SEQUENCE LISTING (5 sequences)

SEQ ID NO: 1 - protein, 6 aa, synthetic construct
  SITE 1: note = Abu
  SITE 1..6: note = Cyclised Abu-Cys via carbon sulfide linkage
  SITE 1: note = Linked to branch Ac-(D)Arg
  SITE 6: note = Linked to branch Tyr(2-ea)-2Nal-Gly(THP)-Glu-Asn-Asn-NH2
XQTWQC

SEQ ID NO: 2 - protein, 6 aa, synthetic construct
  SITE 1: note = Abu
  SITE 1..6: note = Abu-Cys carbon sulfide linkage
  SITE 1: note = Linked to branch Ac-(D)Arg
XQTWQC

SEQ ID NO: 3 - protein, 6 aa, synthetic construct
  SITE 1: note = N-acetyl-L-penicillamine
  SITE 4: note = 7-methyl-L-tryptophan
  SITE 5: note = N-5-acetyl-L-Lysine
  SITE 6: note = L-penicillamine
  SITE 1..6: note = Cyclised Pen-Pen via disulfide bond
  SITE 6: note = Linked to branch Tyr(2-ea)-2Nal-alphaMeLys-Lys(Ac)-Asn-D-Leu-NH2
XNTWKX

SEQ ID NO: 4 - protein, 6 aa, synthetic construct
  SITE 1: note = N-acetyl-L-penicillamine
  SITE 4: note = 7-methyl-L-tryptophan
  SITE 5: note = N-5-acetyl-L-Lysine
  SITE 6: note = L-penicillamine
  SITE 1..6: note = Cyclised Pen-Pen via disulfide bond
  SITE 6: note = Linked to branch Tyr(2-ea)-OH
XNTWKX

SEQ ID NO: 5 - protein, 6 aa, synthetic construct
  SITE 1: note = N-acetyl-L-penicillamine
  SITE 4: note = 7-methyl-L-tryptophan
  SITE 5: note = N-5-acetyl-L-Lysine
  SITE 6: note = L-penicillamine
  SITE 1..6: note = Cyclised Pen-Pen via disulfide bond
  SITE 6: note = Linked to branch Tyr(2-ea)-2Nal-Gly(THP)-Glu-Asn-3Pal-Sarc-NH2
XNTWKX

(19) United States
(12) Patent Application Publication
(10) Pub. No.: US 2024/0218014 A1
(43) Pub. Date: Jul. 4, 2024

(54) SYNTHESIS OF A CYCLIC PEPTIDE

(71) Applicant: Janssen Pharmaceutica NV, Beerse (BE)

(72) Inventors: Pius Bruno Baur, Horgen (CH); Edward Cleator, Whittlesford (GB); Gildas Deniau, Zürich (CH); Frank Eisele, Schaffhausen (CH); Oliver Flögel, Zürich (CH); Anja Huste, Buesingen (DE); Marcel Roman Kehl, Ennetbaden (CH); Markus Löweneck, Dielsdorf (CH); Rolando Ravelo Silva, Bülach (CH); Raffael Vorberg, Rümlang (CH)

(21) Appl. No.: 18/515,672

(22) Filed: Nov. 21, 2023

Related U.S. Application Data
(60) Provisional application No. 63/384,552, filed on Nov. 21, 2022.

(51) Int. Cl. C07K 1/02; C07K 1/06; C07K 7/64

(52) U.S. Cl. CPC C07K 1/026; C07K 1/062; C07K 1/063; C07K 1/065; C07K 7/64

(57) ABSTRACT

Processes for performing peptide synthesis, particularly for cyclic peptide synthesis are generally described. Reaction intermediates of the peptide synthesis are also described.

### FIELD OF THE INVENTION

The present invention relates to processes and intermediates for preparing monocyclic peptides. The monocyclic peptides are useful as inhibitors of the interleukin-23 receptor (IL-23R).

### INCORPORATION OF SEQUENCE LISTING

The sequence listing in ST.26 XML format entitled P086982WO_ST26_sequence_listing.xml, created on Nov. 20, 2023, comprising 14938 bytes, prepared according to 37 CFR 1.822 to 1.824, submitted concurrently with the filing of this application, is incorporated herein by reference in its entirety.

### BACKGROUND

The interleukin-23 (IL-23) cytokine has been implicated as playing a crucial role in the pathogenesis of autoimmune inflammation and related diseases and disorders, such as multiple sclerosis, asthma, rheumatoid arthritis, psoriasis, and inflammatory bowel diseases (IBDs), e.g., ulcerative colitis and Crohn's disease. Peptide inhibitors that bind to IL-23R and inhibit the binding of IL-23 to IL-23R have been identified (see, e.g., US Patent Application Publication No. US2013/0029907). Efficient synthesis of these peptides represents a contemporary challenge. In solid phase peptide synthesis, an amino acid or peptide is bound, usually via the C-terminus, to a solid support. New amino acids are added to the bound amino acid or peptide via coupling reactions. Though solid phase peptide synthesis has been widely used, the process is laborious and requires purification of the reaction products by chromatography after each step, resulting in high cost, slow process and difficulties in scale-up. Accordingly, improved processes and systems are needed for a more efficient peptide synthesis.

### SUMMARY

The present invention provides, inter alia, processes and intermediates for preparing a monocyclic peptide or a salt thereof. In particular, the present invention provides a process for making a monocyclic compound, comprising coupling a monocyclic peptide fragment with a linear chain peptide fragment, wherein the monocyclic peptide fragment is a peptide containing between 4 and 11 amino acid residues; wherein the monocyclic peptide fragment comprises a ring containing between 4 and 8 amino acid residues; wherein the linear chain peptide fragment is a peptide containing between 4 and 10 amino acid residues; and wherein an amide bond is formed between the monocyclic peptide fragment and the linear chain peptide fragment. The present invention is suitable for the synthesis of IL-23 receptor peptide inhibitors.

The present invention also provides a monocyclic peptide fragment which is a compound of Formula (III):

R¹-X3a-X4a-X5a-X6a-X7a-X8a-X9a-X10a-R³ (III)
wherein
R¹ is H, or C₁₋₂₀ alkanoyl;
R³ is OH or OP²;
P² is a carboxyl protecting group;
X3a is absent or any amino acid residue;
X4a is Abu, Cys, (D)Cys, alpha-MeCys, (D)Pen, Pen, or Pen(sulfoxide);
X5a is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys;
X6a is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val;
X7a is unsubstituted Trp, or Trp substituted with cyano, halo, alkyl, substituted or unsubstituted aryl, haloalkyl, hydroxy, or alkoxy;
X8a is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1Nal, 2Nal, or Trp;
X9a is Abu, Cys, (D)Cys, alpha-MeCys, (D)Pen, Pen, or Pen(sulfoxide);
X10a is unsubstituted Phe, or Phe substituted with halo, alkyl, haloalkyl, hydroxy, alkoxy, carboxy, carboxamido, 2-aminoethoxy, or 2-acetylaminoethoxy; and the compound is cyclized via a Pen-Pen disulfide bond between X4a and X9a; or the compound is cyclized via a Abu-Cys or Abu-Pen thioether bond between X4a and X9a.

The present invention also provides a linear peptide fragment which is a compound of Formula (IV):

R²-X11a-X12a-X13a-X14a-X15a-X16a-R⁴ (IV)
wherein
R² is H;
R⁴ is NHP¹, or NH₂;
P¹ is an amino protecting group;
X11a is 2Nal, unsubstituted Trp, or Trp substituted with cyano, halo, alkyl, haloalkyl, hydroxy, alkoxy, Phe(2-Me), Phe(3-Me), Phe(4-Me), Phe(3,4-dimethoxy), or 1Nal;
X12a is 4-amino-4-carboxy-tetrahydropyran (Gly(THP)), alpha-MeLys, alpha-MeLeu, alpha-MeArg, alpha-MePhe, alpha-MeLeu, alpha-MeLys, alpha-MeAsn, alpha-MeTyr, Ala, or cyclohexylAla, Lys, or 2-aminoisobutyric acid;
X13a is 2-aminoisobutyric acid, Glu, Cit, Gln, Lys(Ac), alpha-MeArg, alpha-MeGlu, alpha-MeLeu, alpha-MeLys, alpha-Me-Asn, alpha-MeLys(Ac), Dab(Ac), Dap(Ac), homo-Lys(Ac), or Lys; or X13a is Lys, pegylated Lys, b-homoGlu, or Lys(Y2-Ac), wherein Y2 is an amino acid residue;
X14a is Asn, 2Nal, 2-aminoirobutyric acid, Arg, Cit, Asp, Phe, Gly, Lys, Leu, Ala, (D)Ala, beta-Ala, His, Thr, n-Leu, Gln, Ser, (D)Ser, Tic, Trp, alpha-MeGln, alpha-MeAsn, alpha-MeLys(Ac), Dab(Ac), Dap(Ac), homo-Lys(Ac), or Lys(Ac);
X15a is Ala, beta-Ala, Arg, Asn, Asp, Cit, Cys, Glu, Gln, Gly, substituted or unsubstituted His, (D)His, Ile, Lue, (D)Lue, Lys, (D)Lys, Met, 2Pal, 3Pal, or 4Pal, Phe, Pro, 5-Pyal, 2Quin, 3Quin, Ser, Thr, Trp, Tyr, Val; Leu; and
X16a is absent or Sarc, aMeLeu, (D)NMeTyr, His, (D)Thr, bAla, Pro, or (D)Pro.

The present invention also provides a compound of Formula (VI):

R²-X7a-X8a-X9a-X10a-R⁵ (VI)
wherein R² is H;
R⁵ is NH₂, or NHP¹;
P¹ is an amino protecting group;
X7a is unsubstituted Trp, or Trp substituted with cyano, halo, alkyl, substituted or unsubstituted aryl, haloalkyl, hydroxy, or alkoxy;
X8a is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1-Nal, 2Nal, or Trp;
X9a is Abu, Cys, (D)Cys, alpha-MeCys, (D)Pen, Pen, or Pen(sulfoxide); and
X10a is unsubstituted Phe, or Phe substituted with halo, alkyl, haloalkyl, hydroxy, alkoxy, carboxy, carboxamido, 2-aminoethoxy, or 2-acetylaminoethoxy.

The present invention also provides a compound of Formula (V):

R¹-X3a-X4a-X5a-X6a-R³ (V)
wherein R¹ is H, or C₁₋₂₀ alkanoyl;
R³ is OH or OP²;
P² is a carboxyl protecting group;
X3a is absent or any amino acid residue;
X4a is Abu, Cys, (D)Cys, alpha-MeCys, (D)Pen, Pen, or Pen(sulfoxide);
X5a is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys; and
X6a is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val.

### DETAILED DESCRIPTION

The present invention provides a process for preparing a monocyclic peptide, or a salt thereof, via a convergent solution phase synthesis, in which a monocyclic peptide fragment is coupled with a linear peptide fragment. The process avoids the need for a solid support. Also disclosed are process steps for preparing the monocyclic peptide fragment and the linear peptide fragment in a convergent method, which can be carried out in the solution phase. Intermediates for preparing the monocyclic peptide, monocyclic peptide fragment, and linear peptide fragment are also provided.

Fragments of the monocyclic peptide are coupled together by forming amide bonds between the peptide fragments to form larger fragments. The coupling of the peptide fragments is carried out using any suitable amide coupling reagent, for example using amide coupling reagents described in “Peptide Coupling Reagents, more than a Letter Soup”, A. El-Faham, F. Albericio; Chem. Rev. 2011, 111, 6657-6602. Non-limiting examples of coupling agents include carbodiimides coupling agents, a carbodiimide coupling agent with an additive (activated esters), aminium-based coupling reagents, uronium-based reagents, phosphonium-based coupling agents, halogenating reagents and mixed anhydride reagents. Also useful for preparing amide bonds are acylazoles, acyl azides, acid halides, organophosphorus reagents, organosulfur reagents, triazine coupling agents, and pyridinium coupling reagents, such as those described in “Peptide Coupling Reagents, more than a Letter Soup”, A. El-Faham, F. Albericio; Chem. Rev. 2011, 111, 6657-6602.

The amide bonds can be formed using a coupling reagent selected from carbodiimide coupling reagents, carbodiimides in the presence of an additive, aminium coupling reagents, uronium coupling reagents, phosphonium coupling reagents, halogenating reagents (e.g. chlorinating reagents), acylazoles, acyl azides, acid halides, organophosphorus reagents, organosulfur reagents, triazine coupling reagents, pyridinium coupling reagents, mixed anhydride reagents, activated esters and enzymatic biocatalysis systems. Carbodiimide coupling reagents include N,N′-Diisopropylcarbodiimide (DIC), N,N′-Dicyclohexylcarbodiimide (DCC), 1,1′-carbonyldiimidazole (CDI), N-ethyl-N′-dimethylaminopropylcarbodiimide (EDC), N-ethyl-N′(3-dimethylaminopropyl)carbodiimidehydrochloride (EDCI), N-cyclohexyl-N′-isopropylcarbodiimide (CIC), N-tert-butyl-N′-methylcarbodiimide (BMC), N-tert-butyl-N′-ethylcarbodiimide (BEC), N,N′-dicyclopentylcarbodiimide (CPC), bis(4-(2,2-dimethyl-1,3-dioxolyl))methylcarbodiimide (BDDC), N-ethyl-N′-phenylcarbodiimide (PEC), N-phenyl-N′-isopropylcarbodiimide (PIC). Aminium coupling reagents include O-(Benzotriazol-1-yl)-N,N,N′,N′-tetramethyluroniumtetrafluoroborate (TBTU) or 1-[Bis(dimethylamino)methylene]-1H-1,2,3-triazolo[4,5-b]pyridinium 3-oxide hexafluorophosphate (HATU), O(benzotriazol-1-yl)-N,N,N′,N′-tetramethyluronium hexafluorophosphate (HBTU), 3-(diethylphosphoryloxy)-1,2,3-benzotriazin-4(3H)-one (DEPBT), O-(6-chlorobenzotriazol-1-yl)-N,N,N′,N′-tetramethyluronium hexafluorophosphate (HCTU), and O-(7-azabenzotriazol-1-yl)-N,N,N′,N′-tetramethyluronium tetrafluoroborate (TATU). Uronium-based reagents include O-(5-norbornene-2,3-dicarboximido)-N,N,N′,N′-tetramethyluronium tetrafluoroborate (TNTU), and O—(N-succinimidyl)-1,1,3,3-tetramethyluronium tetrafluoroborate (TSTU), O-(3,4-dihydro-4-oxo-1,2,3-benzotriazine-3-yl)-N,N,N′,N′-tetramethyluronium tetrafluoroborate (TDBTU)), O-(1,2-dihydro-2-oxo-1-pyridyl-N,N,N′,N′-tetramethyluronium tetrafluoroborate (TPTU), or O-[(ethoxycarbonyl)cyano-methyleneamino]-N,N,N′,N′-tetramethyluronium tertafluoroborate (TOTU). Phosphonium coupling agents include (7-Azabenzotriazol-1-yloxy)tripyrrolidinophosphonium hexafluorophosphate (PyAOP), benzotriazol-1-yloxy-tris(dimethylamino)-phosphonium hexafluorophosphate (BOP), (benzotriazol-1-yloxy)tripyrrolidinophosphonium hexafluorophosphate (PyBOP), bromotripyrrolidinophosphonium hexafluorophosphate (PyBrOP), bis(2-oxo-3-oxazolidinyl)phosphinic chloride (BOP—Cl), 1-cyano-2-ethoxy-2-oxoethylideneaminooxy-tris-pyrrolidino-phosphonium hexafluorophosphate (PyOxim), and 6-chloro-benzotriazole-1-yloxy-trispyrrolidinophosphonium hexafluorophosphate (PyClock). Other regents including, but are not limited to 3-(diethylphosphoryloxy)-1,2,3-benzotriazin-4(3H)-one (DEPBT), N,N,N′,N′-tertamethylchloroformamidium hexafluorophosphate (TCFH), or propylphosphonic anhydride solution. Anhydride coupling reagents include symmetric anhydrides, mixed carbonic anhydrides, N-carboxy anhydrides, and urethane-protected N-carboxy anhydrides. Additives for use with a carbodiimide coupling reagent include ethyl cyanohydroxyiminoacetate (Oxyma Pure), 1-hydroxy-7-azabenzotriazole (HOAt) hydroxybenzotriazole (HOBt), 2-hydroxypyridine-N-oxide (HOPO), N-Hydroxysuccinimide (HOSu), 5-(hydroxyimino)-1,3-dimethylpyrimidine-2,4,6(1H,3H,5H)-trione (Oxyma B), hexafluoroisopropanol (HFIP) and 4-nitrophenylalcohol.

A base may be used in the amide bond forming steps. The base can be a tertiary amine base. Suitable bases include bases selected from N-Methylmorpholine (NMM), Di-isopropylethylamine (DIPEA), 1,4-diazabicyclo[2.2.2]octane (DABCO), diethylamine (DEA), triethylamine (TEA) and 1,8-Diazabicyclo[5.4.0]undec-7-ene (DBU).

Suitable solvents for the amide coupling reactions include, but are not limited to, a solvent selected from acetonitrile (MeCN), methyltetrahydrofuran, for example 2-methyltetrahydrofuran (MeTHF), dimethylsulfoxide (DMSO), ethyl acetate (EtOAc), dimethyl formamide (DMF), tetrahydrofuran (THF), dimethyl acetamide (DMA), N-methyl-2-pyrrolidine (NMP), water, and alkyl alcohols such as methanol, ethanol, and 2-propanol.

### General Synthetic Scheme

The following scheme (Scheme A) may be used as a reference to the notation used throughout the following description.

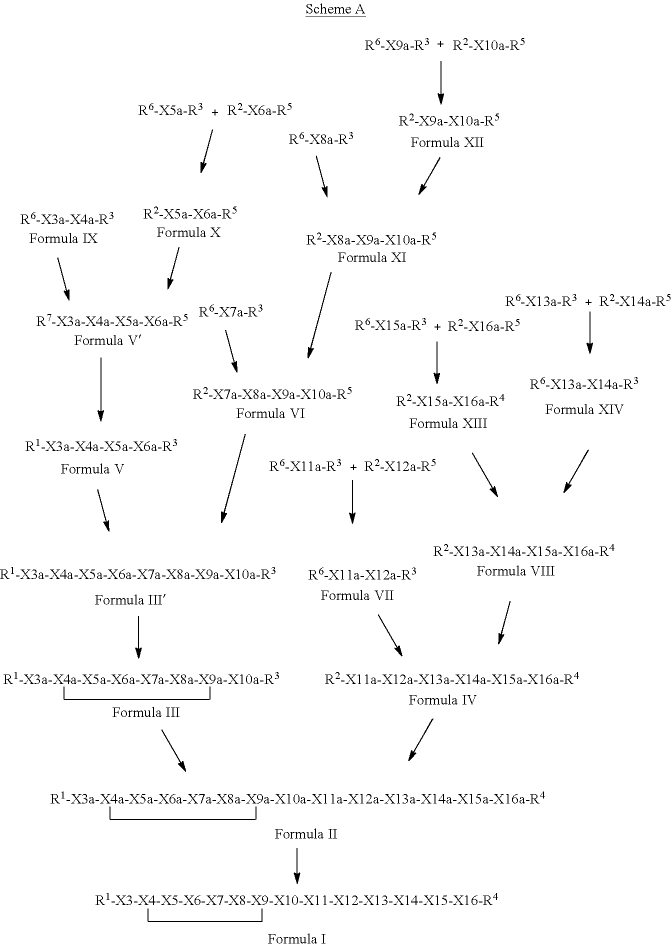

As used in Scheme A and elsewhere herein, X1a, X2a, X3a, X4a, X5a, X6a, X7a, X8a, X9a, X10a, X11a, X12a, X13a, X14a, X15a, X16a, X17a, X18a and X19a designate amino acid residues which are optionally protected by suitable protecting groups. The bridge used in Formula III, II, and I refers to the cyclization of the amino acid residues at the X4a and X9a or X4 and X9 positions. An amino acid residue is a divalent group having the structure—NHCRC(O)— where R is the amino acid side chain. The amino acid side chains can contain reactive functional groups, such as amino groups, hydroxy groups, carboxyl groups and thiol groups, which can be masked during the coupling reaction of peptide fragments using suitable protecting groups. For example, when X4a is “Pen” this includes a Pen amino acid residue which is protected by suitable protecting groups, such as Pen(Trt) and Pen(Acm). Suitable protecting groups are described in “Amino Acid-Protecting Groups”, A. Isidro-Llobet, M. Alvarez, F. Albericio; Chem. Rev. 2009, 109, 2455-2504. The protecting groups may be removed at a convenient subsequent stage using methods known from the art. X1, X2, X3, X4, X5, X6, X7, X8, X9, X10, X11, X12, X13, X14, X15, X16, X17, X18 and X19 designate amino acid residues which are not protected. For example, when X4 is “Pen” this designates a Pen amino acid residue which is not protected (or has been deprotected).

Amino acid residues include amino acid residues selected from Gly, Ala, beta-Ala, Leu, Met, Phe, Phe substituted with halo, alkyl, haloalkyl, hydroxy, alkoxy, cyano, cycloalkyl, carboxy, carboxamido, 2-aminoethoxy (2-ea), or 2-acetylaminoethoxy, Trp, Trp substituted with cyano, halo, alkyl, haloalkyl, hydroxy, substituted or unsubstituted aryl, or alkoxy, Lys, Gln, beta-homoGln, Pro, Val, Ile, Cys, (D)Cys, alpha-MeCys, (D)Pen, Pen, or Pen(sulfoxide), Cit, Tyr, His, (D)His, Arg, (D)Arg Asn, Glu, Ser, alpha-MeSer, alpha-MeGln, alpha-MeLys, alpha-MeLeu, alpha-MeAsn, alpha-MeThr, Lys(Ac), alpha-MeLys(Ac), alpha-MeArg, alpha-MePhe, alpha-MeTyr, Dab(Ac), Dap(Ac), homo-Lys(Ac), Asp, Thr, Sarc, Aib, Dab, Dap, gamma-Glu, Gaba, beta-Pro, Abu, 1Nal, 2Nal, Lys(b-Ala), Lys(Gly), Lys(Benzyl, Ac), Lys(butyl, Ac), Lys(isobutyl, Ac), Lys(propyl, Ac), Lys(PEG2PEG2gEC18OH), Phe (2ea), Phe(2-Me), Phe(3-Me), Phe(4-Me), Phe(3,4-dimethoxy), 2Quin, 3Quin, 4-amino-4-carboxy-tetrahydropyran (Gly(THP)), Acvc, cyclohexylAla, 2Pal, 3Pal, or 4Pal, 5Pyal.

In any of the formulae disclosed herein, for example any of Formula II-Formula XIV, X3a is absent or any amino acid residue. In some embodiments, X3a is absent. In some embodiments, X3a is Arg or D(Arg). In some embodiments, X3a is (D)Arg. Functional groups on the side chain of X3a may be optionally protected with a suitable protecting group.

In any of the formulae disclosed herein, for example any of Formula II-Formula XIV, X4a is Abu, Cys, (D)Cys, alpha-MeCys, (D)Pen, Pen, or Pen(sulfoxide). In some embodiments, X4a is Abu. In some embodiments, X4a is Cys or (D)Cys. In some embodiments, X4a is Pen or (D)Pen. In some embodiments, X4a is Pen. Functional groups on the side chain of X4a may be optionally protected with a suitable protecting group.

In any of the formulae disclosed herein, for example any of Formula II-Formula XIV, X5a is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys. Functional groups on the side chain of X5a may be optionally protected with a suitable protecting group.

In any of the formulae disclosed herein, for example any of Formula II-Formula XIV, X6a is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val. Functional groups on the side chain of X6a may be optionally protected with a suitable protecting group.

In any of the formulae disclosed herein, for example any of Formula II-Formula XIV, X7a is unsubstituted Trp, or Trp substituted with cyano, halo, alkyl, substituted or unsubstituted aryl, haloalkyl, hydroxy, or alkoxy. In any of the formulae disclosed herein, for example any of Formula II-Formula XIV, X7a is unsubstituted Trp, or Trp substituted with cyano, halo, alkyl, phenyl or phenyl substituted by NHC(O)CH₃, haloalkyl, hydroxy, or alkoxy. Functional groups on the side chain of X7a may be optionally protected with a suitable protecting group.

In any of the formulae disclosed herein, for example any of Formula II-Formula XIV, X8a is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1-Nal, 2Nal, or Trp. Functional groups on the side chain of X8a may be optionally protected with a suitable protecting group.

In any of the formulae disclosed herein, for example any of Formula II-Formula XIV, X9a is Abu, Cys, (D)Cys, alpha-MeCys, (D)Pen, Pen, or Pen(sulfoxide). Functional groups on the side chain of X9a may be optionally protected with a suitable protecting group.

In any of the formulae disclosed herein, for example any of Formula II-Formula XIV, X10a is unsubstituted Phe, or Phe substituted with halo, alkyl, haloalkyl, hydroxy, alkoxy, carboxy, carboxamido, 2-aminoethoxy, or 2-acetylaminoethoxy. Functional groups on the side chain of X10a may be optionally protected with a suitable protecting group.

In any of the formulae disclosed herein, for example any of Formula II-Formula XIV, X11a is 2Nal, unsubstituted Trp, or Trp substituted with cyano, halo, alkyl, haloalkyl, hydroxy, alkoxy, Phe(2-Me), Phe(3-Me), Phe(4-Me), Phe(3,4-dimethoxy), or 1Nal. Functional groups on the side chain of X11a may be optionally protected with a suitable protecting group.

In any of the formulae disclosed herein, for example any of Formula II-Formula XIV, X12a is 4-amino-4-carboxy-tetrahydropyran (Gly(THP)), alpha-MeLys, alpha-MeLeu, alpha-MeArg, alpha-MePhe, alpha-MeLeu, alpha-MeLys, alpha-MeAsn, alpha-MeTyr, Ala, or cyclohexylAla, Lys, or 2-aminoisobutyric acid. Functional groups on the side chain of X12a may be optionally protected with a suitable protecting group.

In any of the formulae disclosed herein, for example any of Formula II-Formula XIV, X13a is 2-aminoisobutyric acid, Glu, Cit, Gln, Lys(Ac), alpha-MeArg, alpha-MeGlu, alpha-MeLeu, alpha-MeLys, alpha-Me-Asn, alpha-MeLys(Ac), Dab(Ac), Dap(Ac), homo-Lys(Ac), or Lys; or X13a is Lys, pegylated Lys, b-homoGlu, or Lys(Y2-Ac), wherein Y2 is an amino acid residue. Functional groups on the side chain of X13a may be optionally protected with a suitable protecting group.

In any of the formulae disclosed herein, for example any of Formula II-Formula XIV, X14a is Asn, 2Nal, 2-aminoirobutyric acid, Arg, Cit, Asp, Phe, Gly, Lys, Leu, Ala, (D)Ala, beta-Ala, His, Thr, n-Leu, Gln, Ser, (D)Ser, Tic, Trp, alpha-MeGln, alpha-MeAsn, alpha-MeLys(Ac), Dab(Ac), Dap(Ac), homo-Lys(Ac), or Lys(Ac). Functional groups on the side chain of X14a may be optionally protected with a suitable protecting group.

In any of the formulae disclosed herein, for example any of Formula II-Formula XIV, X15a is Ala, beta-Ala, Arg, Asn, Asp, Cit, Cys, Glu, Gln, Gly, substituted or unsubstituted His, (D)His, Ile, Lue, (D)Lue, Lys, (D)Lys, Met, 2Pal, 3Pal, or 4Pal, Phe, Pro, 5-Pyal, 2Quin, 3Quin, Ser, Thr, Trp, Tyr, Val, Leu. Functional groups on the side chain of X15a may be optionally protected with a suitable protecting group.

In any of the formulae disclosed herein, for example any of Formula II-Formula XIV, X16a is absent or Sarc, aMeLeu, (D)NMeTyr, His, (D)Thr, bAla, Pro, or (D)Pro. Functional groups on the side chain of X16a may be optionally protected with a suitable protecting group.

In any of the formulae disclosed herein, X17a is absent or is Lys(PEG2PEG2gEC18OH). Functional groups on the side chain of X17a may be optionally protected with a suitable protecting group.

Suitable amino protecting groups include benzyl (Bn), trityl (Trt), 4-methyltrityl (Mtt), β-methoxyethoxytrityl (MEM), 2-nitrophenylsulfonyl (Nps), 2-(4-nitrophenyl)sulfonylethoxycarbonyl (Nsc), benzothiazole-2-sulfonyl (Bts), dithiasuccinoyl (Dts), nitrobenzenesulfonyl (Ns), 2-(2-nitrophenyl)propoxycarbonyl (NPPOC), 2-(3,4-methylenedioxy-6-nitrophenyl)propoxycarbonyl (MNPPOC), methylsulfonylethoxycarbonyl (Msc) 9-fluorenylmethyloxycarbonyl (Fmoc), 2,7-di-tert-butyl-Fmoc (Fmoc*), 2-fluoro-Fmoc (Fmoc(2F)), 2-monoisooctyl-Fmoc (mio-Fmoc), benzyloxycarbonyl (Cbz), 2,2,2-trichloroethoxycarbonyl (Troc), 2-(trimethylsilyl)ethoxycarbonyl (Teoc), 2-(4-trifluoromethylphenylsulfonyl)ethoxycarbonyl (Tsc), tert-butoxycarbonyl (BOC), 1-adamantyloxycarbonyl (Adoc), 2-adamantyloxycarbonyl (2-Adoc), 2,4-dimethylpent-3-yloxycarbonyl (Doc), cyclohexyloxycarbonyl (Hoc), 1,1-dimethyl-2,2,2-trichloroethoxycarbonyl (TcBOC), formyl, acetyl (Ac), trifluoroacetyl (TFA), p-toluenesulfonyl (Ts), vinyl, 2-chloroethyl, 2-phenylsulfonylethyl, allyl, 2-nitrobenzyl, 4-nitrobenzyl, diphenyl-4-pyridylmethyl, N′,N′-dimethylhydrazinyl, methoxymethyl, tert-butoxymethyl (Bum), benzyloxymethyl (BOM), 2-tetrahydropyranyl (THP), tri(C₁₋₄ alkyl)silyl (e.g., tri(isopropyl)silyl), 1,1-diethoxymethyl, α,α-dimethyl-3,5-dimethoxybenzyloxycarbonyl (Ddz), 2-(p-biphenylyl)-2-propyloxycarbonyl (Bpoc), 1,1-dioxonaptho[1,2-b]thiophene-2-methyloxycarbonyl (α-Nsmoc), 3,3-dioxonaptho[2,1-b]thiophene-2-methyloxycarbonyl (β-Nsmoc), 1-(4,4-dimethyl-2,6-dioxocyclohex-1-ylidene)ethyl (Dde), 1-(4,4-dimethyl-2,6-dioxocyclohex-1-ylidene)isovaleryl (ivDde), 2-(phenyl(methyl)sulfonio)ethoxycarbonyl (Pms), N-ethanesulfonylethoxycarbonyl (Esc), 2-(4-sulfophenylsulfonyl)ethoxycarbonyl (Sps), allyloxycarbonyl (Alloc), propargyloxycarbonyl (Poc), 9-(4-bromophenyl)-9-fluorenyl (BrPhF), azidomethyloxycarbonyl (Azoc), N-tetrachlorophthaloyl (TCP), phenyldithioethyloxycarbonyl (Phdec), 2-pyridyldithioethyloxycarbonyl (Pydec) or N-pivaloyloxymethyl (POM).

Suitable carboxyl protecting groups are selected from C₁₋₆ alkyl, such as tert-butyl (tBu), methyl (Me), ethyl (Et), propyl and cyclohexyl, allyl, 1,1-dimethylallyl (Dma), phenacyl (Pac), p-nitrobenzyl (p-NB) trityl (Tr), 2-chlorotrityl (2-Cl-Trt), 2,4-dimethoxybenzyl (Dmb), 9-fluorenylmethyl (Fm), phenyl, cyclohexyl, benzyl (Bn), 3,4-ethylenedioxy-2-thenyl (EDOTₙ), 4-(N-(1-(4,4-diemthyl-2,6-dioxocyclohexylidene)-3-methylbutyl)amino)benzyl (Dmab), trimethylsilylethyl (TMSE), 2-(trimethylsilyl)isopropyl (Tmsi), 2,2,2-trichloroethyl (Tce), carbamoylmethyl (Cam), 4,5-dimethoxy-2-nitrobenzyloxycarbonyl (Dmnb), pentamine cobalt (III), β-menthyl (Men), β-3-menthylpent-3-yl (Mpe), or 2-phenylisopropyl (2-Ph(iPr)). In instances where the carboxyl protecting group also acts a leaving group in the amide coupling, the carboxyl protecting group can be p-nitrobenzyl.

Suitable thiol protecting groups include p-methyl benzyl (Meb), p-methoxybenzyl (Mob), trityl (Tr), monomethoxytrityl (Mmt), 2,4,6-trimethoxybenzyl (Tmob), 9-xanthenyl (Xan), 2,2,4,6,7-pentamethyl-5-dihydrobenzofuranylmethyl (Pmbf), benzyl (Bn), tert-butyl (tBu), 1-adamantyl (1-Ada), 9-fluorenylmethyl (Fm), 2-(2,4-dinitrophenyl)ethyl (Dnpe), 9-fluorenylmethyloxycarbonyl (Fmoc), acetamidomethyl (Acm), phenylacetamidomethyl (PhAcm), tert-butylmercapto (S(tBu)), 3-nitro-2-pyridinesulfenyl (Npys), 2-pyridinesulfenyl (S-Pyr), allyloxycarbonyl (Alloc), N-allyloxycarbonyl-N-[2,3,5,6-tetrafluoro-4-(phenylthio)phenyl)aminomethyl (Fsam), o-nitrobenzyl (o-NB), 4-Picolyl, ninhydrin (Nin).

Suitable hydroxyl protecting groups include benzyl (Bn), cyclohexyl, tert-butyl (tBu), trityl (Trt), tert-butyldimethylsilyl (TBDMS), pseudoprolines, tert-butyldiphenylsilyl (TBDPS), 4,5-dimethoxy-2-nitrobenzyloxycarbonyl (Dmnb), propargyloxycarbonyl (Poc).

### Abbreviations

Herein and throughout the application, the following abbreviations may be used.
Ac acetyl
Acm acetamidomethyl
ACN or MeCN acetonitrile
AcOH acetic acid
1-Ada 1-adamantyl
Adoc 1-adamantyloxycarbonyl
2-Adoc 2-adamantyloxycarbonyl
Alloc allyloxycarbonyl
API active pharmaceutical ingredient
Azoc azidomethyloxycarbonyl
BDDC bis[[4-(2,2-dimethyl-1,3-dioxolyl)]methylcarbodiimide
BEC N-tert-butyl,N′-ethylcarbodiimide
BMC N-tert-butyl,N′-methylcarbodiimide
Bn benzyl
Boc or BOC tert-butyloxycarbonyl
BOP benzotriazol-1-yloxy-tris(dimethylamino)-phosphonium
hexafluorophosphate
BOP—Cl bis(2-oxo-3-oxazolidinyl)phosphinic chloride
Bpoc 2-(p-biphenylyl)-2-propyloxycarbonyl
BOM benzyloxymethyl
BrPhF 9-(4-bromophenyl)-9-fluorenyl
BSA bis(trimethylsilyl)acetamide
Bts benzothiazole-2-sulfonyl
Bum tert-butoxymethyl
Bz benzoyl
Cam carbamoylmethyl
Cbz or Z benzyloxycarbonyl
CDI 1,1′-carbonyldiimidazole
CIC N-cyclohexyl,N′-isopropylcarbodiimide
2-Cl-Trt 2-chlorotrityl
CPC N,N′-dicyclopentylcarbodiimide
DABCO 1,8-Diazabicyclo[2.2.2]octane
DBU 1,8 diazabicyclo[5,4,0]undec-7-ene
DCC N—N′-Dicyclohexylcarbodiimide
DCHA dicyclohexylamine
DCM dichloromethane
Dde 1-(4,4-dimethyl-2,6-dioxocyclohex-1-ylidene)ethyl
ivDde 1-(4,4-dimethyl-2,6-dioxocyclohex-1-ylidene)isovaleryl
Ddz α,α-dimethyl-3,5-dimethoxybenzyloxycarbonyl
DEA diethylamine
DEPBT 3-(diethylphosphoryloxy)-1,2,3-benzotriazin-4(3H)-one
DIC diisopropylcarbodiimide
DIEA, DIPEA N,N-diisopropylethylamine
DMA (solvent) dimethyl acetamide
Dma 1,1-dimethylallyl
Dmab 4-(N-[1-(4,4-dimethyl-2,6-dioxocyclohexylidene)-3-methylbutyl)amino)benzyl
Dmb 2,4-dimethoxybenzyl
DMF N,N-dimethylformamide
Dmnb 4,5-dimethoxy-2-nitrobenzyloxycarbonyl
DMSO dimethylsulfoxide
Dnpe 2-(2,4-dinitrophenyl)ethyl
Doc 2,4-dimethylpent-3-yloxycarbonyl
DODT 3,6-dioxa-1,8-octanedithiol
Dts dithiasuccinoyl
EDC N-ethyl-N′-dimethylaminopropylcarbodiimide
EDCI N-ethyl-N′-(3-dimethylaminopropyl)carbodiimide
Esc N-ethanesulfonylethoxycarbonyl
EDOTₙ 3,4-ethylenedioxy-2-thenyl
ESI-MS electrospray ionization-mass spectrometry
Et ethyl
EtOAc ethyl acetate
EtOH ethanol
Fm fluorenylmethyl
Fmoc 9-fluorenylmethoxycarbonyl
Fmoc* 2,7-di-tert-butyl-Fmoc
Fmoc(2F) 2-fluoro-Fmoc
mio-Fmoc 2-monoisooctyl-Fmoc
Fsam N-allyloxycarbonyl-N-[2,3,5,6-tetrafluoro-4-(phenylthio)phenyl)aminomethyl
HATU 1-[Bis(dimethylamino)methylene]-1H-1,2,3-triazolo[4,5-b]pyridinium 3-oxide hexafluorophosphate
HBTU O(benzotriazol-1-yl)-N,N,N′,N′-tetramethyluronium
hexafluorophosphate
HCTU O-(6-chlorobenzotriazol-1-yl)-N,N,N′,N′-tetramethyluronium hexafluorophosphate
HFIP hexafluoroisopropanol
HOAt 1-hydroxy-7-azabenzotriazole
HOBt hydroxybenzotriazole
Hoc cyclohexyloxycarbonyl
HOPO 2-hydroxypyridine-N-oxide
HOSu N-hydroxysuccinimide
HPLC high performance liquid chromatography
HV high vacuum
IBD inflammatory bowel disease
IL-23 interleukin-23
IPA isopropyl alcohol
IPAc isopropylacetate
IPC in-process control
IPE isopropyl ether
Me methyl
Meb p-methyl benzyl
MEM β-methoxyethoxytrityl
Men β-menthyl
MeOH methanol
MeTHF 2-methyl tetrahydrofuran
Mmt monomethoxytrityl
MNPPOC 2-(3,4-methylenedioxy-6-nitrophenyl)propoxycarbonyl
Mob p-methoxybenzyl
MOM methoxymethyl
Moz p-methylbenzyloxycarbonyl
Mpe β-3-menthylpent-3-yl
Mr relative molecular mass
Msc methylsulfonylethoxycarbonyl
MTBE methyl tert-butylether
Mtt 4-methyltrityl
m/z mass-to-charge ratio
NB nitrobenzyl
Nin ninhydrin
NMM N-methyl morpholine
NMP N-methyl pyrrolidine
NMR nuclear magnetic resonance
NPPOC 2-(2-nitrophenyl)propoxycarbonyl
Nps 2-nitrophenylsulfonyl
Npys 3-nitro-2-pyridinesulfenyl
Ns nitrobenzenesulfonyl
Nsc 2-(4-nitrophenyl)sulfonylethoxycarbonyl
α-Nsmoc 1,1-dioxonaptho[1,2-b]thiophene-2-methyloxycarbonyl
β-Nsmoc 3,3-dioxonaptho[2,1-b]thiophene-2-methyloxycarbonyl
OEt ethoxy
OHe hexoxy
OMe methoxy
OPac oxo-phenylacetyl
Oxyma B 5-(hydroxyimino)-1,3-dimethylpyrimidine-2,4,6-(1H,3H,5H)-trione
Oxyma Pure ethyl 2-cyano-2-(hydroxyimino)acetate
Pac phenacyl
Pbf 2,2,4,6,7-pentamethyldihydrobenzofuran-5-sulfanoyl
PEC N-ethyl,N-phenylcarbodiimide
PhAcm phenylacetamidomethyl
Phdec phenyldithioethyloxycarbonyl
2-Ph(iPr) 2-phenylisopropyl
PIC N-phenyl, N-isopropylcarbodiimide
PivCl pivaloyl chloride
Pmbf 2,2,4,6,7-pentamethyl-5-dihydrobenzofuranylmethyl
Pms 2-(phenyl(methyl)sulfonio)ethoxycarbonyl
Poc propargyloxycarbonyl
POM N-pivaloyloxymethyl
1-PrOH 1-propanol
PyAOP (7-Azabenzotriazol-1-yloxy)tripyrrolidino-phosphonium
hexafluorophosphate
PyBOP (benzotriazol-1-yloxy)tripyrrolidinophosphonium
hexafluorophosphate
PyBrOP bromotripyrrolidinophosphonium hexafluorophosphate
Pydec 2-pyridyldithioethyloxycarbonyl
rt or RT room temperature
Sps 2-(4-sulfophenylsulfonyl)ethoxycarbonyl
S-Pyr 2-pyridinesulfenyl
S(tBu) tert-butylmercapto
TATU O-(7-azabenzotriazol-1-yl)-N,N,N′,N′-tetramethyluronium tetrafluoroborate
TBDMS tert-butyldimethylsilyl
TBDPS tert-butyldiphenylsilyl
TBS tert-butyl dimethyl silyl
TBTU N,N,N′N′-tetramethyl-O-(benzotriazole-1-yl)aminium tetrafluoroborate
tBu tert-butyl
TcBOC 1,1-dimethyl-2,2,2-trichloroethoxycarbonyl
Tee 2,2,2-trichloroethyl
TCFH N,N,N′,N′-tertamethylchloroformamidium
hexafluorophosphate
TCP N-tetrachlorophthaloyl
TDBPTU O-(3,4-dihydro-4-oxo-1,2,3-benzotriazine-3-yl)-N,N,N′,N′-tetramethyluronium tetrafluoroborate
TEA triethylamine
Teoc 2-(trimethylsilyl)ethoxycarbonyl
TES triethylsilyl
TFA trifluoroacetyl
THF tetrahydrofuran
THP 2-tetrahydropyranyl
TIS triisopropylsilane
Tmob 2,4,6-trimethoxybenzyl
TMS trimethylsilyl
TMSE trimethylsilylethyl
Tmsi 2-(trimethylsilyl)isopropyl
TNTU O-(5-norbornene-2,3-dicarboximido)-N,N,N′,N′-tetramethyluronium tetrafluoroborate
TOTU O-[(ethoxycarbonyl)cyano-methyleneamino]-N,N,N′,N′-tetramethyluronium tertafluoroborate
TPP thiamine pyrophosphate
TPTU O-(1,2-dihydro-2-oxo-1-pyridyl-N,N,N′,N′-tetramethyluronium tetrafluoroborate
Tr triphenyl methyl
Troc 2,2,2-trichloroethoxycarbonyl
TrtOH triphenylmethanol
Ts p-toluenesulfonyl
Tsc 2-(4-trifluoromethylphenylsulfonyl)ethoxycarbonyl
TSTU O—(N-succinimidyl)-1,1,3,3-tetramethyluronium
tetrafluoroborate
UPLC ultra performance liquid chromatography
v/v volume for volume
w/w weight for weight
Xan 9-xanthenyl

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

| Abbreviation | Definition |
|---|---|
| (1-Me)His | (1-Methyl)Histidine |
| (D)aMeTyr | (D)-alpha-Me-Tyrosine |
| (D)NMeTyr | NMe(D)Tyr or N—Me-(D)Tyrosine |
| 1Nal | L-1-Naphthylalanine |
| 2-Nal or 2Nal | L-2-Naphthylalanine |
| 2-Pal or 2Pal | L-2-Pyridylalanine |
| | 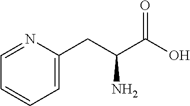 [SMILES: N[C@@H](Cc1ccccn1)C(=O)O] |
| 3-Pal or 3Pal | L-3-Pyridylalanine |
| | 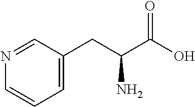 [SMILES: N[C@@H](Cc1cccnc1)C(=O)O] |
| 4-amino-4-carboxy- tetrahydropyran or Gly(THP) or THPGly | 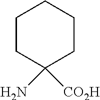 |
| 4-Pal or 4Pal | L-4-Pyridylalanine |
| | 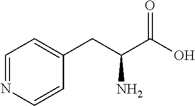 [SMILES: N[C@@H](Cc1ccncc1)C(=O)O] |
| 5Pyal | 5-pyrimidine-alanine |
| | 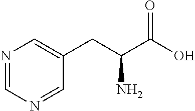 [SMILES: N[C@@H](Cc1cncnc1)C(=O)O] |
| Abu | 2-Aminobutyric acid |
| Ac— | Acetyl |
| Acvc | 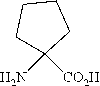 |
| | 1-aminocyclopentanecarboxylic acid |
| Aib | 2-aminoisobutyric acid |
| a-MeAsn, alpha-MeAsn | α-Methyl-L-Asparagine |
| a-MeGln, alpha-MeGln | α-Methyl-L-Glutamine |
| aMeGlu or αMeGlu | alpha-methyl Glutamic Acid |
| Cit | L-Citrulline |
| CONH₂ | Carboxamide |
| COOH | Carboxylic acid |
| cyclohexylAla | (2- or beta-)-cyclohexyl-L-Alanine |
| Dab | L-Diaminobutyric acid |
| Dab(Ac) | N-γ-acetyl-L-diaminobutyric acid |
| Dap | L-Diaminopropionic acid |
| Dap(Ac) | N-γ-acetyl-L-diaminopropionic acid |
| hLys(Ac) or homo-Lys(Ac), | homo-L-Lysine |
| Hphe | Homophenylalanine |
| hPhe(3,4-dimethoxy) | 3,4-dimethoxy-L-homophenylalanine |
| Hse | Homoserine |
| iPr or i-Pr | Iso-Propyl |
| Lys(Ac) | N-ϵ-acetyl-L-Lysine |
| Lys(Benzyl, Ac) | N^{ϵ}-acetyl-N^{ϵ}-benzyl-L-Lysine or Lys(N-acetyl-N-benzyl) |
| | 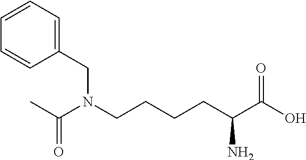 [SMILES: CC(=O)N(CCCC[C@H](N)C(=O)O)Cc1ccccc1] |
| Lys(butyl, Ac) | N^{ϵ}-acetyl-N^{ϵ}-butyl-L-Lysine or Lys(N-acetyl-N-butyl) |
| | 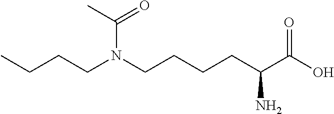 [SMILES: CCCCN(CCCC[C@H](N)C(=O)O)C(C)=O] |
| Lys(isobutyl, Ac) | N^{ϵ}-acetyl-N^{ϵ}-isobutyl-L-Lysine or Lys(N-acetyl-N- |
| | isobutyl) |
| | 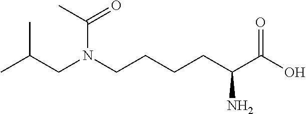 [SMILES: CC(=O)N(CCCC[C@H](N)C(=O)O)CC(C)C] |
| Lys(propyl, Ac) | N^{ϵ}-acetyl-N^{ϵ}-propyl-L-Lysine or Lys(N-acetyl-N-propyl) |
| | 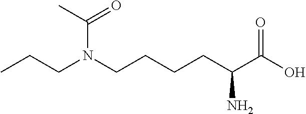 [SMILES: CCCN(CCCC[C@H](N)C(=O)O)C(C)=O] |
| Lys(b-Ala) | β-Alanyl-L-lysine |
| Lys(Gly) | Glycyl-L-lysine |
| Lys(PEG2PEG2gEC18OH) | 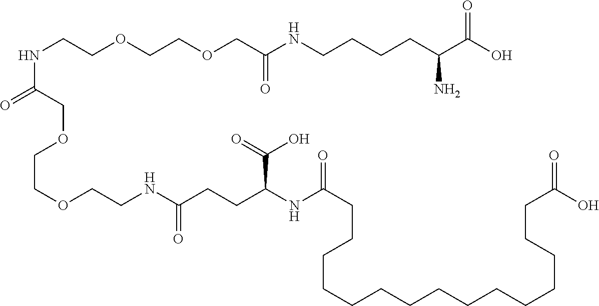 [SMILES: N[C@@H](CCCCNC(=O)COCCOCCNC(=O)COCCOCCNC(=O)CC[C@H](NC(=O)CCCCCCCCCCCCCCCC(=O)O)C(=O)O)C(=O)O] |
| Pen | L-Penicillamine |
| Pen(sulfoxide) | L-Penicillamine(sulfoxide) |
| Phe | L-phenylalanine |
| Phe(2-Me) | 2-methyl-L-phenylalanine |
| Phe(3-Me) | 3-methyl-L-phenylalanine |
| Phe(3,4-dimethoxy) | 3,4-dimethoxy-L-phenylalanine |
| Phe(4-Me) | 4-methyl-L-phenylalanine |
| 2Quin | (S)-2-amino-3-(quinolin-2-yl)propanoic acid or 2- |
| | quinolinylalanine |
| Quin or 3Quin or 3-Quin | 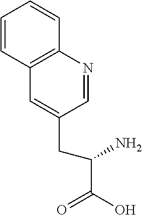 [SMILES: N[C@@H](Cc1cnc2ccccc2c1)C(=O)O] |
| | (S)-2-amino-3-(quinolin-3-yl)propanoic acid or |
| | 3-quinolinylalanine |
| Sarc or NmeGly | Sarcosine or N-methylglycine |
| W(7-Me) | 7-Methyl-Tryptophan |
| Tyr(2-ea) or Tyr(2ea) | 4-[2-aminoethoxy]-L-phenylalanine |
| | 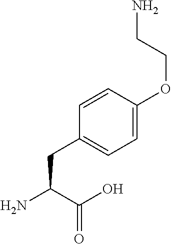 [SMILES: NCCOc1ccc(C[C@H](N)C(=O)O)cc1] |
| | (phenylanlanine substituted by 2-aminoethoxy) |
| Trp(7-Me) | 7-methyl-L-tryptophan |
| β-Ala, beta-Ala or bA | L-β-Alanine |
| β-Glu | L-β-Glutamic acid |
| γ-Glu or gamma-Glu | L-γ-Glutamic acid |
| Gaba | γ-Aminobutyric acid |
| β-Pro or beta-Pro | L-β-Proline |
| βhGln or b-hGln, or beta- | L-β-homoglutamine |
| homoGln | |
| α-MeArg, a-MeArg, or alpha- | alpha-methyl-L-Arginine |
| MeArg | |
| α-MeCys, alpha-MeCys, or a- | alpha-methyl-L-Cysteine |
| MeCys | |
| α-MeLeu, a-MeLeu, alpha- | alpha-methyl-L-Leucine |
| MeLeu | |
| α-MeLys(Ac), a-MeLys(Ac), | ϵ-acetyl-alpha-methyl-L-Lysine |
| or alpha-MeLys(Ac) | |
| α-MeLys, a-MeLys, or alpha- | alpha-methyl-L-Lysine |
| MeLys | |
| α-MeOrn | alpha-methyl-L-Ornithine |
| α-MePhe or a-MePhe or a- | alpha-methyl-L-Phenylalanine |
| Me-Phe | |
| α-MeSer or a-MeSer or a- | alpha-methyl-L-Serine |
| Me-Ser | |
| α-MeThr or a-MeThr or a- | alpha-methyl-L-Threonine |
| Me-Thr | |
| α-MeTrp | alpha-methyl-L-Tryptophan |
| α-MeTyr | alpha-methyl-L-Tyrosine |

### Definitions

Unless otherwise defined herein, scientific and technical terms used in this application shall have the meanings that are commonly understood by those of ordinary skill in the art.

As used herein, the following terms have the meanings ascribed to them unless specified otherwise.

“A,” “an,” or “a(n)” is an indefinite article when used in reference to a group of substituents or “substituent group” herein, mean at least one.

“About” when referring to a value includes the stated value +/−10% of the stated value. For example, about 50% includes a range of from 45% to 55%, while about 20 molar equivalents includes a range of from 18 to 22 molar equivalents. Accordingly, when referring to a range, “about” refers to each of the stated values +/−10% of the stated value of each end of the range. For instance, a ratio of from about 1 to about 3 (weight/weight) includes a range of from 0.9 to 3.3. In some embodiments, reference to about a value or parameter includes a description of that value or parameter per se. For example, reference to about 20 molar equivalents includes and describes 20 molar equivalents per se.

As used in the specification and in the claims, “comprise(s),” “comprising,” “include(s),” “having,” “has,” “can,” “contain(s),” and variants thereof, as used herein, are intended to be open-ended transitional phrases, terms, or words that require the presence of the named features, groups, ingredients, or steps and do not exclude the presence of additional features, groups, ingredients, or steps. The term “comprise(s),” “comprising,” “include(s),” “having,” “has,” “can,” or “contain(s),” can include embodiments encompassed by the term “consisting essentially of” or “consisting of.”

The terms “peptide,” “polypeptide,” and “protein” are used interchangeably herein and typically refer to a molecule comprising a chain of two or more amino acids (e.g., L-amino acids, D-amino acids, modified amino acids, amino acid analogs, amino acid mimetics, etc.).

The term “L-amino acid,” as used herein, refers to the “L” isomeric form of an amino acid, and conversely the term “D-amino acid” refers to the “D” isomeric form of an amino acid (e.g., (D)Asp or D-Asp; (D)Phe or D-Phe). Amino acid residues in the D isomeric form can be substituted for any L-amino acid residue, as long as the desired function is retained by the peptide. D-amino acids may be indicated as customary in lower case when referred to using single-letter abbreviations. L-amino acids may be indicated by either conventional three-letter, capitalized one-letter, or amino acid designations described in the abbreviations. For example, D-arginine can be represented as “arg” or “r.” Alternatively, “D” or a lower case “d” in front of an amino acid can be used to indicate that it is of the D isomeric form, for example D-lysine can be represented by dK. For another example, L-arginine can be represented as “Arg” or “R”.

One of skill in the art will appreciate that certain amino acids and other chemical moieties are modified when bound to another molecule. For example, an amino acid side chain may be modified when it forms an intramolecular bridge with another amino acid side chain, e.g., one or more hydrogen may be removed or replaced by the bond.

The term “NH₂,” as used herein, can refer to a free amino group present at the amino terminus of a polypeptide. The term “OH,” as used herein, can refer to a free carboxy group present at the carboxy terminus of a peptide. Further, the term “Ac,” as used herein, refers to Acetyl protection through acylation of the C- or N-terminus of a polypeptide. In certain peptides shown herein, the NH₂ located at the C-terminus of the peptide indicates an amino group.

The term “carboxy,” as used herein, refers to —CO₂H.

The term “cyclized,” as used herein, refers to one part of a polypeptide molecule being linked to another part of the polypeptide molecule to form a closed ring, such as by forming a disulfide bridge or thioether bond.

The term “subunit,” as used herein, refers to one of a pair of polypeptide monomers that are joined to form a dimer peptide composition.

The term “salt” or “pharmaceutically acceptable salt,” as used herein, represents salts or zwitterionic forms of the peptides or compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethanesulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. A salt may suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such as calcium or magnesium ions, as well as substituted ammonium ions, such as ions of the type N(R1)(R2)(R3)(R4)+, where R1, R2, R3 and R4 independently will typically designate hydrogen, optionally substituted C₁₋₆-alkyl or optionally substituted C₂₋₆alkenyl. Examples of relevant C₁₋₆-alkyl groups include methyl, ethyl, 1-propyl and 2-propyl groups. Examples of C₂₋₆alkenyl groups of possible relevance include ethenyl, 1-propenyl and 2-propenyl. Other examples of pharmaceutically acceptable salts are described in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salts: Properties, Selection, and Use by Stahl and Wermuth (Wiley-VCH, 2002). Other suitable base salts are formed from bases which form non-toxic salts. Representative examples include the aluminum, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts.

By “pharmaceutically acceptable” it is meant the carrier(s), diluent(s) or excipient(s) must be compatible with the other components or ingredients of the compositions of the present invention, i.e., that which is useful, safe, non-toxic, acceptable for pharmaceutical use. In accordance with the present invention pharmaceutically acceptable means approved or approvable as is listed in the U.S. Pharmacopoeia or other generally recognized pharmacopoeia for use in animals, and more particularly, in humans.

The term “alkyl” includes a straight chain or branched, noncyclic or cyclic, saturated aliphatic hydrocarbon containing from 1 to 24 carbon atoms. Representative saturated straight chain alkyls include, but are not limited to, methyl, ethyl, n-propyl, n-butyl, n-pentyl, n-hexyl, and the like, while saturated branched alkyls include, without limitation, isopropyl, sec-butyl, isobutyl, tert-butyl, isopentyl, and the like. Representative saturated cyclic alkyls include, but are not limited to, cyclopropyl, cyclobutyl, cyclopentyl, cyclohexyl, and the like, while unsaturated cyclic alkyls include, without limitation, cyclopentenyl, cyclohexenyl, and the like.

“Halo” or “halogen” refers to bromo (Br), chloro (Cl), fluoro (F) or iodo (I) substituents.

The term “haloalkyl” includes alkyl structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

An “alkoxy” group refers to a (alkyl)O-group, where alkyl is as defined herein.

An “aryl” group refers to monocyclic, bicyclic (fused), and tricyclic (fused or spiro) hydrocarbon ring systems having a total of five to fourteen ring carbon atoms, wherein at least one ring in the system is aromatic and wherein each ring in the system contains 3 to 7 ring carbon atoms. For example, “aryl” can be “phenyl”. Substituted aryl includes aryl substituted by NHC(O)CH₃. Substituted phenyl includes phenyl substituted by NHC(O)CH₃.

Unless otherwise noted, optional substituents include one or more group selected from acetyl, NHC(O)CH₃, alkyl, alkenyl, alkynyl, alkoxy, amino, nitro, CN, hydroxyl, halo, cycloalkyl, heterocycloalkyl, aryl, heteroaryl, and fluoroalkyl.

In general, a “PEGn” associated with the number n, comprises the formula: —[O—CH₂CH₂]n-, where n is the number of ethylene oxide units. For example, PEG2 refers to—[O—CH₂CH₂]₂—.

Substituents are those that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

Abbreviation “(v/v)” refers to the phrase “volume for volume”, i.e., the proportion of a particular substance within a mixture, as measured by volume or a volume amount of a component of the composition disclosed herein relative to the total volume amount of the composition. Accordingly, the quantity is unitless and represents a volume percentage amount of a component relative to the total volume of the composition. For example, a 2% (v/v) solvent mixture can indicate 2 mL of one solvent is present in 100 mL of the solvent mixture.

Abbreviation “(w/w)” refers to the phrase “weight for weight”, i.e., the proportion of a particular substance within a mixture, as measured by weight or mass or a weight amount of a component of the composition disclosed herein relative to the total weight amount of the composition. Accordingly, the quantity is unitless and represents a weight percentage amount of a component relative to the total weight of the composition. For example, a 2% (w/w) solution can indicate 2 grams of solute is dissolved in 100 grams of solution.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991 or in “Amino Acid-Protecting Groups”, A. Isidro-Llobet, M. Alvarez, F. Albericio; Chem. Rev. 2009, 109, 2455-2504. The protecting groups may be removed at a convenient subsequent stage using methods known from the art.

Protected amino acids or peptide fragments may be reacted with each other using coupling reagents such as those described in “Peptide Coupling Reagents, More than a Letter Soup”, A. El-Faham, A. Albericio; Chem. Rev. 2011, 111, 6557-6602 or “Large-Scale Applications of Amide Coupling Reagents for the Synthesis of Pharmaceuticals”, J. R. Dunetz, J. Magano, G. A. Weisenburger; Org. Process Res. Dev. 2016, 20, 140-177.

The disulfide bond can be formed using methods as described in “Disulfide Bond Formation in Peptides”, L. Chen, I. Annis, G. Barany; Current Protocols in Protein Science, 2001, 18.6.1-18.6.19.

### Preparation of the Monocyclic Peptide

Solid phase peptide synthesis (SPPS) involves linking a starting peptide chain to a solid support and subsequently extending by a series of elongation cycles with washing and purification such as chromatography between the cycles. The limitation of the SPPS equipment size and the need of chromatography purification often makes it cost prohibitive to produce a peptide in manufacture scale of multi-10 kg quantities. In contrast, the process of preparing a monocyclic peptide as described herein utilizes a solution phase chemistry, telescoping synthesis, and efficient purification methods such as extraction and crystallization of key intermediates to achieve multi-10 kg production. In particular, the process descried herein provides a more cost-effective approach than SPPS to the large-scale manufacture of a monocyclic peptide having 8-20 amino acid residues.

The present invention provides a process for preparing a monocyclic peptide, or a salt thereof. The process comprises coupling a monocyclic peptide fragment with a linear chain peptide fragment, wherein the monocyclic peptide fragment is a peptide containing between 4 and 11 amino acid residues; wherein the monocyclic peptide fragment comprises a ring containing between 4 and 8 amino acid residues; wherein the linear chain peptide fragment is a peptide containing between 4 and 10 amino acid residues; and wherein an amide bond is formed between the monocyclic peptide fragment and the linear chain peptide fragment.

The process of the present invention is advantageous in that the coupling of the monocyclic peptide fragment with the linear chain peptide fragment can be carried out in a solution phase without using a solid support.

A further advantage of the process of the present invention is that it can be carried out on a large scale which is suitable for commercial manufacture. In some embodiments, the process yields the monocyclic peptide in an amount of greater than 1 Kg, specifically in an amount of greater than 10 Kg, in an among of greater than 20 Kg, in an amount of greater than 50 Kg, more specifically in an amount of between 10 Kg and 60 Kg.

Certain intermediates in the process can be isolated to enhance the overall purity and yield of the monocyclic peptide. In some embodiments, the process further comprises isolating and/or crystallizing one or more intermediates. In some embodiments, the process further comprises isolating and/or crystalizing one or more compounds selected from Formula I, II, III, III′, IV, V, V′, VI, VII, VIII, IX, X, XI, XII, XIII, and XIV. In some embodiments, the intermediates can be selected from compounds of Formula (IV), Formula (V) and Formula (VI). In some embodiments, the process further comprises isolating and/or crystalizing compounds of formula (I), (II), and (III).

In some embodiments, the amide bond is formed between the amino acid residue at the C-terminus of the monocyclic peptide fragment and the amino acid residue at the N-terminus of the linear chain peptide fragment. In an alternative embodiment, the amide bond is formed between the amino acid residue at the N-terminus of the monocyclic peptide fragment and the amino acid residue at the C-terminus of the linear chain peptide fragment.

The coupling of the monocyclic peptide fragment and the linear peptide fragment is carried out using any suitable amide coupling reagent. Non-limiting examples of coupling agents include carbodiimides coupling agents, a carbodiimide coupling agent with an additive (activated esters), aminium-based coupling reagents, uronium-based reagents, phosphonium-based coupling agents, halogenating reagents, acylazoles, acyl azides, acid halides, organophosphorus reagents, organosulfur reagents, triazine coupling reagents, pyridinium coupling reagents, mixed anhydride reagents and activated esters.

Carbodiimide coupling reagents include N,N′-Diisopropylcarbodiimide (DIC), N,N′-Dicyclohexylcarbodiimide (DCC), 1,1′-carbonyldiimidazole (CDI), and N-ethyl-N′-dimethylaminopropylcarbodiimide (EDC), N-ethyl-N′(3-dimethylaminopropyl)carbodiimidehydrochloride (EDCI), N-cyclohexyl-N′-isopropylcarbodiimide (CIC), N-tert-butyl-N′-methylcarbodiimide (BMC), N-tert-butyl-N′-ethylcarbodiimide (BEC), N,N′-dicyclopentylcarbodiimide (CPC), bis(4-(2,2-dimethyl-1,3-dioxolyl))methylcarbodiimide (BDDC), N-ethyl-N′-phenylcarbodiimide (PEC), and N-phenyl-N′-isopropylcarbodiimide (PIC).

Aminium coupling reagents include O-(Benzotriazol-1-yl)-N,N,N′,N′-tetramethyluroniumtetrafluoroborate (TBTU) or 1-[Bis(dimethylamino)methylene]-1H-1,2,3-triazolo[4,5-b]pyridinium 3-oxide hexafluorophosphate (HATU), O(benzotriazol-1-yl)-N,N,N′,N′-tetramethyluronium hexafluorophosphate (HBTU), 3-(diethylphosphoryloxy)-1,2,3-benzotriazin-4(3H)-one (DEPBT), O-(6-chlorobenzotriazol-1-yl)-N,N,N′,N′-tetramethyluronium hexafluorophosphate (HCTU), and O-(7-azabenzotriazol-1-yl)-N,N,N′,N′-tetramethyluronium tetrafluoroborate (TATU).

Uronium-based reagents include O-(5-norbornene-2,3-dicarboximido)-N,N,N′,N′-tetramethyluronium tetrafluoroborate (TNTU), and O—(N-succinimidyl)-1,1,3,3-tetramethyluronium tetrafluoroborate (TSTU), O-(3,4-dihydro-4-oxo-1,2,3-benzotriazine-3-yl)-N,N,N′,N′-tetramethyluronium tetrafluoroborate (TDBTU)), O-(1,2-dihydro-2-oxo-1-pyridyl-N,N,N′,N′-tetramethyluronium tetrafluoroborate (TPTU), or O-[(ethoxycarbonyl)cyano-methyleneamino]-N,N,N′,N′-tetramethyluronium tertafluoroborate (TOTU).

Phosphonium coupling agents include (7-Azabenzotriazol-1-yloxy)tripyrrolidinophosphonium hexafluorophosphate (PyAOP), benzotriazol-1-yloxy-tris(dimethylamino)-phosphonium hexafluorophosphate (BOP), (benzotriazol-1-yloxy)tripyrrolidinophosphonium hexafluorophosphate (PyBOP), bromotripyrrolidinophosphonium hexafluorophosphate (PyBrOP), bis(2-oxo-3-oxazolidinyl)phosphinic chloride (BOP—Cl), 1-cyano-2-ethoxy-2-oxoethylideneaminooxy-tris-pyrrolidino-phosphonium hexafluorophosphate (PyOxim), and 6-chloro-benzotriazole-1-yloxy-trispyrrolidinophosphonium hexafluorophosphate (PyClock).

Anhydride coupling reagents include symmetric anhydrides, mixed carbonic anhydrides, N-carboxy anhydrides, and urethane-protected N-carboxy anhydrides.

Other regents including, but are not limited to 3-(diethylphosphoryloxy)-1,2,3-benzotriazin-4(3H)-one (DEPBT), N,N,N′,N′-tertamethylchloroformamidium hexafluorophosphate (TCFH), or propylphosphonic anhydride solution.

In some embodiments, the amide bond is formed using a carbodiimide coupling reagent in the presence of an additive selected from ethyl cyanohydroxyiminoacetate (Oxyma Pure), 1-hydroxy-7-azabenzotriazole (HOAt) hydroxybenzotriazole (HOBt), 2-hydroxypyridine-N-oxide (HOPO), N-Hydroxysuccinimide (HOSu), 5-(hydroxyimino)-1,3-dimethylpyrimidine-2,4,6(1H,3H,5H)-trione (Oxyma B), hexafluoroisopropanol (HFIP) and 4-nitrophenylalcohol.

In some embodiments, a base, such as a tertiary amine base, may be used in the amide bond forming step. Suitable bases include bases selected from N-Methylmorpholine (NMM), Di-isopropylethylamine (DIPEA), 1,4-diazabicyclo[2.2.2]octane (DABCO), diethylamine (DEA), triethylamine (TEA) and 1,8-Diazabicyclo[5.4.0]undec-7-ene (DBU).

In some embodiments, the coupling is carried out in the presence of a solvent selected from acetonitrile (MeCN), methyltetrahydrofuran (MeTHF), dimethylsulfoxide (DMSO), tetrahydrofuran (THF), water, and 2-propanol.

In some embodiments, the amide bond is formed using a coupling reagent selected from carbodiimide coupling reagents, and carbodiimides in the presence of an additive. In some embodiments, the amide bond is formed using N,N′-diisopropylcarbodiimide and 5-(hydroxyamino)-1,3-dimethylpyrimidine-2,4,6(1H,3H,5H)-trione (Oxyma B). In some embodiments the solvent for the coupling reaction comprises 2-methyltetrahydrofuran.

In some embodiments, provided is a process for preparing a monocyclic peptide, or a salt thereof, comprising coupling a monocyclic peptide fragment with a linear chain peptide fragment, wherein the monocyclic peptide fragment is a peptide of Formula (III-A):

R¹-X1a-X2a-X3a-X4a-X5a-X6a-X7a-X8a-X9a-R³ (III-A);
wherein each of X1a, X2a, and X3a is independently absent or is an amino acid residue;
each of X4a, X5a, X6a, X7a, X8a, and X9a is an amino acid residue;
R¹ is H, or C₁₋₂₀ alkanoyl;
R³ is OH or OP²; and
the peptide of Formula (III-A) is cyclized via a bond between two amino acid residues to form a ring containing between 4 and 8 amino acid residues; and
wherein the linear peptide fragment is of Formula (IV-A):

R²-X10a-X11a-X12a-X13a-X14a-X15a-X16a-X17a-X18a-X19a-R⁴ (IV-A);
wherein each of X10a, X11a, X12a, X13a, X14a, and X15a is an amino acid residue;
each of X16a, X17a, X18a, and X19a is independently absent or is an amino acid residue;
R² is H;
R⁴ is NHP¹, OP², NH₂, or OH;
P¹ is an amino protecting group;
each P² is independently a carboxyl protecting group;
and wherein the amide bond is formed between X9a of Formula (III-A) and X10a of Formula (VI-A).

In instances where R³ is OP², the carboxyl protecting group (P²) can be removed in situ during the coupling.

In some embodiments, provided is a process for preparing a monocyclic peptide, or a pharmaceutically acceptable salt thereof, comprising coupling a monocyclic peptide fragment with a linear chain peptide fragment, wherein the monocyclic peptide fragment is a peptide of Formula (III-A):

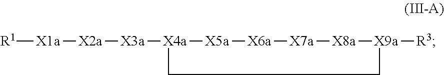

wherein each of X1a, X2a, and X3a is independently absent or is an amino acid residue;
each of X4a, X5a, X6a, X7a, X8a, and X9a is an amino acid residue;
R¹ is H, or C₁₋₂₀ alkanoyl;
R³ is OH or OP²; and
the peptide of Formula (III-A) is cyclized via a bond between X4a and X9a; and
wherein the linear peptide fragment is of Formula (IV-A):

R²-X10a-X11a-X12a-X13a-X14a-X15a-X16a-X17a-X18a-X19a-R⁴ (IV-A);
wherein each of X10a, X11a, X12a, X13a, X14a, and X15a is an amino acid residue;
each of X16a, X17a, X18a, and X19a is independently absent or is an amino acid residue;
R² is H;
R⁴ is NHP¹, OP², NH₂, or OH;
P¹ is an amino protecting group;
each P² is independently a carboxyl protecting group;
and wherein the amide bond is formed between X9a of Formula (III-A) and X10a of Formula (VI-A).

In instances where R³ is OP², the carboxyl protecting group (P²) can be removed in situ during the coupling.

In another embodiment, provided is a process for preparing a monocyclic peptide, or a pharmaceutically acceptable salt thereof, wherein the monocyclic peptide fragment is a peptide of Formula (III-B):

R¹-X1a-X2a-X3a-X4a-X5a-X6a-X7a-X8a-X9a-X10a-R³ (III-B);
wherein each of X1a, X2a, and X3a is independently absent or is an amino acid residue;
each of X4a, X5a, X6a, X7a, X8a, X9a and X10a is an amino acid residue;
R¹ is H, or C₁₋₂₀ alkanoyl;
R³ is OH or OP²; and
the peptide of Formula (III-B) is cyclized via a bond between two amino acid residues to form a ring containing between 4 and 8 amino acid residues; and
wherein the linear peptide fragment is of Formula (IV-B):

R²-X11a-X12a-X13a-X14a-X15a-X16a-X17a-X18a-X19a-R⁴ (IV-B);
wherein each of X11a, X12a, X13a, X14a, and X15a is an amino acid residue;
each of X16a, X17a, X18a, and X19a is independently absent or is an amino acid residue;
R² is H; and
R⁴ is NHP¹, OP², NH₂, or OH;
P¹ is an amino protecting group;
each P² is independently a carboxyl protecting group;
and wherein the amide bond is formed between X10a of Formula (III-B) and X11a of Formula (VI-B).

In instances where R³ is OP², the carboxyl protecting group (P²) can be removed in situ during the coupling.

In another embodiment, provided is a process for preparing a monocyclic peptide, or a pharmaceutically acceptable salt thereof, wherein the monocyclic peptide fragment is a peptide of Formula (III-B):

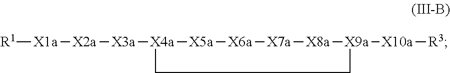

wherein each of X1a, X2a, and X3a is independently absent or is an amino acid residue;
each of X4a, X5a, X6a, X7a, X8a, X9a and X10a is an amino acid residue;
R¹ is H, or C₁₋₂₀ alkanoyl;
R³ is OH or OP²; and
the peptide of Formula (III-B) is cyclized via a bond between X4a and X9a; and
wherein the linear peptide fragment is of Formula (IV-B):

R²-X11a-X12a-X13a-X14a-X15a-X16a-X17a-X18a-X19a-R⁴ (IV-B);
wherein each of X11a, X12a, X13a, X14a, and X15a is an amino acid residue;
each of X16a, X17a, X18a, and X19a is independently absent or is an amino acid residue;
R² is H; and
R⁴ is NHP¹, OP², NH₂, or OH;
P¹ is an amino protecting group;
each P² is independently a carboxyl protecting group;
and wherein the amide bond is formed between X10a of Formula (III-B) and X11a of Formula (VI-B).

In instances where R³ is OP², the carboxyl protecting group (P²) can be removed in situ during the coupling.

In a further embodiment, provided is a process for preparing a monocyclic peptide, or a pharmaceutically acceptable salt thereof, wherein the monocyclic peptide fragment is a peptide of Formula (III-C):

R¹-X1a-X2a-X3a-X4a-X5a-X6a-X7a-X8a-X9a-X10a-X11a-R³ (III-C);
wherein each of X1a, X2a, and X3a is independently absent or is an amino acid residue;
each of X4a, X5a, X6a, X7a, X8a, X9a, X10a, and X11a is an amino acid residue;
R¹ is H, or C₁₋₂₀ alkanoyl;
R³ is OH or OP²; and
the peptide of Formula (III-C) is cyclized via a bond between two amino acid residues to form a ring containing between 4 and 8 amino acid residues; and
wherein the linear peptide fragment is of Formula (IV-C):

R²-X12a-X13a-X14a-X15a-X16a-X17a-X18a-X19a-R⁴ (IV-C);
wherein each of X12a, X13a, X14a, and X15a is an amino acid residue;
wherein each of X16a, X17a, X18a, and X19a is independently absent or is an amino acid residue;
R² is H; and
R⁴ is NHP¹, OP², NH₂ or OH;
P¹ is an amino protecting group;
each P² is independently a carboxyl protecting group; and
wherein the amide bond is formed between X11a of Formula (III-C) and X12a of Formula (IV-C).

In instances where R³ is OP², the carboxyl protecting group (P²) can be removed in situ during the coupling.

In a further embodiment, provided is a process for preparing a monocyclic peptide, or a pharmaceutically acceptable salt thereof, wherein the monocyclic peptide fragment is a peptide of Formula (III-C):

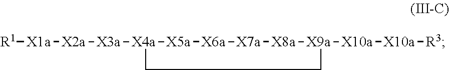

wherein each of X1a, X2a, and X3a is independently absent or is an amino acid residue;
each of X4a, X5a, X6a, X7a, X8a, X9a, X10a, and X11a is an amino acid residue;
R¹ is H, or C₁₋₂₀ alkanoyl;
R³ is OH or OP²; and
the peptide of Formula (III-C) is cyclized via a bond between X4a and X9a; and
wherein the linear peptide fragment is of Formula (IV-C):

R²-X12a-X13a-X14a-X15a-X16a-X17a-X18a-X19a-R⁴ (IV-C);
wherein each of X12a, X13a, X14a, and X15a is an amino acid residue;
wherein each of X16a, X17a, X18a, and X19a is independently absent or is an amino acid residue;
R² is H; and
R⁴ is NHP¹, OP², NH₂ or OH;
P¹ is an amino protecting group;
each P² is independently a carboxyl protecting group;
and wherein the amide bond is formed between X11a of Formula (III-C) and X12a of Formula (VI-C).

In instances where R³ is OP², the carboxyl protecting group (P²) can be removed in situ during the coupling.

Functional groups on the side chain of each amino acid selected from X1a-X19a is independently optionally protected with a suitable protecting group.

P¹ is any suitable amino protecting group. As will be readily recognised by the skilled person, the protecting group P¹ at the N-terminus of a peptide should be deprotected prior to or during the amide bond forming step. In some embodiments, P¹ is an amino protecting group selected from benzyl (Bn), trityl (Trt), 4-methyltrityl (Mtt), β-methoxyethoxytrityl (MEM), 2-nitrophenylsulfonyl (Nps), 2-(4-nitrophenyl)sulfonylethoxycarbonyl (Nsc), benzothiazole-2-sulfonyl (Bts), dithiasuccinoyl (Dts), nitrobenzenesulfonyl (Ns), 2-(2-nitrophenyl)propoxycarbonyl (NPPOC), 2-(3,4-methylenedioxy-6-nitrophenyl)propoxycarbonyl (MNPPOC), methylsulfonylethoxycarbonyl (Msc), 9-fluorenylmethyloxycarbonyl (Fmoc), 2,7-di-tert-butyl-Fmoc (Fmoc*), 2-fluoro-Fmoc (Fmoc(2F)), 2-monoisooctyl-Fmoc (mio-Fmoc), benzyloxycarbonyl (Cbz), 2,2,2-trichloroethoxycarbonyl (Troc), 2-(trimethylsilyl)ethoxycarbonyl (Teoc), 2-(4-trifluoromethylphenylsulfonyl)ethoxycarbonyl (Tsc), tert-butoxycarbonyl (BOC), 1-adamantyloxycarbonyl (Adoc), 2-adamantyloxycarbonyl (2-Adoc), 2,4-dimethylpent-3-yloxycarbonyl (Doc), cyclohexyloxycarbonyl (Hoc), 1,1-dimethyl-2,2,2-trichloroethoxycarbonyl (TcBOC), formyl, acetyl (Ac), trifluoroacetyl (TFA), p-toluenesulfonyl (Ts), vinyl, 2-chloroethyl, 2-phenylsulfonylethyl, allyl, 2-nitrobenzyl, 4-nitrobenzyl, diphenyl-4-pyridylmethyl, N′,N′-dimethylhydrazinyl, methoxymethyl, tert-butoxymethyl (Bum), benzyloxymethyl (BOM), 2-tetrahydropyranyl (THP), tri(C₁₋₄ alkyl)silyl (e.g., tri(isopropyl)silyl), 1,1-diethoxymethyl, α,α-dimethyl-3,5-dimethoxybenzyloxycarbonyl (Ddz), 2-(p-biphenylyl)-2-propyloxycarbonyl (Bpoc), 1,1-dioxonaptho[1,2-b]thiophene-2-methyloxycarbonyl (α-Nsmoc), 3,3-dioxonaptho[2,1-b]thiophene-2-methyloxycarbonyl (β-Nsmoc), 1-(4,4-dimethyl-2,6-dioxocyclohex-1-ylidene)ethyl (Dde), 1-(4,4-dimethyl-2,6-dioxocyclohex-1-ylidene)isovaleryl (ivDde), 2-(phenyl(methyl)sulfonio)ethoxycarbonyl (Pms), N-ethanesulfonylethoxycarbonyl (Esc), 2-(4-sulfophenylsulfonyl)ethoxycarbonyl (Sps), allyloxycarbonyl (Alloc), propargyloxycarbonyl (Poc), 9-(4-bromophenyl)-9-fluorenyl (BrPhF), azidomethyloxycarbonyl (Azoc), N-tetrachlorophthaloyl (TCP), phenyldithioethyloxycarbonyl (Phdec), 2-pyridyldithioethyloxycarbonyl (Pydec) or N-pivaloyloxymethyl (POM).

P² is any suitable carboxyl protecting group. As will be readily recognised by the skilled person, the protecting group P² at the C-terminus of a peptide should be deprotected prior to or during the amide bond forming step. In some embodiments, P² is a carboxyl protecting group selected from t-butyl (tBu), methoxy (Ome), ethoxy (Oet), allyl, 1,1-dimethylallyl (Dma), phenacyl (Pac), p-nitrobenzyl (p-NB) trityl (Tr), 2-chlorotrityl (2-Cl-Trt), 2,4-dimethoxybenzyl (Dmb), 9-fluorenylmethyl (Fm), phenyl, cyclohexyl, benzyl (Bn), 3,4-ehtlyenedioxy-2-thenyl (EDOTₙ), 4-(N-[1-(4,4-diemthyl-2,6-dioxocyclohexylidene)-3-methylbutyl)amino)benzyl (Dmab), trimethylsilylethyl (TMSE), 2-(trimethylsilyl)isopropyl (Tmsi), 2,2,2-trichloroethyl (Tee), carbamoylmethyl (Cam), 4,5-dimethoxy-2-nitrobenzyloxycarbonyl (Dmnb), pentamine cobalt (III), β-menthyl (Men), β-3-menthylpent-3-yl (Mpe), or 2-phenylisopropyl (2-Ph(iPr)).

The Monocyclic Peptide Compound

The process of the present invention allows for the large-scale preparation of a monocyclic peptide. The monocyclic peptide in its final deprotected form may be useful as an inhibitor of the interleukin-23 receptor (IL-23R).

The monocyclic peptide is a peptide containing between 8 and 21 amino acid residues.

In some embodiments, the monocyclic peptide has a relative molecular mass (RMM) of greater than 1000.

In some embodiments, each of the amino acid residues in the compound are independently selected from naturally occurring and non-naturally occurring amino acids, as well as amino acid analogs and amino acid mimetics that function in a manner similar to the naturally occurring amino acids.

In some embodiments, each amino acid residues are independently selected from Gly, Ala, beta-Ala, Leu, Met, Phe, Phe substituted with halo, alkyl, haloalkyl, hydroxy, alkoxy, cyano, cycloalkyl, carboxy, carboxamido, 2-aminoethoxy, or 2-acetylaminoethoxy, Trp, Trp substituted with cyano, halo, alkyl, substituted and unsubstituted aryl, haloalkyl, hydroxy, or alkoxy, Lys, Gln, beta-homoGln, Pro, Val, Ile, Cys, (D)Cys, alpha-MeCys, (D)Pen, Pen, or Pen(sulfoxide), Cit, Tyr, His, (D)His, Arg, Asn, Glu, Ser, alpha-MeSer, alpha-MeGln, alpha-MeLys, alpha-MeLeu, alpha-MeAsn, alpha-MeThr, Lys(Ac), alpha-MeLys(Ac), alpha-MeArg, alpha-MePhe, alpha-MeTyr Dab(Ac), Dap(Ac), homo-Lys(Ac), Asp, Thr, Sarc, Aib, Dab, Dap, gamma-Glu, Gaba, beta-Pro, Abu, 1Nal, 2Nal, Lys(b-Ala), Lys(Gly), Lys(Benzyl, Ac), Lys(butyl, Ac), Lys(isobutyl, Ac), Lys(propyl, Ac), Lys(PEG2PEG2gEC18OH), Phe(2-Me), Phe(3-Me), Phe(4-Me), Phe(3,4-dimethoxy), 2Quin, 3Quin, 4-amino-4-carboxy-tetrahydropyran (Gly(THP)), Acvc, cyclohexylAla, 2Pal, 3Pal, or 4Pal, 5Pyal. Phe substituted with 2-aminoethoxy is sometimes referred to herein as Tyr(2ea).

As will be readily recognised by the skilled person, it may be desirable to protect thiol, amino, carboxyl and hydroxyl groups which are present on the side chains of the amino acid residues. The groups on each of the amino acid residues are optionally protected with a suitable amino acid protecting group.

In any of the formulae disclosed herein, for example any of Formula II-Formula XIV, each amino group on the side chain of each amino acid residue is independently protected with Pg¹, wherein each Pg¹ is an amino protecting group independently selected from the group consisting of benzyl (Bn), trityl (Trt), 4-methyltrityl (Mtt), β-methoxyethoxytrityl (MEM), 2-nitrophenylsulfonyl (Nps), 2-(4-nitrophenyl)sulfonylethoxycarbonyl (Nsc), benzothiazole-2-sulfonyl (Bts), dithiasuccinoyl (Dts), nitrobenzenesulfonyl (Ns), 2-(2-nitrophenyl)propoxycarbonyl (NPPOC), 2-(3,4-methylenedioxy-6-nitrophenyl)propoxycarbonyl (MNPPOC), methylsulfonylethoxycarbonyl (Msc), 9-fluorenylmethyloxycarbonyl (Fmoc), 2,7-di-tert-butyl-Fmoc (Fmoc*), 2-fluoro-Fmoc (Fmoc(2F)), 2-monoisooctyl-Fmoc (mio-Fmoc), benzyloxycarbonyl (Cbz), 2,2,2-trichloroethoxycarbonyl (Troc), 2-(trimethylsilyl)ethoxycarbonyl (Teoc), 2-(4-trifluoromethylphenylsulfonyl)ethoxycarbonyl (Tsc), tert-butoxycarbonyl (BOC), 1-adamantyloxycarbonyl (Adoc), 2-adamantyloxycarbonyl (2-Adoc), 2,4-dimethylpent-3-yloxycarbonyl (Doc), cyclohexyloxycarbonyl (Hoc), 1,1-dimethyl-2,2,2-trichloroethoxycarbonyl (TcBOC), formyl, acetyl (Ac), trifluoroacetyl (TFA), p-toluenesulfonyl (Ts), vinyl, 2-chloroethyl, 2-phenylsulfonylethyl, allyl, 2-nitrobenzyl, 4-nitrobenzyl, diphenyl-4-pyridylmethyl, N′,N′-dimethylhydrazinyl, methoxymethyl, tert-butoxymethyl (Bum), benzyloxymethyl (BOM), 2-tetrahydropyranyl (THP), tri(C₁₋₄ alkyl)silyl (e.g., tri(isopropyl)silyl), 1,1-diethoxymethyl, α,α-dimethyl-3,5-dimethoxybenzyloxycarbonyl (Ddz), 2-(p-biphenylyl)-2-propyloxycarbonyl (Bpoc), 1,1-dioxonaptho[1,2-b]thiophene-2-methyloxycarbonyl (α-Nsmoc), 3,3-dioxonaptho[2,1-b]thiophene-2-methyloxycarbonyl (β-Nsmoc), 1-(4,4-dimethyl-2,6-dioxocyclohex-1-ylidene)ethyl (Dde), 1-(4,4-dimethyl-2,6-dioxocyclohex-1-ylidene)isovaleryl (ivDde), 2-(phenyl(methyl)sulfonio)ethoxycarbonyl (Pms), N-ethanesulfonylethoxycarbonyl (Esc), 2-(4-sulfophenylsulfonyl)ethoxycarbonyl (Sps), allyloxycarbonyl (Alloc), propargyloxycarbonyl (Poc), 9-(4-bromophenyl)-9-fluorenyl (BrPhF), azidomethyloxycarbonyl (Azoc), N-tetrachlorophthaloyl (TCP), phenyldithioethyloxycarbonyl (Phdec), 2-pyridyldithioethyloxycarbonyl (Pydec) or N-pivaloyloxymethyl (POM).

In any of the formulae disclosed herein, for example any of Formula II-Formula XIV, each carboxyl group on the side chain of each amino acid residue is independently protected with Pg², wherein each Pg² is a carboxyl protecting group independently selected from the group consisting of tert-butyl (tBu), methoxy (Ome), ethoxy (Oet), allyl, 1,1-dimethylallyl (Dma), phenacyl (Pac), p-nitrobenzyl (p-NB), trityl (Tr), 2-chlorotrityl (2-Cl-Trt), 2,4-dimethoxybenzyl (Dmb), 9-fluorenylmethyl (Fm), phenyl, cyclohexyl, benzyl (Bn), 3,4-ethlyenedioxy-2-thenyl (EDOTₙ), 4-(N-(1-(4,4-dimethyl-2,6-dioxocyclohexylidene)-3-methylbutyl)amino)benzyl (Dmab), trimethylsilylethyl (TMSE), 2-(trimethylsilyl)isopropyl (Tmsi), 2,2,2-trichloroethyl (Tce), carbamoylmethyl (Cam), 4,5-dimethoxy-2-nitrobenzyloxycarbonyl (Dmnb), hydroxyami cobalt (III), β-menthyl (Men), β-3-menthylpent-3-yl (Mpe), or 2-phenylisopropyl (2-Ph(iPr));

In any of the formulae disclosed herein, for example any of Formula II-Formula XIV, each hydroxyl group on the side chain of each amino acid residue is independently protected with Pg³, wherein each Pg³ is a hydroxyl protecting group independently selected from the group consisting of benzyl (Bn), cyclohexyl, tert-butyl (tBu), trityl (Trt), tert-butyldimethylsilyl (TBDMS), pseudoprolines, tert-butyldiphenylsilyl (TBDPS), 4,5-dimethoxy-2-nitrobenzyloxycarbonyl (Dmnb), propargyloxycarbonyl (Poc).

In any of the formulae disclosed herein, for example any of Formula II-Formula XIV, each thiol group on the side chain of each amino acid residue is independently protected with Pg⁴, wherein each thiol protecting group is independently selected from the group consisting of p-methyl benzyl (Meb), p-methoxybenzyl (Mob), trityl (Tr), monomethoxytrityl (Mmt), 2,4,6-trimethoxybenzyl (Tmob), 9-xanthenyl (Xan), 2,2,4,6,7-pentamethyl-5-dihydrobenzofuranylmethyl (Pmbf), benzyl (Bn), tert-butyl (tBu), 1-adamantyl (1-Ada), 9-fluorenylmethyl (Fm), 2-(2,4-dinitrophenyl)ethyl (Dnpe), 9-fluorenylmethyloxycarbonyl (Fmoc), acetamidomethyl (Acm), phenylacetamidomethyl (PhAcm), tert-butylmercapto (S(tBu)), 3-nitro-2-pyridinesulfenyl (Npys), 2-pyridinesulfenyl (S-Pyr), allyloxycarbonyl (Alloc), N-allyloxycarbonyl-N-[2,3,5,6-tetrafluoro-4-(41ydroxyamino)phenyl)aminomethyl (Fsam), o-nitrobenzyl (o-NB), 4-Picolyl, ninhydrin (Nin).

In some embodiments, amino functional groups on each of the amino acid side chains are protected with a protecting group independently selected from Fmoc, Cbz and Boc. In some embodiments, thiol functional groups on each of the amino acid side chains are protected with a protecting group independently selected from Trt and Acm. In some embodiments, carboxylic acid functional groups on each of the amino acid side chains are protected with a protecting group which is tert-butyl.

In some embodiments, the monocyclic peptide is a compound of Formula (IIa):

R¹-X1a-X2a-X3a-X4a-X5a-X6a-X7a-X8a-X9a-X10a-X11a-X12a-X13a-X14a-X15a-X16a-X17a-X18a-X19a-R⁴ (IIa)
wherein
R¹ is H, or C₁₋₂₀ alkanyol;
R⁴ is NHP¹, or NH₂;
P¹ is any amino protecting group;
each of X1a, X2a, X3a, X18a, and X19a is independently absent or any amino acid residue;
   X4a is Abu, Cys, (D)Cys, alpha-MeCys, (D)Pen, Pen, or Pen(sulfoxide);
   X5a is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys;
   X6a is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val;
   X7a is unsubstituted Trp, or Trp substituted with cyano, halo, alkyl, substituted or unsubstituted aryl, haloalkyl, hydroxy, or alkoxy;
   X8a is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1Nal, 2Nal, or Trp;
   X9a is Abu, Cys, (D)Cys, alpha-MeCys, (D)Pen, Pen, or Pen(sulfoxide);
   X10a is unsubstituted Phe, or Phe substituted with halo, alkyl, haloalkyl, hydroxy, alkoxy, carboxy, carboxamido, 2-aminoethoxy, or 2-acetylaminoethoxy;
   X11a is 2Nal, unsubstituted Trp, or Trp substituted with cyano, halo, alkyl, haloalkyl, hydroxy, alkoxy, Phe(2-Me), Phe(3-Me), Phe(4-Me), Phe(3,4-dimethoxy), or 1Nal;
   X12a is 4-amino-4-carboxy-tetrahydropyran (Gly(THP)), alpha-MeLys, alpha-MeLeu, alpha-MeArg, alpha-MePhe, alpha-MeLeu, alpha-MeLys, alpha-MeAsn, alpha-MeTyr, Ala, cyclohexylAla, Lys, or 2-aminoisobutyric acid;
   X13a is 2-aminoisobutyric acid, Glu, Cit, Gln, Lys(Ac), alpha-MeArg, alpha-MeGlu, alpha-MeLeu, alpha-MeLys, alpha-Me-Asn, alpha-MeLys(Ac), Dab(Ac), Dap(Ac), homo-Lys(Ac), or Lys; or X13a is Lys, pegylated Lys, b-homoGlu, or Lys(Y2-Ac), wherein Y2 is an amino acid residue;
   X14a is Asn, 2Nal, 2-aminoisobutyric acid, Arg, Cit, Asp, Phe, Gly, Lys, Leu, Ala, (D)Ala, beta-Ala, His, Thr, n-Leu, Gln, Ser, (D)Ser, Tic, Trp, alpha-MeGln, alpha-MeAsn, alpha-MeLys(Ac), Dab(Ac), Dap(Ac), homo-Lys(Ac), or Lys(Ac);
   X15a is Ala, beta-Ala, Arg, Asn, Asp, Cit, Cys, Glu, Gln, Gly, substituted or unsubstituted His, (D)His, Ile, Lue, (D)Lue, Lys, (D)Lys, Met, 2Pal, 3Pal, or 4Pal, Phe, Pro, 5Pyal, 2Quin, 3Quin, Ser, Thr, Trp, Tyr, Val; Leu;
   X16a is absent or Sarc, aMeLeu, (D)NmeTyr, His, (D)Thr, bAla, Pro, or (D)Pro;
   X17a is absent or Lys(PEG2PEG2gEC18OH); and wherein the monocyclic peptide fragment is cyclized via a bond between to amino acids to form a ring containing 4 and 8 amino acid residues.

In some embodiments, the amino, carboxyl, hydroxyl and thiol groups on the side chains of each of X1a-X19a are independently protected with a suitable protecting group. In some embodiments, each amino group on the side chain of each of X1a-X19a is independently protected with Pg¹ as defined above; each carboxyl group on the side chain of each of X1a-X19a is independently protected with Pg² as defined above; each hydroxyl group on the side chain of each of X1a-X19a is independently protected with Pg³ as defined above; and each thiol group on the side chain of each of X1a-X19a is independently protected with Pg⁴ as defined above.

In some embodiments, the monocyclic peptide is a compound of Formula (II):

R¹-X3a-X4a-X5a-X6a-X7a-X8a-X9a-X10a-X11a-X12a-X13a-X14a-X15a-X16a-R⁴ (II).

In some embodiments, the amino, carboxyl, hydroxyl and thiol groups on the side chains of each of X3a-X16a are independently protected with a suitable protecting group. In some embodiments, each amino group on the side chain of each of X3a-X16a is independently protected with Pg¹ as defined above; each carboxyl group on the side chain of each of X3a-X16a is independently protected with Pg² as defined above; each hydroxyl group on the side chain of each of X3a-X16a is independently protected with Pg³ as defined above; and each thiol group on the side chain of each of X3a-X16a is independently protected with Pg⁴ as defined above.

In some embodiments, the monocyclic peptide is a compound of Formula (Ia), (Ib), (Ic), (Id), or (Je):

R¹-X4a-X5a-X6a-[Trp]-X8a-X9a-[Phe]-[2Nal]-X12a-X13a-X14a-X15a-X16a-R⁴ (Ia),

R¹-X4a-X5a-X6a-[Trp]-X8a-X9a-[Phe]-X11a-X12a-X13a-X14a-[Pal]-X16a-R⁴ (Ib),

R¹-X4a-X5a-X6a-X7a-X8a-X9a-[Phe]-[2Nal]-X12a-X13a-X14a-[Pal]-X16a-R⁴ (Ic),

R¹-X4a-X5a-X6a-[Trp]-X8a-X9a-X10-[2Nal]-X12a-X13a-X14a-[Pal]-X16a-R⁴ (Id);

or

R¹-X4a-X5a-X6a-[Trp]-X8a-X9a-[Phe]-[2Nal]-X12a-X13a-X14a-[Pal]-X16a-R⁴ (Ie)
wherein Trp is unsubstituted Trp, or Trp substituted with cyano, halo, alkyl, haloalkyl, alkoxy, substituted or unsubstituted aryl, or substituted or unsubstituted heteroaryl;
wherein Phe is unsubstituted Phe, or Phe substituted with halo, alkyl, haloalkyl, hydroxy, alkoxy, cyano, cycloalkyl, carboxy, carboxamido, 2-aminoethoxy, or 2-acetylaminoethoxy;
wherein 2Nal is unsubstituted 2Nal;
wherein Pal is 2Pal, 3Pal, or 4Pal.

In some embodiments, the monocyclic peptide compound is Ac-(D)Arg-Abu-Gln-Thr-Trp-Gln-Cys-Tyr(2ea)-2Nal-Gly(THP)-Glu-Asn-Asn-NH₂ (SEQ ID NO. 1); the monocyclic peptide fragment is Ac-(D)Arg-Abu-Gln-Thr-Trp-Gln-Cys-OH (SEQ ID NO. 2), and the linear peptide fragment is H-Tyr(2ea)-2Nal-Gly(THP)-Glu-Asn-Asn-NH₂.

In some embodiments, the monocyclic peptide compound has the following structure:

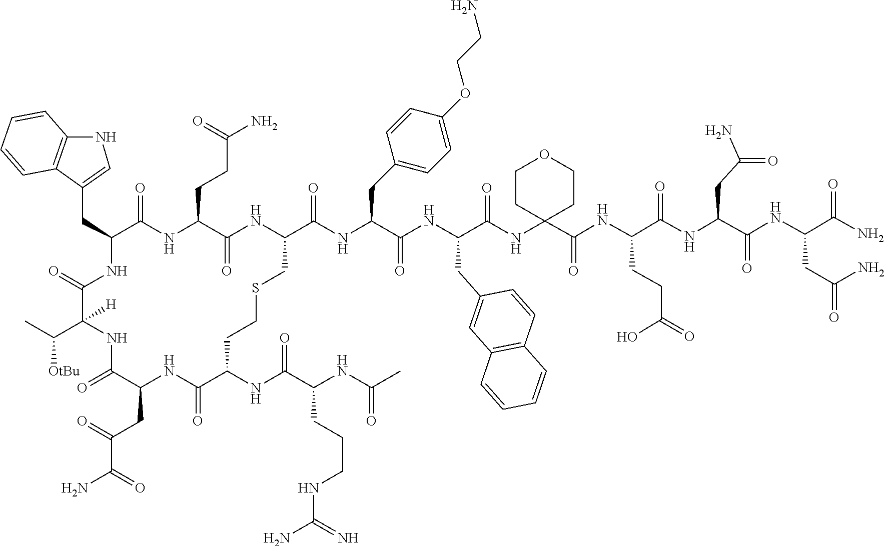 [SMILES: CC(=O)N[C@H](CCCNC(=N)N)C(=O)N[C@H]1CCSC[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](CC(N)=O)C(N)=O)CCOCC2)NC(=O)[C@H](CCC(N)=O)NC(=O)[C@H](Cc2c[nH]c3ccccc23)NC(=O)[C@H]([C@@H](C)OC(C)(C)C)NC(=O)[C@H](CC(=O)C(N)=O)NC1=O]

In some embodiments, the monocyclic peptide compound is Ac-Pen-Asn-Thr-Trp(7-Me)-Lys (Ac)-Pen-Tyr(2ea)-2Nal-alphaMeLys-Lys(Ac)-Asn-D-Leu-NH₂ (SEQ ID NO. 3); the monocyclic peptide fragment is Ac-Pen-Asn-Thr-Trp(7-Me)-Lys (Ac)-Pen-Tyr(2ea)—OH (SEQ ID NO. 4), and the linear peptide fragment is H-2Nal-alphaMeLys-Lys(Ac)-Asn-D-Leu-NH₂.

In some embodiments, the monocyclic peptide compound has the following

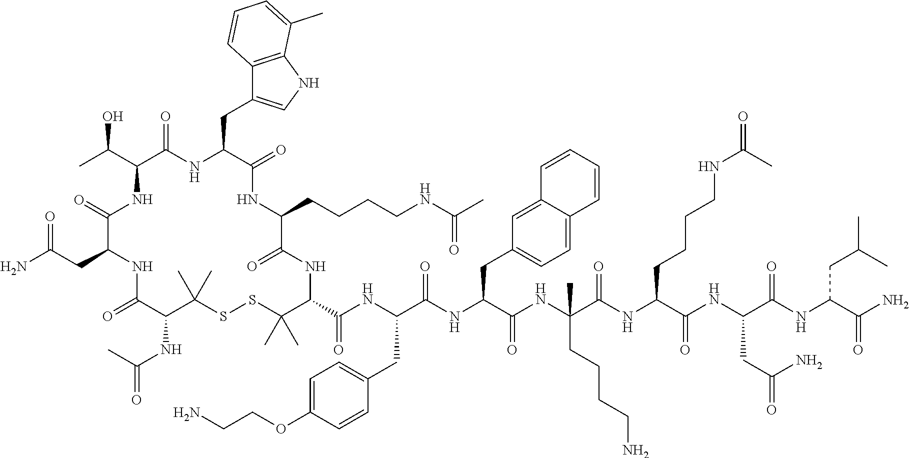 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)N[C@@](C)(CCCCN)C(=O)N[C@@H](CCCCNC(C)=O)C(=O)N[C@@H](CC(N)=O)C(=O)N[C@H](CC(C)C)C(N)=O)NC1=O]

In some embodiments, the monocyclic peptide compound is Ac-Pen-Asn-Thr-Trp(7-Me)-Lys(Ac)-Pen-Tyr(2ea)-2Nal-Gly(THP)-Glu-Asn-3Pal-DSarc-NH₂ (SEQ ID NO. 5); the monocyclic peptide fragment is Ac-Pen-Asn-Thr-Trp(7-Me)-Lys(Ac)-Pen-Tyr(2ea)-OH (SEQ ID NO. 4), and the linear peptide fragment is H-2Nal-Gly(THP)-Glu-Asn-3Pal-Sarc-NH₂.

In some embodiments, the monocyclic peptide compound has the following structure:

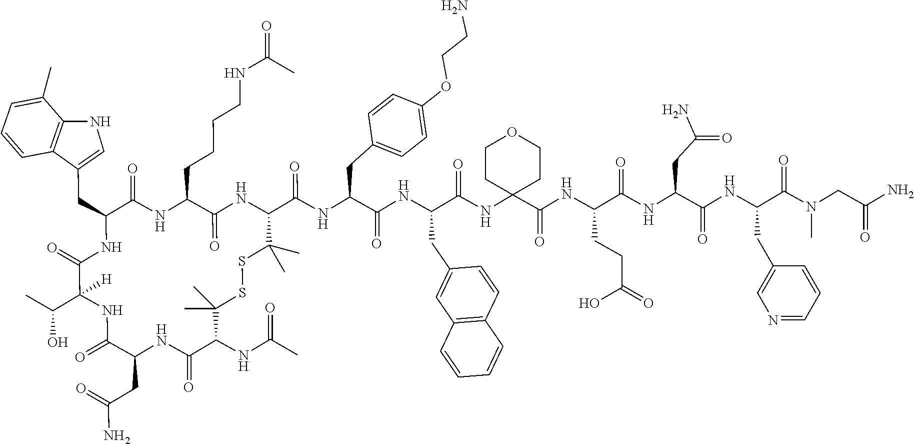 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

Protected Monocyclic Peptide Compound

To arrive at the final active pharmaceutical ingredient (API), it may be necessary to carry out a final global deprotection step to remove the protecting groups on the amino acid residues, for example the protecting groups on the side chains of each of amino acid residues X1a-X19a. In some embodiments, global deprotection is carried out using acidic conditions.

In some embodiments, the process of preparing the monocyclic peptide compound involves a deprotection step of a compound of Formula (II), wherein the compound of Formula (II) is:

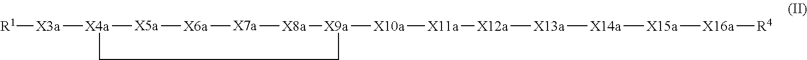

wherein
R¹ is H, or C₁₋₂₀ alkanoyl;
R⁴ is NHP¹;
P¹ is an amino protecting group;
X3a is absent or any amino acid residue;
X4a is Abu, Cys, (D)Cys, alpha-MeCys, (D)Pen, Pen, or Pen(sulfoxide);
X5a is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys;
X6a is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val;
X7a is unsubstituted Trp, or Trp substituted with cyano, halo, alkyl, substituted or unsubstituted aryl, haloalkyl, hydroxy, or alkoxy;
X8a is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1Nal, 2Nal, or Trp;
X9a is Abu, Cys, (D)Cys, alpha-MeCys, (D)Pen, Pen, or Pen(sulfoxide);
X10a is unsubstituted Phe, or Phe substituted with halo, alkyl, haloalkyl, hydroxy, alkoxy, carboxy, carboxamido, 2-aminoethoxy, or 2-acetylaminoethoxy; X11a is 2Nal, unsubstituted Trp, or Trp substituted with cyano, halo, alkyl, haloalkyl, hydroxy, alkoxy, Phe(2-Me), Phe(3-Me), Phe(4-Me), Phe(3,4-dimethoxy), or 1Nal;
X12a is 4-amino-4-carboxy-tetrahydropyran (Gly(THP)), alpha-MeLys, alpha-MeLeu, alpha-MeArg, alpha-MePhe, alpha-MeLeu, alpha-MeLys, alpha-MeAsn, alpha-MeTyr, Ala, or cyclohexylAla, Lys, or 2-aminoisobutyric acid;
X13a is 2-aminoisobutyric acid, Glu, Cit, Gln, Lys(Ac), alpha-MeArg, alpha-MeGlu, alpha-MeLeu, alpha-MeLys, alpha-Me-Asn, alpha-MeLys(Ac), Dab(Ac), Dap(Ac), homo-Lys(Ac), or Lys; or X13a is Lys, pegylated Lys, b-homoGlu, or Lys(Y2-Ac), wherein Y2 is an amino acid residue;
X14a is Asn, 2Nal, 2-aminoirobutyric acid, Arg, Cit, Asp, Phe, Gly, Lys, Leu, Ala, (D)Ala, beta-Ala, His, Thr, n-Leu, Gln, Ser, (D)Ser, Tic, Trp, alpha-MeGln, alpha-MeAsn, alpha-MeLys(Ac), Dab(Ac), Dap(Ac), homo-Lys(Ac), or Lys(Ac);
X15a is Ala, beta-Ala, Arg, Asn, Asp, Cit, Cys, Glu, Gln, Gly, substituted or unsubstituted His, (D)His, Ile, Lue, (D)Lue, Lys, (D)Lys, Met, 2Pal, 3Pal, or 4Pal, Phe, Pro, 5-Pyal, 2Quin, 3Quin, Ser, Thr, Trp, Tyr, Val; Leu;
X16a is absent or Sarc, aMeLeu, (D)NmeTyr, His, (D)Thr, bAla, Pro, or (D)Pro;
and the compound is cyclized via a Pen-Pen disulfide bond between X4a and X9a; or the compound is cyclized via a Abu-Cys or Abu-Pen thioether bond between X4a and X9a; wherein the side chains of each of the amino acid residues are independently optionally protected by suitable protecting groups.

In some embodiments, the amino, carboxyl, hydroxyl and thiol groups on the side chains of each of X3a-X19a are independently protected with a protecting group. Amino protecting groups can be independently selected from Fmoc, Cbz, and Boc. The carboxyl protecting groups can be independently selected from Methyl, tert-butyl, trityl (Tr), 2,4-dimethoxybenzyl (Dmb), 9-fluorenylmethyl (Fm), and benzyl (Bn). The hydroxyl protecting groups can be independently selected from tert-butyl. The thiol protecting groups can be independently selected from trityl (Tr), or acetamidomethyl (Acm).

In some embodiments, P¹ is Fmoc, Cbz, or Boc; and/or R¹ is C(O)CH₃; and/or R⁴ is NH₂.

In some embodiments, the compound of Formula (III) is Ac-Pen-Asn-Thr-Trp(7-Me)-Lys(Ac)-Pen-Tyr(2ea)-OH, wherein the hydroxyl and amino groups on Thr and Tyr(2ea) are protected; and the compound of Formula (IV) is H-2Nal-Gly(THP)-Glu-Asn-3Pal-Sarc-NHP¹, wherein the carboxyl group on Glu is protected; and the compound of Formula (II) is Ac-Pen-Asn-Thr-Trp(7-Me)-Lys(Ac)-Pen-Tyr(2ea)-2Nal-Gly(THP)-Glu-Asn-3Pal-Sarc-NHP¹, wherein the carboxyl and amino groups on each Thr, Tyr(2ea), and Glu are protected.

In some embodiments, the compound of Formula (II) is deprotected to form a compound of Formula (I):

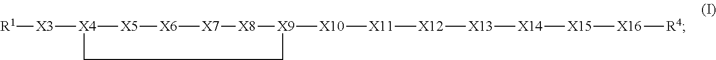

wherein R¹ is H, or C₁₋₂₀ alkanoyl;
R⁴ is NH₂;
X3 is absent or any amino acid residue;
X4 is Abu, Cys, (D)Cys, alpha-MeCys, (D)Pen, Pen, or Pen(sulfoxide);
X5 is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys;
X6 is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val;
X7 is unsubstituted Trp, or Trp substituted with cyano, halo, alkyl, haloalkyl, hydroxy, or alkoxy;
X8 is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1Nal, 2Nal, or Trp;
X9 is Abu, Cys, (D)Cys, alpha-MeCys, (D)Pen, Pen, or Pen(sulfoxide);
X10 is unsubstituted Phe, or Phe substituted with halo, alkyl, haloalkyl, hydroxy, alkoxy, carboxy, carboxamido, 2-aminoethoxy, or 2-acetylaminoethoxy; and
X11 is 2Nal, unsubstituted Trp, or Trp substituted with cyano, halo, alkyl, haloalkyl, hydroxy, alkoxy, Phe(2-Me), Phe(3-Me), Phe(4-Me), Phe(3,4-dimethoxy), or 1Nal;
X12 is 4-amino-4-carboxy-tetrahydropyran (Gly(THP)), alpha-MeLys, alpha-MeLeu, alpha-MeArg, alpha-MePhe, alpha-MeLeu, alpha-MeLys, alpha-MeAsn, alpha-MeTyr, Ala, cyclohexylAla, Lys, or 2-aminoisobutyric acid;
X13 is 2-aminoisobutyric acid, Glu, Cit, Gln, Lys(Ac), alpha-MeArg, alpha-MeGlu, alpha-MeLeu, alpha-MeLys, alpha-Me-Asn, alpha-MeLys(Ac), Dab(Ac), Dap(Ac), homo-Lys(Ac), or Lys; or X13 is Lys, pegylated Lys, b-homoGlu, or Lys(Y2-Ac), wherein Y2 is an amino acid residue;
X14 is Asn, 2Nal, 2-aminoirobutyric acid, Arg, Cit, Asp, Phe, Gly, Lys, Leu, Ala, (D)Ala, beta-Ala, His, Thr, n-Leu, Gln, Ser, (D)Ser, Tic, Trp, alpha-MeGln, alpha-MeAsn, alpha-MeLys(Ac), Dab(Ac), Dap(Ac), homo-Lys(Ac), or Lys(Ac);
X15 is Ala, beta-Ala, Arg, Asn, Asp, Cit, Cys, Glu, Gln, Gly, substituted or unsubstituted His, (D)His, Ile, Lue, (D)Lue, Lys, (D)Lys, Met, 2Pal, 3Pal, or 4Pal, Phe, Pro, 5-Pyal, 2Quin, 3Quin, Ser, Thr, Trp, Tyr, Val, Leu;
X16 is absent or Sarc, aMeLeu, (D)NmeTyr, His, (D)Thr, bAla, Pro, or (D)Pro;
and the compound is cyclized via a Pen-Pen disulfide bond between X4 and X9; or the compound is cyclized via a Abu-Cys or Abu-Pen thioether bond between X4 and X9.

In some embodiments, X4a is (D)Pen, Pen, or Pen(sulfoxide); and X9a is (D)Pen, Pen, or Pen(sulfoxide).

In some embodiments, deprotection is performed using acid. Acids include acetic acid.

In some embodiments, the compound of Formula (I) is Ac-Pen-Asn-Thr-Trp(7-Me)-Lys(Ac)-Pen-Tyr(2ea)-2Nal-Gly(THP)-Glu-Asn-3Pal-Sarc-NH₂. Ac-Pen-Asn-Thr-Trp(7-Me)-Lys(Ac)-Pen-Tyr(2ea)-2-Nal-Gly(THP)-Glu-Asn-3-Pal-Sarc-NH₂ has the following structure:

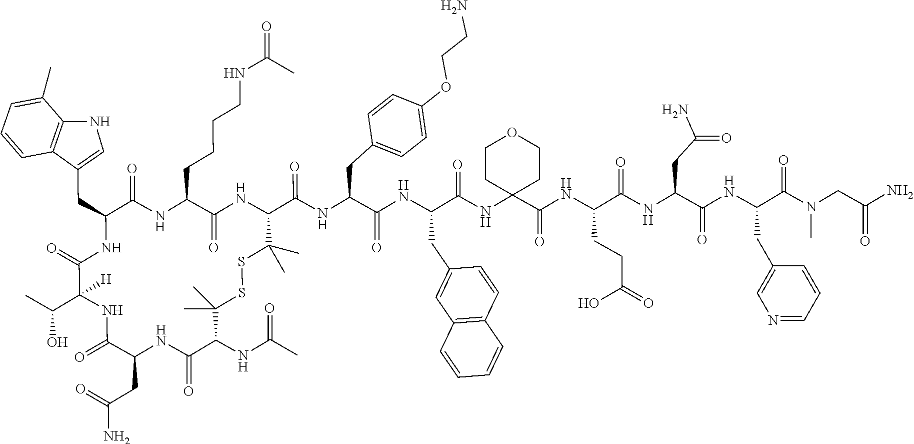 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

In some embodiments, the protected precursor to the compound of Formula (I), wherein the compound of Formula (I) is Ac-Pen-Asn-Thr-Trp(7-Me)-Lys(Ac)-Pen-Tyr(2ea)-2-Nal-Gly(THP)-Glu-Asn-3-Pal-Sarc-NH₂, is a compound of Formula (II) which has the following structure:

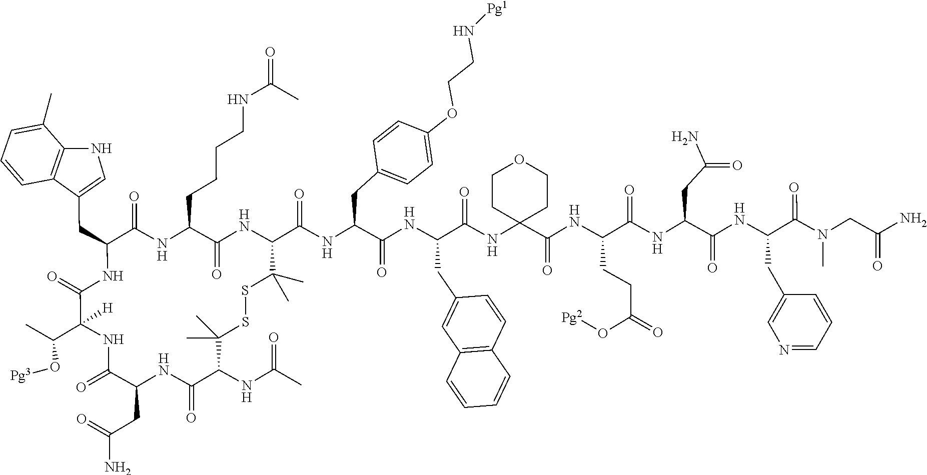

wherein Pg¹, Pg² and Pg³ are protecting groups as described above. In some embodiments Pg¹ is Boc, Pg² is tert-butyl, and Pg³ is Boc.

Linear Chain Peptide Fragment

In some embodiments, the linear chain fragment is a peptide having a relative molecular mass (RMM) of between 500 and 3000.

In some embodiments, the linear chain peptide fragment is a peptide containing 5 or 6 amino acid residues. In another embodiment, the linear chain peptide fragment is a peptide containing 6 residues.

In some embodiments, the linear chain peptide fragment is an unbranched peptide. An unbranched peptide is a peptide consisting of a chain of amino acid residues which does not include further amino acid residues branching off the chain of amino acids. A peptide consisting of a chain of amino acid residues which is cyclized through the amino acid side chains at the terminal positions is also referred to as unbranched.

In some embodiments, the linear peptide fragment is a peptide of Formula (IV-A), (IV-B), or (IV-C):

R²-X10a-X11a-X12a-X13a-X14a-X15a-X16a-X17a-X18a-X19a-R⁴ (IV-A);

R²-X11a-X12a-X13a-X14a-X15a-X16a-X17a-X18a-X19a-R⁴ (IV-B);

R²-X12a-X13a-X14a-X15a-X16a-X17a-X18a-X19a-R⁴ (IV-C);
wherein each of X10a, X11a, X12a, X13a, X14a, and X15a is an amino acid residue;
each of X16a, X17a, X18a, and X19a is independently absent or is an amino acid residue;
R² is H;
R⁴ is NHP¹, OP², NH₂ or OH;
P¹ is an amino protecting group; and
P² is a carboxyl protecting group.

In some embodiments, the amino, carboxyl, hydroxyl and thiol groups on the side chains of each of X10a-X19a are independently protected with a suitable protecting group.

In some embodiments, the linear chain fragment is a compound of Formula (IV):

R²-X11a-X12a-X13a-X14a-X15a-X16a-R⁴ (IV)
wherein
R² is H;
R⁴ is NHP¹, or NH₂;
P¹ is an amino protecting group;
X11a is 2Nal, unsubstituted Trp, or Trp substituted with cyano, halo, alkyl, haloalkyl, hydroxy, alkoxy, Phe(2-Me), Phe(3-Me), Phe(4-Me), Phe(3,4-dimethoxy), or 1Nal;
X12a is 4-amino-4-carboxy-tetrahydropyran (Gly(THP)), alpha-MeLys, alpha-MeLeu, alpha-MeArg, alpha-MePhe, alpha-MeLeu, alpha-MeLys, alpha-MeAsn, alpha-MeTyr, Ala, cyclohexylAla, Lys, or 2-aminoisobutyric acid;
X13a is 2-aminoisobutyric acid, Glu, Cit, Gln, Lys(Ac), alpha-MeArg, alpha-MeGlu, alpha-MeLeu, alpha-MeLys, alpha-Me-Asn, alpha-MeLys(Ac), Dab(Ac), Dap(Ac), homo-Lys(Ac), or Lys; or X13a is Lys, pegylated Lys, b-homoGlu, or Lys(Y2-Ac), wherein Y2 is an amino acid residue;
X14a is Asn, 2Nal, 2-aminoirobutyric acid, Arg, Cit, Asp, Phe, Gly, Lys, Leu, Ala, (D)Ala, beta-Ala, His, Thr, n-Leu, Gln, Ser, (D)Ser, Tic, Trp, alpha-MeGln, alpha-MeAsn, alpha-MeLys(Ac), Dab(Ac), Dap(Ac), homo-Lys(Ac), or Lys(Ac);
X15a is Ala, beta-Ala, Arg, Asn, Asp, Cit, Cys, Glu, Gln, Gly, substituted or unsubstituted His, (D)His, Ile, Lue, (D)Lue, Lys, (D)Lys, Met, 2Pal, 3Pal, or 4Pal, Phe, Pro, 5-Pyal, 2Quin, 3Quin, Ser, Thr, Trp, Tyr, Val, Leu;
X16a is absent or Sarc, aMeLeu, (D)NmeTyr, His, (D)Thr, bAla, Pro, or (D)Pro.

In some embodiments, the amino, carboxyl, hydroxyl and thiol groups on the side chains of each of X11a-X16a are independently protected with a suitable protecting group.

In some embodiments, the linear chain peptide fragment is R²-2Nal-Gly(THP)-Glu-Asn-3Pal-Sarc-R⁴.

In some embodiments the linear chain peptide fragment is a compound of Formula (IV) having the following structure:

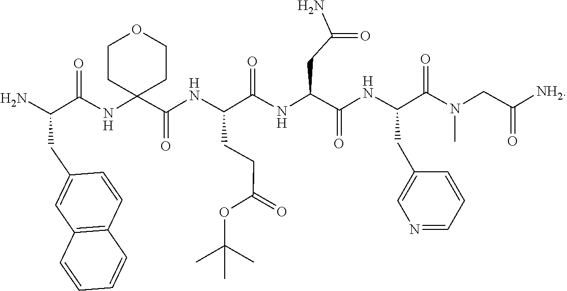 [SMILES: CN(CC(N)=O)C(=O)[C@H](Cc1cccnc1)NC(=O)[C@H](CC(N)=O)NC(=O)[C@H](CCC(=O)OC(C)(C)C)NC(=O)C1(NC(=O)[C@@H](N)Cc2ccc3ccccc3c2)CCOCC1]

In some embodiments, the linear chain peptide fragment is prepared by deprotecting a protected precursor to the linear chain peptide fragment. The protected precursor to the linear chain peptide fragment is a compound produced by reacting a compound of Formula (VII) with a compound of Formula (VIII), wherein R⁴ is NHP¹. In some embodiments, the protected precursor to the linear chain peptide fragment is a compound having the following structure:

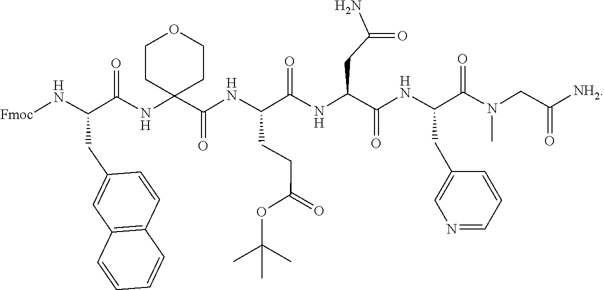

### Preparation of the Linear Chain Peptide Fragment

The process for preparing the monocyclic peptide may further comprise a step of preparing the linear chain peptide fragment. The preparation of the linear chain peptide fragment is a convergent synthesis which can be carried out in a solution phase without using a solid support.

In some embodiments, the linear chain peptide fragment is a compound of Formula (IV) which may be produced by reacting a compound of Formula (VII)

R⁶-X11a-X12a-R³ (VII)
with a compound of Formula (VIII)

R²-X13a-X14a-X15a-X16a-R⁴ (VIII)
to form an amide bond between X12a and X13a;
wherein
R² is H;
R³ is OH or OP²;
R⁴ is NH₂, or NHP¹;
R⁶ is H or P¹;
each P¹ is an amino protecting group; and
each P² is independently a carboxyl protecting group.

In instances where R³ is OP², the carboxyl protecting group (P²) can be removed in situ during the coupling.

In some embodiments, the amide bond is formed using a carbodiimide coupling reagent, optionally in the presence of an additive.

In some embodiments, the amide bond is formed in the presence of N-ethyl-N′-dimethylaminopropylcarbodiimide (EDCI) and 1-hydroxy-7-azabenzotriazole (HOAt).

In some embodiments, R⁴ is NH₂; and/or R⁶ is P¹ and P¹ is Fmoc.

In some embodiments, when R⁶ is P¹, reacting a compound of Formula (VII) with a compound of Formula (VIII) provides a protected precursor to the compound of Formula (IV). Deprotection of the terminal amino group to remove P¹ provides a compound of Formula (IV).

In some embodiments, the process of preparing the linear peptide fragment further comprises preparing a compound of Formula (VII), comprising reacting a compound of Formula R⁶-X11a-R³ with a compound of R²-X12a-R⁵ to form an amide bond between X11a and X12a, wherein R² is H, R³ is OH, R⁵ is OH or OP², R⁶ is P¹, P¹ is an amino protecting group and P² is a carboxyl protecting group.

In some embodiments of the compound of Formula (VII):
R³ is OH or OP²;
R⁶ is H or P¹;
P¹ is an amino protecting group; and
P² is a carboxyl protecting group;
X11a is 2Nal, unsubstituted Trp, or Trp substituted with cyano, halo, alkyl, haloalkyl, hydroxy, alkoxy, Phe(2-Me), Phe(3-Me), Phe(4-Me), Phe(3,4-dimethoxy), or 1Nal;
X12a is 4-amino-4-carboxy-tetrahydropyran (Gly(THP)), alpha-MeLys, alpha-MeLeu, alpha-MeArg, alpha-MePhe, alpha-MeLeu, alpha-MeLys, alpha-MeAsn, alpha-MeTyr, Ala, cyclohexylAla, Lys, or 2-aminoisobutyric acid.

In some embodiments, the amino, carboxyl, hydroxyl and thiol groups on the side chains of each of X11a-X12a are independently protected with a suitable protecting group.

In some embodiments, the compound of Formula (VII) is R⁶-2Nal-Gly(THP)-R³.

In some embodiments, the compound of Formula (VII) has the following structure:

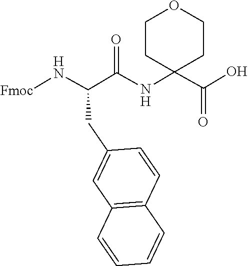

In some embodiments of the compound of Formula (VIII):
R² is H;
R⁵ is NH₂, or NHP¹;
P¹ is an amino protecting group;
X13a is 2-aminoisobutyric acid, Glu, Cit, Gln, Lys(Ac), alpha-MeArg, alpha-MeGlu, alpha-MeLeu, alpha-MeLys, alpha-Me-Asn, alpha-MeLys(Ac), Dab(Ac), Dap(Ac), homo-Lys(Ac), or Lys; or X13a is Lys, pegylated Lys, b-homoGlu, or Lys(Y2-Ac), wherein Y2 is an amino acid residue;
X14a is Asn, 2Nal, 2-aminoirobutyric acid, Arg, Cit, Asp, Phe, Gly, Lys, Leu, Ala, (D)Ala, beta-Ala, His, Thr, n-Leu, Gln, Ser, (D)Ser, Tic, Trp, alpha-MeGln, alpha-MeAsn, alpha-MeLys(Ac), Dab(Ac), Dap(Ac), homo-Lys(Ac), or Lys(Ac);
X15a is Ala, beta-Ala, Arg, Asn, Asp, Cit, Cys, Glu, Gln, Gly, substituted or unsubstituted His, (D)His, Ile, Lue, (D)Lue, Lys, (D)Lys, Met, 2Pal, 3Pal, or 4Pal, Phe, Pro, 5-Pyal, 2Quin, 3Quin, Ser, Thr, Trp, Tyr, Val, Leu;
X16a is absent or Sarc, aMeLeu, (D)NmeTyr, His, (D)Thr, bAla, Pro, or (D)Pro.

In some embodiments, the amino, carboxyl, hydroxyl and thiol groups on the side chains of each of X13a-X16a are independently protected with a suitable protecting group.

In some embodiments, the compound of Formula (VIII) is R²-Glu-Asn-3Pal-Sarc-R⁵.

In some embodiments, the compound of Formula (VIII) has the following structure:

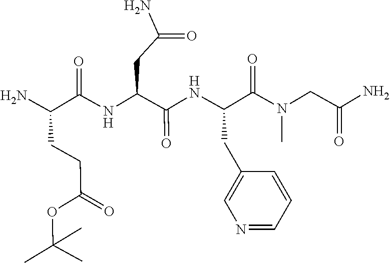 [SMILES: CN(CC(N)=O)C(=O)[C@H](Cc1cccnc1)NC(=O)[C@H](CC(N)=O)NC(=O)[C@@H](N)CCC(=O)OC(C)(C)C]

In some embodiments, the compound of Formula (VIII) is prepared by deprotecting a protected precursor to the compound of Formula (VIII). In some embodiments, the protected precursor to the compound of Formula (VIII) has the following structure:

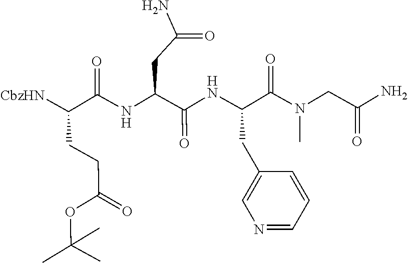 [SMILES: CN(CC(N)=O)C(=O)[C@H](Cc1cccnc1)NC(=O)[C@H](CC(N)=O)NC(=O)[C@H](CCC(=O)OC(C)(C)C)NC(=O)OCc1ccccc1]

### Preparation of the Compound of Formula (VIII)

The process for preparing the compound of Formula (IV) may further comprise a step of preparing the compound of Formula (VIII).

In some embodiments, the process of preparing the compound of Formula (VIII) comprises reacting a compound of Formula (XIII)

R²-X15a-X16a-R⁴ (XIII)
with a compound of Formula (XIV):

R⁶-X13a-X14a-R³ (XIV)
to form an amide bond between X14a and X15a; wherein
R² is H;
R³ is OH or OP²;
R⁴ is NH₂, or NHP¹;
R⁶ is H or P¹;
P¹ is an amino protecting group and P² is a carboxyl protecting group.

In instances where R³ is OP², the carboxyl protecting group (P²) can be removed in situ during the coupling.

In some embodiments, the amide bond is formed using a carbodiimide coupling reagent, optionally in the presence of an additive.

In some embodiments, the amide bond is formed in the presence of N-ethyl-N′-dimethylaminopropylcarbodiimide (EDCI), 1-hydroxy-7-azabenzotriazole (HOAt) and 1,4-diazabicyclo[2.2.2]octane (DABCO).

In some embodiments, R⁴ is NH₂; and/or R⁶ is P¹ and P¹ is Cbz.

In some embodiments, when R⁶ is P¹, reacting a compound of Formula (XIII) with a compound of Formula (XIV) provides a protected precursor to the compound of Formula (VIII). Deprotection of the terminal amino group to remove P¹ provides a compound of Formula (VIII).

In some embodiments, the compound of Formula (XIII) is prepared by reacting a compound of Formula R⁶-X15a-R³ with a compound of R²-X16a-R⁵ to form an amide bond between X15a and X16a. The amide bond may be formed in the presence of 1-[Bis(dimethylamino)methylene]-1H-1,2,3-triazolo[4,5-b]pyridinium 3-oxide hexafluorophosphate (HATU) and N-Methyl-2-pyrrolidone (NMP).

In some embodiments, the compound of Formula (XIV) is prepared by reacting a compound of Formula R⁶-X13a-R³ with a compound of R²-X14a-R⁵ to form an amide bond between X13a and X14a; wherein R² is H, R³ is OH, R⁵ is OH or OP², and R⁶ is H or P¹, wherein P¹ is an amino protecting group and P² is an carboxyl protecting group. In some embodiments, the amide bond is formed in the presence of pivaloyl chloride.

In some embodiments of the compound of Formula (XIII):
R² is H;
R⁴ is NHP¹, or NH₂;
P¹ is an amino protecting group;
X15a is Ala, beta-Ala, Arg, Asn, Asp, Cit, Cys, Glu, Gln, Gly, substituted or unsubstituted His, (D)His, Ile, Lue, (D)Lue, Lys, (D)Lys, Met, 2Pal, 3Pal, or 4Pal, Phe, Pro, 5Pyal, 2Quin, 3Quin, Ser, Thr, Trp, Tyr, Val; Leu;
X16a is absent or Sarc, aMeLeu, (D)NmeTyr, His, (D)Thr, bAla, Pro, or (D)Pro.

In some embodiments, the amino, carboxyl, hydroxyl and thiol groups on the side chains of each of X15a-X16a are independently protected with a suitable protecting group.

In some embodiments, the compound of Formula (XIII) is R²-3Pal-Sarc-R⁴.

In some embodiments, the compound of Formula (XIII) has the following structure:

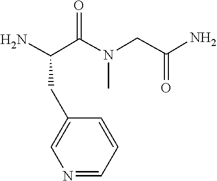 [SMILES: CN(CC(N)=O)C(=O)[C@@H](N)Cc1cccnc1]

In some embodiments of the compound of Formula (XIV):
R³ is OH or OP²;
R⁶ is H or P¹;
P¹ is an amino protecting group;
P² is a carboxyl protecting group;
X13a is 2-aminoisobutyric acid, Glu, Cit, Gln, Lys(Ac), alpha-MeArg, alpha-MeGlu, alpha-MeLeu, alpha-MeLys, alpha-Me-Asn, alpha-MeLys(Ac), Dab(Ac), Dap(Ac), homo-Lys(Ac), or Lys; or X13a is Lys, pegylated Lys, b-homoGlu, or Lys(Y2-Ac), wherein Y2 is an amino acid residue;
X14a is Asn, 2Nal, 2-aminoirobutyric acid, Arg, Cit, Asp, Phe, Gly, Lys, Leu, Ala, (D)Ala, beta-Ala, His, Thr, n-Leu, Gln, Ser, (D)Ser, Tic, Trp, alpha-MeGln, alpha-MeAsn, alpha-MeLys(Ac), Dab(Ac), Dap(Ac), homo-Lys(Ac), or Lys(Ac).

In some embodiments, the amino, carboxyl, hydroxyl and thiol groups on the side chains of each of X13a-X14a are independently protected with a suitable protecting group.

In some embodiments, the compound of Formula (XIV) is R⁶-Glu-Asn-R³.

In some embodiments, the compound of Formula (XIV) has the following structure:

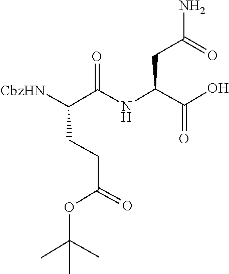 [SMILES: CC(C)(C)OC(=O)CC[C@H](NC(=O)OCc1ccccc1)C(=O)N[C@@H](CC(N)=O)C(=O)O]

Monocyclic Peptide Fragment

In some embodiments, the monocyclic peptide fragment is a peptide having a relative molecular mass (RMM) of between 500 and 3500.

In some embodiments, the monocyclic peptide fragment comprises a ring which is cyclized by a bond between side chains of two amino acid residues.

In some embodiments, the monocyclic peptide fragment comprises a ring which is cyclized via a disulfide-bridge or a thioether bond between side chains of two amino acid residues.

In some embodiments, the monocyclic peptide fragment comprises a ring containing 6 amino acid residues.

In some embodiments, the monocyclic peptide fragment is a peptide containing 7 amino acid residues.

In some embodiments, the monocyclic peptide fragment is a peptide containing 7 amino acid residues, wherein 6 of the amino acid residues form the ring.

In some embodiments, the monocyclic peptide fragment comprises a ring which is cyclized by a bond between the side chain of the amino acid residue at the N-terminus and the side chain of the amino acid residue which is adjacent to the amino acid residue at the C-terminus.

In some embodiments, the monocyclic peptide fragment is prepared by a step of cyclizing a second linear peptide fragment, wherein the second linear peptide fragment is a peptide containing between 4 and 11 amino acid residues. In some embodiments, the step of cyclizing the second linear peptide fragment comprises forming a bond between side chains of two amino acid residues. In some embodiments, the step of cyclizing the second linear peptide fragment comprises forming a bond between the side chain of the amino acid residue at the N-terminus and the side chain of the amino acid residue which is adjacent to the amino acid residue at the C-terminus. In some embodiments, the step of cyclizing the second linear peptide fragment comprises forming a disulfide-bridge or a thioether bond between side chains of two amino acid residues. In some embodiments, the disulfide-bridge is formed in the presence of an oxidising agent. In some embodiments, the disulfide-bridge is formed in the presence of formic acid. In some embodiments, the disulfide-bridge is formed in the presence of formic acid and diiodine. Alternative reagents for forming disulfide bonds include thallium (III) trifluroacetyate, or trans-[Pt(en)₂Cl₂]²⁺.

In some embodiments, the monocyclic peptide fragment is a peptide of Formula (III-A), (III-B) or (III-C):

R¹-X1a-X2a-X3a-X4a-X5a-X6a-X7a-X8a-X9a-R³ (III-A);

R¹-X1a-X2a-X3a-X4a-X5a-X6a-X7a-X8a-X9a-X10a-R³ (III-B);

R¹-X1a-X2a-X3a-X4a-X5a-X6a-X7a-X8a-X9a-X10a-X11a-R³ (III-C);
wherein each of X1a, X2a, and X3a is independently absent or is an amino acid residue;
each of X4a, X5a, X6a, X7a, X8a, X9a, X10a and X11a is an amino acid residue;
R¹ is H, or C₁₋₂₀ alkanoyl;
R³ is OH or OP²;
P² is a carboxyl protecting group.

In some embodiments, the peptide of each of Formulas (III-A), (III-B) or (III-C) is cyclized via a bond between X4a and X9a.

In some embodiments, the amino, carboxyl, hydroxyl and thiol groups on the side chains of each of X1a-X11a are independently protected with a suitable protecting group.

In some embodiments, the monocyclic peptide fragment comprises a ring which is appended by at least one peptide chain containing at least 1 amino acid residue. For example, the monocyclic peptide fragment can contain 7 amino acid residues, where 6 amino acids make up the ring and 1 amino acid residue is appended to the ring.

In some embodiments, the monocyclic peptide fragment is a compound of Formula (I):

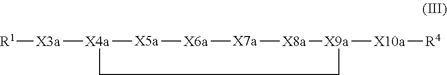

wherein
R¹ is H, or C₁₋₂₀ alkanoyl;
R³ is OH or OP²;
P² is a carboxyl protecting group;
X3a is absent or any amino acid residue;
X4a is Abu, Cys, (D)Cys, alpha-MeCys, (D)Pen, Pen, or Pen(sulfoxide);
X5a is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys;
X6a is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val;
X7a is unsubstituted Trp, or Trp substituted with cyano, halo, alkyl, substituted or unsubstituted aryl, haloalkyl, hydroxy, or alkoxy;
X8a is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1Nal, 2Nal, or Trp;
X9a is Abu, Cys, (D)Cys, alpha-MeCys, (D)Pen, Pen, or Pen(sulfoxide);
X10a is unsubstituted Phe, or Phe substituted with halo, alkyl, haloalkyl, hydroxy, alkoxy, carboxy, carboxamido, 2-aminoethoxy, or 2-acetylaminoethoxy; and the compound is cyclized via a Pen-Pen disulfide bond between X4a and X9a; or the compound is cyclized via a Abu-Cys or Abu-Pen thioether bond between X4a and X9a.

In some embodiments, the amino, carboxyl, hydroxyl and thiol groups on the side chains of each of X3a-X10a are independently protected with a suitable protecting group.

In some embodiments, R¹ is C(O)CH₃.

In some embodiments, the compound of Formula (III) is R¹-Pen-Asn-Thr-Trp(7-Me)-Lys(Ac)-Pen-Tyr(2ea)-R³.

In some embodiments, the compound of Formula (III) has the following structure:

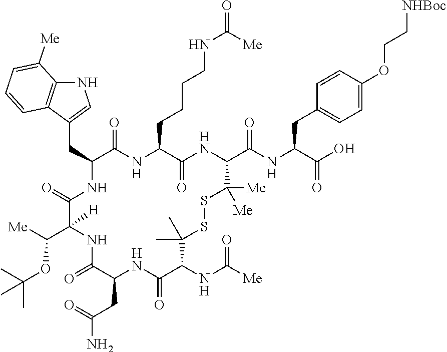 [SMILES: CC(=O)NCCCC[C@@H]1NC(=O)[C@H](Cc2c[nH]c3c(C)cccc23)NC(=O)[C@H]([C@@H](C)OC(C)(C)C)NC(=O)[C@H](CC(N)=O)NC(=O)[C@@H](NC(C)=O)C(C)(C)SSC(C)(C)[C@@H](C(=O)N[C@@H](Cc2ccc(OCCNC(=O)OC(C)(C)C)cc2)C(=O)O)NC1=O]

### Preparation of the Monocyclic Peptide Fragment

The process for preparing the monocyclic peptide may further comprise a step of preparing the monocyclic peptide fragment. The process for preparing the monocyclic peptide fragment may comprise a step of cyclizing a second linear peptide fragment to form the monocyclic peptide fragment, wherein the second linear peptide fragment is a peptide containing between 4 and 11 amino acid residues.

In some embodiments, the step of cyclizing the second linear peptide fragment comprises forming a bond between side chains of two amino acid residues.

In some embodiments, the step of cyclizing the second linear peptide fragment comprises forming a bond between the side chain of the amino acid residue at the N-terminus and the side chain of the amino acid residue which is adjacent to the amino acid residue at the C-terminus.

In some embodiments, the step of cyclizing the second linear peptide fragment comprises forming a disulfide-bridge or a thioether bond between side chains of two amino acid residues. The disulfide-bridge is formed using any suitable disulfide bond forming reaction. The a thioether bond is formed using any suitable thioether bond forming reaction.

In some embodiments, the disulfide bridge is formed in the presence of a oxidizing agent. In some embodiments, the disulfide bridge is formed in the presence of formic acid.

In some embodiments, the disulfide-bridge is formed in the presence of diiodide.

In some embodiments, the process for preparing the monocyclic peptide fragment comprises cyclizing a compound of Formula (III′):

R¹-X3a-X4a-X5a-X6a-X7a-X8a-X9a-X10a-R⁵ (III′)

to form a Pen-Pen disulfide bond between X4a and X9a,
R¹ is H, or C₁₋₂₀ alkanoyl;
R⁵ is OH, or OP²; and P² is a carboxyl protecting group.

In some embodiments, the amino, carboxyl, hydroxyl and thiol groups on the side chains of each of X3a-X10a are independently protected with a suitable protecting group.

In some embodiments, the thiol groups on X4a and X9a are deprotected prior to, or during, the cyclizing of the compound of Formula (III′).

In some embodiments, the Pen-Pen disulfide bond is formed in the presence of an oxidising agent. In some embodiments, the Pen-Pen disulfide bond is formed in the presence of formic acid. In some embodiments, when the thiol groups on X4a and X9a are protected with protecting groups independently selected from acetamidomethyl (Acm) and trityl (Tr), the Pen-Pen disulfide bond is formed in the presence of diiodine and formic acid.

In some embodiments, P² is CH₃ or C(CH₃)₃; and/or R¹ is C(O)CH₃; and/or R⁵ is OH.

In some embodiments, R⁵ is OP². In instances where R⁵ is OP² the carboxyl protecting group (P²) may be removed following the cyclization reaction.

Second Linear Peptide Fragment

The monocyclic peptide fragment comprises a ring which is cyclized by a bond between the side chains of two amino acid residues. The monocyclic peptide fragment is prepared by cyclizing a second linear peptide fragment. The second linear peptide fragment is a peptide of Formula (III′), Formula (III-A′), Formula (III-B′), or Formula (III-C′).

In some embodiments, the second linear peptide fragment is a peptide of Formula (III-A′):

R¹-X1a-X2a-X3a-X4a-X5a-X6a-X7a-X8a-X9a-R⁵ (III-A′);
wherein X1a-X9a are as described above for the monocyclic peptide fragment, and R¹ is H, or C₁₋₂₀ alkanoyl, and R⁵ is OH, or OP².

In some embodiments, the amino, carboxyl, hydroxyl and thiol groups on the side chains of each of X1a-X9a are independently protected with a suitable protecting group.

In instances where R⁵ is OP², the carboxyl protecting group (P²) may be removed following the cyclization reaction.

In some embodiments, the second linear peptide fragment is a peptide of Formula (III-B′):

R¹-X1a-X2a-X3a-X4a-X5a-X6a-X7a-X8a-X9a-X10a-R⁵ (III-B′);
wherein X1a-X10a are as described above for the monocyclic peptide fragment, and R¹ is H, or C₁₋₂₀ alkanoyl, and R⁵ is OH, or OP².

In some embodiments, the amino, carboxyl, hydroxyl and thiol groups on the side chains of each of X1a-X10a are independently protected with a suitable protecting group.

In instances where R⁵ is OP² the carboxyl protecting group (P²) may be removed following the cyclization reaction.

In some embodiments, the second linear peptide fragment is a peptide of Formula (III-C′):

R¹-X1a-X2a-X3a-X4a-X5a-X6a-X7a-X8a-X9a-X10a-X11a-R⁵ (III-C′);
wherein X1a-X10a are as described above for the monocyclic peptide fragment, R¹ is H, or C₁₋₂₀ alkanoyl, and R⁵ is OH, or OP².

In some embodiments, the amino, carboxyl, hydroxyl and thiol groups on the side chains of each of X1a-X11a are independently protected with a suitable protecting group.

In instances where R⁵ is OP² the carboxyl protecting group (P²) may be removed following the cyclization reaction.

In some embodiments R¹ is —C(O)CH₃.

In some embodiments, the second linear peptide fragment is a peptide of Formula (III′).

R¹-X3a-X4a-X5a-X6a-X7a-X8a-X9a-X10a-R⁵ (III′)
wherein
R¹ is H, or C₁₋₂₀ alkanoyl;
R⁵ is OH or OP²;
P² is a carboxyl protecting group;
X3a is absent or any amino acid residue;
X4a is Abu, Cys, (D)Cys, alpha-MeCys, (D)Pen, Pen, or Pen(sulfoxide);
X5a is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys;
X6a is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val;
X7a is unsubstituted Trp, or Trp substituted with cyano, halo, alkyl, substituted or unsubstituted aryl, haloalkyl, hydroxy, or alkoxy;
X8a is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1Nal, 2Nal, or Trp;
X9a is Abu, Cys, (D)Cys, alpha-MeCys, (D)Pen, Pen, or Pen(sulfoxide);
X10a is unsubstituted Phe, or Phe substituted with halo, alkyl, haloalkyl, hydroxy, alkoxy, carboxy, carboxamido, 2-aminoethoxy, or 2-acetylaminoethoxy.

In some embodiments, the amino, carboxyl, hydroxyl and thiol groups on the side chains of each of X3a-X10a are independently protected with a suitable protecting group.

In some embodiments in which the second linear peptide fragment is a peptide of Formula (III′), R¹ is C(O)CH₃ and R⁵ is OH.

In instances where R⁵ is OP² the carboxyl protecting group (P²) may be removed following the cyclization reaction.

In some embodiments, the second linear peptide fragment is R¹-Pen-Asn-Thr-Trp(7-Me)-Lys(Ac)-Pen-Tyr(2ea)-R⁵.

In some embodiments, the second linear peptide fragment is a compound having the following structure:

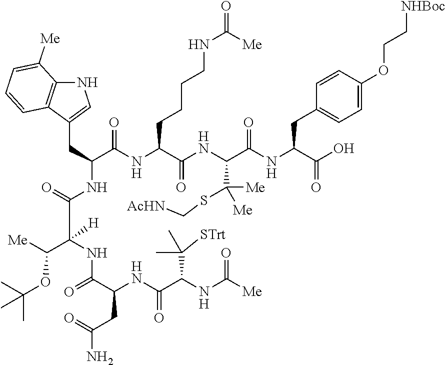

In some embodiments, the compound of Formula (III′) is prepared by deprotecting a protected precursor to the compound of Formula (III′). In some embodiments, the protected precursor to the compound of Formula (III′) has the following structure:

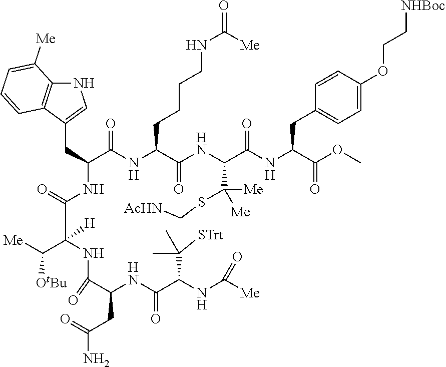

### Preparation of the Second Linear Peptide Fragment (Formula III′)

The second linear peptide fragment is prepared by coupling two smaller peptide fragments.

The process for preparing the monocyclic peptide fragment may further comprise a step of preparing the second linear peptide fragment.

In some embodiments, the second linear peptide fragment is a peptide of Formula (III′) and the step of preparing this compound comprises reacting a compound of Formula (V)

R¹-X3a-X4a-X5a-X6a-R³ (V)
with a compound of Formula (VI)

R²-X7a-X8a-X9a-X10a-R⁵ (VI)
to form an amide bond between X6a and X7a;
wherein
R¹ is H, or C₁₋₂₀ alkanoyl;
R² is H;
R³ is OH or OP²;
R⁵ is OH, or OP²; and each P² is independently a carboxyl protecting group.

In instances where R³ is OP², the carboxyl protecting group (P²) can be removed in situ during the coupling.

In some embodiments, the amide bond is formed using a carbodiimide coupling reagent, optionally in the presence of an additive. In some embodiments, the carbodiimide is 5-(hydroxyamino)-1,3-dimethylpyrimidine-2,4,6(1H,3H,5H)-trione (Oxyma B).

In the embodiments, the amide bond is formed in a solvent which is 2-methyl tetrahydrofuran (2-MeTHF).

In some embodiments, the amide bond is formed in the presence of N,N′-diisopropylcarbodiimide (DIC) and 5-(hydroxyamino)-1,3-dimethylpyrimidine-2,4,6(1H,3H,5H)-trione (Oxyma B).

In some embodiments, the amide bond is formed in the solution phase at a temperature of between 5 and 25° C.

In some embodiments, R¹ is C(O)CH₃; and/or R⁵ is OP²; and/or P² is a C₁₋₆ alkyl; and/or P² is CH₃.

In some embodiments, when R⁵ is OP², reacting a compound of Formula (V) with a compound of Formula (VI) provides a protected precursor to the compound of Formula (III′). Deprotection of the terminal carboxyl group to remove P² provides a compound of Formula (III′).

In some embodiments of the compound of Formula (V):
R¹ is H, or C₁₋₂₀ alkanoyl;
R³ is OH or OP²;
P² is a carboxyl protecting group;
X3a is absent or any amino acid residue;
X4a is Abu, Cys, (D)Cys, alpha-MeCys, (D)Pen, Pen, or Pen(sulfoxide);
X5a is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys;
X6a is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val.

In some embodiments, the amino, carboxyl, hydroxyl and thiol groups on the side chains of each of X3a-X6a are independently protected with a suitable protecting group.

In some embodiments of the compound of Formula (V), R¹ is C(O)CH₃.

In some embodiments, the compound of Formula (V) is R¹-Pen-Asn-Thr-R³.

In some embodiments, the compound of Formula (V) is a compound having the following structure:

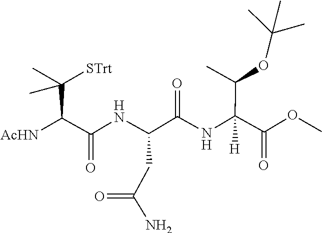

In some embodiments, the compound of Formula (V) is prepared by deprotecting a protected precursor to the compound of Formula (V). In some embodiments, the protected precursor to the compound of Formula (V) has the following structure:

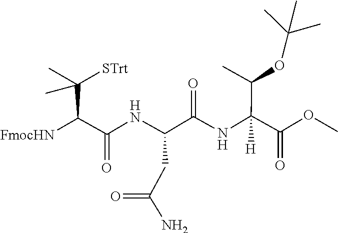

In some embodiments of the compound of Formula (VI):
R² is H;
R⁵ is NH₂, or NHP¹;
P¹ is an amino protecting group;
X7a is unsubstituted Trp, or Trp substituted with cyano, halo, alkyl, substituted or unsubstituted aryl, haloalkyl, hydroxy, or alkoxy;
X8a is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1Nal, 2Nal, or Trp;
X9a is Abu, Cys, (D)Cys, alpha-MeCys, (D)Pen, Pen, or Pen(sulfoxide);
X10a is unsubstituted Phe, or Phe substituted with halo, alkyl, haloalkyl, hydroxy, alkoxy, carboxy, carboxamido, 2-aminoethoxy, or 2-acetylaminoethoxy.

In some embodiments, the amino, carboxyl, hydroxyl and thiol groups on the side chains of each of X7a-X10a are independently protected with a suitable protecting group.

In some embodiments, the compound of Formula (VI) is R²-Trp(7-Me)-Lys(Ac)-Pen-Tyr(2ea)-R⁵.

In some embodiments, the compound of Formula (VI) is a compound having the following structure:

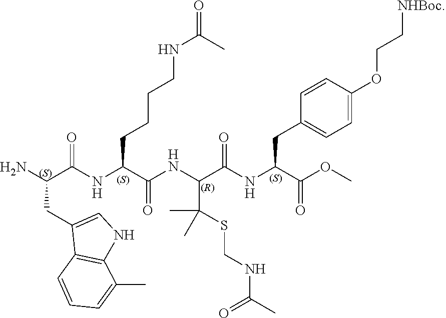

In some embodiments, the protected precursor to the compound of Formula (VI) is a compound having the following structure:

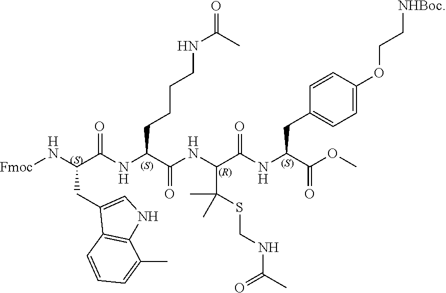

### Preparation of the Compound of Formula (V)

The process for preparing the monocyclic peptide fragment may comprise a step of preparing the second linear peptide fragment, which may further comprise a step of preparing the compound of Formula (V).

In some embodiments, the step of preparing a compound of Formula (III) further comprises converting a compound of Formula (V′):

R⁷-X3a-X4a-X5a-X6a-R⁵ (V′)
to a compound of Formula (V):

R¹-X3a-X4a-X5a-X6a-R³ (V)
wherein
R¹ is H, or C₁₋₂₀ alkanoyl;
R³ is OH or OP²
R⁵ is OH or OP²; and
R⁷ is H or P¹;
P¹ is an amino protecting group and each P² is independently a carboxyl protecting group.

In some embodiments, R⁷ is Fmoc, and/or R¹ is C(O)CH₃.

In some embodiments, converting a compound of Formula (V′) to a compound of Formula (V) comprises (i) deprotection of the terminal amino group of the compound of Formula (V′) to form a compound of having formula H-X3a-X4a-X5a-X6a-R³, followed by (ii) contacting the compound of Formula H-X3a-X4a-X5a-X6a-R³ with acetic anhydride to form the compound of Formula (V′).

In some embodiments, the process for preparing a compound of Formula (V) from Formula (V′) may further comprise steps for preparing a compound of Formula (V′).

In some embodiments of the compound of Formula (V′):
R⁵ is OH or OP²;
R⁷ is H or P¹;
P¹ is an amino protecting group;
P² is a carboxyl protecting group;
X3a is absent or any amino acid residue;
X4a is Abu, Cys, (D)Cys, alpha-MeCys, (D)Pen, Pen, or Pen(sulfoxide);
X5a is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys;
X6a is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val.

In some embodiments, the amino, carboxyl, hydroxyl and thiol groups on the side chains of each of X3a-X6a are independently protected with a suitable protecting group.

In some embodiments, the compound of Formula (V′) is R⁷—Pen-Asn-Thr-R⁵.

In some embodiments, the compound of Formula (V′) has the following structure:

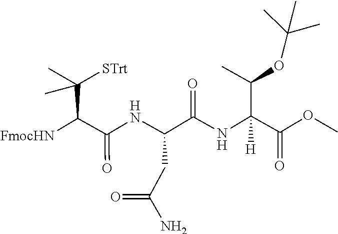

In some embodiments, the compound of Formula (V′) is prepared by reaction a compound of Formula (IX)

R⁶-X3a-X4a-R³ (IX)
with a compound of Formula (X)

R²-X5a-X6a-R⁵ (X)
to form an amide bond between X4a and X5a;
wherein R² is H; R³ is OH, or OP², R⁵ is OH, or OP²; R⁶ is H or P¹, P¹ is an amino protecting group and each P² is independently a carboxyl protecting group.

In instances where R³ is OP², the carboxyl protecting group (P²) can be removed in situ during the coupling.

In some embodiments, the compound of Formula (IX) is R⁶-X4a-R³, wherein R³ is OH and R⁶ is P¹ and P¹ is an amino protecting group.

In some embodiments, the amide bond is formed using an aminium coupling reagent.

In some embodiments, the amide bond is formed in the presence of O-(Benzotriazol-1-yl)-N,N,N′,N′-tetramethyluroniumtetrafluoroborate (TBTU) and di-isopropylethylamine (DIPEA).

In some embodiments, R⁵ is OP² and P² is C₁₋₆ alkyl; and/or R⁶ is P¹ and P¹ is Fmoc.

In some embodiments, when R⁶ is P¹, reacting a compound of Formula (IX) with a compound of Formula (X) provides a protected precursor to the compound of Formula (V). Deprotection of the terminal amino group to remove P¹ provides a compound having formula R⁷-X3a-X4a-X5a-X6a-R⁵, wherein R⁷ is H or P¹.

In some embodiments of the compound of Formula (IX):
R³ is OH or OP²;
R⁶ is H or P¹;
P¹ is an amino protecting group;
P² is a carboxyl protecting group;
X3a is absent or any amino acid residue;
X4a is Abu, Cys, (D)Cys, alpha-MeCys, (D)Pen, Pen, or Pen(sulfoxide).

In some embodiments, the amino, carboxyl, hydroxyl and thiol groups on the side chains of each of X3a-X4a are independently protected with a suitable protecting group.

In some embodiments, the compound of Formula (IX) is R⁶—Pen-R³.

In some embodiments, the compound of Formula (IX) has the following structure:

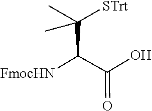

In some embodiments of the compound of Formula (X):
R² is H;
R⁵ is NH₂, or NHP¹;
P¹ is an amino protecting group;
X5a is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys;
X6a is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val.

In some embodiments, the amino, carboxyl, hydroxyl and thiol groups on the side chains of each of X5a-X6a are independently protected with a suitable protecting group.

In some embodiments, the compound of Formula (X) is R²—Asn-Thr-R⁵.

In some embodiments, the compound of Formula (X) has the following structure:

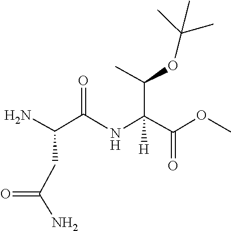 [SMILES: COC(=O)[C@@H](NC(=O)[C@@H](N)CC(N)=O)[C@@H](C)OC(C)(C)C]

In some embodiments, the compound of Formula (X) is prepared by deprotecting a protected precursor to the compound of Formula (X). In some embodiments, the protected precursor to the compound of Formula (X) has the following structure:

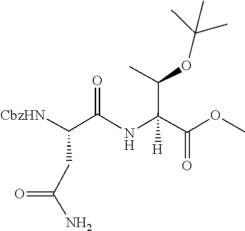 [SMILES: COC(=O)[C@@H](NC(=O)[C@H](CC(N)=O)NC(=O)OCc1ccccc1)[C@@H](C)OC(C)(C)C]

### Preparation of the Compound of Formula (X)

The process for preparing the compound of Formula (V) may further comprise a step of preparing the compound of Formula (X).

In some embodiments, the process of preparing the compound of Formula (X) comprises reacting a compound of Formula R⁶-X5a-R³ with a compound of Formula R²-X6a-R⁵ to form an amide bond between X5a and X6a, wherein R² is H, R³ is OH, R⁵ is OP², R⁶ is P¹, P¹ is an amino protecting group and P² is a carboxyl protecting group.

In some embodiments, the amide bond is formed using a carbodiimide coupling reagent, optionally in the presence of an additive.

In some embodiments, the amide bond is formed in the presence of N-ethyl-N′-dimethylaminopropylcarbodiimide (EDCI) and 1-hydroxy-7-azabenzotriazole (HOAt).

In some embodiments, R⁵ is OP² and P² is C₁₋₆ alkyl; and/or R⁶ is P¹ and P¹ is Cbz.

In some embodiments, when R⁶ is P¹, reacting a compound of Formula R⁶-X5a-R³ with a compound of Formula R²-X6a-R⁵ provides a protected precursor to the compound of Formula (X). Deprotection of the terminal amino group to remove P¹ provides a compound of Formula (X).

### Preparation of the Compound of Formula (VI)

The process for preparing the monocyclic peptide fragment may further comprise a step of preparing the compound of Formula (VI).

In some embodiments, the process of preparing the compound of Formula (VI) comprises reacting a compound of Formula (XI):

R²-X8a-X9a-X10a-R⁵ (XI)
with R⁶-X7a-R³ to form an amide bond between X7a and X8a; wherein
R² is H;
R³ is OH or OP²;
R⁵ is OH, or P²;
R⁶ is H or P¹; and
each P¹ is an amino protecting group; and
each P² is independently a carboxyl protecting group.

In instances where R³ is OP², the carboxyl protecting group (P²) can be removed in situ during the coupling.

In some embodiments, the amide bond is formed using a carbodiimide coupling reagent, optionally in the presence of an additive.

In some embodiments, the amide bond is formed in the presence of N-ethyl-N′-dimethylaminopropylcarbodiimide (EDCI) and ethyl cyanoglyoxylate-2-oxime (Oxyma Pure).

In some embodiments, R⁵ is P² and P² is C₁₋₆ alkyl; and/or R⁶ is P¹ and P¹ Fmoc.

In some embodiments, when R⁶ is P¹, reacting a compound of Formula (XI) with a compound of Formula R⁶-X7a-R³ provides a protected precursor to the compound of Formula (VI). Deprotection of the terminal amino group to remove P¹ provides a compound of Formula (VI).

In some embodiments of the compound of Formula (XI):
R² is H;
R⁵ is NH₂, or NHP¹;
P¹ is an amino protecting group;
X8a is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1Nal, 2Nal, or Trp;
X9a is Abu, Cys, (D)Cys, alpha-MeCys, (D)Pen, Pen, or Pen(sulfoxide);
X10a is unsubstituted Phe, or Phe substituted with halo, alkyl, haloalkyl, hydroxy, alkoxy, carboxy, carboxamido, 2-aminoethoxy, or 2-acetylaminoethoxy.

In some embodiments, the amino, carboxyl, hydroxyl and thiol groups on the side chains of each of X8a-X10a are independently protected with a suitable protecting group.

In some embodiments, the compound of Formula (XI) is R²-Lys(Ac)-Pen-Tyr(2ea)-R⁵.

In some embodiments, the compound of Formula (XI) has the following structure:

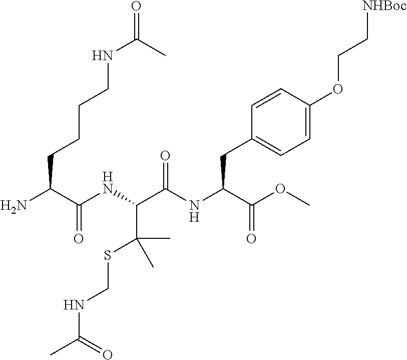 [SMILES: COC(=O)[C@H](Cc1ccc(OCCNC(=O)OC(C)(C)C)cc1)NC(=O)[C@@H](NC(=O)[C@@H](N)CCCCNC(C)=O)C(C)(C)SCNC(C)=O]

In some embodiments, the compound of Formula (XI) is prepared by deprotecting a protected precursor to the compound of Formula (XI). In some embodiments, the protected precursor to the compound of Formula (XI) has the following structure:

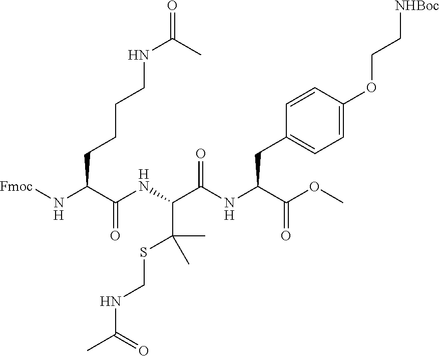

### Preparation of the Compound of Formula (XI)

The process for preparing the compound of Formula (VI) may further comprise a step of preparing the compound of Formula (XI).

In some embodiments, the process of preparing the compound of Formula (XI) comprises reacting a compound of Formula (XII):

R²-X9a-X10a-R⁵
with R⁶-X8a-R³ to form an amide bond between X8a and X9a; wherein.
R² is H;
R³ is OH or OP²;
R⁵ is OH, or P²;
R⁶ is H or P¹; and
each P¹ is an amino protecting group; and
each P² is independently a carboxyl protecting group.

In instances where R³ is OP², the carboxyl protecting group (P²) can be removed in situ during the coupling.

In some embodiments, the amide bond is formed using a carbodiimide coupling reagent, optionally in the presence of an additive.

In some embodiments, the amide bond is formed in the presence of N-ethyl-N′-dimethylaminopropylcarbodiimide (EDCI) and ethyl cyanohydroxyiminoacetate (Oxyma Pure).

In some embodiments, R⁵ is P² and P² is C₁₋₆ alkyl; and/or R⁶ is Fmoc.

In some embodiments, when R⁶ is P¹, reacting a compound of Formula (XII) with a compound of Formula R⁶-X8a-R³ provides a protected precursor to the compound of Formula (XI). Deprotection of the terminal amino group to remove P¹ provides a compound of Formula (XI).

In some embodiments, the process of preparing the compound of Formula (XI) further comprises a step of preparing the compound of Formula (XII), comprising reacting a compound of Formula R⁶-X9a-R³ with a compound of R²-X10a-R⁵ to form an amide bond between X9a and X10a, wherein R² is H, R³ is OH, R⁵ is OH or P², R⁶ is H or P¹, P¹ is an amino protecting group and P² is a carboxyl protecting group. The amide bond may be formed using a carbodiimide coupling reagent, optionally in the presence of an additive. The amide bond may be formed in the presence of N-ethyl-N′-dimethylaminopropylcarbodiimide (EDCI) and ethyl cyanohydroxyiminoacetate (Oxyma Pure). In some embodiments, R⁵ is P¹ and P¹ is C₁₋₆ alkyl; and/or R⁶ is Fmoc.

In some embodiments, when R⁶ is P¹, reacting a compound of Formula R⁶-X9a-R³ with a compound of R²-X10a-R⁵ provides a protected precursor to the compound of Formula (XII). Deprotection of the terminal amino group to remove P¹ provides a compound of Formula (XII).

In some embodiments of the compound of Formula (XII):
R² is H;
R⁵ is NH₂, or NHP¹;
P¹ is an amino protecting group;
X9a is Abu, Cys, (D)Cys, alpha-MeCys, (D)Pen, Pen, or Pen(sulfoxide);
X10a is unsubstituted Phe, or Phe substituted with halo, alkyl, haloalkyl, hydroxy, alkoxy, carboxy, carboxamido, 2-aminoethoxy, or 2-acetylaminoethoxy.

In some embodiments, the amino, carboxyl, hydroxyl and thiol groups on the side chains of each of X9a-X10a are independently protected with a suitable protecting group.

In some embodiments, the compound of Formula (XII) is R²—Pen-Tyr(2ea)-R⁵.

In some embodiments, the compound of Formula (XII) has the following structure:

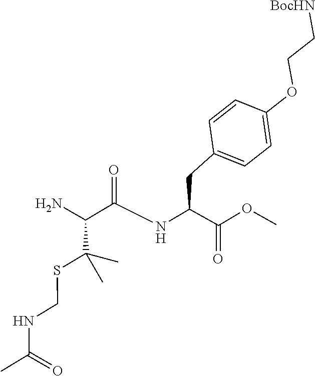 [SMILES: COC(=O)[C@H](Cc1ccc(OCCNC(=O)OC(C)(C)C)cc1)NC(=O)[C@@H](N)C(C)(C)SCNC(C)=O]

In some embodiments, the compound of Formula (XII) is prepared by deprotecting a protected precursor to the compound of Formula (XII). In some embodiments, the protected precursor to the compound of Formula (XII) has the following structure:

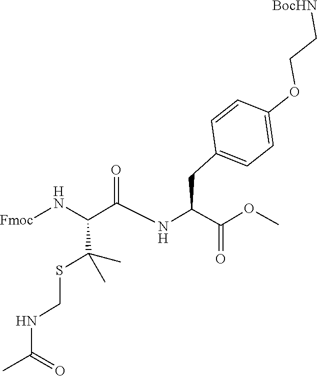

### Further Embodiments of the Invention

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

In the synthesis method described herein, the peptide elongation is carried out in solution and the synthesis schemes allow for the large-scale production of peptides, reduce the use of excess reagents and solvents, ease in purifying reaction intermediates, thus meeting the principles of green chemistry.

In one embodiment the invention relates to a compound of Formula (A-V), (A-VI), or (A-VII)

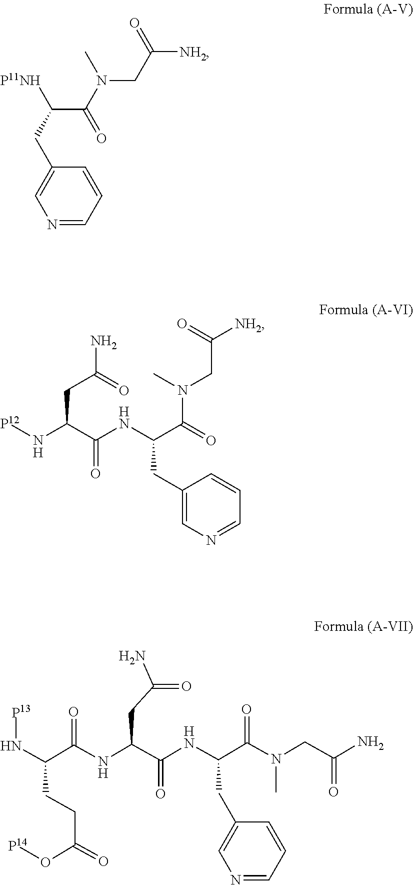

wherein:
P¹¹ is H, Fmoc, Cbz, or BOC;
P¹² is H, Fmoc, Cbz, or BOC;
P¹³ is H, Fmoc, Cbz, or BOC;
P¹⁴ is H, or C₍₁₋₄₎ alkyl;
   or a pharmaceutically acceptable salt thereof.

In one embodiment the invention relates to a compound of Formula (A-II), (A-III), or (A-IV)

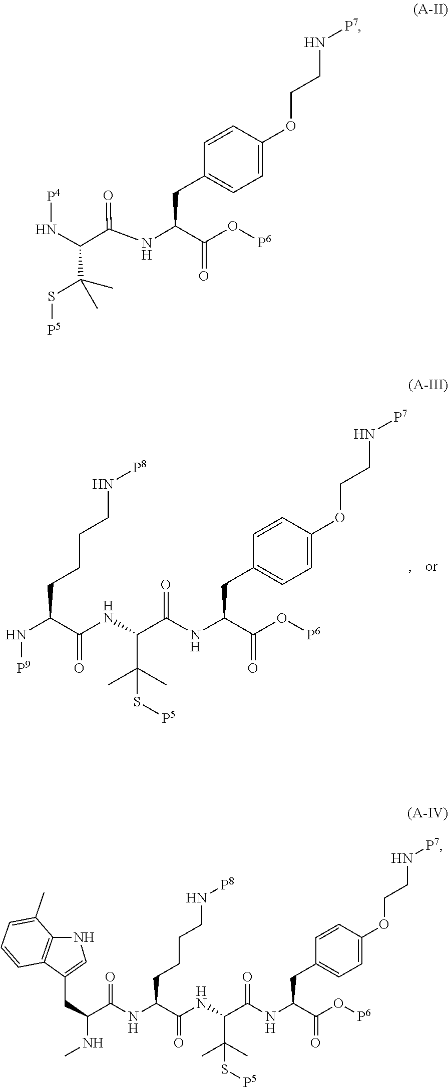

wherein:
P⁴ is H, Fmoc, Cbz, or BOC;
P⁵ is H, trityl, or Acm;
P⁶ is H, or C₍₁₋₄₎ alkyl;
P⁷ is H, Fmoc, Cbz, or BOC;
P⁸ is H or Ac;
P⁹ is H, Fmoc, Cbz, or BOC;
P¹⁰ is H, Fmoc, Cbz, or BOC;
   or a pharmaceutically acceptable salt thereof.

In one embodiment the invention relates to a compound of Formula (A-I)

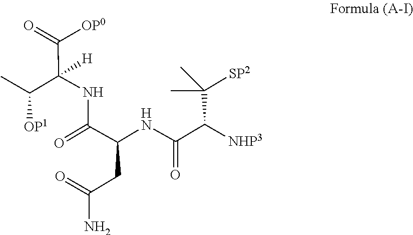

wherein:
P⁰ is H, or C₍₁₋₄₎ alkyl;
P¹ is H, tert-butyl, Bn, or Bz;
P² is H, trityl, or Acm;
P³ is H, Fmoc, Cbz, or BOC;
   or a pharmaceutically acceptable salt thereof.

In some embodiments, is the compound of formula (A-I) is

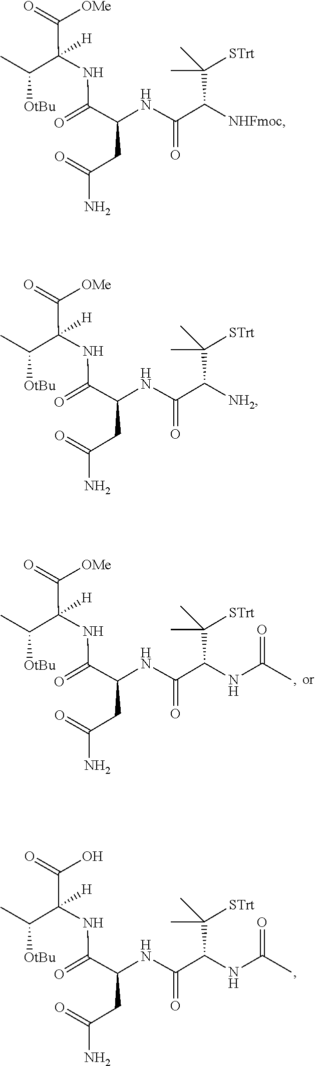

or a pharmaceutically acceptable salt thereof.

Some embodiments include a Formula (A-II)

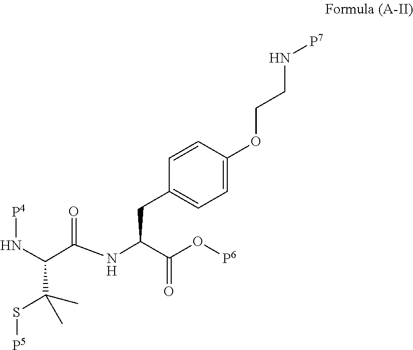

wherein:
P⁴ is H, Fmoc, Cbz, or BOC;
P⁵ is H, trityl, or Acm;
P⁶ is H, or C₍₁₋₄₎ alkyl;
P⁷ is H, Fmoc, Cbz, or BOC;
   or a pharmaceutically acceptable salt thereof.

In some embodiments, the compound of formula (A-II) is

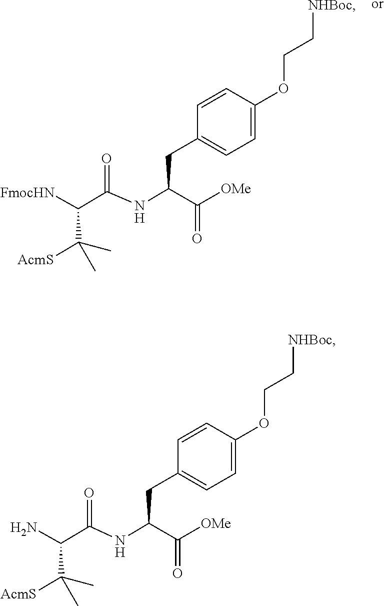

or a pharmaceutically acceptable salt thereof.

In one embodiment, the compound has a structure of Formula (A-III)

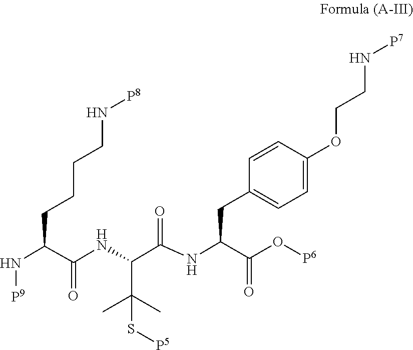

wherein:
P⁵ is H, trityl, or Acm;
P⁶ is H, or C₍₁₋₄₎ alkyl;
P⁷ is H, Fmoc, Cbz, or BOC;
P¹ is H or Ac;
P⁹ is H, Fmoc, Cbz, or BOC;
   or a pharmaceutically acceptable salt thereof.

In some embodiments, the compound of formula (A-III) is

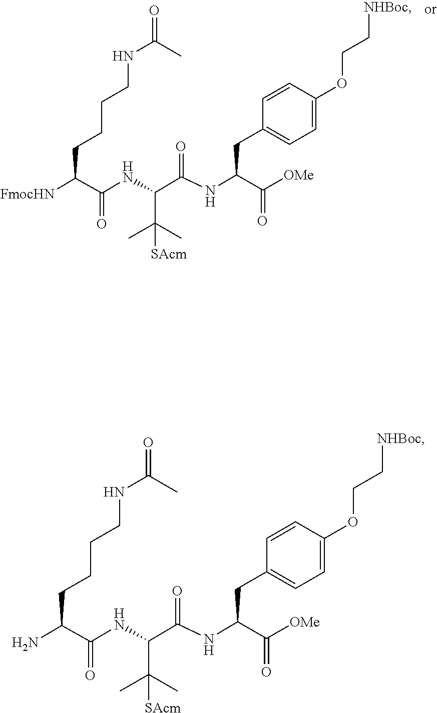

or a pharmaceutically acceptable salt thereof.

In one embodiment, the compound has a structure of Formula (A-IV)

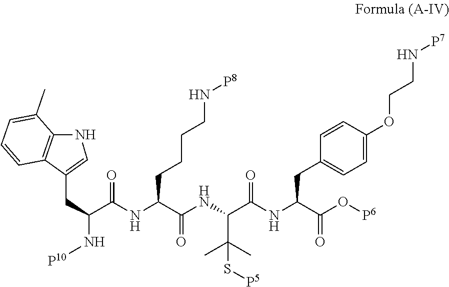

wherein:
P⁶ is H, or C₍₁₋₄₎ alkyl;
P⁷ is H, Fmoc, Cbz, or BOC;
P⁸ is H or Ac;
P¹⁰ is H, Fmoc, Cbz, or BOC;
   or a pharmaceutically acceptable salt thereof.

In one embodiment the compound of formula (A-IV) is

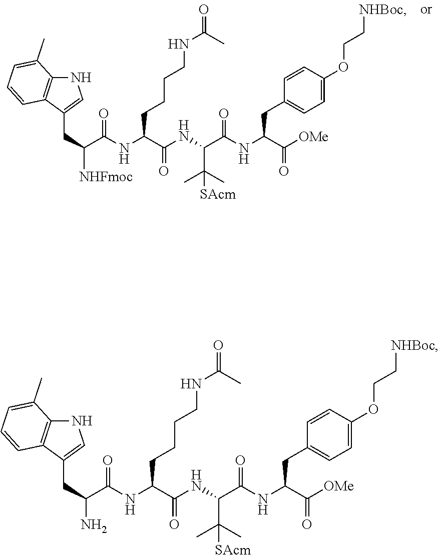

or a pharmaceutically acceptable salt thereof.

In one embodiment, the compound has a structure of Formula (A-V)

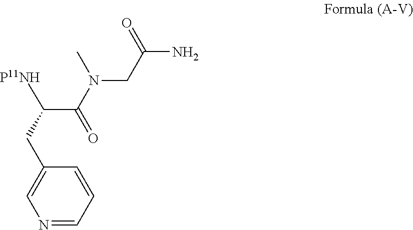

wherein:
P¹¹ is H, Fmoc, Cbz, or BOC;
   or a pharmaceutically acceptable salt thereof.

In some embodiments, the compound of formula (A-V) is

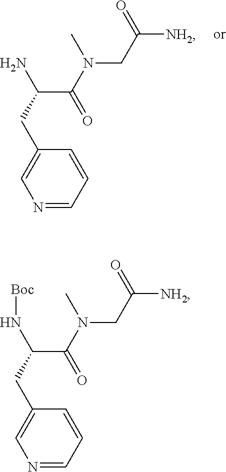 [SMILES: CN(CC(N)=O)C(=O)[C@@H](N)Cc1cccnc1 ; "or" ; CN(CC(N)=O)C(=O)[C@H](Cc1cccnc1)NC(=O)OC(C)(C)C]

or a pharmaceutically acceptable salt thereof.

In one embodiment, the compound has a structure of Formula (A-VI)

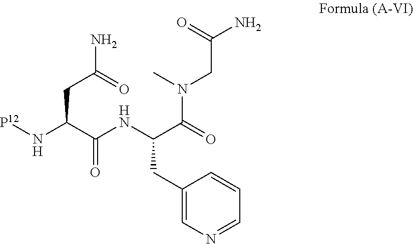

wherein:
P¹² is H, Fmoc, Cbz, or BOC;
   or a pharmaceutically acceptable salt thereof.

In some embodiments, the compound of formula (A-VI) is

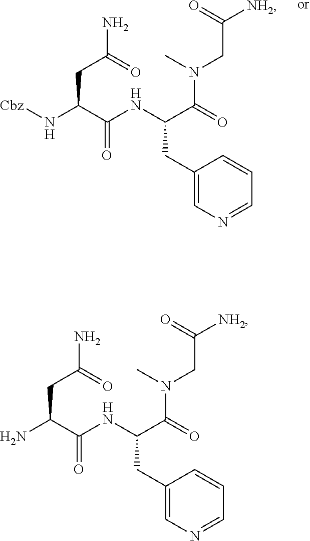 [SMILES: CN(CC(N)=O)C(=O)[C@H](Cc1cccnc1)NC(=O)[C@H](CC(N)=O)NC(=O)OCc1ccccc1 ; "or" ; CN(CC(N)=O)C(=O)[C@H](Cc1cccnc1)NC(=O)[C@@H](N)CC(N)=O]

or a pharmaceutically acceptable salt thereof.

In one embodiment the compound has a structure of Formula (A-VII)

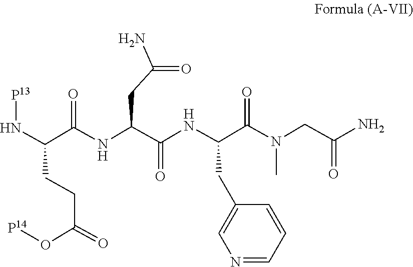

wherein:
P¹³ is H, Fmoc, Cbz, or BOC;
P¹⁴ is H, or C₍₁₋₄₎ alkyl;
   or a pharmaceutically acceptable salt thereof.

In some embodiments, the compound of formula (A-VII) is

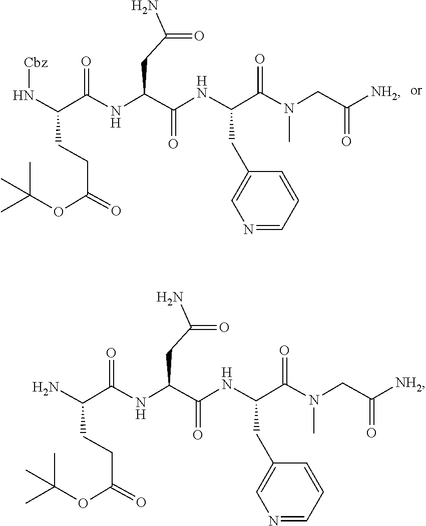 [SMILES: CN(CC(N)=O)C(=O)[C@H](Cc1cccnc1)NC(=O)[C@H](CC(N)=O)NC(=O)[C@H](CCC(=O)OC(C)(C)C)NC(=O)OCc1ccccc1 ; "or" ; CN(CC(N)=O)C(=O)[C@H](Cc1cccnc1)NC(=O)[C@H](CC(N)=O)NC(=O)[C@@H](N)CCC(=O)OC(C)(C)C]

or a pharmaceutically acceptable salt thereof.

In one embodiment the compound has the structure of Formula (A-VIII)

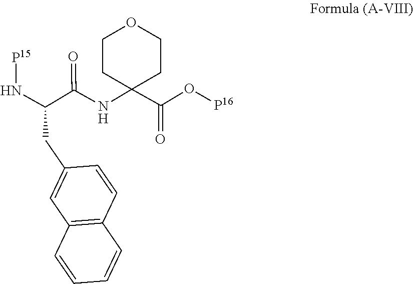

wherein:
P¹⁵ is H, Fmoc, Cbz, or BOC;
P¹⁶ is H, or C₍₁₋₄₎ alkyl;
   or a pharmaceutically acceptable salt thereof.

In some embodiments, the compound of formula (A-VIII) is

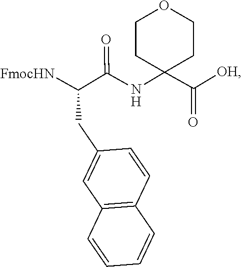

or a pharmaceutically acceptable salt thereof.

In one embodiment the compound has a structure of Formula (A-IX)

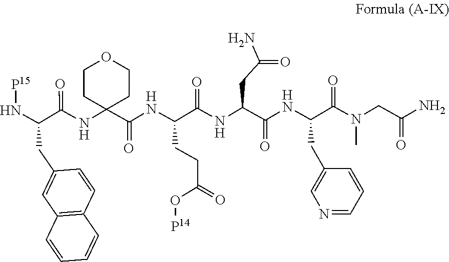

wherein:
P¹⁵ is H, Fmoc, Cbz, or BOC;
P¹⁴ is H, or C₍₁₋₄₎ alkyl;
   or a pharmaceutically acceptable salt thereof.

In some embodiments, the compound of formula (A-IX) is

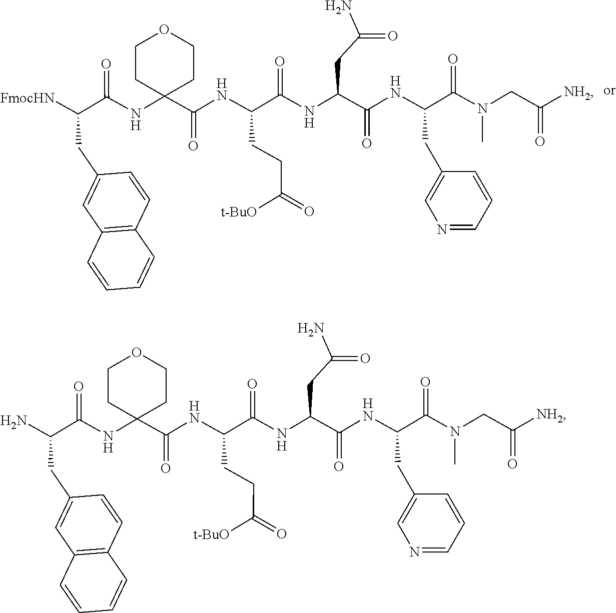

or a pharmaceutically acceptable salt thereof.

In one embodiment the compound has a structure of Formula (A-X)

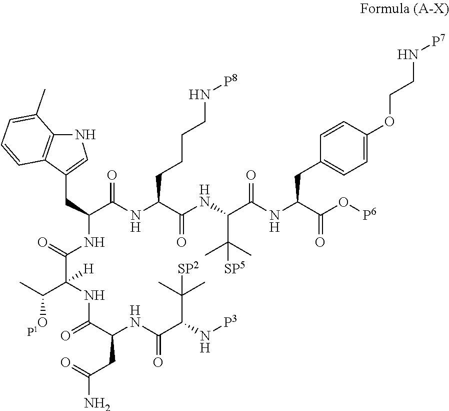

wherein:
P¹ is H, tert-butyl, Bn, or Bz;
P² is H, trityl, or Acm;
P³ is H, Fmoc, Cbz, or BOC;
P⁵ is H, trityl, or Acm;
P⁶ is H, or C₍₁₋₄₎ alkyl;
P⁷ is H, Fmoc, Cbz, or BOC;
P⁸ is H or Ac;
   or a pharmaceutically acceptable salt thereof.

In some embodiments, the compound of formula (A-X) is

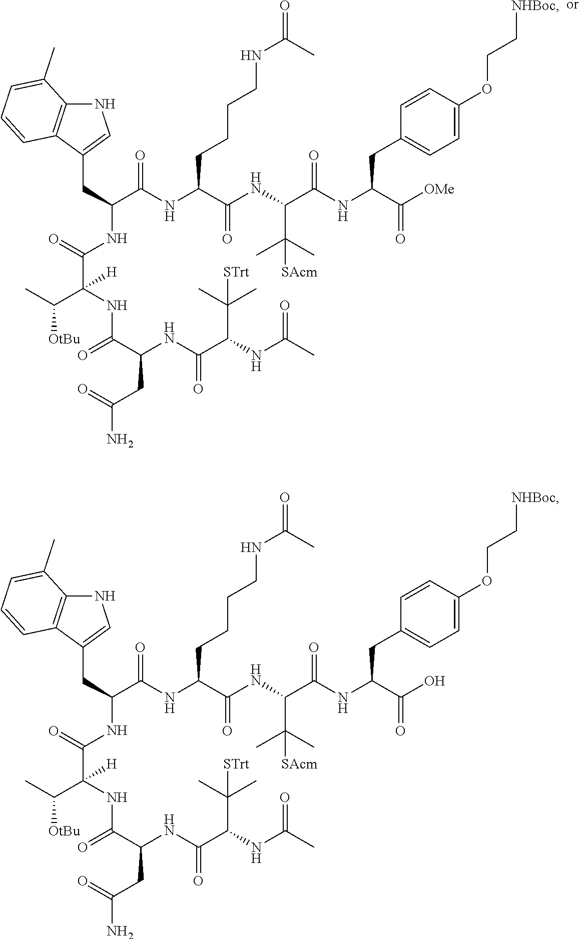

or a pharmaceutically acceptable salt thereof.

In one embodiment the compound has a structure of Formula (A-XI)

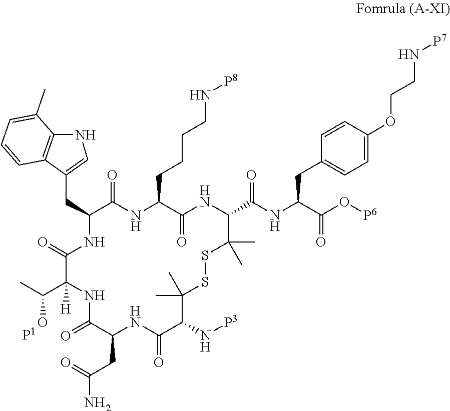

wherein:
P¹ is H, tert-butyl, Bn, or Bz;
P³ is H, Fmoc, Cbz, or BOC;
P⁶ is H, or C₍₁₋₄₎ alkyl;
P⁷ is H, Fmoc, Cbz, or BOC;
P¹ is H or Ac;
   or a pharmaceutically acceptable salt thereof.

In some embodiments, the compound of formula (A-XI) is

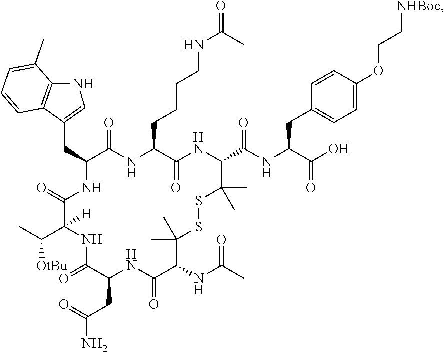 [SMILES: CC(=O)NCCCC[C@@H]1NC(=O)[C@H](Cc2c[nH]c3c(C)cccc23)NC(=O)[C@H]([C@@H](C)OC(C)(C)C)NC(=O)[C@H](CC(N)=O)NC(=O)[C@@H](NC(C)=O)C(C)(C)SSC(C)(C)[C@@H](C(=O)N[C@@H](Cc2ccc(OCCNC(=O)OC(C)(C)C)cc2)C(=O)O)NC1=O]

or a pharmaceutically acceptable salt thereof.

Some embodiments relate to a process of preparing compound of compound 26, wherein Ac-[1-7]—OH-cyclic is reacted with H-[8-13]-NH2:

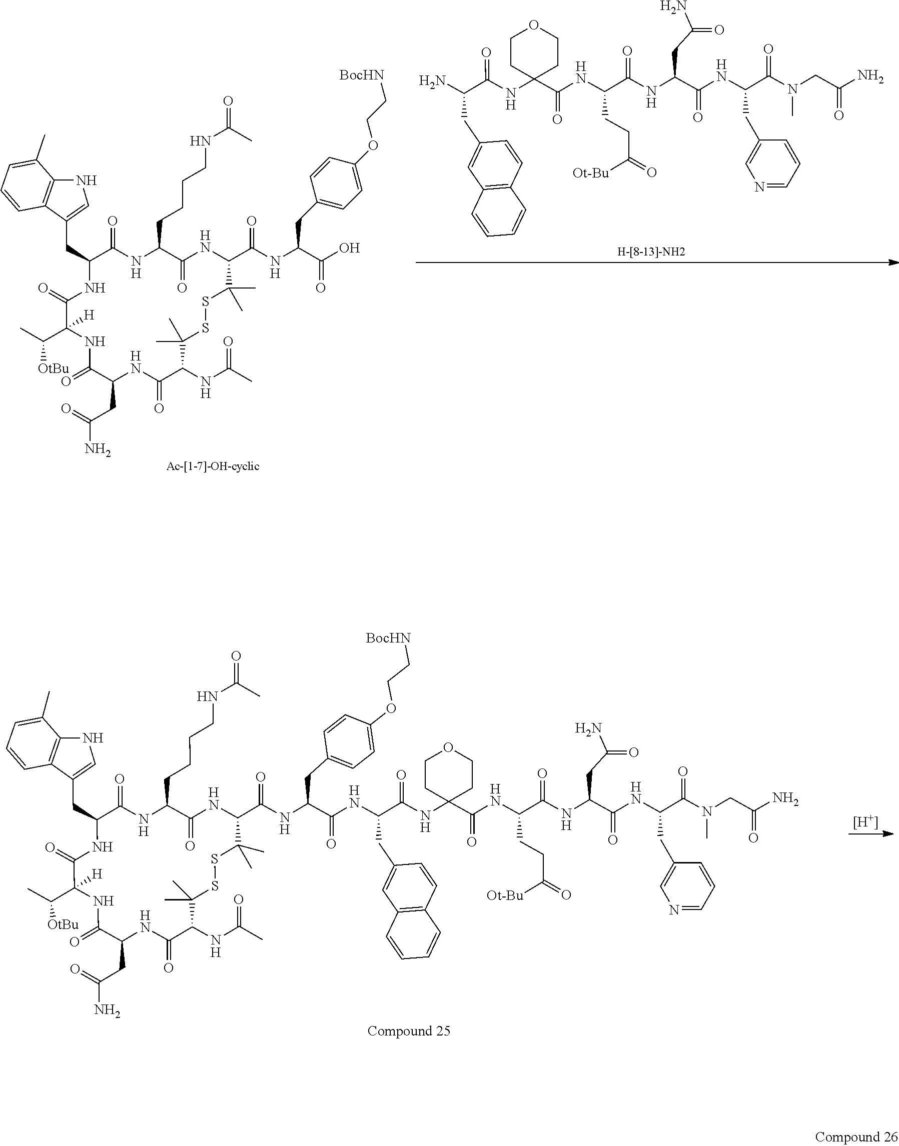

in the presence of diisopropylcarbodiimide and Oxyma-B to yield compound 25 followed by acid mediated removal of the tert-butyl groups from threonine and glutamic acid and the butyloxycarbonyl group from aminoethoxy phenyl alanine to form compound 26.

In some embodiments, H[8-13]NH2 is formed by reaction of Fmoc-2-Nal-THPGly-OH with H-Glu(OtBu)-Asn-3-Pal-Sar-NH2.

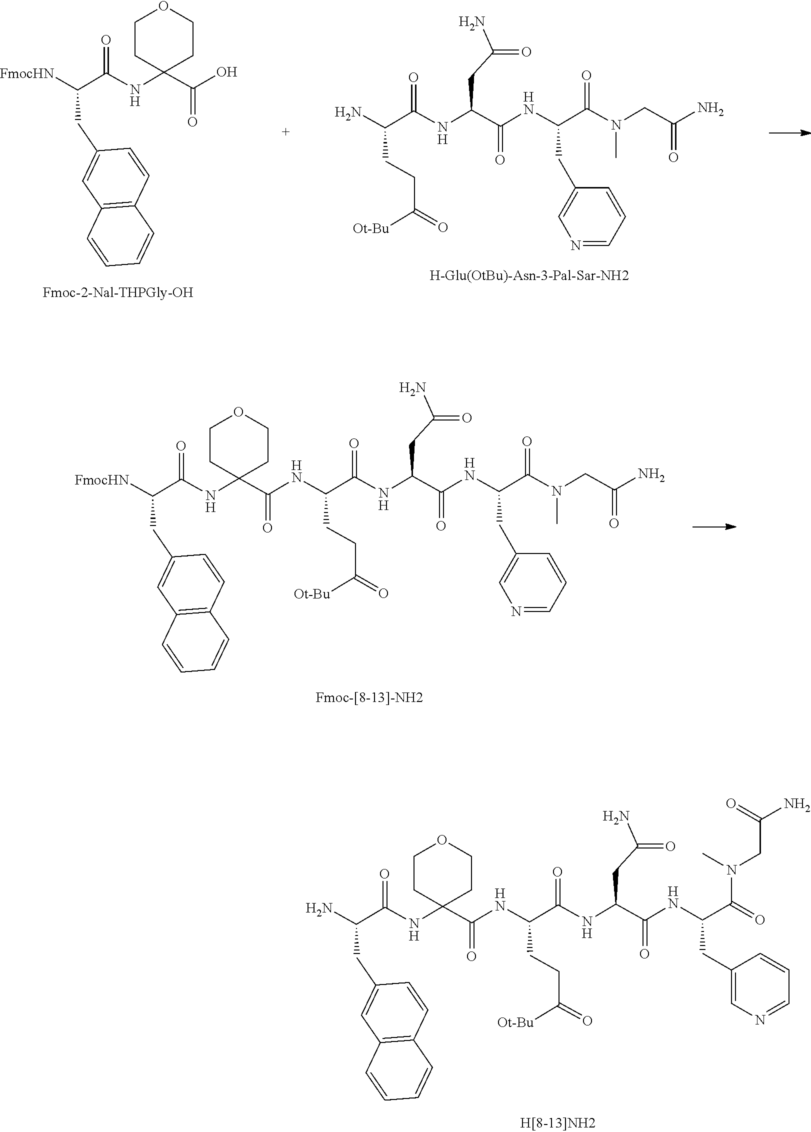

in the presence of (7-azabenzotriazol-1-yloxy)tripyrrolidinophosphonium hexafluorophosphate and diisopropylethylamine, to form Fmoc[8-13]NH2, followed by reaction with 1,8-diazabicyclo[5.4.0]undec-7-ene.

In some embodiments, Fmoc-2-Nal-THPGly-OH is formed by reaction of H-THPGly-OH with Fmoc-2-Nal-OH

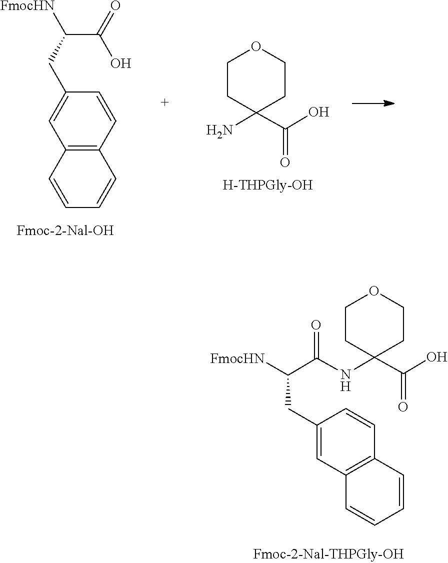

in the presence of N,O-bis(trimethylsilyl)acetamide.

In some embodiments, wherein H-Glu(OtBu)-Asn-3-Pal-Sar-NH2 if formed by reaction of Z-Glu(OtBu)-OH reaction with N-hydroxysuccinimide and diisopropylcarbodiimide, followed by H-Asn-3-Pal-Sar-NH2 to form Cbz-Glu(OtBu)-Asn-3-Pal-Sar-NH2, followed by catalytic hydrogenation.

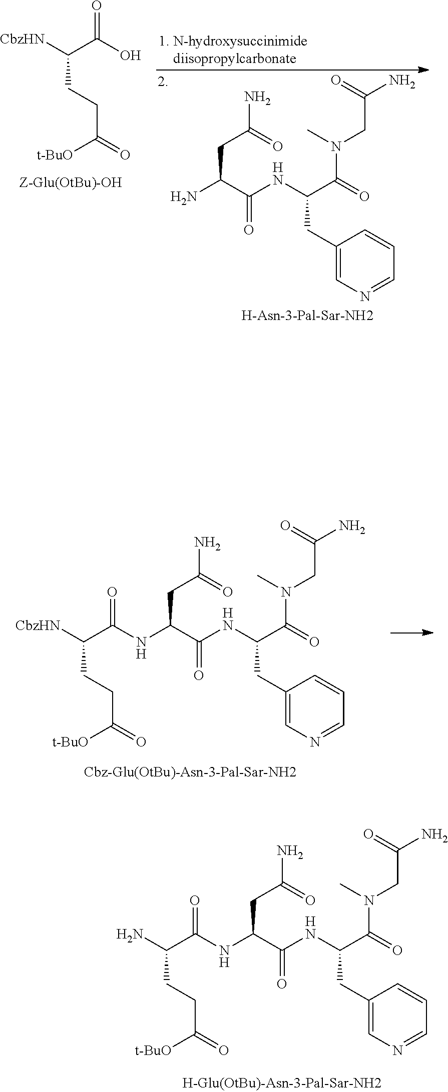 [SMILES: CC(C)(C)OC(=O)CC[C@H](NC(=O)OCc1ccccc1)C(=O)O ; CN(CC(N)=O)C(=O)[C@H](Cc1cccnc1)NC(=O)[C@@H](N)CC(N)=O ; CN(CC(N)=O)C(=O)[C@H](Cc1cccnc1)NC(=O)[C@H](CC(N)=O)NC(=O)[C@H](CCC(=O)OC(C)(C)C)NC(=O)OCc1ccccc1 ; CN(CC(N)=O)C(=O)[C@H](Cc1cccnc1)NC(=O)[C@H](CC(N)=O)NC(=O)[C@@H](N)CCC(=O)OC(C)(C)C]

In some embodiments, H-Asn-3-Pal-Sar-NH2 is formed by reaction of Z-Asn-OH with N-hydroxysuccinimide and diisopropylcarbodiimide, followed by H-3-Pal-Sar-NH2 to form Cbz-[11-13]-NH2, followed by catalytic hydrogenation

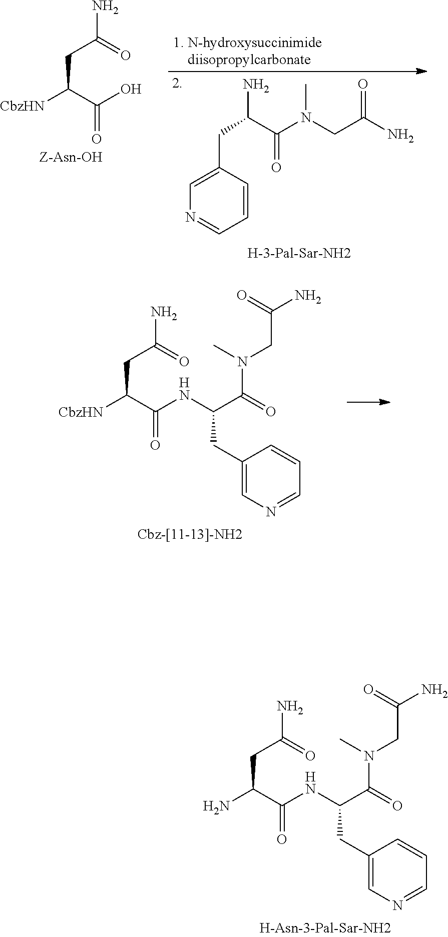 [SMILES: NC(=O)C[C@H](NC(=O)OCc1ccccc1)C(=O)O ; CN(CC(N)=O)C(=O)[C@@H](N)Cc1cccnc1 ; CN(CC(N)=O)C(=O)[C@H](Cc1cccnc1)NC(=O)[C@H](CC(N)=O)NC(=O)OCc1ccccc1 ; CN(CC(N)=O)C(=O)[C@H](Cc1cccnc1)NC(=O)[C@@H](N)CC(N)=O]

In some embodiments, H-3-Pal-Sar-NH2 is formed by reaction of Boc-3-Pal-OH with pivaloyl chloride in the presence of pyridine and N-methyl morpholine, followed by reaction with H-Sar-NH2 to form Boc-3-Pal-Sar-NH2, followed by acid mediated removal the butyloxycarbonyl group.

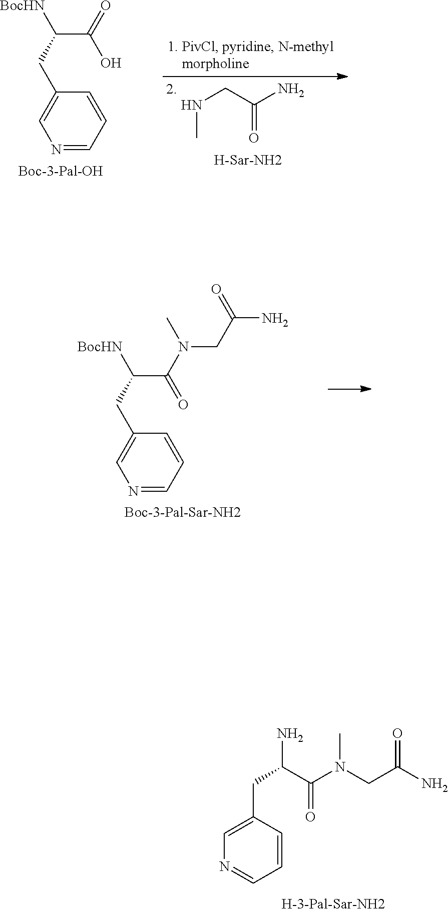

Some embodiments relate to a process of preparing Ac-[1-7]-OMe wherein Ac-Pen(Trt)-Asn-Thr(tBu)-OH is reacted with H-7Me-Trp-Lys(Ac)-Pen(Acm)-Tyr(2-Boc-ea)-OMe in the presence of diisopropylcarbodiimide and Oxyma B, followed by reaction with iodine in the presence of potassium iodide.

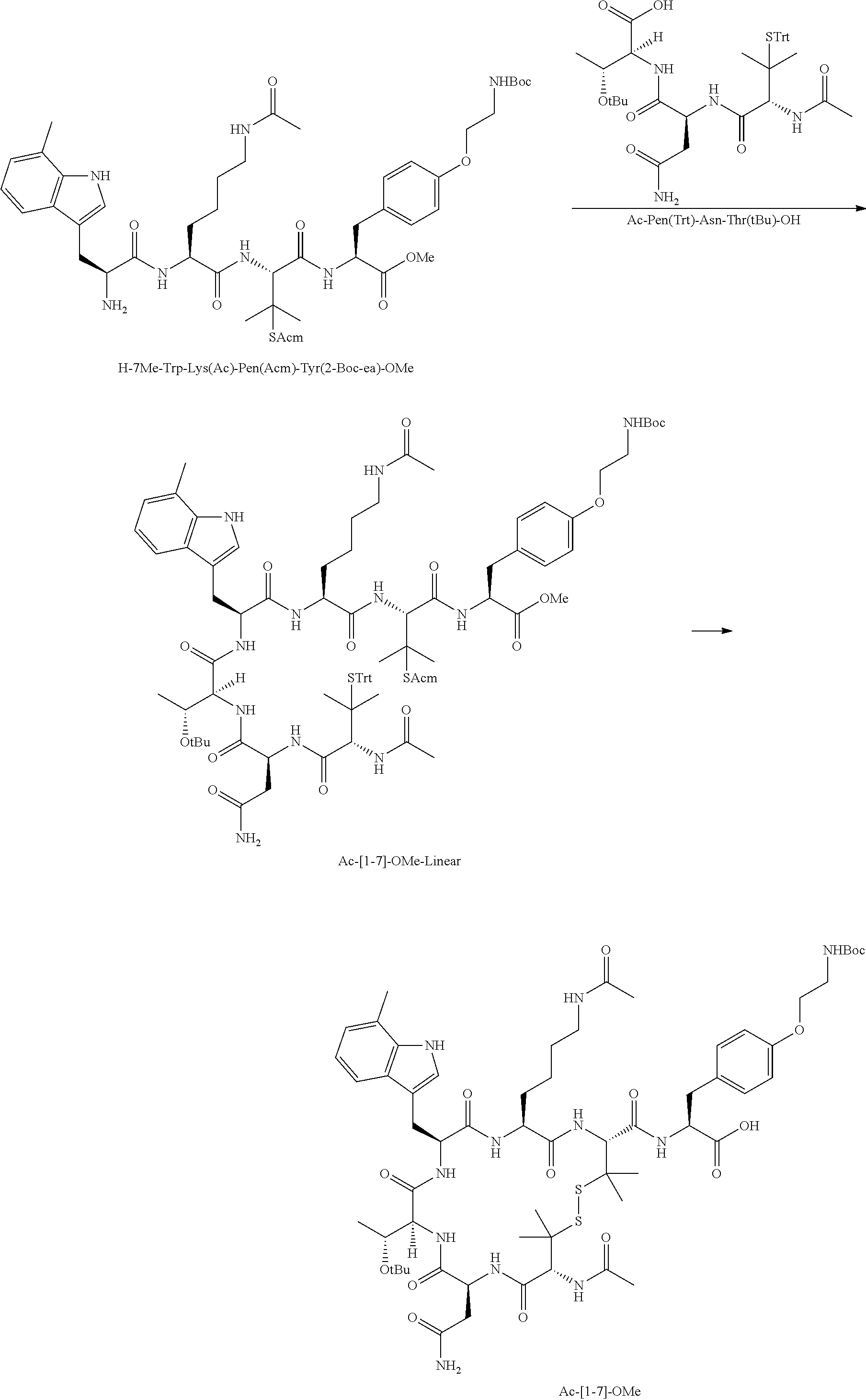

In some embodiments, H-7Me-Trp-Lys(Ac)-Pen(Acm)-Tyr(2-Boc-ea)-OMe is formed by reaction of H-Lys(Ac)-Pen(Acm)-Tyr(2-Boc-ea)-OMe with Fmoc-Trp(7Me)-OH in the presence of N,N,N′N′-tetramethyl-O-(benzotriazole-1-yl)uronium tetrafluoroborate and diisopropylethylamine to form Fmoc-7Me-Trp-Lys(Ac)-Pen(Acm)-Tyr(2-Boc-ea)-OMe followed by reaction with 1,8 diazabicyclo[5,4,0]undec-7-ene.

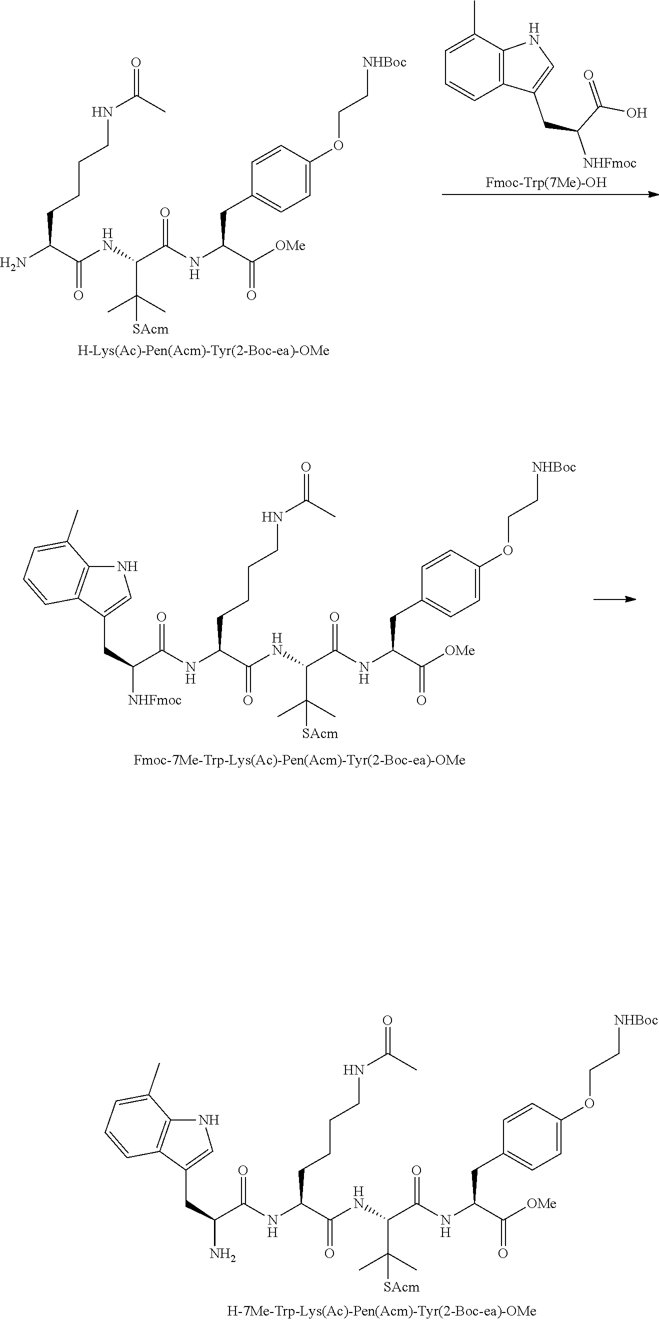

In some embodiments, H-Lys(Ac)-Pen(Acm)-Tyr(2-Boc-ea)-OMe is formed by reaction of H-Pen(Acm)-Tyr(2-Boc-ea)-OMe with Fmoc-Lys(Ac)—OH in the presence of N,N,N′N′-tetramethyl-O-(benzotriazole-1-yl)uronium tetrafluoroborate and diisopropylethylamine to form Fmoc-Lys(Ac)-Pen(Acm)-Tyr(2-Boc-ea)-OMe followed by reaction with 1,8 diazabicyclo[5,4,0]undec-7-ene.

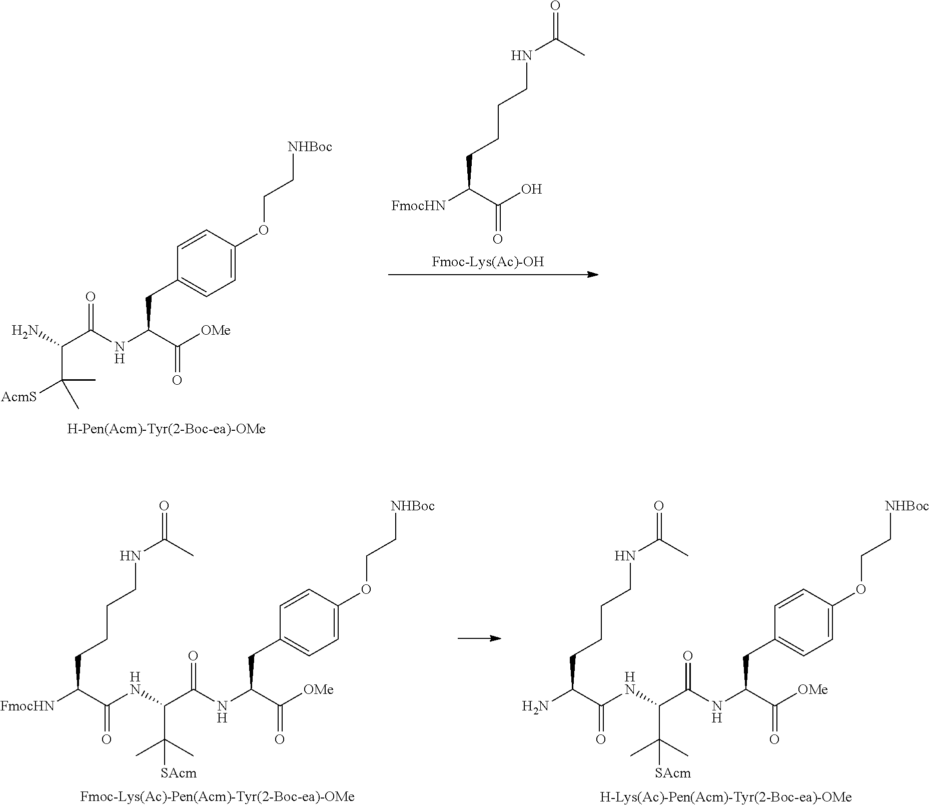

In some embodiments, H-Pen(Acm)-Tyr(2-Boc-ea)-OMe is formed by reaction of H-Tyr(2-Boc-ea)-OMe with Fmoc-Pen(Acm)-OH in the presence of Oxyma Pure and diisopropylcarbodiimide to form Fmoc-Pen(Acm)-Tyr(2-Boc-ea)-OMe followed by reaction with 1,8 diazabicyclo[5,4,0]undec-7-ene.

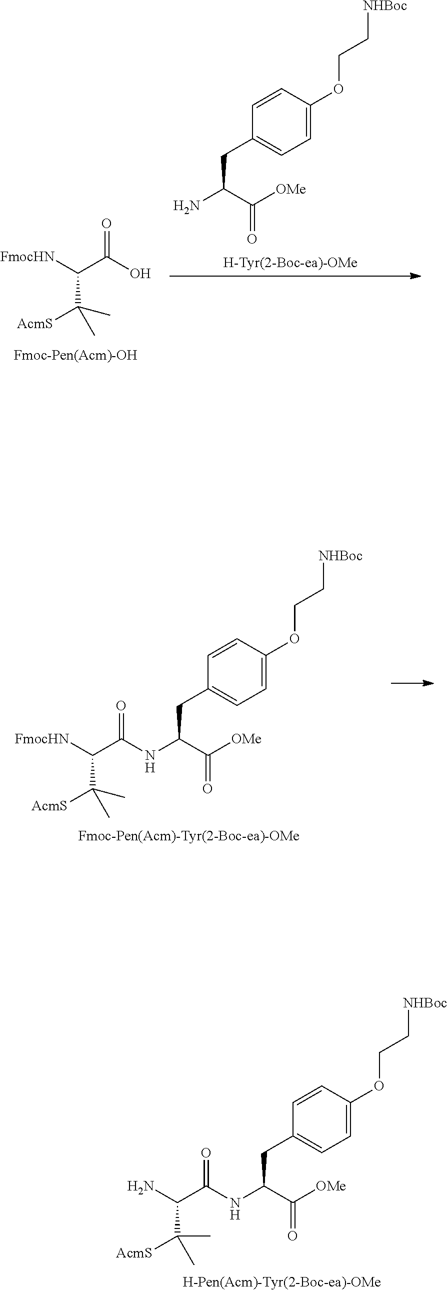

In some embodiments, Ac-Pen(Trt)-Asn-Thr(tBu)-OH is formed by reaction of H-Asn-Thr(tBu)-OMe with Fmoc-Pen(Trt)-OH in the presence of N,N,N′N′-tetramethyl-O-(benzotriazole-1-yl)uronium tetrafluoroborate and diisopropylethylamine to form Fmoc-Pen(Trt)-Asn-Thr(tBu)-OMe followed by reaction with 1,8 diazabicyclo[5,4,0]undec-7-ene to form H-Pen(Trt)-Asn-Thr(tBu)-OMe followed by reaction with acetic anhydride to form Ac-Pen(Trt)-Asn-Thr(tBu)-OMe, followed by reaction with lithium hydroxide.

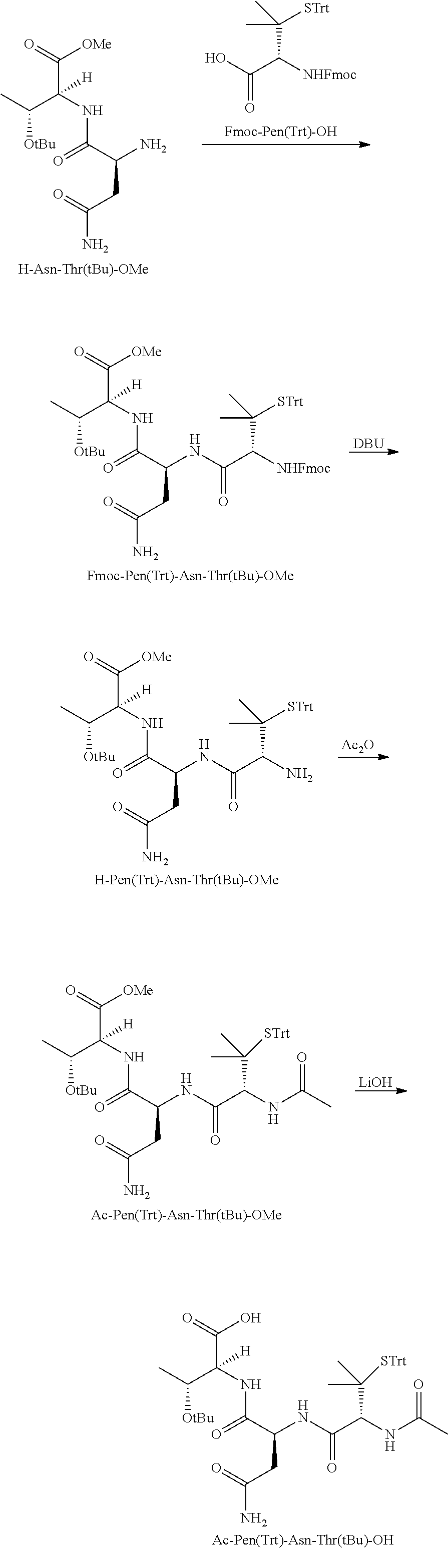

Some embodiments relate to a process of preparing (A-XII), by reaction of (A-XI) with (A-IX)

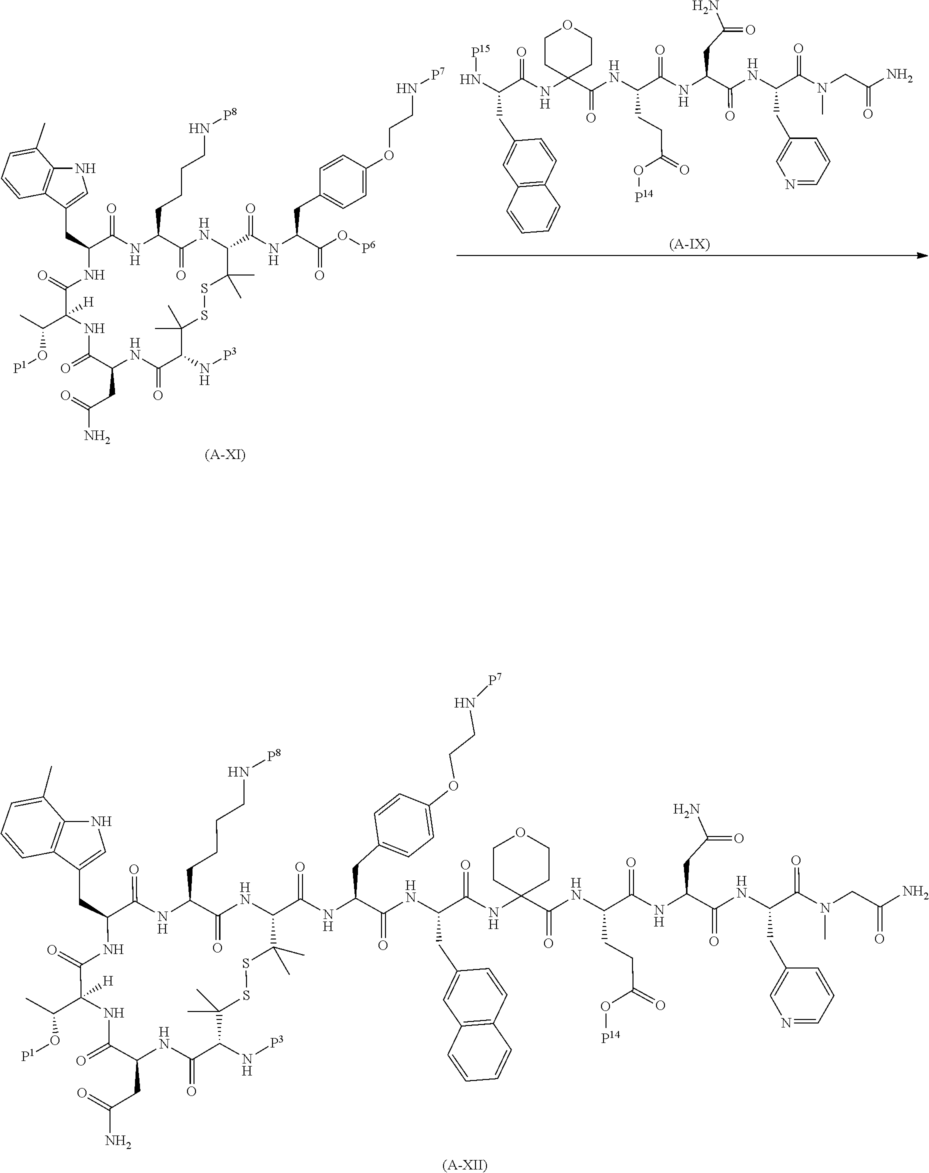

wherein:
P¹ is H, tert-butyl, Bn, or Bz;
P³ is H, Fmoc, Cbz, or BOC;
P⁶ is H;
P⁷ is H, Fmoc, Cbz, or BOC;
P⁸ is H or Ac;
P¹⁴ is H, or C₍₁₋₄₎ alkyl;
P¹⁵ is H;
   or a pharmaceutically acceptable salt thereof.

Some embodiments relate to a process of preparing (A-IX) by reaction of (A-VII) with (A-VIII) in the presence of reagents selected from
Oxyma B and diisopropylcarbodiimide,
Oxyma Pure and diisopropylcarbodiimide,
(7-azabenzotriazol-1-yloxy)tripyrrolidinophosphonium hexafluorophosphate and diisopropylethylamine,
pivaloyl chloride, pyridine and N-methyl morpholine, with or without N, O bis(trimethylsilyl)acetamide,
N,N,N′N′-tetramethyl-O-(benzotriazole-1-yl)uronium tetrafluoroborate and diisopropylethylamine,
N-hydroxysuccinimide and diisopropylcarbodiimide, or
Oxyma Pure and N-ethyl-N′-(3-dimethylaminopropyl)carbodiimide

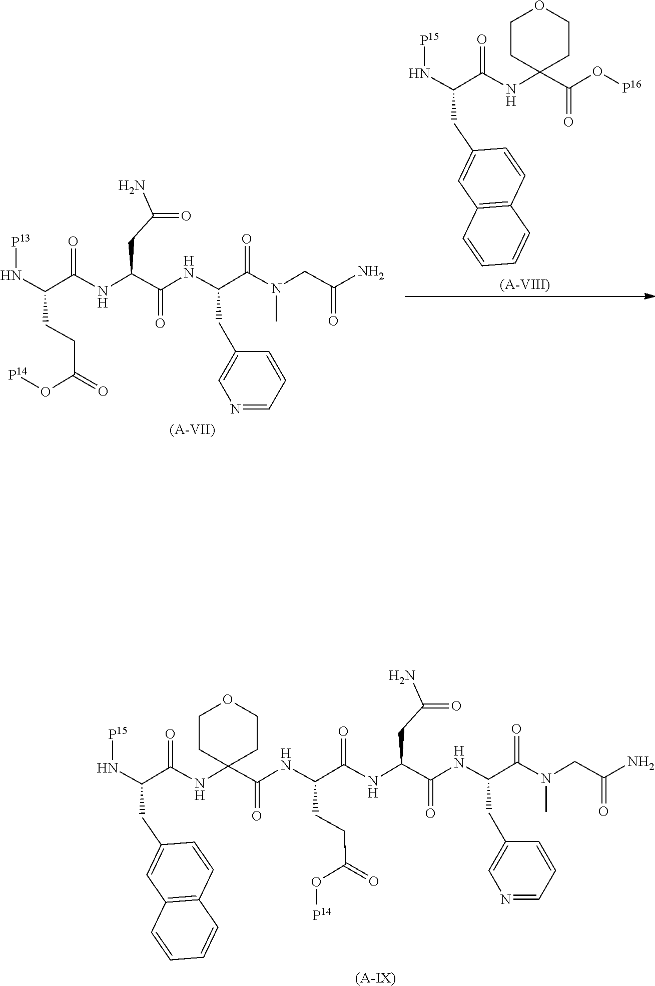

wherein:
P¹³ is H;
P¹⁴ is H, or C₍₁₋₄₎alkyl;
P¹⁵ is H, Fmoc, Cbz, or BOC;
P¹⁶ is H;
or a pharmaceutically acceptable salt thereof.

Some embodiments relate to a process of preparing (A-VIII) by reaction of (A-VIII-A) with (A-VIII-B) in the presence of reagents selected from
Oxyma B and diisopropylcarbodiimide,
Oxyma Pure and diisopropylcarbodiimide,
(7-azabenzotriazol-1-yloxy)tripyrrolidinophosphonium hexafluorophosphate and diisopropylethylamine,
pivaloyl chloride, pyridine and N-methyl morpholine, with or without N, O bis(trimethylsilyl)acetamide,
N,N,N′N′-tetramethyl-O-(benzotriazole-1-yl)uronium tetrafluoroborate and diisopropylethylamine,
N-hydroxysuccinimide and diisopropylcarbodiimide, or
Oxyma Pure and N-ethyl-N′-(3-dimethylaminopropyl)carbodiimide

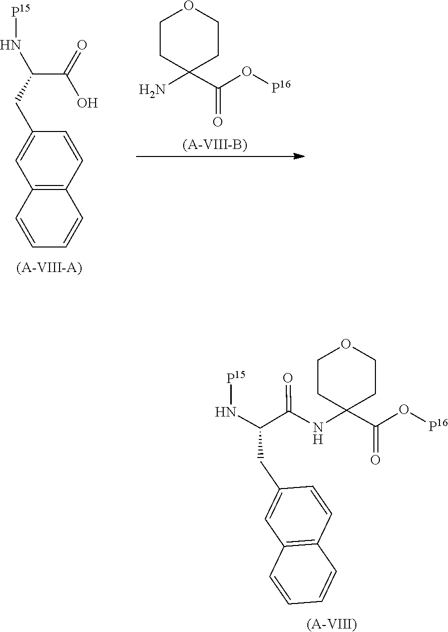

wherein:
P¹⁵ is H, Fmoc, Cbz, or BOC;
P¹⁶ is H, or C₍₁₋₄₎ alkyl;
   or a pharmaceutically acceptable salt thereof.

Some embodiments relate to a process of preparing (A-VII) by reaction of (A-VI) with (A-VII-A) in the presence of reagents selected from
Oxyma B and diisopropylcarbodiimide,
Oxyma Pure and diisopropylcarbodiimide,
(7-azabenzotriazol-1-yloxy)tripyrrolidinophosphonium hexafluorophosphate and diisopropylethylamine,
pivaloyl chloride, pyridine and N-methyl morpholine, with or without N, O bis(trimethylsilyl)acetamide,
N,N,N′N′-tetramethyl-O-(benzotriazole-1-yl)uronium tetrafluoroborate and diisopropylethylamine,
N-hydroxysuccinimide and diisopropylcarbodiimide, or
Oxyma Pure and N-ethyl-N′-(3-dimethylaminopropyl)carbodiimide

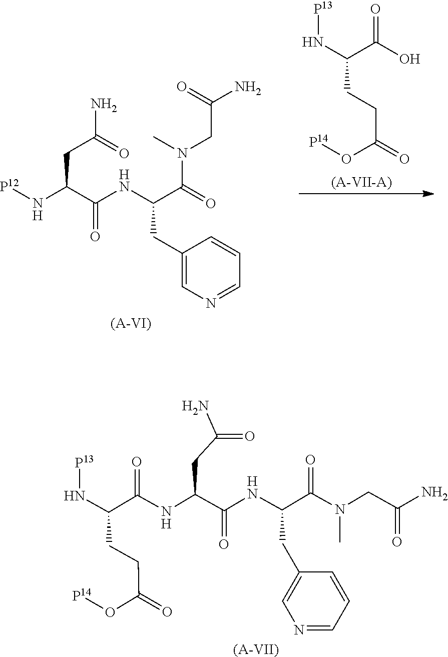

wherein:
P¹² is H;
P¹³ is H, Fmoc, Cbz, or BOC;
P¹⁴ is H, or C₍₁₋₄₎ alkyl;
   or a pharmaceutically acceptable salt thereof.

Some embodiments relate to a process of preparing (A-VI) by reaction of (A-V) with (A-VI-A) in the presence of reagents selected from
Oxyma B and diisopropylcarbodiimide,
Oxyma Pure and diisopropylcarbodiimide,
(7-azabenzotriazol-1-yloxy)tripyrrolidinophosphonium hexafluorophosphate and diisopropylethylamine,
pivaloyl chloride, pyridine and N-methyl morpholine, with or without N, O bis(trimethylsilyl)acetamide,
N,N,N′N′-tetramethyl-O-(benzotriazole-1-yl)uronium tetrafluoroborate and diisopropylethylamine,
N-hydroxysuccinimide and diisopropylcarbodiimide, or
Oxyma Pure and N-ethyl-N′-(3-dimethylaminopropyl)carbodiimide

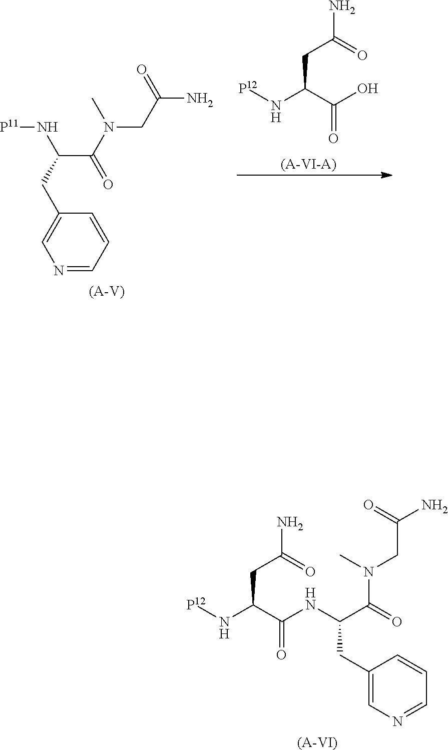

wherein:
P¹¹ is H;
P¹² is H, Fmoc, Cbz, or BOC;
   or a pharmaceutically acceptable salt thereof.

Some embodiments relate to a process of preparing (A-V) by reaction of H-Sar-NH2 with (A-V-A) in the presence of reagents selected from
Oxyma B and diisopropylcarbodiimide,
Oxyma Pure and diisopropylcarbodiimide,
(7-azabenzotriazol-1-yloxy)tripyrrolidinophosphonium hexafluorophosphate and diisopropylethylamine,
pivaloyl chloride, pyridine and N-methyl morpholine, with or without N, O bis(trimethylsilyl)acetamide,
N,N,N′N′-tetramethyl-O-(benzotriazole-1-yl)uronium tetrafluoroborate and diisopropylethylamine,
N-hydroxysuccinimide and diisopropylcarbodiimide, or
Oxyma Pure and N-ethyl-N′-(3-dimethylaminopropyl)carbodiimide

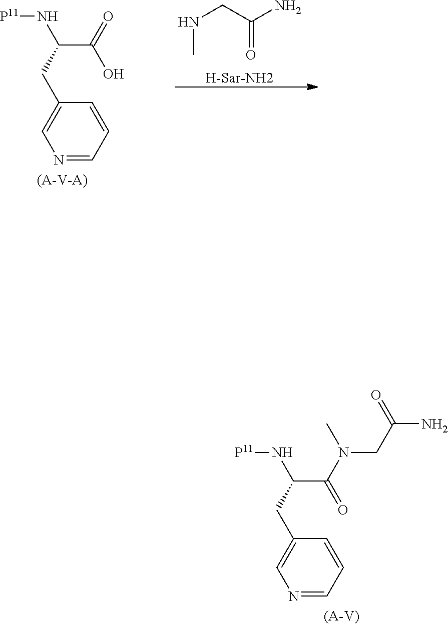

wherein:
P¹¹ is H, Fmoc, Cbz, or BOC;
   or a pharmaceutically acceptable salt thereof.

Some embodiments relate to a process of preparing (A-X) by reaction of (A-I) with (A-IV) in the presence of reagents selected from
Oxyma B and diisopropylcarbodiimide,
Oxyma Pure and diisopropylcarbodiimide,
(7-azabenzotriazol-1-yloxy)tripyrrolidinophosphonium hexafluorophosphate and diisopropylethylamine,
pivaloyl chloride, pyridine and N-methyl morpholine, with or without N, O bis(trimethylsilyl)acetamide,
N,N,N′N′-tetramethyl-O-(benzotriazole-1-yl)uronium tetrafluoroborate and diisopropylethylamine,
N-hydroxysuccinimide and diisopropylcarbodiimide, or
Oxyma Pure and N-ethyl-N′-(3-dimethylaminopropyl)carbodiimide

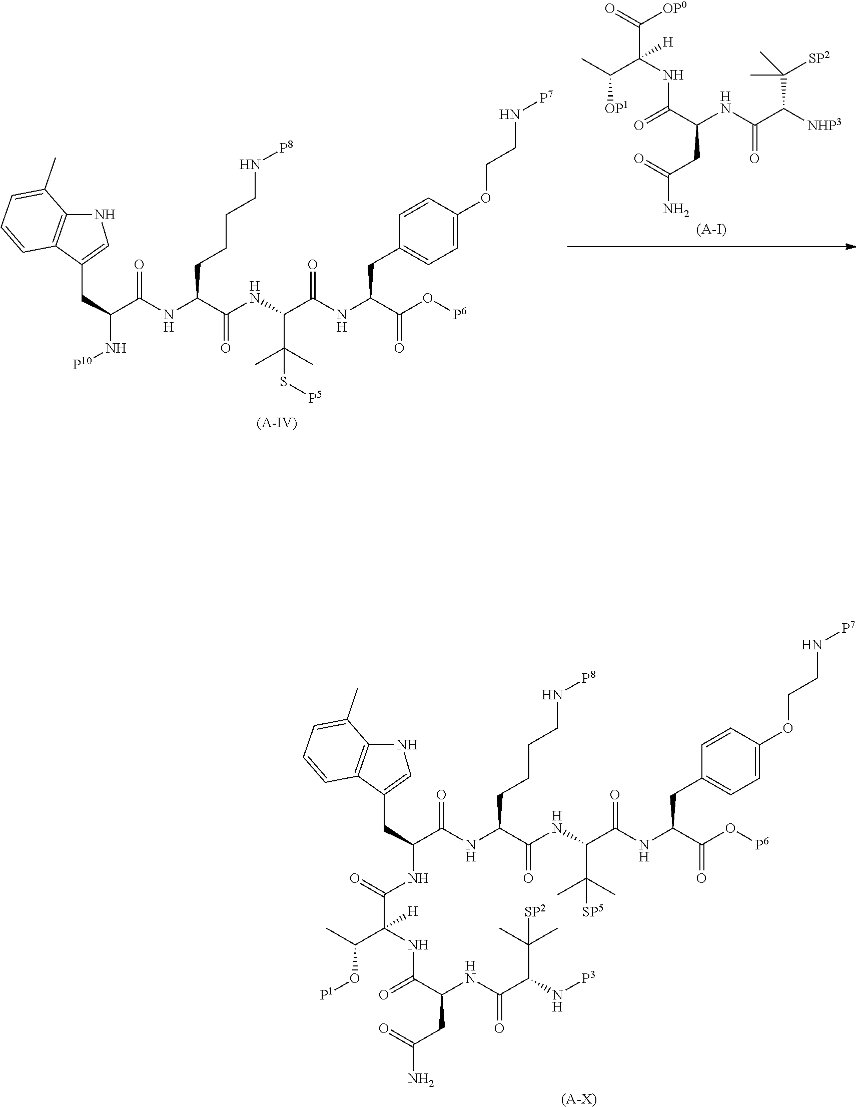

wherein:
P⁰ is H;
P¹ is H, tert-butyl, Bn, or Bz;
P² is H, trityl, or Acm;
P³ is H, Fmoc, Cbz, or BOC;
P⁵ is H, trityl, or Acm;
P⁶ is H, or C₍₁₋₄₎alkyl;
P⁷ is H, Fmoc, Cbz, or BOC;
P⁸ is H or Ac;
P¹⁰ is H;
   or a pharmaceutically acceptable salt thereof.

Some embodiments relate to a process of preparing (A-IV) by reaction of (A-III) with (A-IV-A) in the presence of reagents selected from
Oxyma B and diisopropylcarbodiimide,
Oxyma Pure and diisopropylcarbodiimide,
(7-azabenzotriazol-1-yloxy)tripyrrolidinophosphonium hexafluorophosphate and diisopropylethylamine,
pivaloyl chloride, pyridine and N-methyl morpholine, with or without N, O bis(trimethylsilyl)acetamide,
N,N,N′N′-tetramethyl-O-(benzotriazole-1-yl)uronium tetrafluoroborate and diisopropylethylamine,
N-hydroxysuccinimide and diisopropylcarbodiimide, or
Oxyma Pure and N-ethyl-N′-(3-dimethylaminopropyl)carbodiimide

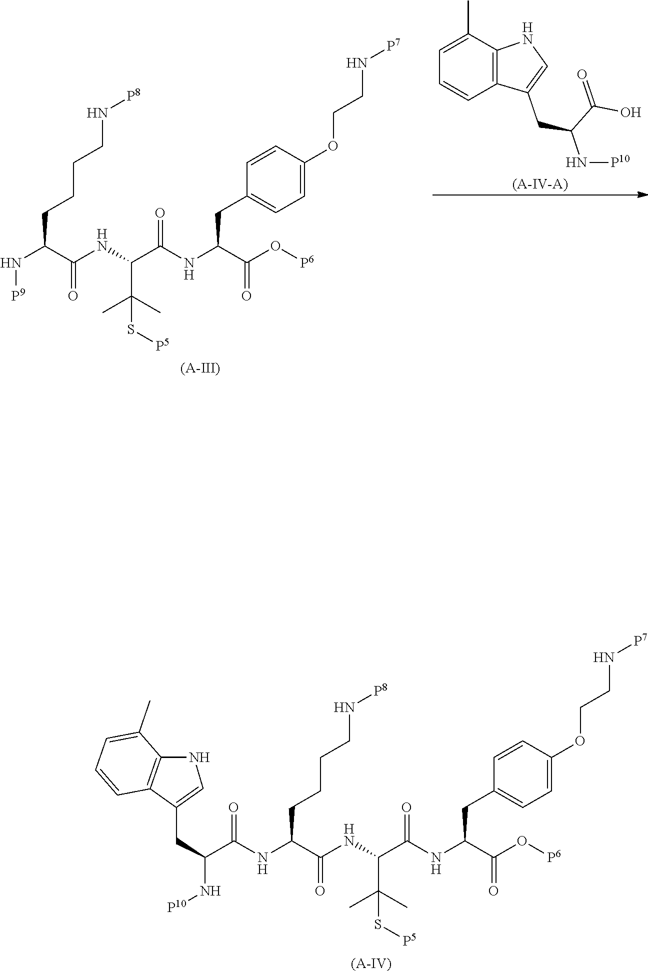

wherein:
P⁵ is H, trityl, or Acm;
P⁶ is H, or C₍₁₋₄₎ alkyl;
P⁷ is H, Fmoc, Cbz, or BOC;
P⁸ is H or Ac;
P⁹ is H;
P¹⁰ is H, Fmoc, Cbz, or BOC;
or a pharmaceutically acceptable salt thereof.

Some embodiments relate to a process of preparing (A-III) by reaction of (A-II) with (A-III-A) in the presence of reagents selected from
Oxyma B and diisopropylcarbodiimide,
Oxyma Pure and diisopropylcarbodiimide,
(7-azabenzotriazol-1-yloxy)tripyrrolidinophosphonium hexafluorophosphate and diisopropylethylamine,
pivaloyl chloride, pyridine and N-methyl morpholine, with or without N, O bis(trimethylsilyl)acetamide,
N,N,N′N′-tetramethyl-O-(benzotriazole-1-yl)uronium tetrafluoroborate and diisopropylethylamine,
N-hydroxysuccinimide and diisopropylcarbodiimide, or
Oxyma Pure and N-ethyl-N′-(3-dimethylaminopropyl)carbodiimide

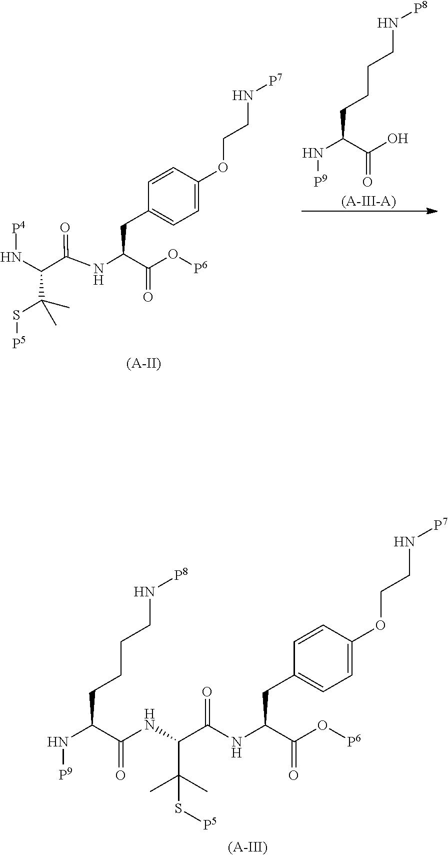

wherein
P⁴ is H;
P⁵ is H, trityl, or Acm;
P⁶ is H, or C₍₁₋₄₎ alkyl;
P⁷ is H, Fmoc, Cbz, or BOC;
P⁸ is H or Ac;
P⁹ is H, Fmoc, Cbz, or BOC;
   or a pharmaceutically acceptable salt thereof.

Some embodiments relate to a process of preparing (A-II) by reaction of (A-II-A) with (A-II-B) in the presence of reagents selected from
Oxyma B and diisopropylcarbodiimide,
Oxyma Pure and diisopropylcarbodiimide,
(7-azabenzotriazol-1-yloxy)tripyrrolidinophosphonium hexafluorophosphate and diisopropylethylamine,
pivaloyl chloride, pyridine and N-methyl morpholine, with or without N, O bis(trimethylsilyl)acetamide,
N,N,N′N′-tetramethyl-O-(benzotriazole-1-yl)uronium tetrafluoroborate and diisopropylethylamine,
N-hydroxysuccinimide and diisopropylcarbodiimide, or
Oxyma Pure and N-ethyl-N′-(3-dimethylaminopropyl)carbodiimide

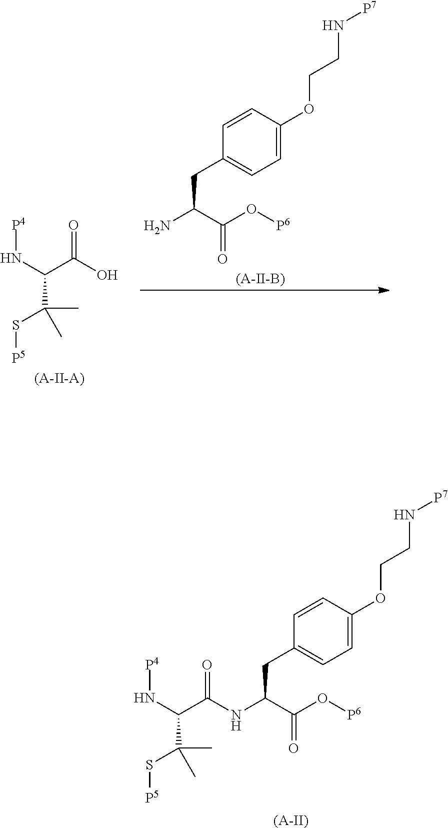

wherein:
P⁴ is H, Fmoc, Cbz, or BOC;
P⁵ is H, trityl, or Acm;
P⁶ is H, or C₍₁₋₄₎ alkyl;
P⁷ is H, Fmoc, Cbz, or BOC;
   or a pharmaceutically acceptable salt thereof.

Some embodiments relate to a process of preparing (A-I) by reaction of (A-I-A) with (A-I-B) in the presence of reagents selected from
Oxyma B and diisopropylcarbodiimide,
Oxyma Pure and diisopropylcarbodiimide,
(7-azabenzotriazol-1-yloxy)tripyrrolidinophosphonium hexafluorophosphate and diisopropylethylamine,
pivaloyl chloride, pyridine and N-methyl morpholine, with or without N, O bis(trimethylsilyl)acetamide,
N,N,N′N′-tetramethyl-O-(benzotriazole-1-yl)uronium tetrafluoroborate and diisopropylethylamine,
N-hydroxysuccinimide and diisopropylcarbodiimide, or
Oxyma Pure and N-ethyl-N′-(3-dimethylaminopropyl)carbodiimide

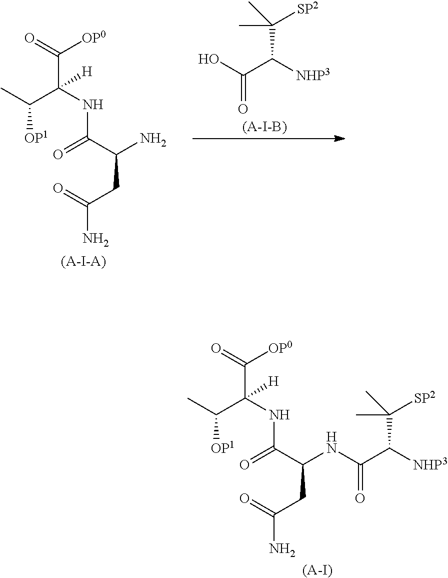

wherein:
P⁰ is H, or C₍₁₋₄₎alkyl;
P¹ is H, tert-butyl, Bn, or Bz;
P² is H, trityl, or Acm;
P³ is H, Fmoc, Cbz, or BOC;
   or a pharmaceutically acceptable salt thereof.

A product of Compound 26 or a pharmaceutically acceptable salt thereof made by a process comprising the steps of:

(a) reacting Fmoc-2-Nal-THPGly-OH with H-Glu(OtBu)-Asn-3-Pal-Sar-NH2

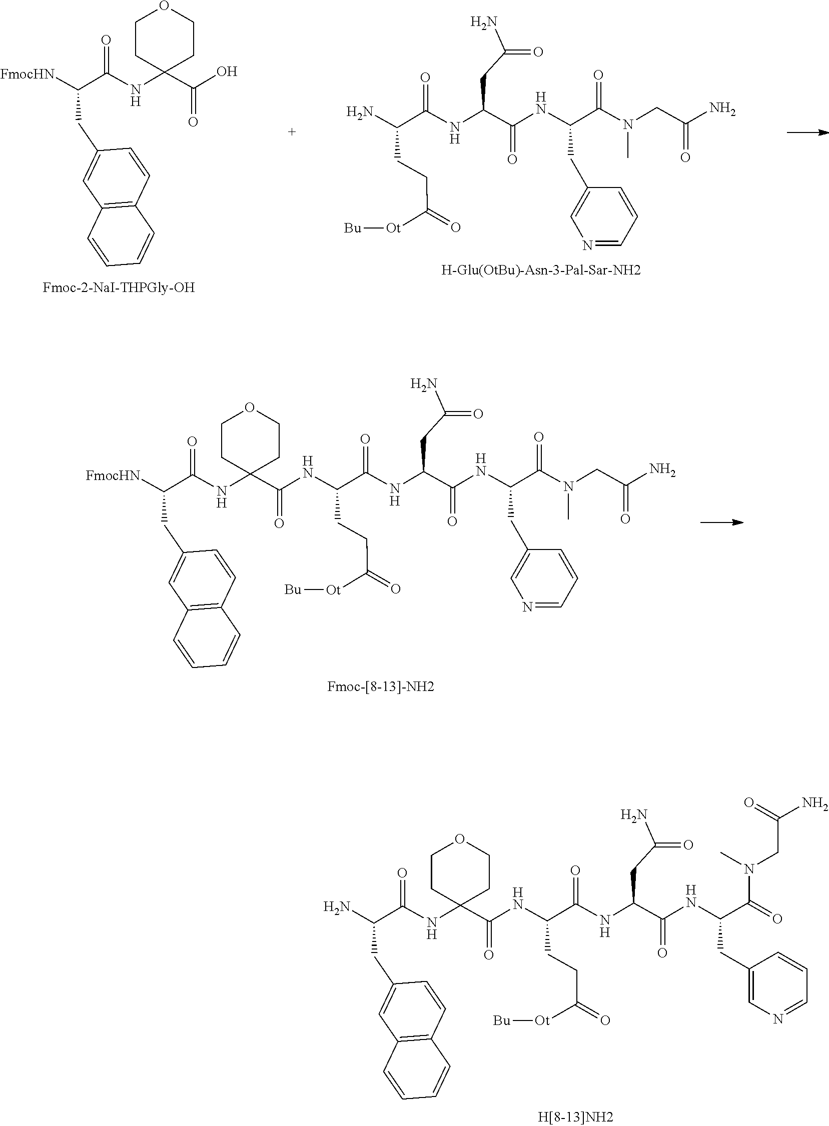

in the presence of (7-azabenzotriazol-1-yloxy)tripyrrolidinophosphonium hexafluorophosphate and diisopropylethylamine, to form Fmoc[8-13]NH2, followed by reaction with 1,8-diazabicyclo[5.4.0]undec-7-ene to form H[8-13]NH2;
(b) reacting Ac-Pen(Trt)-Asn-Thr(tBu)-OH with H-7Me-Trp-Lys(Ac)-Pen(Acm)-Tyr(2-Boc-ea)-OMe in the presence of diisopropylcarbodiimide and Oxyma B, followed by reaction with iodine in the presence of potassium iodide.

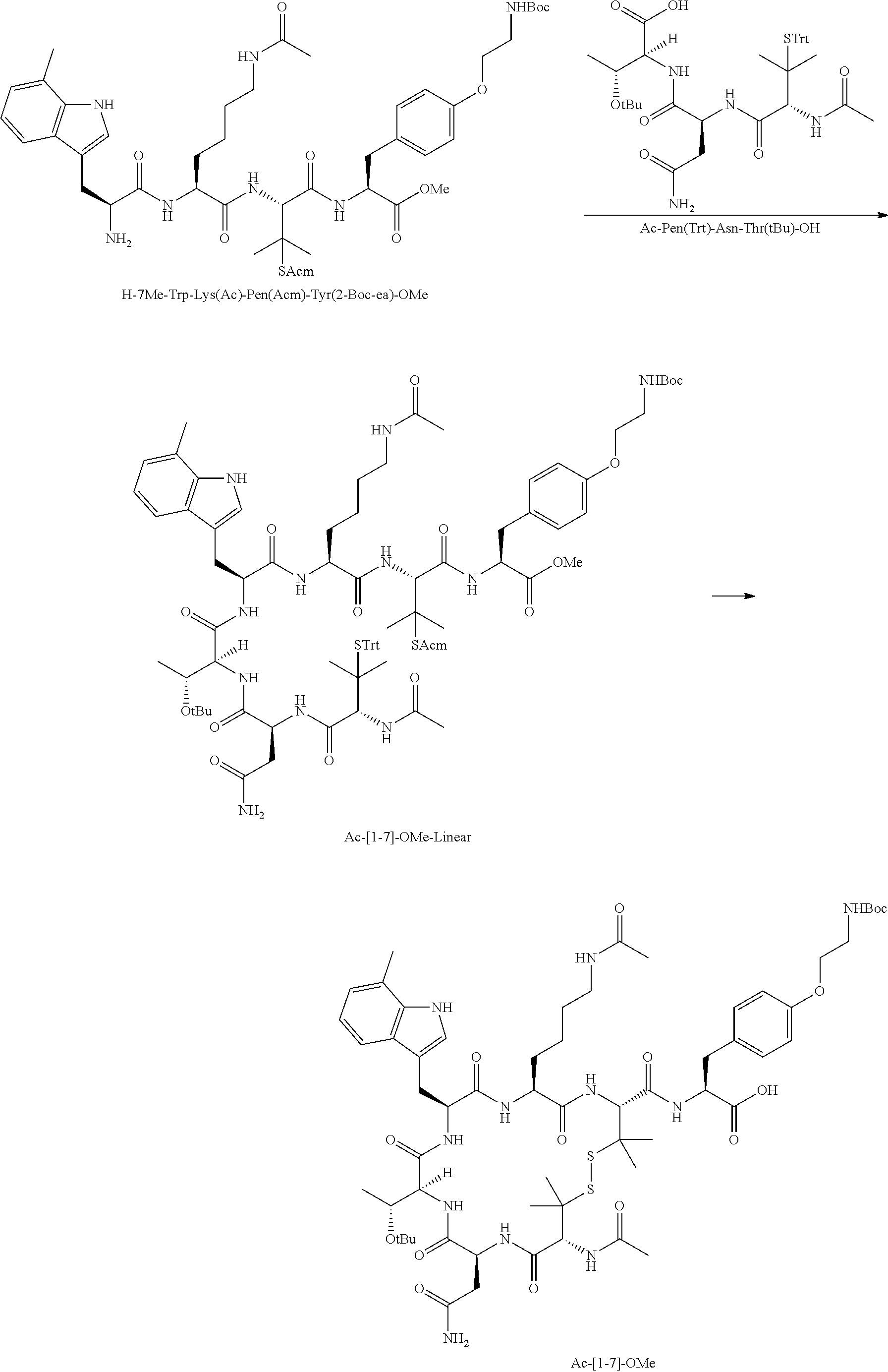

to form Ac-[1-7]-OMe;
(c) reacting Ac-[1-7]—OH-cyclic with H-[8-13]-NH2:

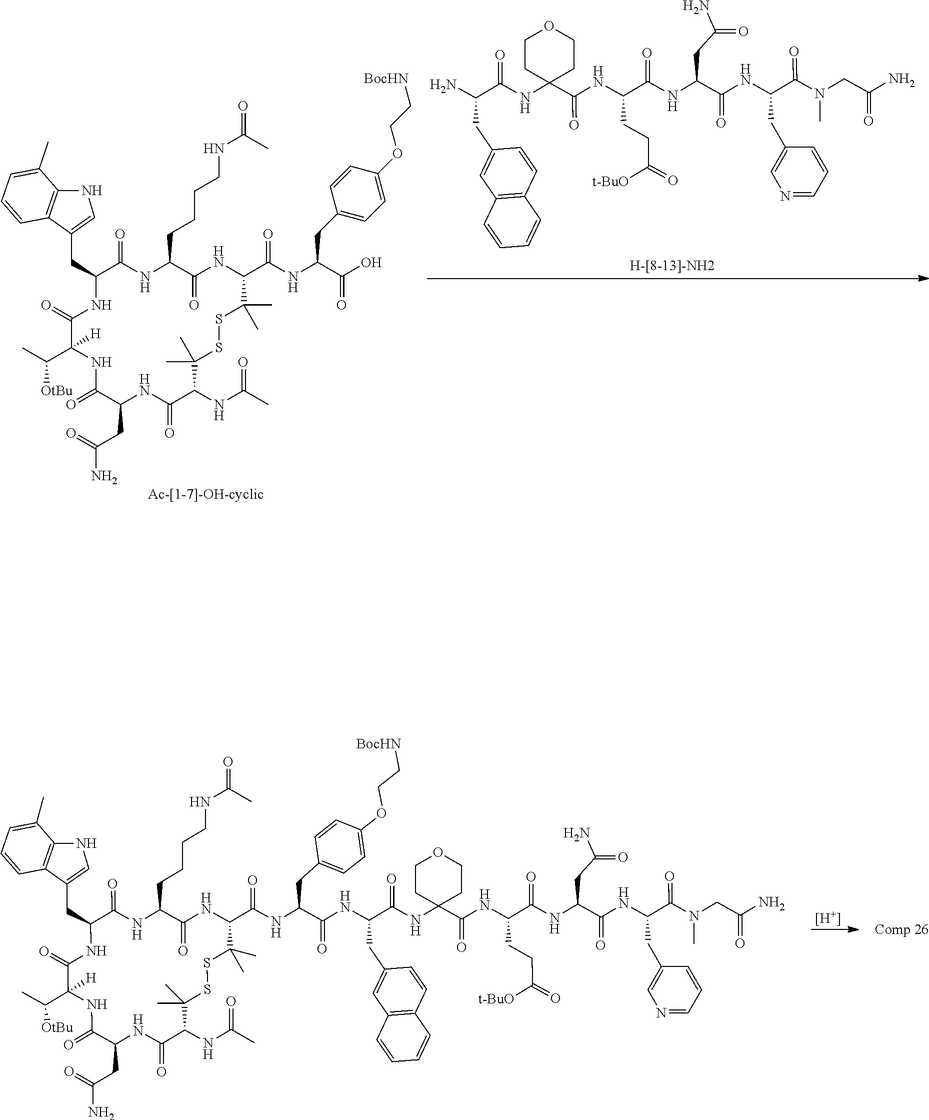

In some embodiments, the reaction described above occur in the presence of diisopropylcarbodiimide and Oxyma-B, followed by acid mediated removal of the tert-butyl groups from threonine and glutamic acid and the butyloxycarbonyl group from aminoethoxy phenyl alanine; to form the product of Compound 26 or a pharmaceutically acceptable salt thereof.

### EXAMPLES

The invention will now be described in greater detail by way of specific examples.

The following reaction schemes may aid in understanding the reactions discussed throughout the following examples.

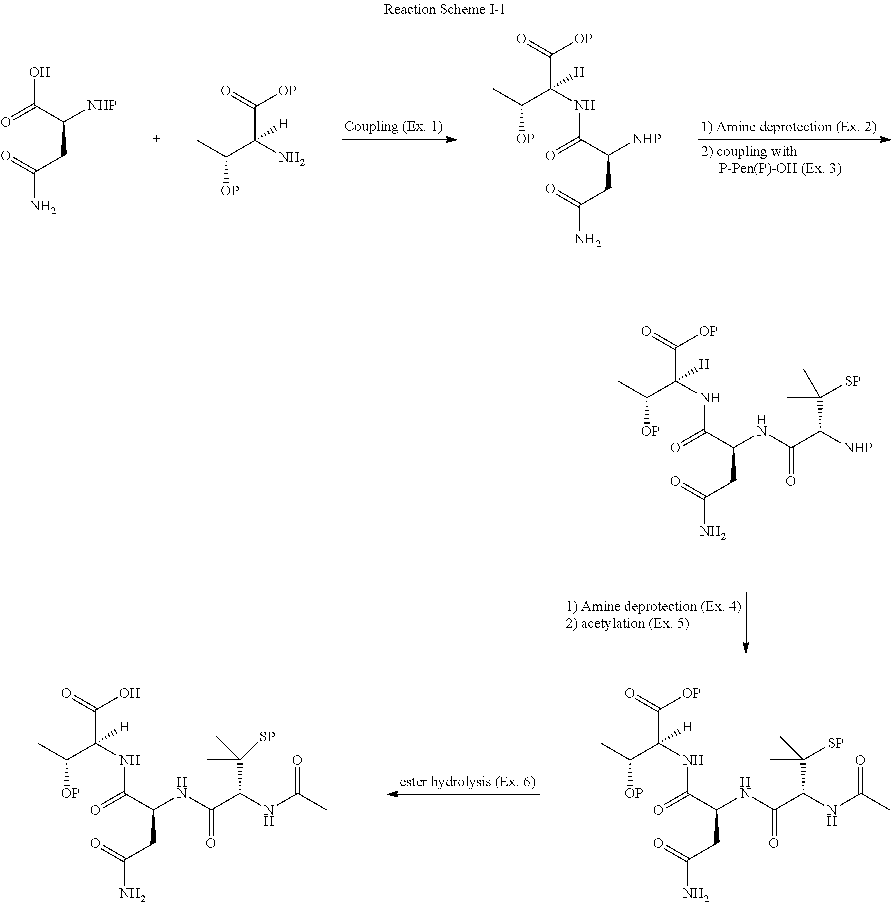

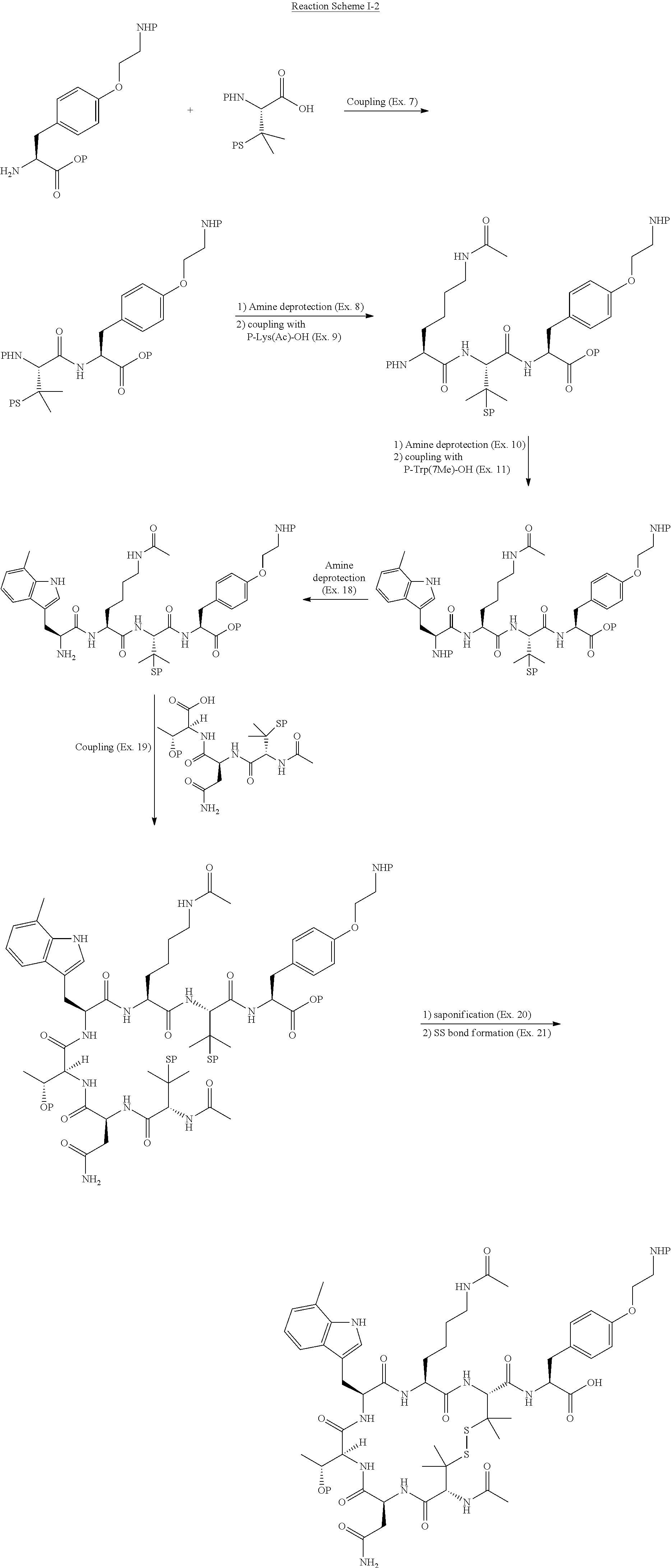

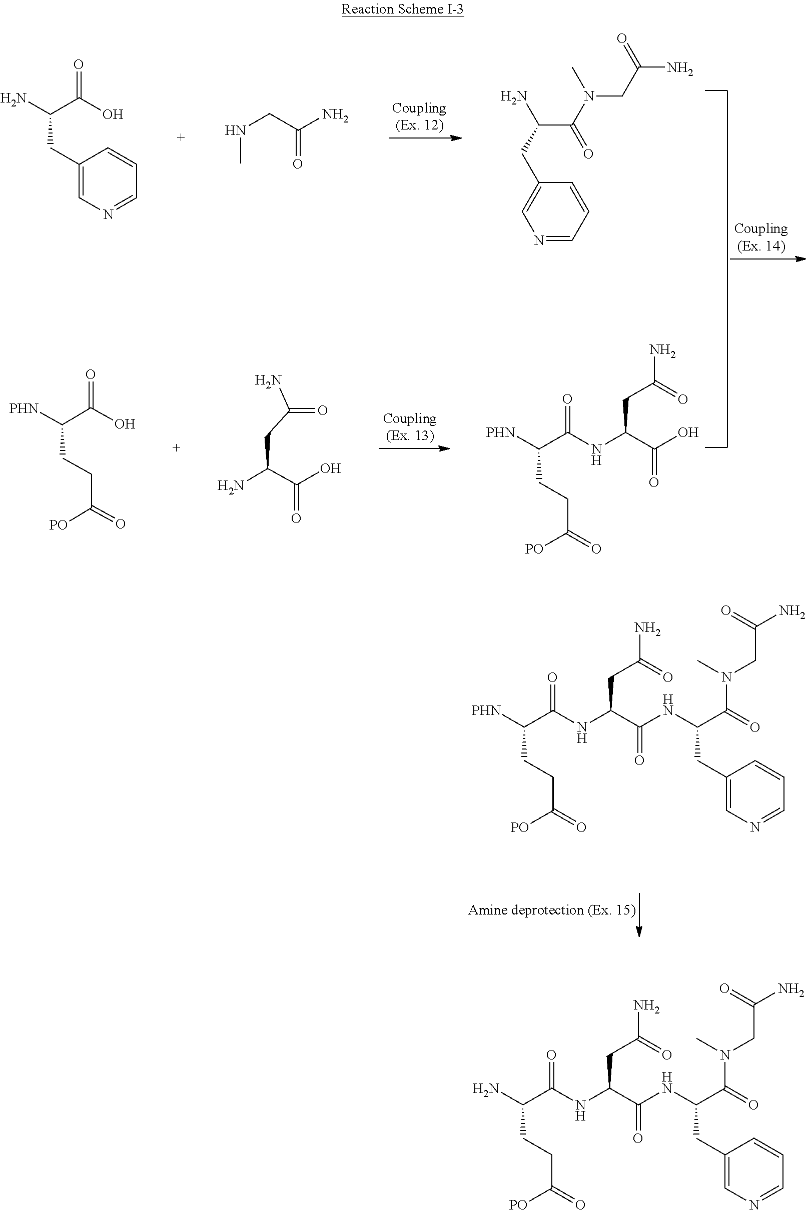

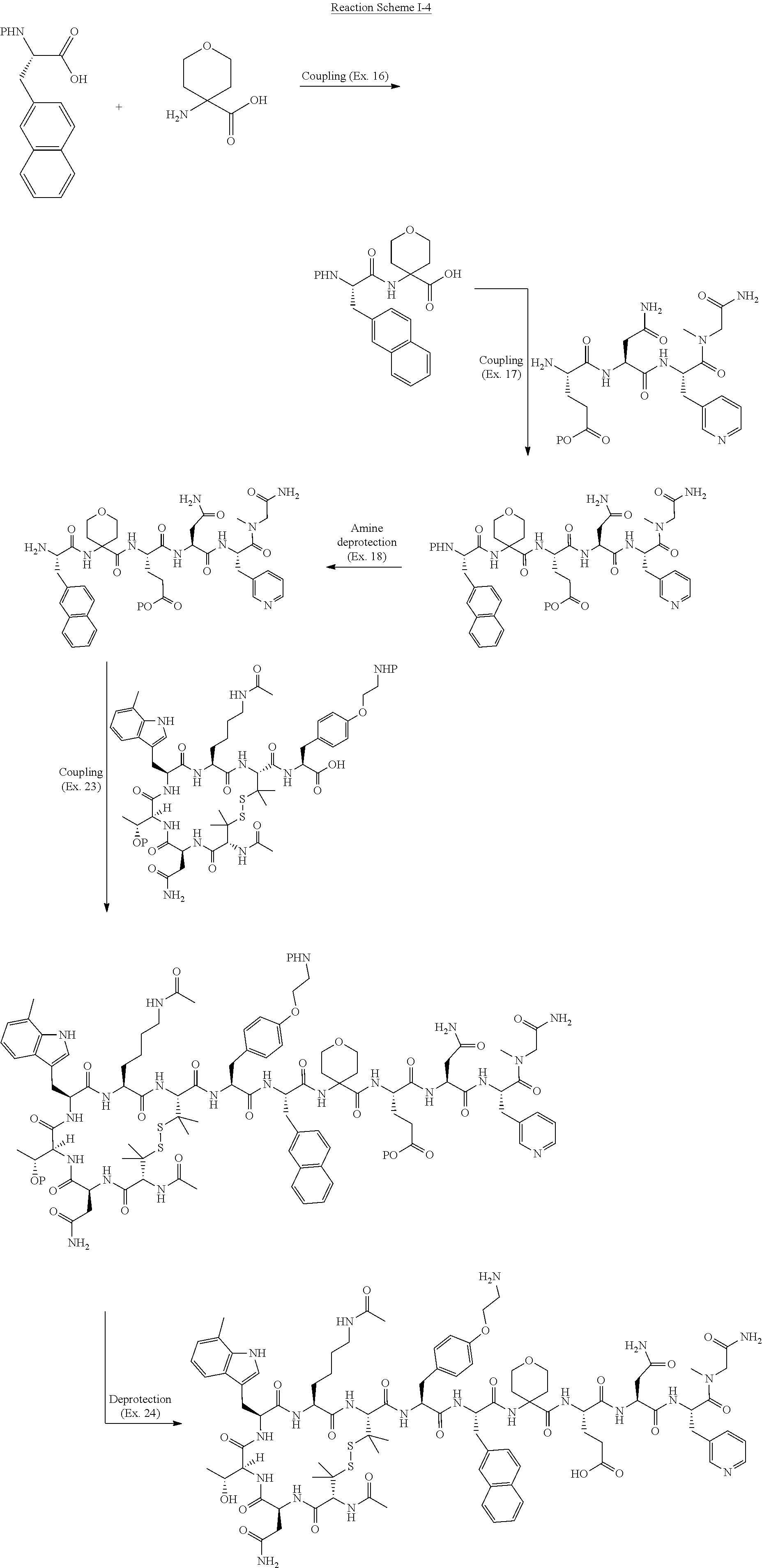

### Example I-1: Synthesis of Cbz-Asn-Thr(tBu)-OMe

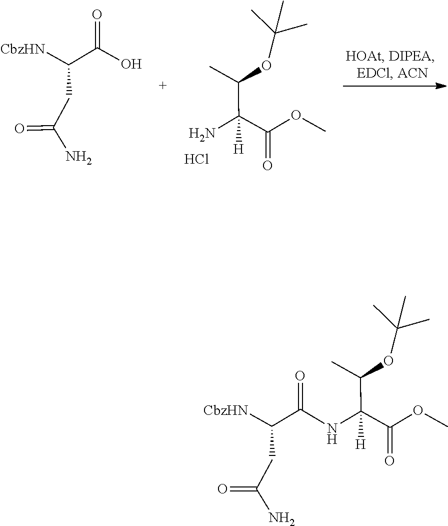 [SMILES: NC(=O)C[C@H](NC(=O)OCc1ccccc1)C(=O)O ; Cl ; COC(=O)[C@@H](N)[C@@H](C)OC(C)(C)C ; COC(=O)[C@@H](NC(=O)[C@H](CC(N)=O)NC(=O)OCc1ccccc1)[C@@H](C)OC(C)(C)C]

To a solution of Cbz-Asn-OH (203.1 kg, 750 mol, 1.1 eq.), H-Thr(tBu)-OMe*HCl (160.1 kg, 681 mol, 1.0 eq.) and MeCN (3.5 vol., 560 L, 443 kg) were added. HOAt (48 kg, 341 mol, 0.5 eq.) was added portion wise over 2.5 hours, then DIPEA (202 kg, 1498 mol, 2.2 eq.) was added dropwise over 2.1 hours. EDCI (163 kg, 817 mol, 1.2 eq.) was then added portion wise over 4 hours at −5.5° C., then the mixture was stirred at this temperature for 8 hours, then at 5-15° C. for 16 hours.

IPAc (12.5 vol., 1931 L, 1680 kg) and 5% citric acid (5 vol., 770 L, 770 kg) were added to the reaction mixture at 5-15° C., then the organic phase was separated out and washed with 5% NaHCO₃ (5 vol., 770 L, 770 kg) twice and with 23% aq. NaCl (3 vol., 473 L, 473 kg) once. The organic phase was concentrated to 9 vol. (vs H-Thr(tBu)-OMe*HCl) under reduced pressure at 40° C., then IPAc (3 vol. 462 L, 402 kg) was added and the mixture was once again concentrated to 9 vol. (vs H-Thr(tBu)-OMe*HCl). n-Heptane was added (16 vol., 2600 L, 1768 kg) dropwise to the solution for 5 hours at 25-35° C. The mixture was then stirred for 1 hour at 25-35° C., cooled to −5-5° C. over 5 hours, then stirred at −5-5° C. for 10.5 hours. More n-heptane (2 vol., 300 L, 204 kg) was added dropwise for 1 hour at −5-5° C., then the mixture was stirred at −5-5° C. for 4.5 hours and filtered. The filter cake was washed with pre-cooled IPAc/n-heptane (v/v) (2.4 vol., 371 kg) and the product was dried under reduced pressure at 40° C. for 17 hours to give 275.1 kg (90.0% yield, 98.8% UPLC purity) of the desired product (Cbz-Asn-Thr(tBu)-OMe).

### Example I-2: Catalytic Hydrogenolysis to Give H-Asn-Thr(tBu)-OMe

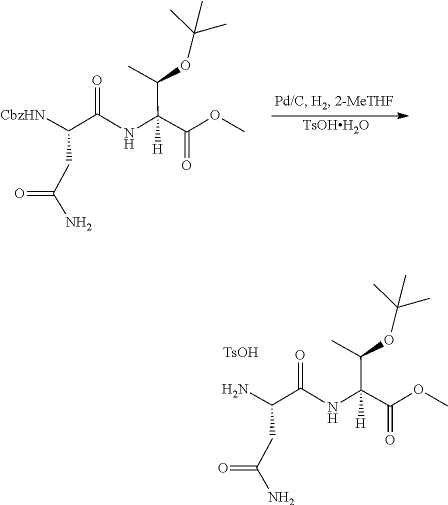 [SMILES: COC(=O)[C@@H](NC(=O)[C@H](CC(N)=O)NC(=O)OCc1ccccc1)[C@@H](C)OC(C)(C)C ; "TsOH·H2O" ; "TsOH" ; COC(=O)[C@@H](NC(=O)[C@@H](N)CC(N)=O)[C@@H](C)OC(C)(C)C]

Cbz-Asn-Thr(tBu)-OMe (266.8 kg, 609.8 mol, 1.0 eq.) and 2-MeTHF (7.5 vol., 1981 L, 1704 kg) were added to a hydrogenation reactor. Wet Pd/C (26.75 kg, 10 wt %) was added and the gas atmosphere was swapped for N₂ three times, then H₂ three times. Hydrogen pressure (40 psi) was applied, and the reaction mixture was stirred at 24-27° C. for 4 hours.

The mixture was filtered through a celite filter and the filter cake was washed with 2-MeTHF (2 vol., 533 L, 458 kg). The product was transferred into solution by adding 4-methylbenzenesulfonic acid (117.6 kg, 609.8 mol, 1.0 eq.) in 2-MeTHF (1.0 vol., 260 L, 223 kg), giving a 2916 kg solution (90.4% yield, 89.9% HPLC purity) of the desired product (H-Asn-Thr(tBu)-OMe).

### Example I-3: Synthesis of Fmoc-Pen(Trt)-Asn-Thr(tBu)-OMe

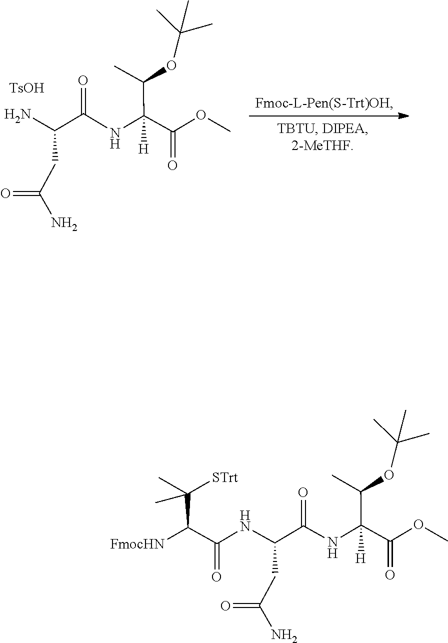

In a 10000 L glass reactor with overhead stirrer were loaded Fmoc-Pen(Trt)-OH (348 kg, 551.9 mol, 1.03 eq), 2-MeTHF (4 vol., 1105 L, 950 kg) and the solution obtained in Example 2 (2916 kg, H-Asn-Thr(tBu)-Ome). The reaction mixture stirred at 20-25° C. for 0.5 hours, then N-ethyl-N-isopropylpropan-2-amide (228 kg, 1764.1 mol, 2.89 eq.) was added dropwise over 1 hour. TBTU was added portion-wise (201 kg, 626 mol, 1.02 eq.) over 3 hours, then the reaction mixture was stirred at 20-25° C. for 20 hours until completion of the reaction.

The reaction mixture was diluted with 5% NaHCO₃ aq. (4.1 vol., 2270 kg), added dropwise at 21-23° C. over 2 hours, then the mixture was stirred for 1 hour at 20-25° C. and separated. The organic phase was washed with 5% NaHCO₃ aq. (4.1 vol., 2276 kg), then with process water (3.2 vol., 1747 kg).

The organic phase was concentrated at 40° C. to 3 vol. (vs Fmoc-Pen(Trt)-Asn-Thr(tBu)-OMe), then 2-MeTHF (5 vol., 2741 L, 2357 kg) was added and the mixture was again concentrated to 3 vol. a total of two times. The wall of the reaction flask was rinsed with 2-MeTHF (0.84 vol., 460 L, 396 kg), EtOH (12 vol., 5643 L, 45000 kg) was added dropwise over 6 hours at 15-25° C., then the mixture was stirred for 3 hours. The solution was cooled to −1-1° C. for 5 hours, then stirred for a further 16 hours.

The resulting mixture was filtered and the filter cake was washed with precooled (0° C.) EtOH/2Me-THF 4:1 mixture (2 vol., 1060 L, 858 kg) and dried at 40° C. under reduced pressure and N₂ flow for 35 hours, yielding 464.5 kg (85.6% yield, 98.6% HPLC purity) of the desired product (Fmoc-Pen(Trt)-Asn-Thr(tBu)-OMe).

### Example I-4: Synthesis of H-Pen(Trt)-Asn-Thr(tBu)-OMe

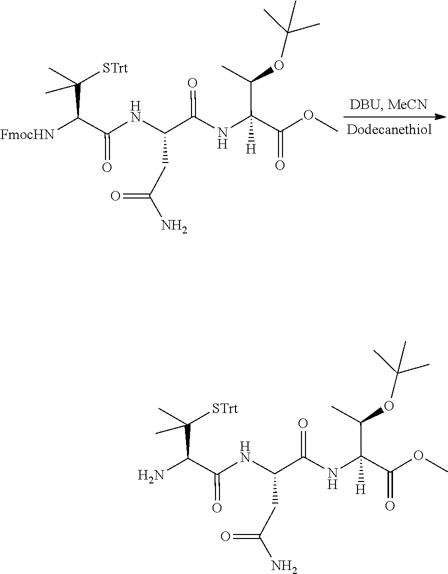

In a 12500 L reactor with overhead stirrer were loaded Fmoc-Pen(Trt)-Asn-Thr(tBu)-OMe (431 kg, 479.4 mol, 1.0 eq.), acetonitrile (5.0 vol., 2168 L, 1704 kg), and dodecanethiol (291 kg, 1438.2 mol, 3.0 eq.). DBU (22.2 kg, 143.8 mol, 0.3 eq.) was added dropwise over 1 hour and the reaction mixture stirred at 15-25° C. for 8 hours until completion of the reaction.

The reaction mixture was allowed to stand for 2 hours and the bottom layer was removed. n-Heptane (5 vol., 2167 L, 1473 kg) was added and the mixture was stirred for 1 hour at 20° C. and allowed to stand for 2 hours. The mixture was separated and the upper layer was removed. n-Heptane (5 vol., 2160 L, 1469 kg) was added and the solution was stirred for 1 hour at 0° C., then allowed to stand for 2 hours. After separation, the upper layer was removed, and to the bottom layer was added n-heptane (5 vol., 2160 L, 1469 kg), then the mixture was stirred at 0° C. for 1 hour and allowed to stand for 2 hours. Further separation occurred and the upper layer was removed, giving a solution (97.2% yield) of the desired product (H-Pen(Trt)-Asn-Thr(tBu)-OMe).

### Example I-5: Synthesis of Ac-Pen(Trt)-Asn-Thr(tBu)-OMe

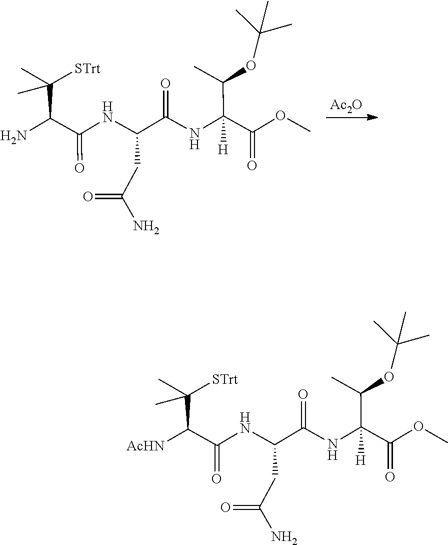

Process water (0.4 vol., 174 kg) was added to the solution from the previous step (H-Pen(Trt)-Asn-Thr(tBu)-Ome), followed by dropwise addition of acetic anhydride (55.6 kg, 527.3 mol, 1.1 eq.) at 0° C. over 2 hours. The mixture was stirred for 1 hour, giving a 2370 kg (97.9% yield, 97.7% UPLC purity) solution of the desired product (Ac-Pen(Trt)-Asn-Thr(tBu)-Ome).

### Example I-6: Synthesis of Ac-Pen(Trt)-Asn-Thr(tBu)-OH

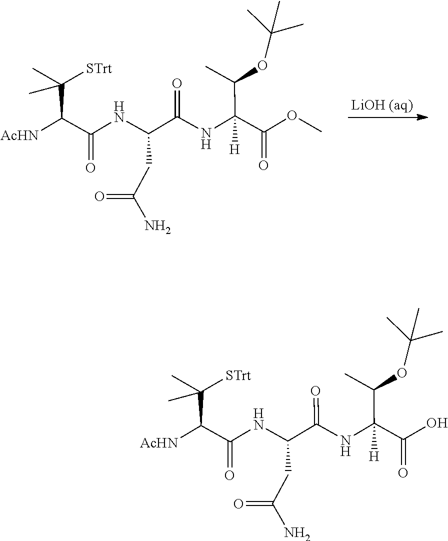

In a 12500 L reactor with overhead stirring were loaded the solution from the previous step (Ac-Pen(Trt)-Asn-Thr(tBu)-OMe) diluted to be homogeneous with MeCN/H₂O and process water (6 vol., 2074 L, 2074 kg) at 0° C. LiOH·H₂O (51.8 kg, 1246 mol, 2.6 eq.) was added dropwise over 4 hours at −5-5° C., then the mixture was stirred at 0° C. for 15 hours.

The reaction mixture was adjusted to pH 4.96 with 8.5% aq. HCl (1.16 vol., 339 kg) at −5-5° C. and 2-MeTHF (8 vol., 2924 L, 2514 kg) was added. The pH was further adjusted to 2.34 using 1.1% aq. HCl (5.8 vol., 1984 kg) and the mixture was stirred for 2 hours and allowed to stand for 1 hour. After separation, the upper layer was collected and to the bottom layer was added 2-MeTHF (5 vol., 1490 kg), before stirring for 2 hours and allowing to stand for 1 hour. The mixture was separated again and all the organic phase was collected. To this, 23% NaCl aq. (5 vol., 1924 kg) was added, the mixture was stirred for two hours, allowed to stand for 2 hours, and the bottom layer was removed after separation.

The organic phase was concentrated at 40° C. to 3 vol., then 2-MeTHF (4.5 vol., 1451 L, 1248 kg) was added. This process was repeated a further four times with the following volumes of 2-MeTHF: 1323 L, 1391 L, 1416 L, 1431 L. MeCN (0.1 vol., 38 L, 30 kg) was then added to the reaction mixture, along with Ac-Pen(Trt)-Asn-Thr(tBu)-OH (1.62 kg), and the solution was stirred for 1 hour. More MeCN (12 vol., 3931 L, 3090 kg) was added, and the reaction was stirred at 40° C. for 2 hours, then at 0° C. for 38 hours.

The resulting mixture was filtered and the cake was washed with 2-MeTFH/MeCN (1/2701 L, 568 kg), then dried at 43° C. under reduced pressure. This process produced 320 kg (90.2% yield, 100% UPLC purity) of the desired product (Ac-Pen(Trt)-Asn-Thr(tBu)-OH).

### Example I-7: Synthesis of Fmoc-Pen(Acm)-Tyr(2-Boc-ea)-OMe

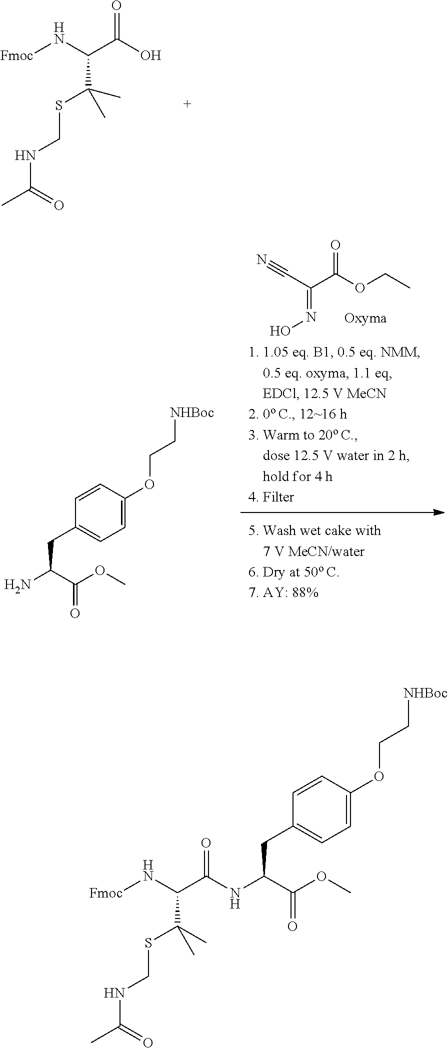

Fmoc-Pen(Acm)-OH (369 kg, 1.0 eq), H-Tyr(2-Boc-ea)-OMe (298 kg, 1.05 eq.) and MeCN (12.5 vol., 4612 L) were added to a 15000 L reactor, and the mixture was cooled to 0° C. N-methylmorpholine (0.5 eq.) and Oxyma Pure (0.5 eq.) were added, followed by portion wise addition of EDCI (1.1 eq.) at 0-5° C. over 2 hours. The resulting mixture was stirred for 16-20 hours at 0° C. until completion of the reaction. Fmoc-Pen(Acm)-Tyr(2-Boc-ea)-OMe (1.5 kg) was added and the mixture was stirred for 12 hours at 0° C., then the mixture was warmed to 20° C. over 1 hour and the slurry was aged for a further 4 hours. Water (12 vol., 4612 L) was added dropwise to the mixture over 2 hours and the solution was left to stand for 4 hours. The reaction mixture was filtered and the filter cake was washed with MeCN/H₂O (1:1 v/v). The wet cake was dried in a vacuum oven under reduced pressure at 45° C. over 48 hours to give 561 kg (88% yield, 99.8% UPLC purity) of the desired product (Fmoc-Pen(Acm)-Tyr(2-Boc-ea)-OMe).

### Example I-8: Synthesis of H-Pen(Acm)-Tyr(2-Boc-ea)-OMe

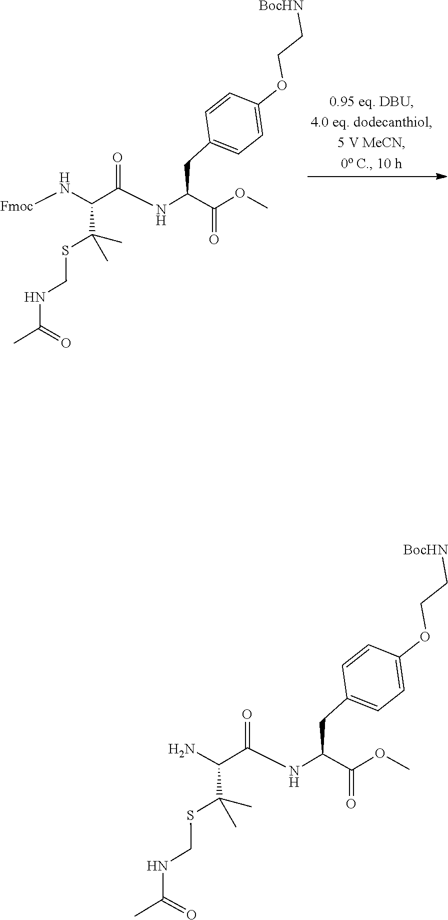

Fmoc-Pen(Acm)-Tyr(2-Boc-ea)-OMe (558 kg, 1.0 eq) was combined with MeCN (5 vol., 2760 L) in an 8000 L reactor and the mixture was cooled to 0° C. Pre-cooled dodecanethiol (4.0 eq.) was added, followed by DBU (0.95 eq.), which was dosed over 1 hour. After 10 hours, the reaction mixture was washed with heptane (3×5 vol.), then diluted with EtOAc (6 vol.) and washed with 15% NH₄Cl aq. (3 vol.) and 25% NaCl aq. (1 vol.), then 10% NaCl aq. (3 vol.). This produced 371.7 kg (94% yield, 99.5% UPLC purity) of the desired product (H-Pen(Acm)-Tyr(2-Boc-ea)-OMe).

### Example I-9: Synthesis of Fmoc-Lys(Ac)-Pen(Acm)-Tyr(2-Boc-ea)-OMe

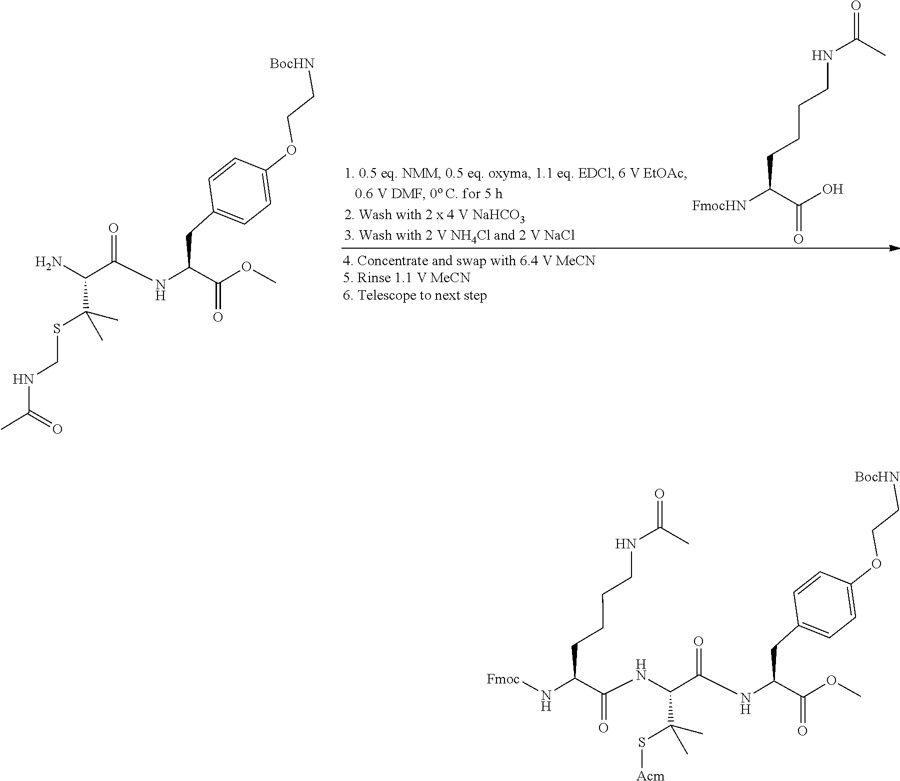

To the solution of H-Pen(Acm)-Tyr(2-Boc-ea)-OMe (371.7 kg) was added Fmoc-Lys(Ac)—OH (0.96 eq. vs Fmoc-Pen(Acm)-Tyr(2-Boc-ea)-OMe from Example 8)) and the solution was cooled to 0° C. N-methylmorpholine (0.5 eq.), ethyl cyanoglyoxylate-2-oxime (0.5 eq.), and EDCI (1.1 eq.) were added and the reaction mixture was stirred for 16-20 hours until the completion of the reaction. The solution was washed twice with 5% NaHCO₃ aq. (2×6 vol.), then NH₄Cl (3 vol.) and 10% NaCl aq. (3 vol.) at 0° C. The solution was concentrated to 3 vol., then MeCN (8 vol.) was added and the resulting solution was concentrated to 5 vol., yielding 641.5 kg (100% yield, 96.7% UPLC purity) of the desired solution (Fmoc-Lys(Ac)-Pen(Acm)-Tyr(2-Boc-ea)-OMe).

### Example I-10: Synthesis of H-Lys(Ac)-Pen(Acm)-Tyr(2-Boc-ea)-OMe

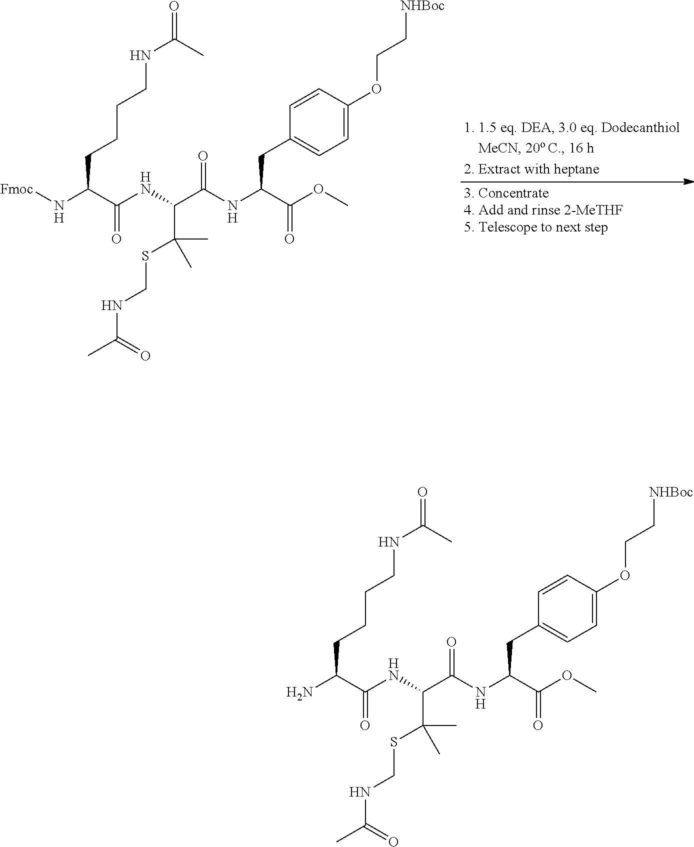

To the solution of Fmoc-Lys(Ac)-Pen(Acm)-Tyr(2-Boc-ea)-OMe (640.5 kg, 1.0 eq) was added diethylamine (1.5 eq.) and dodecanethiol (3 eq.) at 20° C., with MeCN (1 vol.) being used to rinse the pipeline. The resulting solution was stirred for 7-10 hours at 20° C., the reaction mixture was washed three times with heptane (3.5 vol.) and concentrated to 2 vol., before 2-MeTHF (4 vol.) was added. This resulted in a 488 kg (100% yield, 97.2% UPLC purity) of solution of the desired product (H-Lys(Ac)-Pen(Acm)-Tyr(2-Boc-ea)-OMe).

### Example I-11: Synthesis of Fmoc-Trp(7Me)-Lys(Ac)-Pen(Acm)-Tyr(2-Boc-ea)-OMe

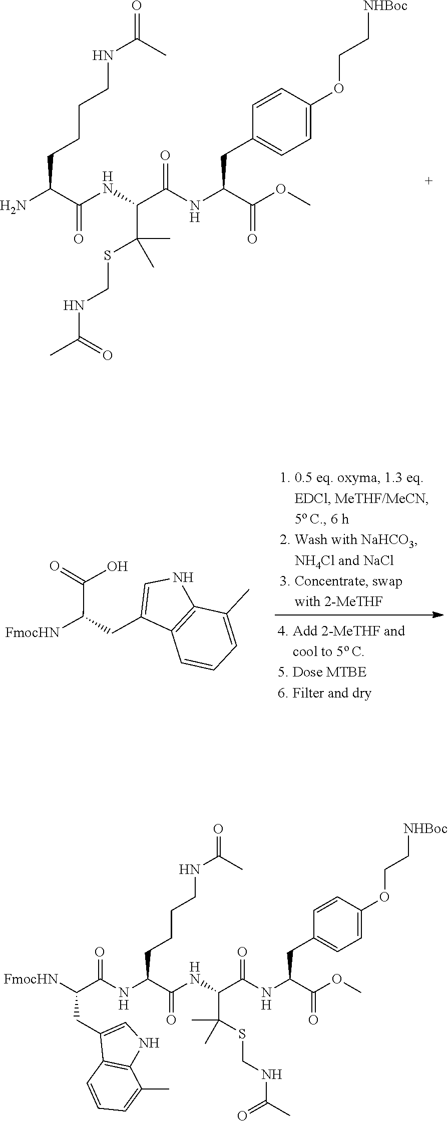

To the solution of H-Lys(Ac)-Pen(Acm)-Tyr(2-Boc-ea)-OMe (496 kg, 0.97 eq.) was added Fmoc-Trp(7Me)-OH (0.97 eq.), Ethyl cyanoglyoxylate-2-oxime (0.5 eq.) and EDCI (1.3 eq.) were added at 0-5° C., before 2-MeTHF (1.2 vol.) was used to rinse the reaction. The resulting mixture was stirred for 10 hours, washed twice with 5% NaHCO₃ (2×6 vol.), then washed with NH₄Cl (6 vol.) and 10% NaCl aq. (6 vol.). The solution was concentrated to 3.5 vol (vs H-Lys(Ac)-Pen(Acm)-Tyr(2-Boc-ea)-OMe), 2-MeTFH (8.6 vol.) was added, and the solution was concentrated to 3.5 vol. again. This operation was repeated until reaching a water content (KF) below 0.3%, then additional 2-MeTHF (3.5 vol.) was added.

The solution was cooled to 5° C., 1 wt % of Fmoc-Trp(7Me)-Lys(Ac)-Pen(Acm)-Tyr(2-Boc-ea)-OMe was added, and the mixture was stirred for 20 hours. MBTE (10.0 vol. vs Fmoc-Trp(7Me)-Lys(Ac)-Pen(Acm)-Tyr(2-Boc-ea)-OMe) was dosed over 5 hours, the mixture was stirred at 5° C. for 4 hours, then stirred at 20° C. for 8 hours. The mixture was filtered, and the filter cake was washed with cold MeTFH/MTBE (2 vol., 1:2 v/v), then with MTBE (2 vol.). The resultant solid was dried at 25° C., yielding 694 kg (87.7% yield, 99.0% UPLC purity) of the desired product (Fmoc-Trp(7Me)-Lys(Ac)-Pen(Acm)-Tyr(2-Boc-ea)-OMe).

### Example I-12: Synthesis of Boc-3Pal-Sarc-NH₂

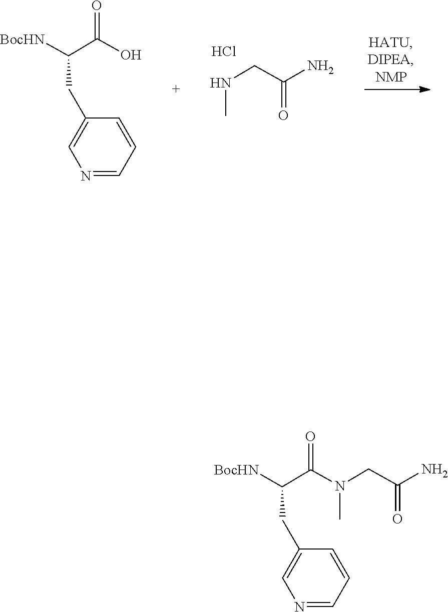

Boc-3Pal-OH (95.1 kg, 357.1 mol, 1.0 eq), NMP (4.0 vol., 380 L, 390 kg) and H-Sarc-NH2-HCl(46.7 kg, 375 mol, 1.05 eq.) were added to a 3000 L reactor at 20-30° C. N,N-diisopropylethylamine (114.1 kg, 892.8 mol 2.5 eq.) was added dropwise over 20 minutes, followed by HATU (152.2 kg, 392.8 mol, 1.1 eq.), which was added in portions over 3.5 hours. The mixture was stirred at 20-30° C. for 2 hours, then EtOAc (10 vol., 951 L, 856 kg) was added over 7 hours, the mixture temperature was adjusted to 10-20° C. over 1 hour, and then stirred for 7 hours. The resulting mixture was filtered and the cake was washed with EtOAc (4 vol., 280.4 L, 342.4 kg) and dried at 45° C. under reduced pressure for 20 hours. This yielded 111 kg (92% yield, 99.7% UPLC purity) of the desired product (Boc-3Pal-Sarc-NH₂).

### Example I-13: Synthesis of Cbz-Glu(tBu)-Asn-H

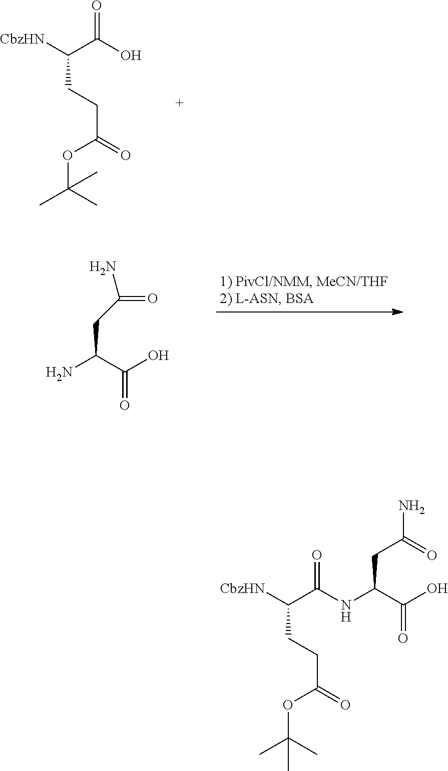 [SMILES: CC(C)(C)OC(=O)CC[C@H](NC(=O)OCc1ccccc1)C(=O)O ; NC(=O)C[C@H](N)C(=O)O ; CC(C)(C)OC(=O)CC[C@H](NC(=O)OCc1ccccc1)C(=O)N[C@@H](CC(N)=O)C(=O)O]

MeCN (6 vol., 762 L, 602 kg), THF (4 vol., 508 L, 452 kg), and Cbz-Glu(tBu)-OH (127 kg, 376.4 mol, 1.0 eq.) were added to a 5000 L reactor at 20-30° C. The reactor was cooled to −15-−5° C., PivCl was added over 20 minutes, then N-methylmorpholine was added over 2.5 hours and the reaction mixture was stirred for a further 2 hours. L-Asn (74.6 kg, 564.6 mol, 1.5 eq.) and N,O-bis(trimethylsilyl)acetamide (153 kg, 752.8 mol, 2.0 eq.) were added over 3 hours, then the mixture was warmed to 15-25° C. and stirred for 14 hours. Process water (20 kg, 1129.2 mol, 3.0 eq.) was added to quench the reaction, then the mixture was stirred for 2 hours.

Following filtration to remove excess L-Asn, the cake was washed with MeCN (3 vol., 381 L, 301 kg), then the filtrate was concentrated to 4 vol. below 45° C. under reduced pressure. MeCN (5 vol., 635 L, 502 kg) was added and the mixture was again concentrated to 4 vol., then the solution temperature was adjusted to 15-25° C. 5% NaHSO₄ aq/(2.5 vol., 318 L, 318 kg) was added over 1 hour followed by product (Cbz-Glu(tBu)-Asn-H) (508 kg, 0.4 wt %). The mixture was stirred for 1 hour, then further 5% NaHSO₄ (9.5 vol., 1207 L, 1207 kg) over 5 hours, then the mixture was stirred for 6 hours.

The suspension was filtered and the cake was washed with process water (4 vol., 508 L, 508 kg), then to the wet cake was added MTBE (10 vol., 1270 L, 953 kg). The slurry was stirred for 4 hours, then filtered. The cake was washed with MTBE (5 vol., 635 L, 470 kg) and dried at 45° C. under vacuum for 32 hours, yielding 141.9 kg (83.5% yield, 98.2% UPLC purity) of the desired product (Cbz-Glu(tBu)-Asn-H).

### Example I-14: Synthesis of Cbz-Glu(tBu)-Asn-3Pal-Sarc-NH2

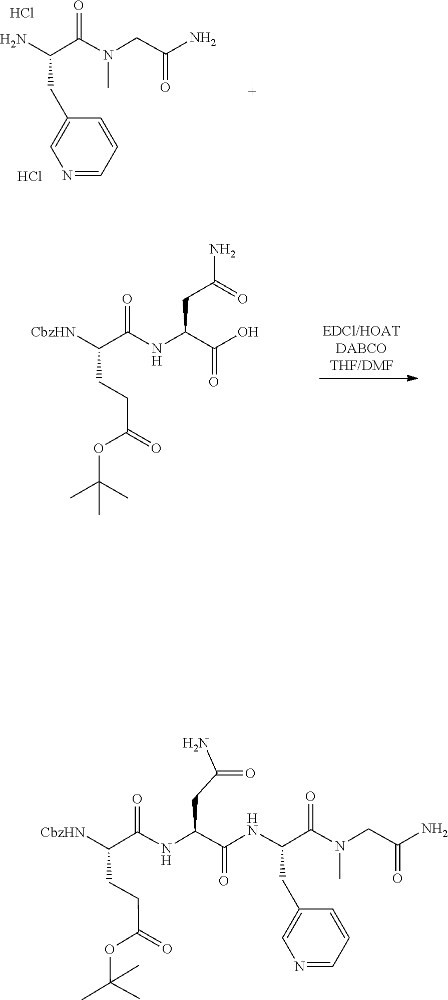 [SMILES: Cl ; CN(CC(N)=O)C(=O)[C@@H](N)Cc1cccnc1 ; Cl ; CC(C)(C)OC(=O)CC[C@H](NC(=O)OCc1ccccc1)C(=O)N[C@@H](CC(N)=O)C(=O)O ; CN(CC(N)=O)C(=O)[C@H](Cc1cccnc1)NC(=O)[C@H](CC(N)=O)NC(=O)[C@H](CCC(=O)OC(C)(C)C)NC(=O)OCc1ccccc1]

Cbz-Glu(tBu)-Asn-H (137.8 kg, 305.3 mol, 1.25 eq.), H-3Pal-Sarc-NH₂ (57.7 kg, 244.2 mol, 1.0 eq.), DMF (3.4 vol., 194 L, 185 kg), THF (15 vol., 866 L, 770 kg), and HOAt (33.2 kg, 244.2 mol, 1.0 eq.) were added to a 5000 L reactor at 20-30° C. The reaction mixture was then adjusted to −15-−5° C. and a solution of DABCO in THF and DMF (DABCO (98.2 kg, 854.7 mol, 3.5 eq.) dissolved in THF (9 vol., 519 L, 462 kg) and DMF (2 vol., 115 L, 110 kg)) was added over 2 hours below −8° C., followed by addition of EDCI (70.2 kg, 366.3 mol, 1.5 eq.) was added in portions over 2.5 hours.

The mixture was stirred at −15-−5° C. for 18 hours, then 7% NaHCO₃ aq. was added at 25° C. until the pH of the mixture reached 5-6. The solution temperature was adjusted to 20-30° C., DMF (8.7 vol., 504 L, 479 kg) was added, and the mixture was distilled to 23 vol. below 40° C. under reduced pressure. EtOH (14 vol., 818 L, 646 kg) was added, and the mixture was again distilled to 23 vol. below 50° C. under reduced pressure, then the temperature was adjusted to 20-30° C. Further EtOH (5 vol., 289 L, 228 kg) was added, and the mixture was again distilled to 23 vol. below 50° C. under reduced pressure, then the temperature was adjusted to 20-30° C. and more EtOH (8.5 vol., 489 L, 387 kg) was added. The mixture was stirred at 45-55° C. for 1 hour, then the temperature was adjusted to 35-45° C., product (577 g, 1 wt %) was added and the mixture was stirred for 3 hours. EtOH (34 vol., 1957 L, 1546 kg) was added over 5 hours, then the mixture was stirred at 35-45° C. for 2 hours. The mixture temperature was adjusted to 15-25° C. over 3 hours, then stirred for 3 hours. Then, the mixture was warmed to 45-55° C. over 1.5 hours, stirred at this temperature for 8 hours, cooled to 15-25° C. over 3 hours and stirred at this temperature for 4 hours, then cooled to 5-15° C. over 3 hours and stirred at this temperature for 10 hours.

The suspension produced was filtered and the cake was washed with EtOH (5.6 vol., 328 L, 259 kg), then dried at 45° C. under reduced pressure for 54 hours. This yielded 126.94 kg (77.5% yield, 100% UPLC purity) of the desired product (Cbz-Glu(tBu)-Asn-3Pal-Sarc-NH2).

### Example I-15: Synthesis of H-Glu(tBu)-Asn-3Pal-Sarc-NH₂

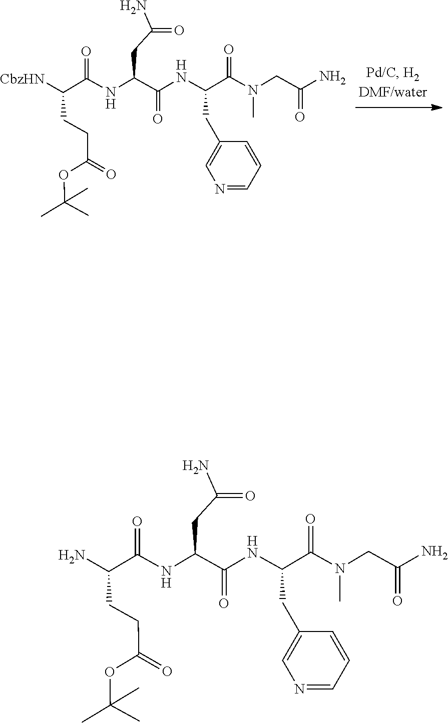 [SMILES: CN(CC(N)=O)C(=O)[C@H](Cc1cccnc1)NC(=O)[C@H](CC(N)=O)NC(=O)[C@H](CCC(=O)OC(C)(C)C)NC(=O)OCc1ccccc1 ; CN(CC(N)=O)C(=O)[C@H](Cc1cccnc1)NC(=O)[C@H](CC(N)=O)NC(=O)[C@@H](N)CCC(=O)OC(C)(C)C]

Cbz-Glu(tBu)-Asn-3Pal-Sarc-NH₂ (119.9 kg, 189.3 mol, 1.0 eq.) was suspended in DMF (4 vol., 480 L, 456 kg) and water (0.25 vol., 30 L, 30 kg) at 20-30° C. Pd/C (12 kg, 10 wt %) was then added and the air in the system was swapped with nitrogen three times by vacuum. The reactor was filled under vacuum with hydrogen to 0.24 MPa at 10-20° C., and the reaction mixture was stirred at 10-20° C. for 19 hours. The reaction suspension was filtered and the cake washed with DMF (1.5 vol., 180 L, 171 kg) in water (0.09 vol., 10.8 L, 10.8 kg), yielding a 661.6 kg solution (94.4% yield, 98.0% UPLC purity) of the desired product (H-Glu(tBu)-Asn-3Pal-Sarc-NH2).

### Example I-16: Synthesis of Fmoc-2Nal-Gly(THP)-OH

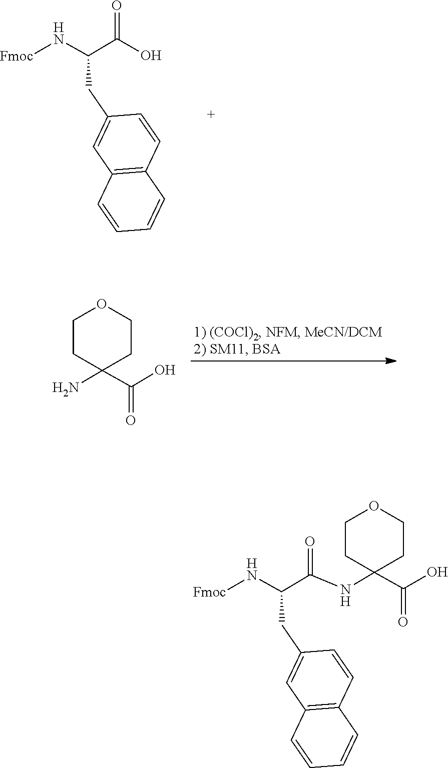

To a solution of Fmoc-2Nal-OH (100.1 kg, 228.8 mol, 1.0 eq.) was added MeCN (5 vol. 500 L, 395 kg), DCM (7 vol., 700 L, 931 kg), and N-formylmorpholine (790 g, 6.87 mol, 0.03 eq.) at 20-30° C. Oxalyl chloride (43.6 kg, 343.2 mol, 1.5 eq.) was added over 2 hours and the mixture was stirred for 2 hours, before being distilled under vacuum to 7 vol. at 25-35° C. MeCN (3 vol., 300 L, 237 kg) was added to the reactor, the temperature was adjusted to 5-15° C., and H-Gly(THP)-OH (46.5 kg, 343.2 mol, 1.5 eq.) was added, followed by BSA (93 kg, 456.4 mol, 2 eq.) within 1 hour. The mixture was stirred for 16 hours, then the reaction was quenched with 5% NaHSO₄ aq. (3.3 vol., 330 L, 330 kg) at 20° C. The solution was distilled in vacuo to 8.3 vol. at 35-45° C., MeCN (5 vol., 500 L, 395 kg) was added, and the mixture was distilled again to 8.3 vol. Further MeCN (5 vol., 500 L, 395 kg) was added, the temperature was adjusted 5-15° C., and Fmoc-2Nal-Gly(THP)-OH (1 kg, 1 wt %) was added. The mixture was stirred for 8 hours, then 5% NaHSO₄ aq. (11.7 vol., 1170 L, 1170 kg) was added over 14 hours and the solution was stirred for a further 5 hours.

The suspension was filtered and the cake was washed with process water (2 vol., 200 L, 200 kg), then mixed with DCM (6 vol., 600 L, 663 kg) at 20-30° C. for 5 hours. The suspension was filtered again, and the cake was washed with DCM (1 vol., 100 L, 133 kg), then dried under reduced pressure at 45° C. for 41 hours. This produced 106.4 kg (82% yield, 100% UPLC purity) of the desired product (Fmoc-2Nal-Gly(THP)-OH).

### Example I-17: Synthesis of Fmoc-2Nal-Gly(THP)-Glu(tBu)-Asn-3Pal-Sarc-NH2

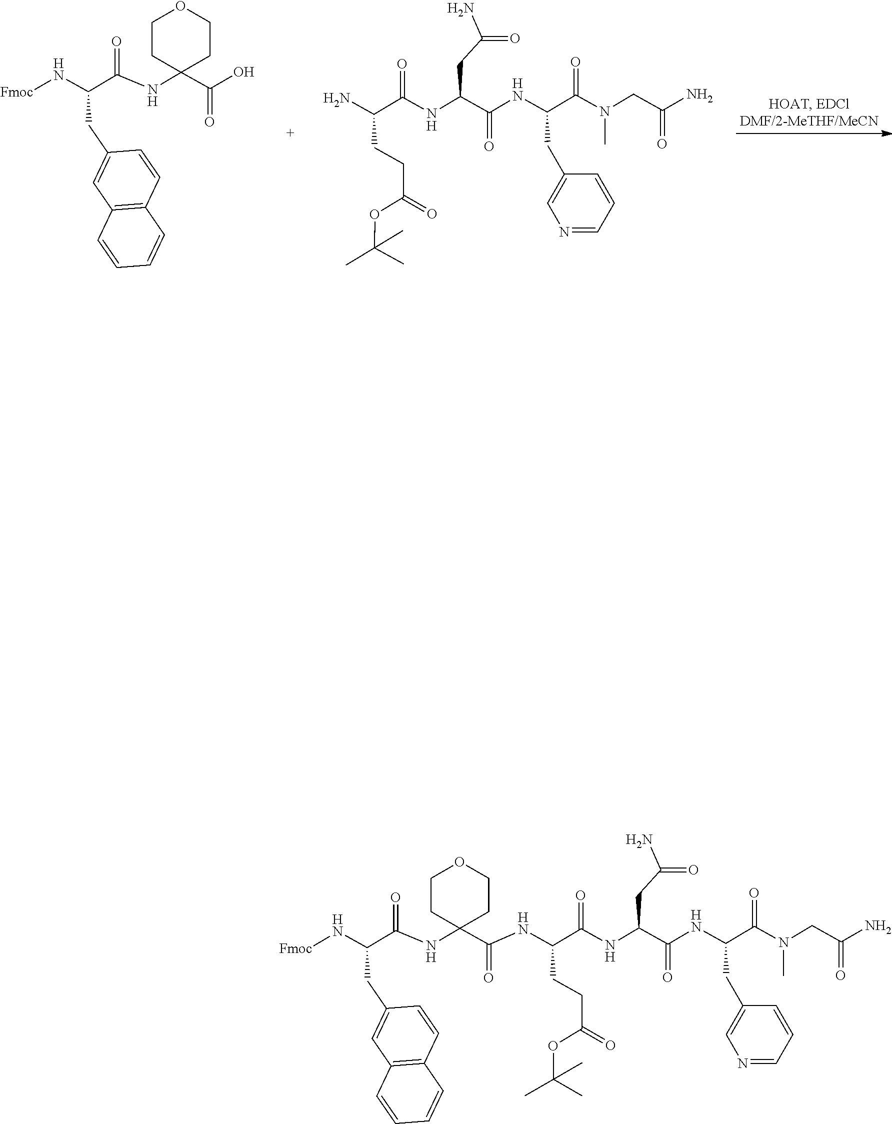

Fmoc-2Nal-Gly(THP)-OH (95.9 kg, 178.8 mol, 1.0 eq.) and H-Glu(tBu)-Asn-3Pal-Sarc-NH2 (106.0 kg, 187.8 mol, 1.2 eq.) were suspended in 2-MeTHF (7 vol., 671 L, 577 kg) and MeCN (2 vol., 192 L, 153 kg) at 20-30° C. The mixture was cooled to 5-15° C., HOAt (12.2 kg, 89.4 mol, 0.5 eq.) was added, EDCI (51.4 kg, 268.2 mol, 1.5 eq.) was added over 1 hour, then the mixture was stirred for 10 hours. The reaction was quenched with 0.72 wt % HCl aq. (5 vol., 500 L, 500 lg) to pH 3.0, the mixture was stirred at 15-25° C. for 1 hour, MBTE (5 vol., 480 L, 355 kg) was added, and the mixture was stirred for 1 hour, then allowed to stand for 1 hour.

After separation, the aqueous layer was put aside and the organic layer was extracted with 0.72 wt % HCl aq. (3 vol., 288 L, 288 kg), stirred for 1 hour at 20° C., and allowed to stand for 1 hour, before being combined with the aqueous layer. The combined aqueous layer was washed with MTBE (5 vol., 480 L, 355 kg) three times, then 2-MeTHF (5 vol., 480 L, 413 kg) was added at 15-25° C. The pH of the resulting solution was adjusted to pH 6.2 with 7% NAHCO₃ aq. (3 vol., 480 L, 480 kg), then NaCl (2.5 vol., 240 kg) was added, and the mixture was stirred for 1 hour and allowed to stand for 1 hour.

After a further separation step, the organic layer was put aside and the aqueous layer was extracted with 2-MeTHF (5 vol., 480 L, 413 kg) twice, then combined with the organic layer. The combined organic layer was washed with 5% LiCl aq. (5 vol., 480 L, 480 kg), then with 5% NaCl aq. (5 vol., 480 L, 480 kg). Fmoc-2Nal-Gly(THP)-Glu(tBu)-Asn-3Pal-Sarc-NH₂ (1.9 kg, 2 wt %) and MeCN (20 vol., 1918 L, 1515 kg) were added, and the mixture was stirred at 20-30° C. for 3 hours, then distilled to 15 vol. under reduced pressure at below 40° C. MeCN (15 vol., 1439 L, 1136 kg) was added and the mixture was stirred at 35-45° C. for 2 hours, then adjusted to 15-25° C. over 4 hours and stirred for 15 hours.

The suspension was filtered and the cake was washed with MeCN (4 vol., 384 L, 303 kg), then dried at 45° C. under reduced pressure for 54 hours, yielding 661.6 kg (80-82% yield, 99.85% UPLC purity) of the desired product (Fmoc-2Nal-Gly(THP)-Glu(tBu)-Asn-3Pal-Sarc-NH2).

### Example I-18: Synthesis of H-Trp(7Me)-Lys(Ac)-Pen(Acm)-Tyr(2-Boc-ea)-OMe

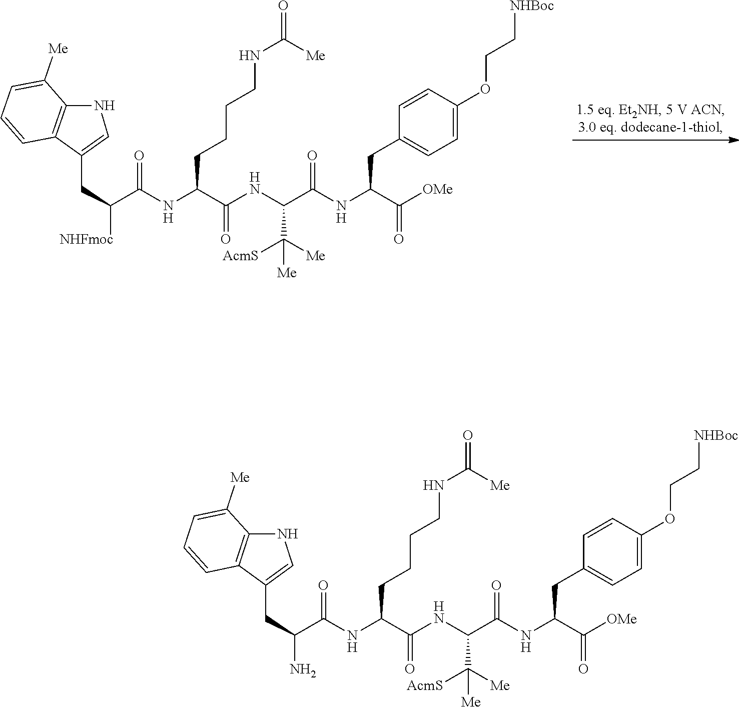

To a solution of Fmoc-Trp(7Me)-Lys(Ac)-Pen(Acm)-Tyr(2-Boc-ea)-OMe (59.31 kg, 1.0 eq.) was added MeCN (202.4 kg), then diethylamine (5.75 kg, 1.5 eq.) was added over 0.38 hours, before further addition of MeCN (61.6 kg). The reactor temperature was adjusted to 35-45° C. over 0.82 hours, the mixture was stirred at this temperature for 4 hours, then the temperature was adjusted to 15-25° C. over 1.3 hours. Dodecane-1-thiol (31.79 kg, 3.0 eq.) was added over 0.62 hours and the mixture was stirred for 4 hours, before addition of n-heptane (1.5 vol., 62.0 kg) followed by stirring for 1 hour. The reaction mixture was allowed to stand for 40 minutes and, following separation, the n-heptane phase was discarded and the MeCN phase was transferred back into the reaction. The addition of n-heptane followed by stirring, standing, and separation was repeated a further 3 times, before the MeCN phase was concentrated to 3.5 vol. over 3.7 hours. 2-MeTHF (12.0 vol., 712.2 kg) was added, the solution was concentrated to 3.5 vol. over 6.3 hours, then further 2-MeTHF (4.0 vol., 201.8 kg) was added, yielding a 402 kg (107.1% yield, 95.0% UPLC purity) solution containing the desired product (H-Trp(7Me)-Lys(Ac)-Pen(Acm)-Tyr(2-Boc-ea)-OMe).

### Example I-19: Synthesis of Ac-Pen(Trt)-Asn-Thr(tBu)-Trp(7Me)-Lys(Ac)-Pen(Acm)-Tyr(2-Boc-ea)-OMe

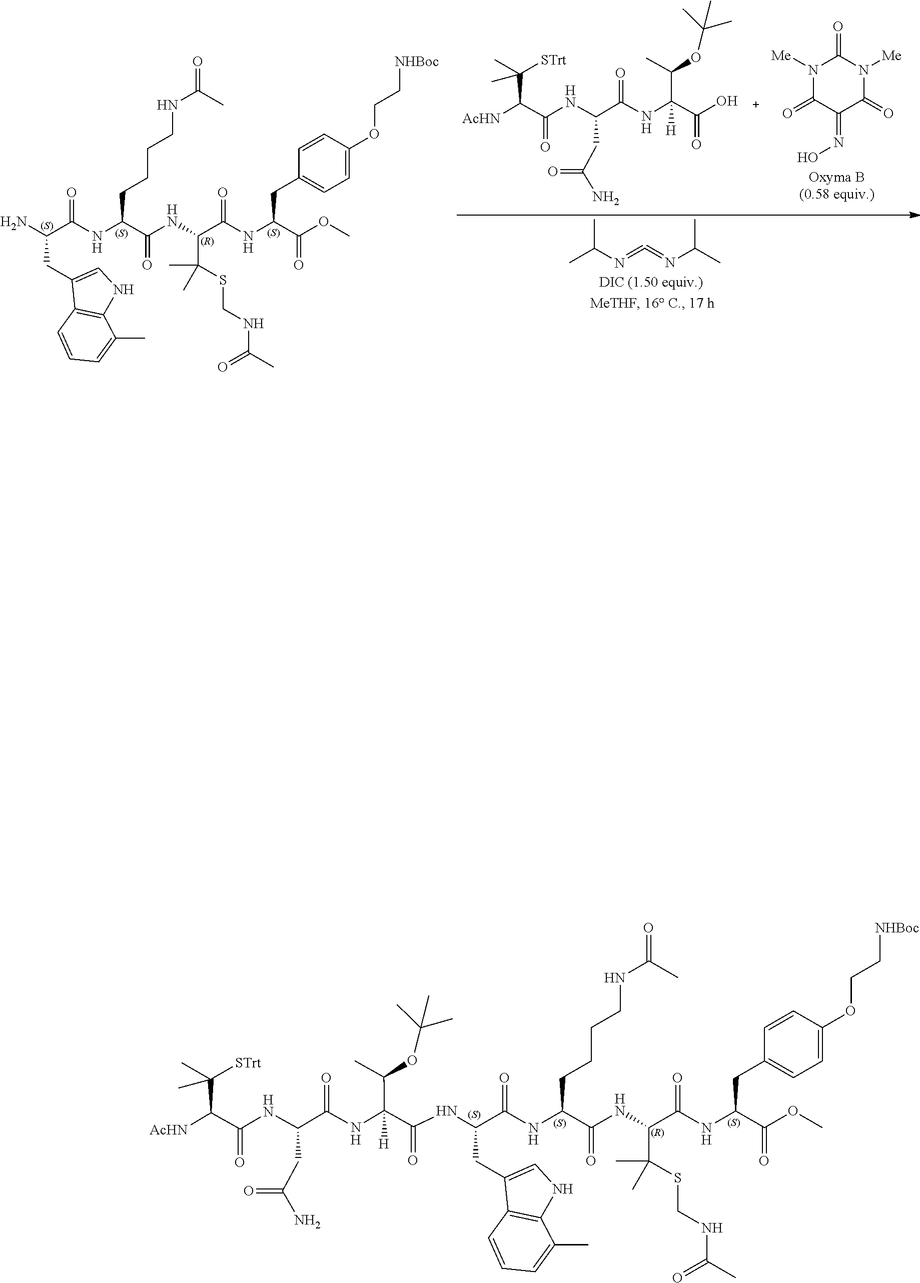

In a 3000 L reactor were loaded Ac-Pen(Trt)-Asn-Thr(tBu)-OH (37.06 kg, 1.0 eq.), H-Trp(7Me)-Lys(Ac)-Pen(Acm)-Tyr(2-Boc-ea)-OMe solution in 2-MeTHF (401.6 kg, 1.0 eq.), 2-MeTHF (14.0 kg) and process water (21.0 kg), followed by anhydrous Oxyma B (5.5 kg, 0.56 eq.) and 2-MeTHF (54.0 kg). The temperature was adjusted to 10-20° C., N,N′-diisopropylcarbodiimide (10.0 kg, 1.5 eq.) was added dropwise, and the mixture was stirred for 10 hours. 2 M HCl (1.2 vol., 56.2 kg) was then added over 1 hour, the mixture was stirred for 1 hour, then 5% NaHCO₃ aq. (5.5 vol., 262.8 kg) was added over 1 hour. The temperature of the reactor was adjusted to 20-30° C., then the mixture was stirred for 1 hour, allowed to stand for 1 hour and, following separation, the aqueous phase was discarded, then this washing step was repeated on the remaining organic phase. 20% NaCl aq. (6.5 vol., 309.2 kg) was added over 1 hour at 10-20° C., then the temperature was adjusted to 20-30° C. and the mixture was stirred, allowed to stand, separated, and the aqueous phase was discarded. The mixture was concentrated to 6.5-7.5 vol. under reduced pressure below 40° C., then 17% H₂O/THF solution (9.5 vol., 453.8 kg) was added and the mixture was stirred for 1 hour at 20-30° C., yielding a 776.4 kg (103.9% yield, 90.2% UPLC purity) H₂O/THF solution of the desired product (Ac-Pen(Trt)-Asn-Thr(tBu)-Trp(7Me)-Lys(Ac)-Pen(Acm)-Tyr(2-Boc-ea)-OMe).

### Example I-20: Synthesis of Ac-Pen(Trt)-Asn-Thr(tBu)-Trp(7Me)-Lys(Ac)-Pen(Acm)-Tyr(2-Boc-ea)-OH-linear

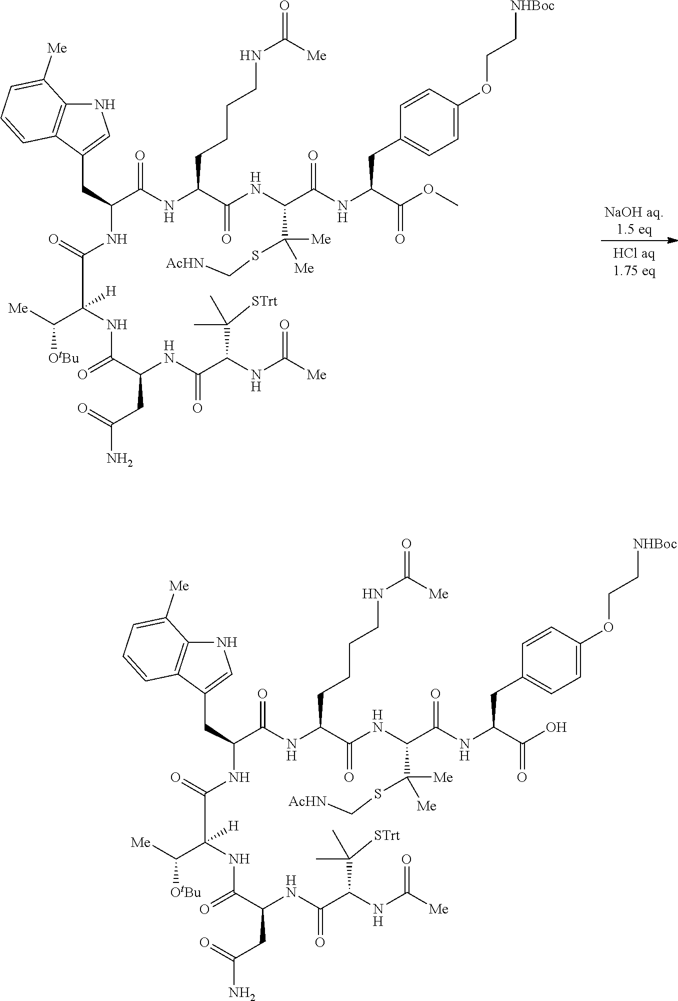

Ac-Pen(Trt)-Asn-Thr(tBu)-Trp(7Me)-Lys(Ac)-Pen(Acm)-Tyr(2-Boc-ea)-OMe in H₂O/THF (776.4 kg, 1.0 eq.) and water (2.53 vol., 211.5 kg) were loaded into a 3000 L reactor at RT. The temperature was adjusted to 0-10° C., a solution of NaOH (1.5 eq., 3.15 kg) in water (0.47 vol., 39.3 kg) was added dropwise over 30 minutes, and the mixture was stirred for 5 hours. 2M HCl (53.4 kg) was added, the mixture was warmed to 15-25° C., and NaCl (0.27 vol., 22.6 kg) was added. Following separation, the organic phase was concentrated to 3 vol. below 35° C. under reduced pressure. 10% process water (3.5 vol., 296 kg) in acetonitrile was added and the mixture was concentrated to 3 vol. below 35° C., a process which was repeated a further two times. 10% process water (4.5 vol., 390 kg) in acetonitrile was added to obtain a 645.8 kg (97.3% yield, 89.9% UPLC purity) solution of the desired product (Ac-Pen(Trt)-Asn-Thr(tBu)-Trp(7Me)-Lys(Ac)-Pen(Acm)-Tyr(2-Boc-ea)-OH-linear).

### Example I-21: Synthesis of Ac-Pen(Trt)-Asn-Thr(tBu)-Trp(7Me)-Lys(Ac)-Pen(Acm)-Tyr(2-Boc-ea)-OH-cyclic

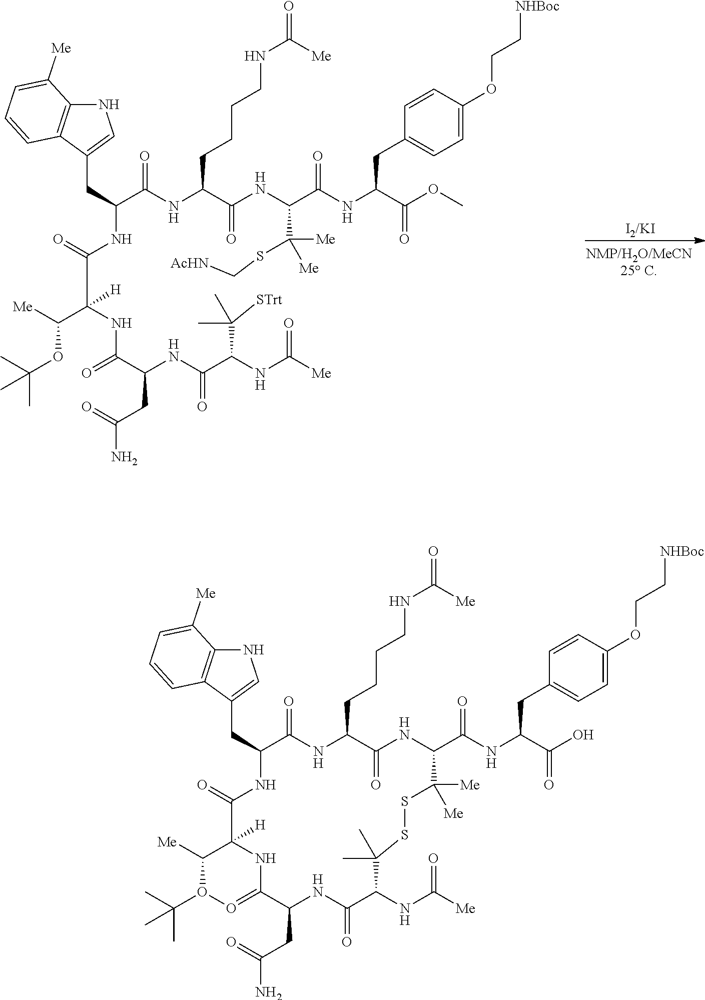

The solution of Ac-Pen(Trt)-Asn-Thr(tBu)-Trp(7Me)-Lys(Ac)-Pen(Acm)-Tyr(2-Boc-ea)-OH-linear (645.4 kg, 1.0 eq.) produced in Example 20 was combined with 2,6-dimethylpyridine (12.4 kg, 2.25 eq.) and process water (0.47 vol., 38.0 kg) in a 2000 L reactor. MeCN (10.0 vol., 807 L, 634 kg), process water (4.3 vol., 355 L, 355 kg), diiodine (29.1 kg, 0.9 eq.), KI (19.05 kg, 2.25 eq.), and formic acid (2.1 kg, 0.9 eq.) were added to a 3000 L reactor and the temperature was adjusted to 20-30° C. The solution from the 2000 L reactor was added dropwise to the 3000 L reactor over 10.5 hours, and the mixture was stirred for 2 hours. 7% NaHCO₃ aq. (0.1 vol., 8 kg) was added, the reaction was quenched with 15.2% Na₂S₂O₃ (1.5 vol., 122 kg), then NaCl (1.0 vol., 81 kg) was added. The mixture was stirred for 2 hours, allowed to stand for 1 hour and, following separation, the aqueous layer was removed. The organic layer was concentrated to 3.5 vol. below 35° C. under vacuum, then 44% MeCN aq. (1.13 vol., 91.4 kg) and EtOAc (3.9 vol., 317.8 L, 286 kg) were added and the mixture was stirred for 5 hours, allowed to stand for 30 minutes and separated, and the aqueous phase was again discarded. 10% NaCl aq. (1.92 vol., 154.6 kg) was added, the mixture was stirred for 30 minutes, allowed to stand for 30 minutes, separated, and the aqueous phase was removed.

The organic layer was concentrated to obtain a 414.0 kg solution with a water content (KF) of 5.7 wt % and the final product (Ac-Pen(Trt)-Asn-Thr(tBu)-Trp(7Me)-Lys(Ac)-Pen(Acm)-Tyr(2-Boc-ea)-OH-cyclic) at 9.2 wt %. MeCN (83.0 L, 65.2 kg) and EtOAc (1.1 L, 1.0 kg) were added, along with 0.31% product (0.25 kg), then the mixture was stirred for 17 hours, before dropwise addition of EtOAc (928.1 L, 835.2 kg) over 4 hours. The solution was cooled to 15-25° C. over 1 hour, then stirred at this temperature for 20 hours.

After filtration, the cake was washed with MeCN/EtOAc/H₂O (ratio: 2.82/17.7/0.51 v/v/v) (1 vol., 81.4 kg) and with EtOAc (0.9 vol., 70.4 kg), then dried at 20-30° C. for 23 hours to obtain 55.75 kg (75.1% yield, 97.1% UPLC purity) of the desired product (Ac-Pen(Trt)-Asn-Thr(tBu)-Trp(7Me)-Lys(Ac)-Pen(Acm)-Tyr(2-Boc-ea)-OH-cyclic) as a solid.

### Example I-22: Synthesis of H-2Nal-Gly(THP)-Glu(tBu)-Asn-3Pal-Sarc-NH2

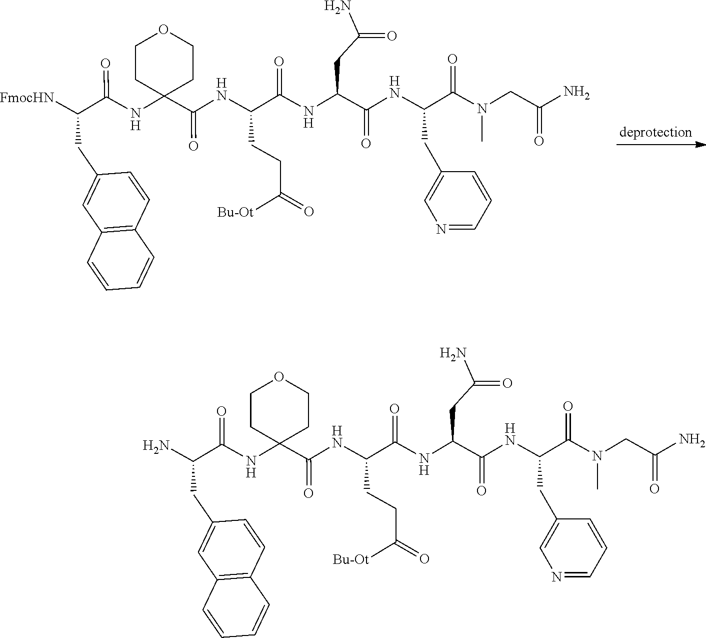

Fmoc-2Nal-Gly(THP)-Glu(tBu)-Asn-3Pal-Sarc-NH₂ (42.03 kg, 1.0 eq.) was dissolved at room temperature in THF (7.86 vol., 296.2 kg)). Dihexylamine (8.80 kg, 1.22 eq.), THF (2.32 vol., 85.8 kg), and process water (1.14 vol., 48.0 kg) were added to the solution, and the mixture was stirred at 35-45° C. for 16 hours. The reactor was cooled to 15-25° C., n-heptane (3.40 vol., 97.2 kg) and process water (1.69 vol., 71.0 kg) were added, then the phases were separated. The aqueous phase was extracted with n-heptane (3.40 vol., 97.2 kg), and the resulting solution was separated again, with the organic layers being combined, yielding a 172.8 kg (99.3% yield, 99.2% HPLC purity) solution of the desired product (H-2Nal-Gly(THP)-Glu(tBu)-Asn-3Pal-Sarc-NH2).

### Example I-23: Synthesis of Ac-Pen-Asn-Thr(tBu)-Trp(7Me)-Lys(Ac)-Pen-Tyr(2-Boc-ea)-2Nal-Gly(THP)-Glu(tBu)-Asn-3Pal-Sarc-NH2

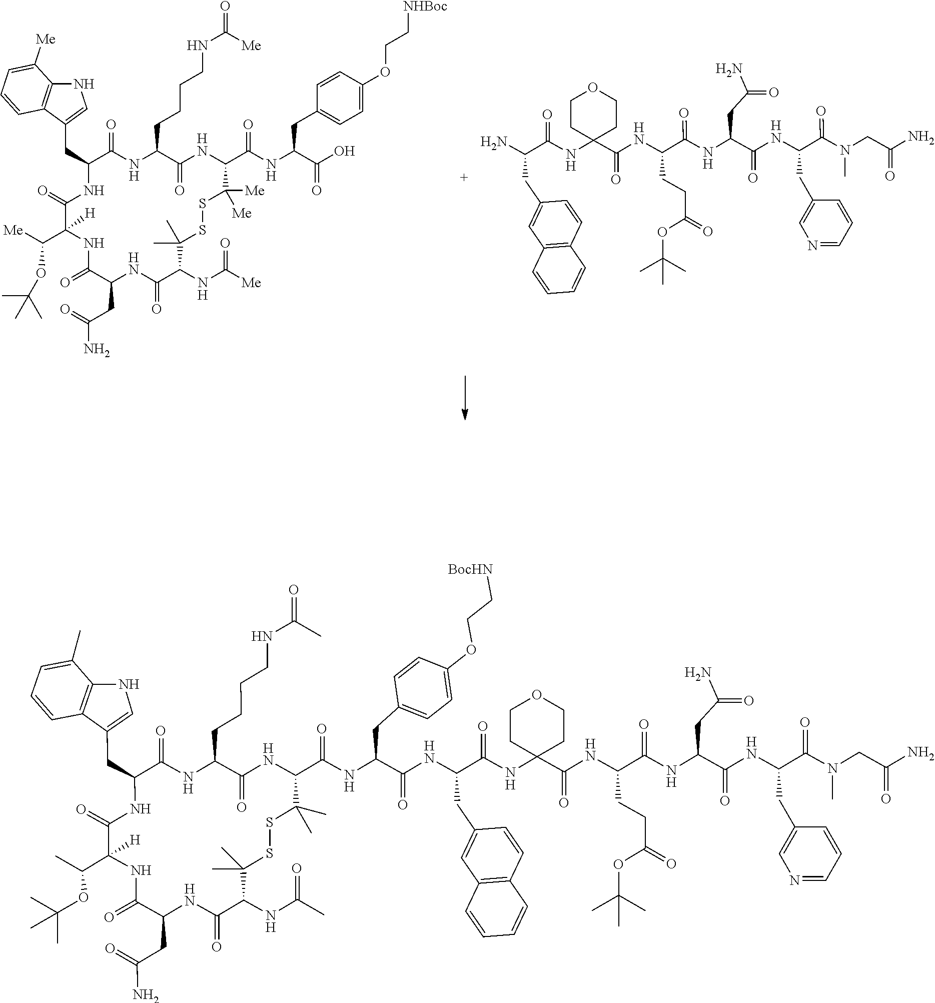

In a 2000 L reactor Ac-Pen(Trt)-Asn-Thr(tBu)-Trp(7Me)-Lys(Ac)-Pen(Acm)-Tyr(2-Boc-ea)-OH-cyclic (46.5 kg, 1.0 eq.) and the H-2Nal-Gly(THP)-Glu(tBu)-Asn-3Pal-Sarc-NH2 solution from the previous step (172.2 kg, 1.05 eq.) were combined with THF (4 vol., 186 L, 165.5 kg). Oxyma B anhydrous (3.40 kg, 0.5 eq.) was added at 20-25° C., along with N,N′-diisopropylcarbodiimide (6.95 kg, 1.5 eq.) which was added dropwise over 30 minutes, before the mixture was stirred for 5 hours. The mixture was cooled to 10-20° C., 2M HCl (18.6 kg) was added and the mixture was stirred for 1 hour, then 5% NaHCO₃ aq. (62.0 kg) was added and the mixture was stirred for 30 minutes. 2-MeTHF (4.1 vol., 189.4 kg) was added and the mixture was stirred for 30 minutes, allowed to stand for 1 hour, and the aqueous phase was removed. 5% NaHCO₃ aq. (62.0 kg) was added, the mixture was stirred for 1 hour, allowed to stand for 1 hour and, following separation, the aqueous phase was removed. As a final washing step, 5% NaCl aq. (42.4 kg) was added, the mixture was stirred for 1 hour, allowed to stand for 1 hour and, following separation, the aqueous phase was removed.

The organic phase was concentrated to 2-3 vol. below 40° C. under vacuum, then IPA (5 vol., 290 kg) was added and the concentration step was repeated, with the IPA and concentration step being performed a total of two times. IPA (1.5 vol., 90.0 kg) was added at 30° C., then further IPA (1.1 vol., 67.2 kg) was added at 35° C., along with the addition of process water (10 vol., 765 kg) over 1.5 hours. Product (Ac-Pen(Trt)-Asn-Thr(tBu)-Trp(7Me)-Lys(Ac)-Pen(Acm)-Tyr(2-Boc-ea)-2Nal-Gly(THP)-Glu(tBu)-Asn-3Pal-Sarc-NH₂) (0.276 kg, 0.3 wt %) were added and the mixture was stirred for 4 hours, then cooled to 20° C. over 10 hours and stirred for a further 6 hours.

After filtration, the cake was washed with 28% IPA aq. (2.7 vol., 206 kg) and EtOAc (0.9 vol., 70 kg), then dried at 40-45° C. over 4 days to obtain two batches (1.30.0 kg, 37.9% yield, 99.1% UPLC purity, and 2. 40.93 kg, 51.4% yield, 99.3% UPLC purity) of the desired product (Ac-Pen(Trt)-Asn-Thr(tBu)-Trp(7Me)-Lys(Ac)-Pen(Acm)-Tyr(2-Boc-ea)-2Nal-Gly(THP)-Glu(tBu)-Asn-3Pal-Sarc-NH2) as a solid.

### Example I-24: Synthesis of Ac-Pen-Asn-Thr-Trp(7Me)-Lys(Ac)-Pen-Tyr(2-ea)-2Nal-Gly(THP)-Glu-Asn-3Pal-Sarc-NH2

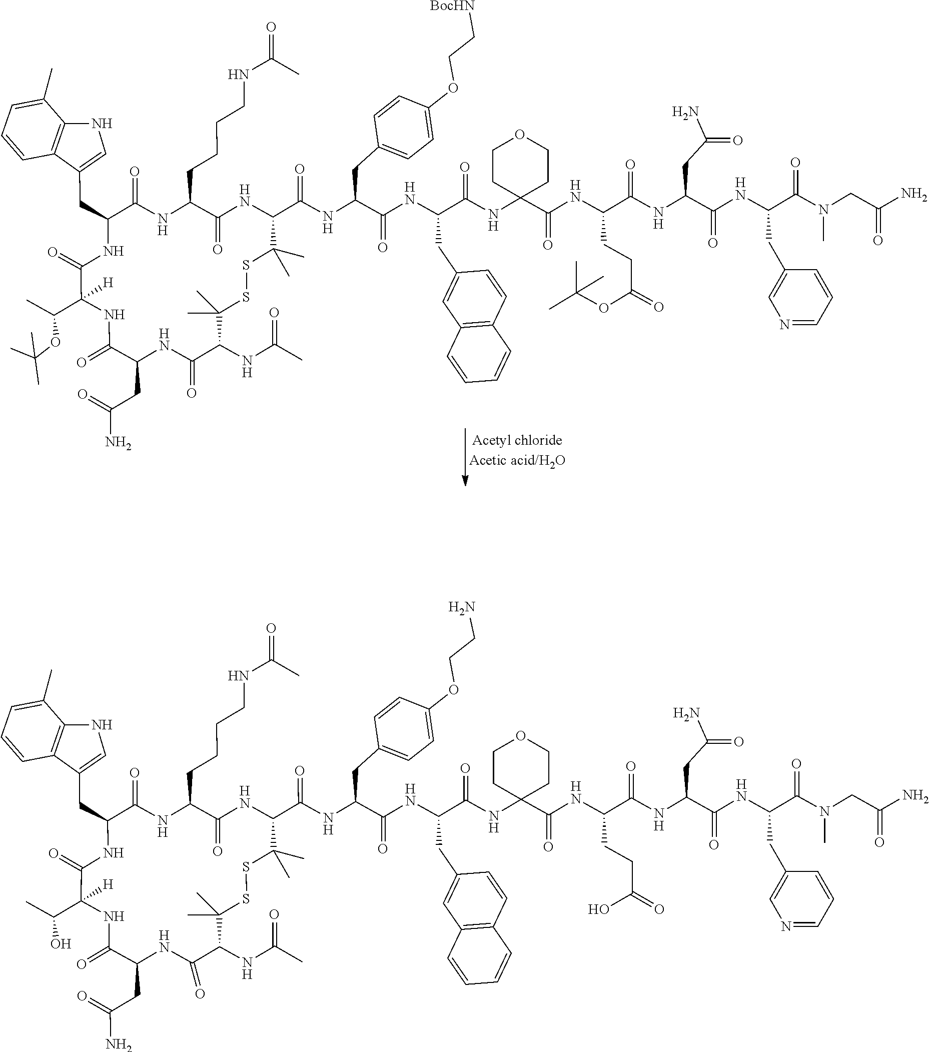

Ac-Pen-Asn-Thr(tBu)-Trp(7Me)-Lys(Ac)-Pen-Tyr(2-Boc-ea)-2Nal-Gly(THP)-Glu(tBu)-Asn-3Pal-Sarc-NH₂ (total of both batches) was loaded in a 3000 L reactor and AcOH (3.6 vol., 262 kg) and process water (0.15 vol., 10 kg) were added at 25° C. The mixture was stirred until a clear solution was obtained, then the temperature was adjusted to 15° C. In a separate reactor AcOH (1.65 vol., 119 kg) and acetyl chloride (61 kg, 24 eq.) were added and cooled to 10° C., followed by addition of 18.9% (H₂O/AcOH) solution (1.4 vol., 93.5 kg) over 2 hours, then the initial Ac-Pen(Trt)-Asn-Thr(tBu)-Trp(7Me)-Lys(Ac)-Pen(Acm)-Tyr(2-Boc-ea)-2Nal-Gly(THP)-Glu(tBu)-Asn-3Pal-Sarc-NH₂ mixture was added to the reactor over 1.2 hours. The pipeline was rinsed with AcOH (0.32 vol., 22 kg) and the mixture was stirred for 1 hour, then 19.4% NaOH aq. (14.6 vol., 1000 kg) was added over 6 hours, followed by product (Ac-Pen-Asn-Thr-Trp(7Me)-Lys(Ac)-Pen-Tyr(2-ea)-2Nal-Gly(THP)-Glu-Asn-3Pal-Sarc-NH2) (0.21 kg, 0.3 wt %). This mixture was stirred at 20° C. for 2 hours, then the pH was adjusted by addition of 19.4% NaOH aq. (2.3 vol., 160 kg), followed by stirring for 16 hours.

The mixture was filtered and the cake was washed with process water (4.1 vol., 280 kg) and EtOAc (1.8 vol., 125 kg), then dried at 40-45° C. under vacuum for 16 hours to obtain 56.1 kg (84.4% yield, 97.8% UPLC purity) of the desired product (Ac-Pen-Asn-Thr-Trp(7Me)-Lys(Ac)-Pen-Tyr(2-ea)-2Nal-Gly(THP)-Glu-Asn-3Pal-Sarc-NH2).

### Example I-25: Synthesis of H-3Pal-Sarc-NH2 (Alternative to Example 14)

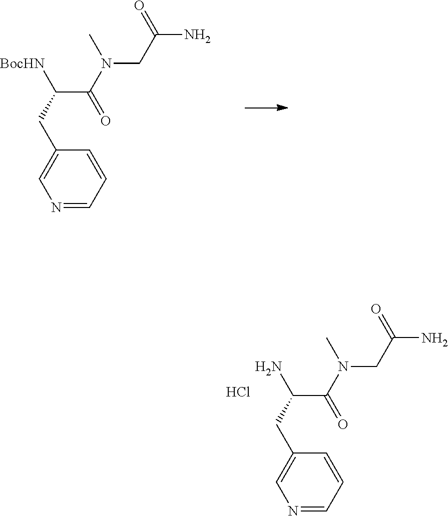 [SMILES: CN(CC(N)=O)C(=O)[C@H](Cc1cccnc1)NC(=O)OC(C)(C)C ; Cl ; CN(CC(N)=O)C(=O)[C@@H](N)Cc1cccnc1]

MeCN (8 vol., 696 L, 687 kg) and 4 M HCl in EtOAc (765.6 kg, 3103.2 mol, 12 eq.) were added to a 5000 L reactor at 20-30° C., and the mixture was stirred for 1 hour. Boc-3Pal-Sarc-NH₂ (87 kg, 258.6 mol, 1.0 eq.) was added in ten portions over 7 hours, then the mixture was stirred for 3 hours. The suspension was filtered, and the cake was washed with MeCN (4 vol., 348 L, 278 kg) and dried in vacuo at 25° C. for 22 h to give 92.8 kg (98.2% yield, 99.6% UPLC purity) of the desired product(H-3Pal-Sarc-NH2).

The following reaction scheme may aid in understanding the reactions discussed throughout the following examples.

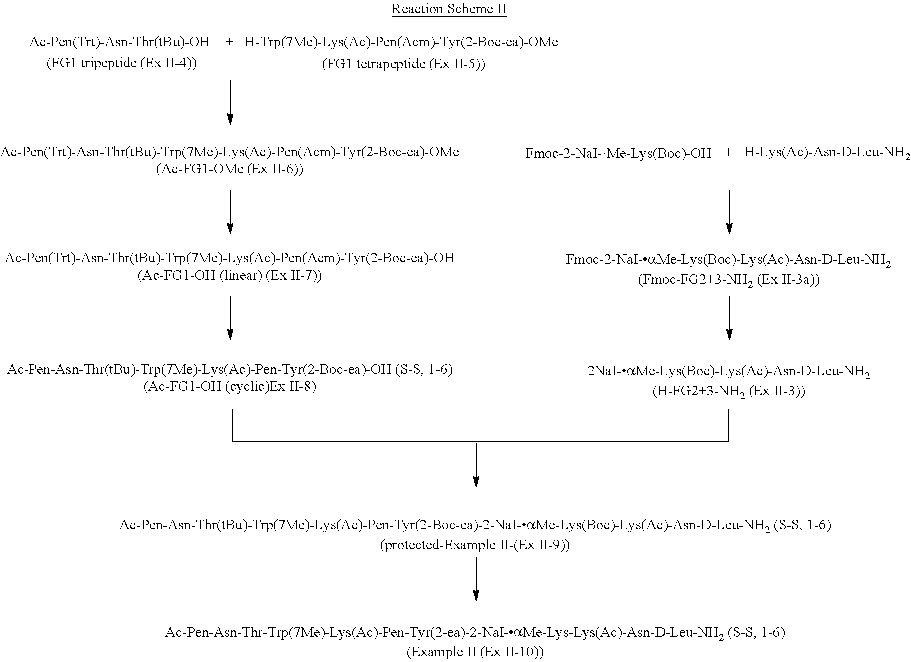

### Example II-1: Synthesis of H-Lys(Ac)-Asn-D-Leu-NH₂ (H-FG3-NH₂)

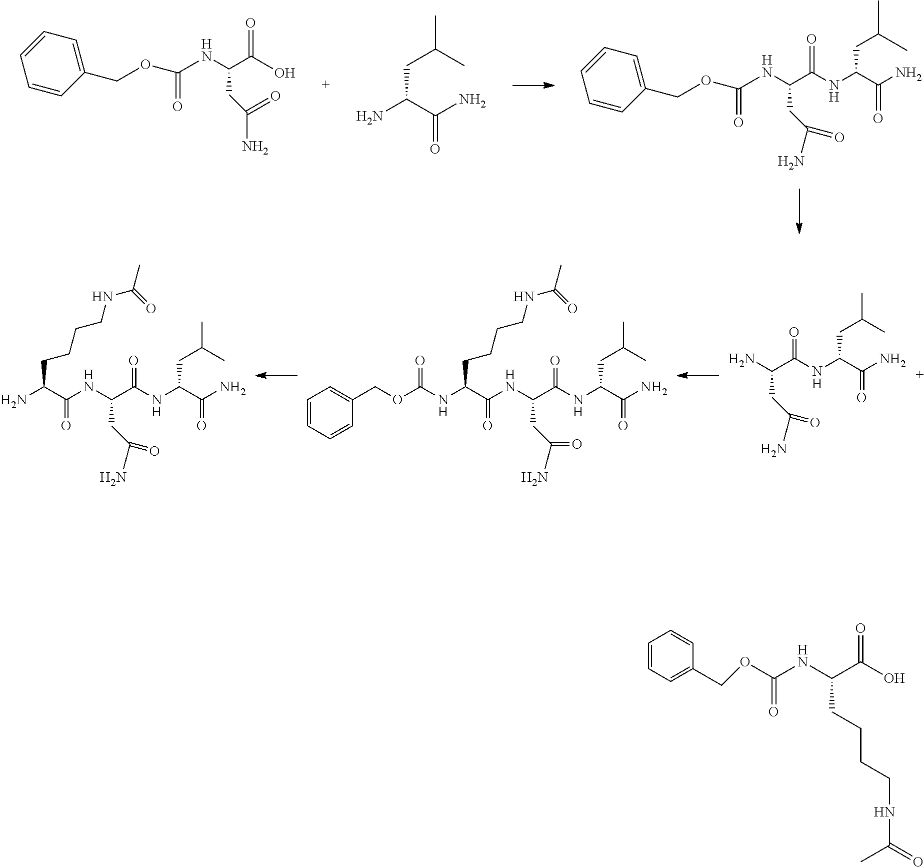

### Example II-1a: Synthesis of Z-Asn-D-Leu-NH₂

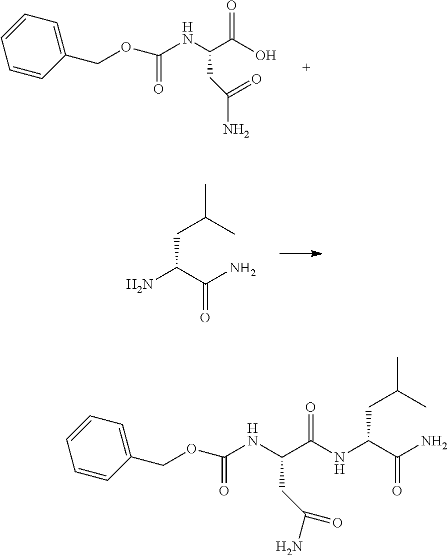

In a 10 L reactor Z-Asn-OH (150.0 g; 1.0 eq.), OxymaPure® (80.1 g; 1.0 eq.) and H-D-Leu-NH₂*HCl (98.6 g; 1.05 eq.) were dissolved in DMF (1.8 L) and cooled to 0-5° C. TEA (172 mL; 2.2 eq.) was added to the mixture followed by EDC*HCl (3×40.5 g; 1.13 eq.) which was added in three portions over one hour. The yellow reaction mixture was stirred for 1 h at 0-5° C. and then allowed to warm to room temperature. After stirring for an additional hour, the mixture was precipitated on 2% aqu. NaHCO₃ (15 L). The resulting yellow suspension was stirred for a few minutes and then filtered. The filter cake was washed with water (6×750 mL) and EtOAc (6×600 mL). The product was dried in a vacuum oven under reduced pressure at 30° C. over the weekend to give 188 g of a white solid Z-Asn-D-Leu-NH2 (88% yield, ESI-MS m/z 379.16 ([M+H]⁺).

### Example II-1b: Synthesis of Asn-D-Leu-NH2

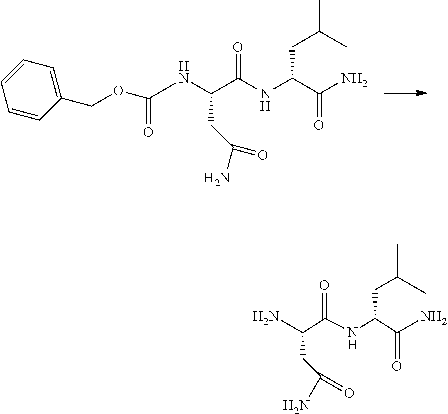 [SMILES: CC(C)C[C@@H](NC(=O)[C@H](CC(N)=O)NC(=O)OCc1ccccc1)C(N)=O ; CC(C)C[C@@H](NC(=O)[C@@H](N)CC(N)=O)C(N)=O]

Z-Asn-D-Leu-NH₂ (230 g, 1.0 eq.) and Pd/C 5% (23 g) were suspended in MeOH/H₂O (95:5, 4.6 L) followed by the addition of HCl 36% (57 mL, 1.1 eq.). The reaction mixture was stirred at room temperature for 3 h under 3.0 bar H₂ pressure. After the reaction was complete, the hydrogenation solution was clear-filtrated and the filter washed with MeOH (2×200 mL). The filtrate was evaporated under reduced pressure at 40° C. and the residual oil co-distilled with EtOH (3×2.5 L). EtOH (2.5 L) was added to the residual oil and the solution was stirred for 1 h at 10° C. IPE (5.75 L) was added to the formed suspension and the mixture was stirred at room temperature for 30 minutes. The suspension was filtrated and the cake washed with IPE (3×575 mL). The product was dried in a vacuum oven at 40° C. for 18 h to give 169 g of a white solid Asn-D-Leu-NH2 (Yield: 99%, ESI-MS: m/z=415.36 [M+H⁺]⁺).

### Example II-1c: Synthesis of Z-Lys(Ac)-Asn-D-Leu-NH2

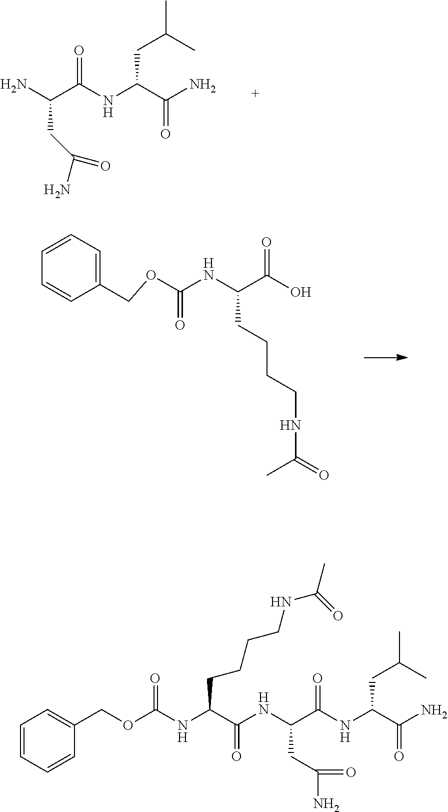

H-Asn-D-Leu-NH₂·HCl (140.0 g, 1.0 eq.) and Z-Lys(Ac)—OH (160.7 g, 1.0 eq.) were suspended in DMF (2.8 L). The thin white suspension was cooled to 0-5° C. and DIPEA (261 mL, 3.0 eq.) was added to the reaction mixture. TBTU (168 g, 1.05 eq.) was added in one portion and the yellow reaction mixture was stirred at 0-5° C. for 30 minutes, then allowed to warm to room temperature and stirred for another hour. The mixture was precipitated onto a 2% NaHCO₃ (17 L) aqueous solution and the formed suspension was stirred for 2.5 h at 10° C. The suspension was filtered and the cake washed with 2% NaHCO₃ (3×300 mL) and water (3×300 mL). The product was dried in a vacuum oven at 50° C. for three days to give 132.0 g of a white solid Z-Lys(Ac)-Asn-D-Leu-NH2 (Yield: 48% ESI-MS m/z=549.33 [M+H]⁺).

### Example II-1d: Synthesis of H-Lys(Ac)-Asn-D-Leu-NH2

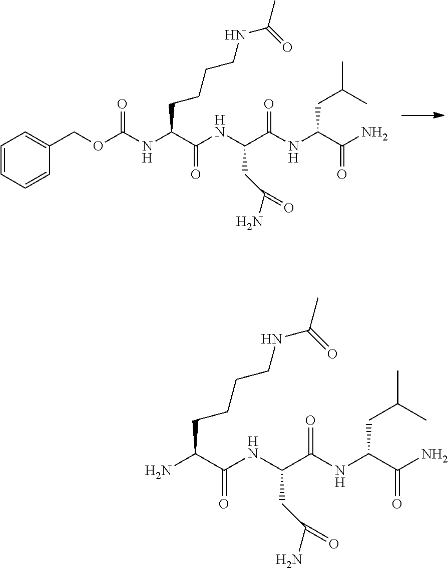 [SMILES: CC(=O)NCCCC[C@H](NC(=O)OCc1ccccc1)C(=O)N[C@@H](CC(N)=O)C(=O)N[C@H](CC(C)C)C(N)=O ; CC(=O)NCCCC[C@H](N)C(=O)N[C@@H](CC(N)=O)C(=O)N[C@H](CC(C)C)C(N)=O]

Z-Lys(Ac)-Asn-D-Leu-NH₂ (153 g, 1.0 eq.) and Pd/C 5% (15.3 g) were suspended in a MeOH/water mixture (5:1, 2.22 L). The suspension was then hydrogenated at 3.5 bar H₂ pressure at room temperature. After 4 h the reaction conversion reached only 50%. The suspension was filtered and the filtrate stored at 0-5° C. overnight. Fresh Pd/C 5% (15.3 g) was added to the filtrate and the suspension was hydrogenated at 3.5 bar H₂ pressure at 40° C. for 4 h. The reaction mixture was then filtered and the filter washed with MeOH (3×100 mL). The filtrate was concentrated under reduced pressure at 50° C. and the residue co-distilled with EtOH (4×1000 mL) to a residue of 700 g. The oil was diluted with EtOAc (3 L) and the resultant white suspension was stirred at room temperature for 30 minutes. The suspension was filtered and washed with EtOAc (3×400 mL). The product was dried in a vacuum oven at 30° C. for 2 days to give 105.7 g of a white solid H-Lys(Ac)-Asn-D-Leu-NH2. (Yield 91%, ESI-MS m/z=415.36 [M+H]⁺).

### Example II-2: Synthesis of Fmoc-2-Nal-αMe-Lys(Boc)-OH (Fmoc-FG2-OH)

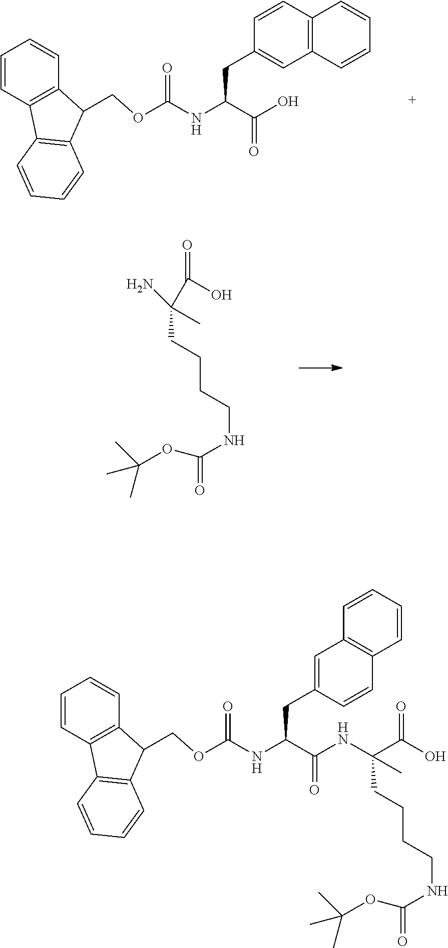 [SMILES: O=C(N[C@@H](Cc1ccc2ccccc2c1)C(=O)O)OCC1c2ccccc2-c2ccccc21 ; CC(C)(C)OC(=O)NCCCC[C@](C)(N)C(=O)O ; CC(C)(C)OC(=O)NCCCC[C@](C)(NC(=O)[C@H](Cc1ccc2ccccc2c1)NC(=O)OCC1c2ccccc2-c2ccccc21)C(=O)O]

PyAOP (65.5 g, 1.1 eq) was dissolved in Me-THF/DMSO (5:1, 1.2 L) and cooled to 0-5° C. with an ice bath. DIPEA (40 mL, 2.0 eq.) and Fmoc-2-Nal-OH (50 g, 1.0 eq.) was added and the yellow suspension was stirred at 0-5° C. for 15 minutes. H-αMeLys(Boc)-OH (35.7, 1.2 eq.) was added in one portion and the reaction mixture was stirred at room temperature for 2 h. The reaction mixture was diluted with MeTHF (1000 mL) and washed with 5% NaHCO₃ (1×1000 mL). The organic layer was then washed with 2% NaHCO₃/DMSO (7:3, 9×800 mL) and 2% NaCl (2×800 mL). The organic layer was concentrated under reduced pressure at 40° C. and the oil co-distilled with MeTHF (2×500 mL). The light yellow oil was dissolved in MeTHF/IPE (1:2, 600 mL) and poured onto pentane (2 L). The suspension was stirred for 15 minutes at room temperature then filtered and the cake washed with pentane/IPE 7:1 (2×400 mL). The product was dried in a vacuum oven at 40° C. for 18 h. The dried material was suspended in IPE (1.1 L) and stirred at room temperature for 1 h. The white suspension was filtered and washed with IPE (2×250 mL). The product was dried in a vacuum oven at 40° C. for 16 h, then suspended in IPE (2000 mL) and stirred at room temperature. After 4 h the off-white suspension was filtered and the filter cake washed with IPE (2×200 mL). The product was dried in a vacuum oven at 40° C. for 18 h. This procedure was repeated a second time and the product was isolated as a white solid, Fmoc-2-Nal-αMe-Lys(Boc)-OH (Fmoc-FG2-OH), (156 g). (Yield 72.2 g, 92%, ESI-MS m/z=680.17 [M+H]⁺).

### Example II-3: Synthesis of 2Nal-αMe-Lys(Boc)-Lys(Ac)-Asn-D-Leu-NH2 (H-FG2+3-NH₂)

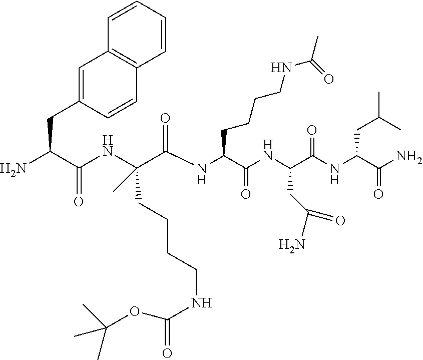 [SMILES: CC(=O)NCCCC[C@H](NC(=O)[C@](C)(CCCCNC(=O)OC(C)(C)C)NC(=O)[C@@H](N)Cc1ccc2ccccc2c1)C(=O)N[C@@H](CC(N)=O)C(=O)N[C@H](CC(C)C)C(N)=O]

### Example II-3a: Synthesis of Fmoc-2Nal-αMe-Lys(Boc)-Lys(Ac)-Asn-D-Leu-NH2 (Fmoc-FG2+3-NH₂)

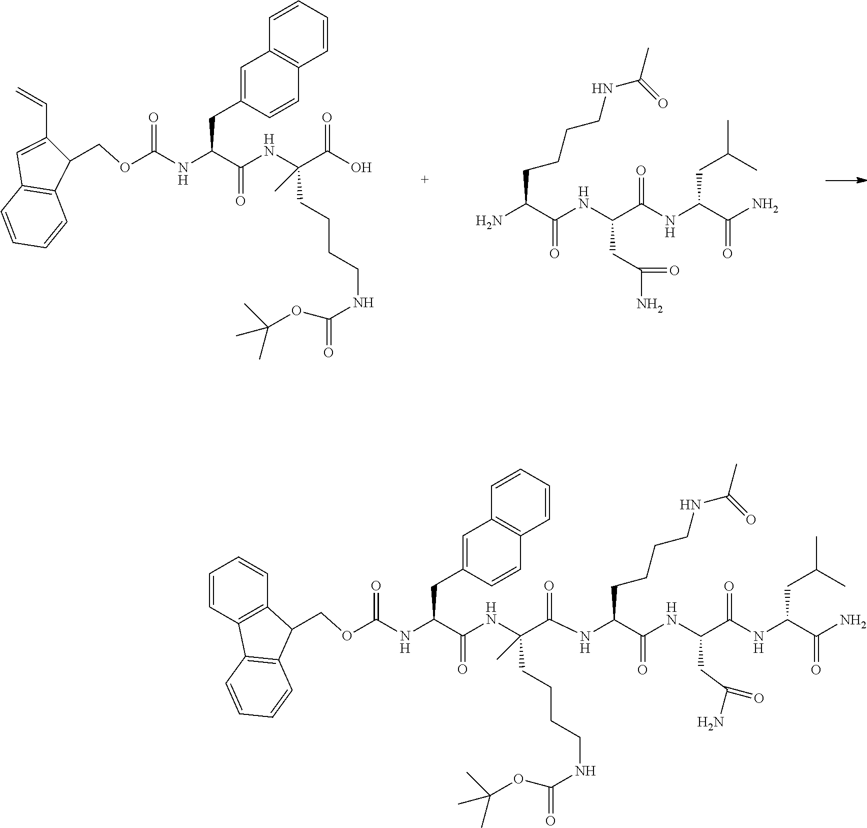 [SMILES: C=CC1=Cc2ccccc2C1COC(=O)N[C@@H](Cc1ccc2ccccc2c1)C(=O)N[C@@](C)(CCCCNC(=O)OC(C)(C)C)C(=O)O ; CC(=O)NCCCC[C@H](N)C(=O)N[C@@H](CC(N)=O)C(=O)N[C@H](CC(C)C)C(N)=O ; CC(=O)NCCCC[C@H](NC(=O)[C@](C)(CCCCNC(=O)OC(C)(C)C)NC(=O)[C@H](Cc1ccc2ccccc2c1)NC(=O)OCC1c2ccccc2-c2ccccc21)C(=O)N[C@@H](CC(N)=O)C(=O)N[C@H](CC(C)C)C(N)=O]

Fmoc-2-αMeLys(Ac)—OH (113.0 g, 1.0 eq) was dissolved in MeTHF/DMSO (4:1, 1700 mL) and the solution cooled to 0-5° C. H-Lys(Ac)-Asn-D-Leu-NH₂ (82.7 g, 1.2 eq.) and PyAOP (95.3 g, 1.1 eq.) were added to the reaction mixture in one portion followed by the addition of DIPEA (57.9 mL, 2.0 eq.). The ice bath was then removed and the reaction was allowed to warm to room temperature and stirred overnight (17 h). The reaction mixture was then washed with 5% NaHCO₃ (2×3400 mL) and 2% (2×2300 mL). The organic layer was filtered and the filtrate evaporated under reduced pressure at 50° C. The residue was co-distilled with EtOAc (2×700 mL), the formed suspension was diluted with EtOAc (800 mL) and stirred at 50° C. for 10 minutes, then at room temperature for 30 minutes. The suspension was filtered and the cake washed with EtOAc (3×380 mL). The product was dried under vacuum at 40° C. for 18 h to give 137 g of the desired product as a white solid, 2Nal-αMe-Lys(Boc)-Lys(Ac)-Asn-D-Leu-NH2 (H-FG2+3-NH2), (Yield 77%, ESI-MS m/z=1077.47 [M+H]⁺).

### Example II-3b: Synthesis of H-2Nal-αMe-Lys(Boc)-Lys(Ac)-Asn-D-Leu-NH2 (H-FG2+3-NH₂)

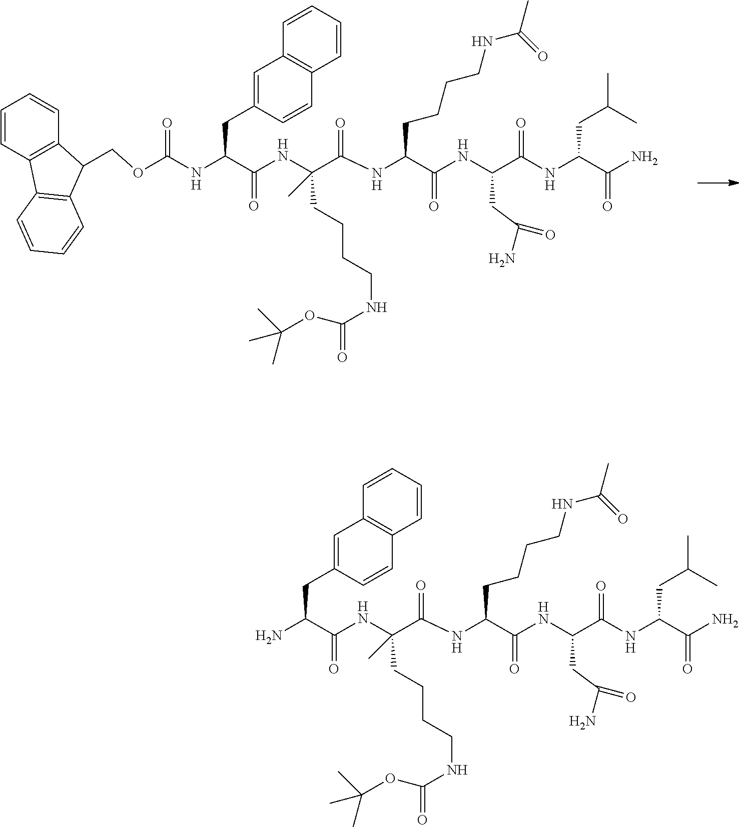 [SMILES: CC(=O)NCCCC[C@H](NC(=O)[C@](C)(CCCCNC(=O)OC(C)(C)C)NC(=O)[C@H](Cc1ccc2ccccc2c1)NC(=O)OCC1c2ccccc2-c2ccccc21)C(=O)N[C@@H](CC(N)=O)C(=O)N[C@H](CC(C)C)C(N)=O ; CC(=O)NCCCC[C@H](NC(=O)[C@](C)(CCCCNC(=O)OC(C)(C)C)NC(=O)[C@@H](N)Cc1ccc2ccccc2c1)C(=O)N[C@@H](CC(N)=O)C(=O)N[C@H](CC(C)C)C(N)=O]

Fmoc-2-αMeLys(Ac)—OH (113.0 g, 1.0 eq) was dissolved in MeTHF/DMSO (4:1, 1700 mL) and the solution cooled to 0-5° C. H-Lys(Ac)-Asn-D-Leu-NH₂ (82.7 g, 1.2 eq.) and PyAOP (95.3 g, 1.1 eq.) were added to the reaction mixture in one portion followed by the addition of DIPEA (57.9 mL, 2.0 eq.). The ice bath was then removed and the reaction was allowed to warm to room temperature and stirred overnight (17 h). The reaction mixture was then washed with 5% NaHCO₃ (2×3400 mL) and 2% (2×2300 mL). The organic layer was filtered and the filtrate evaporated under reduced pressure at 50° C. The residue was co-distilled with EtOAc (2×700 mL), the formed suspension was diluted with EtOAc (800 mL) and stirred at 50° C. for 10 minutes, then at room temperature for 30 minutes. The suspension was filtered and the cake washed with EtOAc (3×380 mL). The product was dried under vacuum at 40° C. for 18 h to give 137 g of the desired product as a white solid, H-2Nal-αMe-Lys(Boc)-Lys(Ac)-Asn-D-Leu-NH2 (H-FG2+3-NH2). (Yield 96%, ESI-MS m/z=854.48 [M+H]⁺).

### Example II-4: Synthesis of Ac-Pen(Trt)-Asn-Thr(tBu)-OH (FG1 tripeptide)

### Example II-4a: Synthesis of Z-Asn-Thr(tBu)-Ome

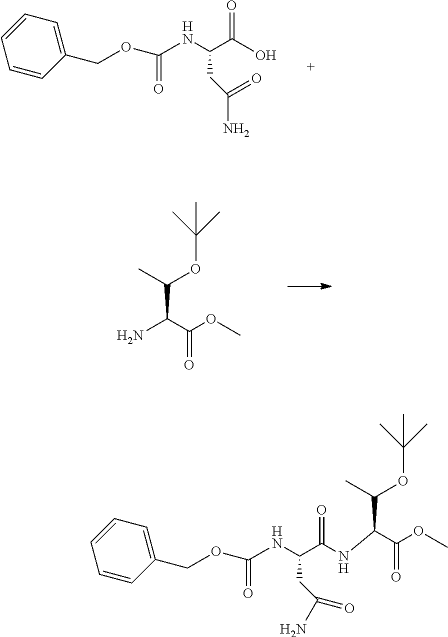 [SMILES: NC(=O)C[C@H](NC(=O)OCc1ccccc1)C(=O)O ; COC(=O)[C@@H](N)C(C)OC(C)(C)C ; COC(=O)[C@@H](NC(=O)[C@H](CC(N)=O)NC(=O)OCc1ccccc1)C(C)OC(C)(C)C]

To a solution of Z-Asn-OH (500.00 g, 1.877 mol, 1.0 eq.), H-Thr(tBu)-OMe (445.06 g, 1.971 mol, 1.05 eq.) and Oxyma Pure (266.85 g, 1.877 mol, 1.0 eq.) in EtOAc:DMSO (1:1, v:v) (3 L) was added triethylamine (572.6 mL, 4.311 mol, 2.2 eq.) and the mixture stirred at 25-30° C. for 10 minutes. EDC×HCl (468.00 g, 2.441 mol, 1.3 eq.) was added in four portions within one hour to the reaction mixture and stirred overnight. IPC-HPLC showed completeness of the reaction.

The reaction mixture was diluted with EtOAc (6 L) and washed with NaHSO₄ (5% aq.) (2×5 L), NaHCO₃ (5% aq.) (4×5 L) and water (5 L). The organic layer was evaporated under reduced pressure at 40° C. (AT) and the residue co-evaporated with EtOAc (3×3 L). The residue (ca. 25% w/v) was cooled to 20° C. and seeded with seed crystals. The product crystallized and the suspension formed was stirred at 0-5° C. for 2 h before the product was separated by filtration. The filter cake was washed with pre-cooled EtOAc (3×1 L) and the product dried under reduced pressure at 30° C. overnight to give 653 g of a white solid, Z-Asn-Thr(tBu)-OMe (Yield 80%, ESI-MS m/z 438.18 [M+H]⁺).

### Example II-4b: Synthesis of Fmoc-Pen(Trt)-Asn-Thr(tBu)-OMe

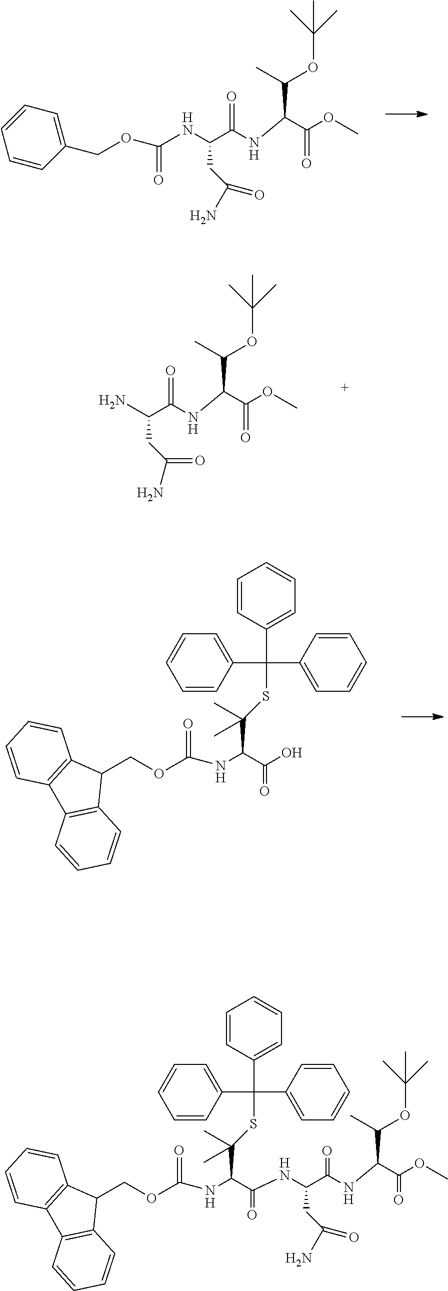 [SMILES: COC(=O)[C@@H](NC(=O)[C@H](CC(N)=O)NC(=O)OCc1ccccc1)C(C)OC(C)(C)C ; COC(=O)[C@@H](NC(=O)[C@@H](N)CC(N)=O)C(C)OC(C)(C)C ; CC(C)(SC(c1ccccc1)(c1ccccc1)c1ccccc1)[C@H](NC(=O)OCC1c2ccccc2-c2ccccc21)C(=O)O ; COC(=O)[C@@H](NC(=O)[C@H](CC(N)=O)NC(=O)[C@@H](NC(=O)OCC1c2ccccc2-c2ccccc21)C(C)(C)SC(c1ccccc1)(c1ccccc1)c1ccccc1)C(C)OC(C)(C)C]

Z-Asn-Thr(tBu)-OMe (330.0 g, 0.754 mol, 1.0 eq.) and EtOAc (2.6 L) were loaded into an hydrogenation reactor and the mixture stirred until a clear solution was obtained. Pd(OH)₂ on activated charcoal (6.6 g, 2% (w:w)) was added to the reaction mixture and the reactor was closed, inertized with nitrogen and the temperature set at 40° C.. Hydrogen pressure (3 bar) was applied and the reaction mixture stirred at this conditions. IPC-HPLC after 5 hours shows quantitative reaction. The reaction mixture was cooled to 25° C. and filtered through a deep filter to remove the catalyst and stored at 4° C. overnight (Mass: 2.46 kg). The concentration of H-Asn-Thr(tBu)-OMe was calculated using a non-qualified standard of H-Asn-Thr(tBu)-OMe and the amount of solution needed taken into the next step without isolation of the intermediate.

In a 15 L glass reactor with overhead stirrer were loaded Fmoc-Pen(Trt)-OH (387.0 g, 0.628 mol, 1.0 eq), TBTU (216.2 g, 0.673 mol, 1.07 eq.), DMSO (0.6 L) and the solution obtained in the step above (2.3 kg, 200.0 g H-Asn-Thr(tBu)-OMe, 0.659 mol, 1.05 eq.). The reaction mixture stirred at 22° C. until a clear solution was formed and DIPEA (305.2 mL, 1.795 mol, 2.0 eq) was added at once. The reaction mixture stirred at 20-25° C. IPC-HPLC after one hour shown completeness of the reaction.

At this point, the reaction mixture was diluted with EtOAc (2.0 L) and washed with NaHCO₃ (5% aq.) (3×3 L) and water (2×3 L). The solvent of the organic phase was evaporated under reduced pressure at 35° C. and the residue co-evaporated with EtOAc (2×2.5 L). The residue was taken in EtOAc (3.0 L) and the product precipitated by adding the product solution slowly to heptane (30 L). The white suspension formed was stirred at 20-25° C. for 30 minutes before the product was separated by filtration. The filter cake was washed with heptane (2×2 L) and dried under vacuum at 35° C. for 18 h to give 545 g of the desired product as a white solid, Fmoc-Pen(Trt)-Asn-Thr(tBu)-OMe. (HPLC-Purity: 98.7%, Yield 96.5%)

### Example II-4c: Synthesis of H-Pen(Trt)-Asn-Thr(tBu)-Ome

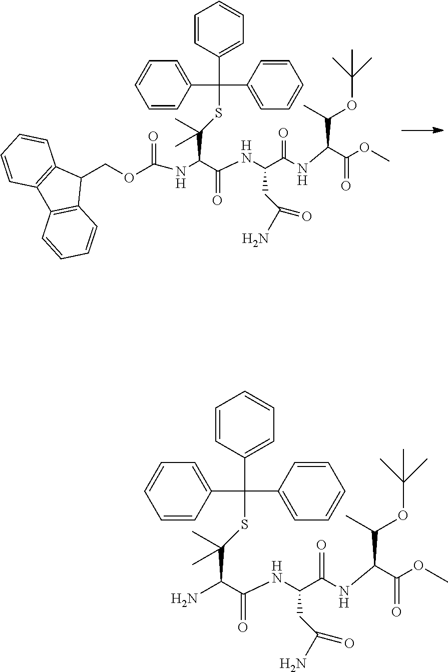 [SMILES: COC(=O)[C@@H](NC(=O)[C@H](CC(N)=O)NC(=O)[C@@H](NC(=O)OCC1c2ccccc2-c2ccccc21)C(C)(C)SC(c1ccccc1)(c1ccccc1)c1ccccc1)C(C)OC(C)(C)C ; COC(=O)[C@@H](NC(=O)[C@H](CC(N)=O)NC(=O)[C@@H](N)C(C)(C)SC(c1ccccc1)(c1ccccc1)c1ccccc1)C(C)OC(C)(C)C]

In a 15 L glass reactor with overhead stirrer were loaded Fmoc-Pen(Trt)-Asn-Thr(tBu)-OMe (500.0 g, 0.556 mol, 1.0 eq.) and EtOAc:DMSO (9:1, v:v) (5.0 L). The reaction mixture was stirred at 22° C. until a clear solution was formed. DBU (24.9 mL, 0.167 mol, 0.3 eq.) was added and the reaction mixture stirred at 20-25° C. IPC-HPLC after 90 minutes shown completeness of the reaction.

At this point, the reaction mixture was diluted with EtOAc (4.0 L) and washed with NaHCO₃ (5% aq.) (4 L) and water (4 L). The solvent of the organic phase was evaporated under reduced pressure at 40° C. and the residue co-evaporated with EtOAc (2×2.0 L).

The residue was taken in EtOAc (2.0 L) and the product precipitated by adding the product solution slowly to heptane (30 L). The white suspension formed was stirred at 20-25° C. for 15 minutes before the product was separated by filtration. The filter cake was washed with heptane (2×2 L) and dried under vacuum at 35° C. for 18 h to give 337 g of the desired product as a white solid, H-Pen(Trt)-Asn-Thr(tBu)-OMe. (Yield 89.5%).

### Example II-4d: Synthesis of Ac-Pen(Trt)-Asn-Thr(tBu)-OMe

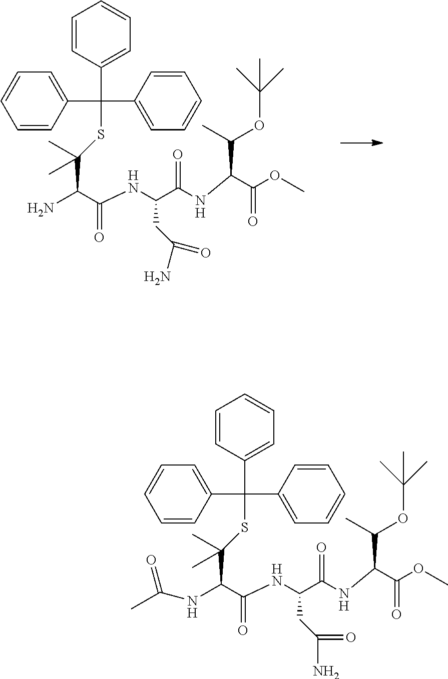

In a 15 L glass reactor with overhead stirrer were loaded H-Pen(Trt)-Asn-Thr(tBu)-OMe (318.2 g, 0.470 mol, 1.0 eq.) and MeOH (3.0 L) and the reaction mixture was stirred at 22° C. until a clear solution was formed. Acetic anhydride (55.6 mL, 0.588 mol, 1.25 eq.) was added and the reaction mixture stirred at 20-25° C. IPC-HPLC after 1 hour shown completeness of the reaction.

At this point, the reaction mixture was concentrated by evaporation of the solvent at 45° C. to a final volume of 0.4 Land the residue was taken up in EtOAc:Me-THF (1:1, v:v) (4.0 L). The organic phase was washed with NaHCO₃ (5% aq.) (2 L) and water (2 L) and the solvent of the organic phase evaporated under reduced pressure at 45° C. (AT) and the residue co-evaporated with EtOAc (2×1.5 L).

The residue was taken in EtOAc (3.0 L) and the product precipitated by adding the product solution slowly to heptane (30 L). The white suspension formed was stirred at 20-25° C. for 15 minutes before the product was separated by filtration. The filter cake was washed with heptane (4×1 L) and dried under vacuum at 35° C. for 18 h to give 328.2 g of the desired product as a white solid, Ac-Pen(Trt)-Asn-Thr(tBu)-OMe. (Yield 97.1%)

### Example II-4e: Synthesis of Ac-Pen(Trt)-Asn-Thr(tBu)-OH (FG1 tripeptide)

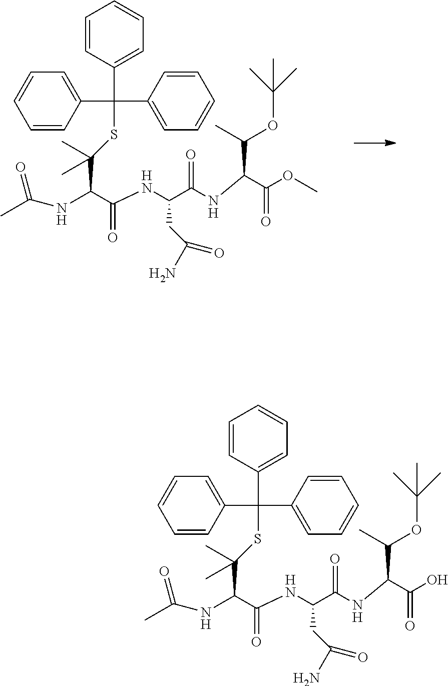

In a 10 L reactor with overhead stirring were loaded Ac-Pen(Trt)-Asn-Thr(tBu)-OMe (310.0 g, 0.431 mol, 1.0 eq.) and THF (1.5 L). The mixture was stirred until a clear solution was obtained. The product solution was diluted with water (1.5 L) and cooled to 5° C. A solution of LiOH×H₂O (25.35 g, 0.603 mol, 1.4 eq.) in water (0.5 L) was added to the reaction mixture within 90 minutes while stirring at 5° C. IPC-HPLC after 5 hours shown almost complete reaction (S.M: 1.2%).

At this point, the reaction mixture was diluted with water (1.5 L) and Me-THF (5 L) and the layers separated (the product remained in the aqueous phase). Me-THF (6 L) was added to the aqueous phase and the pH of the biphasic mixture was adjusted to 4.7 with HCl (10%, ca. 100 mL). The phases were separated and the aqueous phase re-extracted with EtOAc (3.0 L). Both organic phases were mixed and the solvent evaporated under reduced pressure at 35° C. (AT) followed by co-evaporation of the residue with EtOAc (2×3.0 L).

The residue was taken in EtOAc (3.0 L) and the product precipitated by adding the product solution slowly to heptane (35 L). The white suspension formed was stirred at 20-25° C. for 15 minutes before the product was separated by filtration. The filter cake was washed with heptane (2×0.5 L) and dried under vacuum at 35° C. for 18 h to give 252.4 g of the desired product as a white solid, Ac-Pen(Trt)-Asn-Thr(tBu)-OH. (Yield 83.0%, ESI-MS: m/z=1408.74 [2M+H⁺]⁺).

### Example II-5: Synthesis of Fmoc-Trp(7Me)-Lys(Ac)-Pen(acm)-Tyr(2-Boc-ea)-OH (FG1 tetrapeptide)

### Example II-5a: Synthesis of Fmoc-Pen(Acm)-Tyr(2-Boc-ea)-OMe

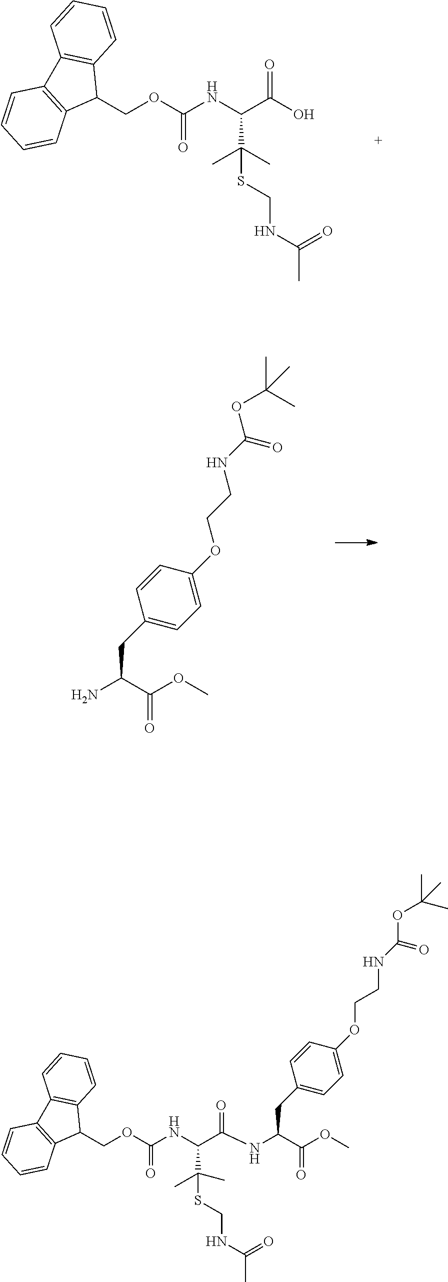 [SMILES: CC(=O)NCSC(C)(C)[C@H](NC(=O)OCC1c2ccccc2-c2ccccc21)C(=O)O ; COC(=O)[C@@H](N)Cc1ccc(OCCNC(=O)OC(C)(C)C)cc1 ; COC(=O)[C@H](Cc1ccc(OCCNC(=O)OC(C)(C)C)cc1)NC(=O)[C@@H](NC(=O)OCC1c2ccccc2-c2ccccc21)C(C)(C)SCNC(C)=O]

Fmoc-Pen(Acm)-OH (0.79 mol, 1.0 eq), H-Tyr(2-Boc-ea)-OMe (0.81 mol, 1.02 eq.) and Oxyma Pure (0.79 mol, 1.0 eq.) were dissolved in EtOAc (3.5 L). DIC (0.87 mol, 1.1 eq.) was added over 2 hours at 23° C. IPC-HPLC after 1 h showed reaction completion. The reaction mixture was filtered and the filter cake was washed with EtOAc. The product solution was washed with NaHSO₄ aq., NaHCO₃ aq. and water. The solvent was evaporated under reduced pressure at 35° and the resulting residue was co-evaporated with EtOAc three times. The residue was taken up in Me-THF (2 L) and the product solution was precipitated by adding the solution to pentane (35 L) over 30 minutes. The white suspension was separated by filtration and the product washed with pentane. The off-white product was dried in a vacuum oven under reduced pressure at 35° C. over the weekend. 637.8 g of an off-white solid, Fmoc-Pen(Acm)-Tyr(2-Boc-ea)-OMe, were obtained (HPLC purity: 93.8% product, Yield 106%, ESI-MS: m/z=763.23 [M+H]⁺).

### Example II-5b: Synthesis of H-Pen(Acm)-Tyr(2-Boc-ea)-OMe

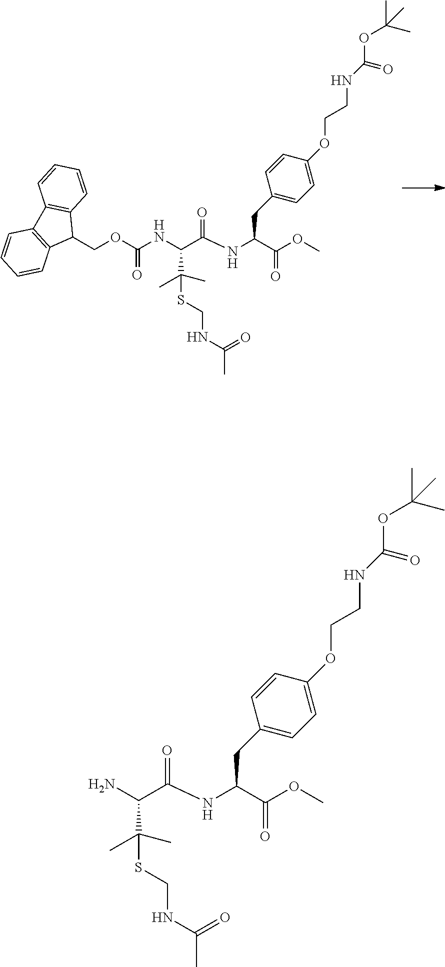

Fmoc-Pen(Acm)-Tyr(2-Boc-ea)-OMe (0.786 mol, 1.0 eq) was dissolved in EtOAc/DMSO (9:1; v:v) (6 L). To this solution, DBU (0.393 mol, 0.5 eq.) was added. The reaction mixture was stirred at r.t. for 2.5 hours. IPC-HPLC after 2 hours showed 0.65% Fmoc-Pen(Acm)-Tyr(2-Boc-ea)-OMe. The organic product layer was diluted with EtOAc and washed with 5% NaHCO₃ aq. and H₂O. The aqueous phases were washed with EtOAc. The organic phases were combined and evaporated at 35° C. The residue was co-distillated twice with EtOAc. The residue was stored at 4-5° C. overnight and directly used as starting material for the coupling with Fmoc-Lys(Ac)—OH.

The H-Pen(Acm)-Tyr(2-Boc-ea)-OMe was diluted in EtOAc/DMSO (8:2; v:v) (3 L). 100 μl (93.0 mg) of the solution were diluted in 10 ml ACN and analyzed by HPLC. Mass of the solution: 4409.5 g. The amount of H-Pen(Acm)-Tyr(2-Boc-ea)-OMe was calculated using a non-qualified standard of the compound. The amount of H-Pen(Acm)-Tyr(2-Boc-ea)-OMe was calculated to be 396.91 g (Yield: 93.3%, HPLC purity 96.98).

### Example II-5c: Synthesis of Fmoc-Lys(Ac)-Pen(Acm)-Tyr(2-Boc-ea)-Ome

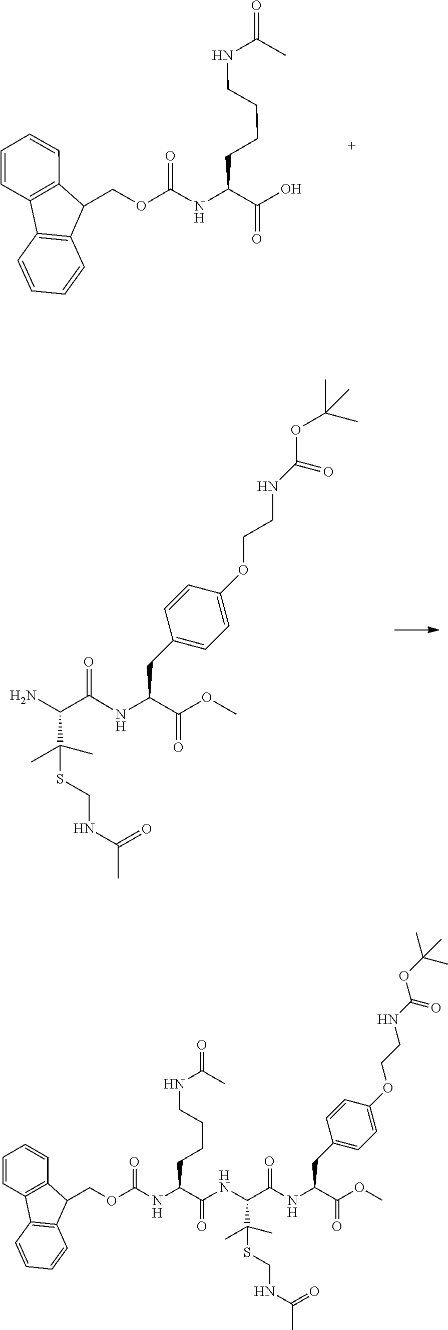 [SMILES: CC(=O)NCCCC[C@H](NC(=O)OCC1c2ccccc2-c2ccccc21)C(=O)O ; COC(=O)[C@H](Cc1ccc(OCCNC(=O)OC(C)(C)C)cc1)NC(=O)[C@@H](N)C(C)(C)SCNC(C)=O ; COC(=O)[C@H](Cc1ccc(OCCNC(=O)OC(C)(C)C)cc1)NC(=O)[C@@H](NC(=O)[C@H](CCCCNC(C)=O)NC(=O)OCC1c2ccccc2-c2ccccc21)C(C)(C)SCNC(C)=O]

To the mixture of H-Pen(Acm)-Tyr(2-Boc-ea)-OMe (0.732 mol, 1.0 eq.) in EtOAc/DMSO (8:2; v:v) (3 L) was added Fmoc-Lys(Ac)—OH (0.637 mol, 0.87 eq.) and TBTU (0.696 mol, 0.95 eq.) and the reaction mixture was stirred for 2 minutes at r.t.. DIPEA (1.391 mol, 1.90 eq.) was added and the reaction mixture was stirred at r.t. IPC-HPLC after 1 h showed 2.1% H-Pen(Acm)-Tyr(2-Boc-ea)-OMe, n.d. Fmoc-Lys(Ac)—OH. Fmoc-Lys(Ac)—OH (18.6 mmol, 0.025 eq.) were added. IPC-HPLC after 2 h showed 0.81% H-Pen(Acm)-Tyr(2-Boc-ea)-OMe. The reaction mixture was diluted with EtOAc and washed twice with 20% NaHSO₄ aq, H₂O, twice with 5% Na₂CO₃ aq. and again with water. Because the HPLC of the organic phase still showed 0.68% H-Pen(Acm)-Tyr(2-Boc-ea)-OMe the organic phase was washed again twice with 20% NaHSO₄ aq, H₂O, twice with 5% Na₂CO₃ aq. and again with water. The HPLC of the organic phase showed 0.60% H-Pen(Acm)-Tyr(2-Boc-ea)-OMe. The water phases were washed with EtOAc. The organic phases was stored at 4-5° C. overnight. The organic phase was evaporated at 35° C. and co-distilled twice with EtOAc. The residue was dissolved in EtOAc. The mixture was slowly added to IPE over 25 minutes. The suspension was filtered and washed three times with IPE. The solid was dried under high vacuum at 35° C. overnight. The white solid was dissolved in EtOAC. The solution was added to IPE over 5 minutes. The suspension was filtered and washed three times with IPE. The solid was dried under high vacuum at 35° C. overnight. 554.9 g of a white solid, Fmoc-Lys(Ac)-Pen(Acm)-Tyr(2-Boc-ea)-OMe, were obtained (HPLC-Purity 97.05% product, ESI-MS: 933.32 m/z [M+H]⁺, Yield 75.6% over both steps).

### Example II-5d: Synthesis of H-Lys(Ac)-Pen(Acm)-Tyr(2-Boc-ea)-OMe

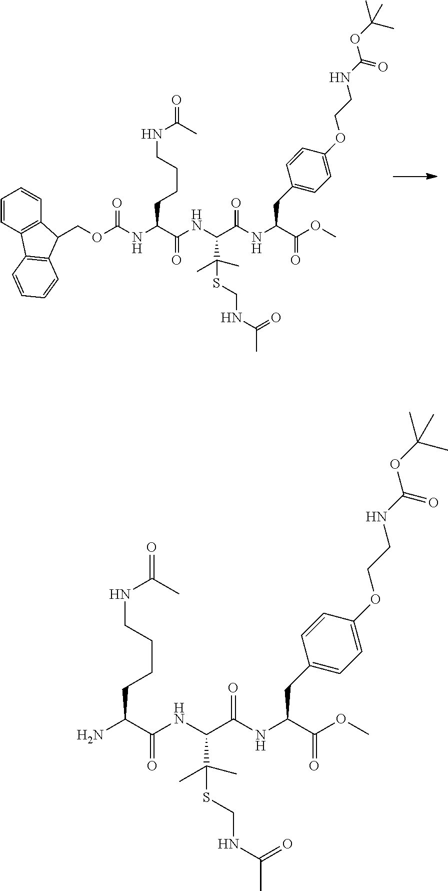 [SMILES: COC(=O)[C@H](Cc1ccc(OCCNC(=O)OC(C)(C)C)cc1)NC(=O)[C@@H](NC(=O)[C@H](CCCCNC(C)=O)NC(=O)OCC1c2ccccc2-c2ccccc21)C(C)(C)SCNC(C)=O ; COC(=O)[C@H](Cc1ccc(OCCNC(=O)OC(C)(C)C)cc1)NC(=O)[C@@H](NC(=O)[C@@H](N)CCCCNC(C)=O)C(C)(C)SCNC(C)=O]

Fmoc-Lys(Ac)-Pen(Acm)-Tyr(2-Boc-ea)-OMe (0.563 mol, 1.0 eq) was dissolved in EtOAc/DMSO (9:1; v:v) (5.2 L). To this solution, DBU (0.281 mol, 0.5 eq.) was added. The reaction mixture was stirred at r.t. for 1.5 hours. IPC-HPLC after 1 hours showed 0.03% Fmoc-Lys(Ac)-Pen(Acm)-Tyr(2-Boc-ea)-OMe. The organic product layer was diluted with EtOAc and washed twice with H₂O.

The aqueous phases were washed twice with EtOAc. The organic phases were combined and evaporated at 40° C. The residue was co-distillated twice with EtOAc. The residue was stored at 4-5° C. overnight and directly used as starting material for the coupling with Fmoc-7-Me-Trp-OH.

The H-Lys(Ac)-Pen(Acm)-Tyr(2-Boc-ea)-OMe was diluted in EtOAc/DMSO (8:2; v:v) (2.6 L). 100 ul (89.3 mg) of the solution were diluted in 10 ml MeOH and analyzed by HPLC. Mass of the solution: 3517 g. The amount of H-Lys(Ac)-Pen(Acm)-Tyr(2-Boc-ea)-OMe was calculated using a non-qualified standard of the compound. The amount of H-Lys(Ac)-Pen(Acm)-Tyr(2-Boc-ea)-OMe was calculated to be 367 g (Yield 91.7%, HPLC purity 97.8%).

### Example II-5e: Synthesis of Fmoc-Trp(7Me)-Lys(Ac)-Pen(Acm)-Tyr(2-Boc-ea)-OMe

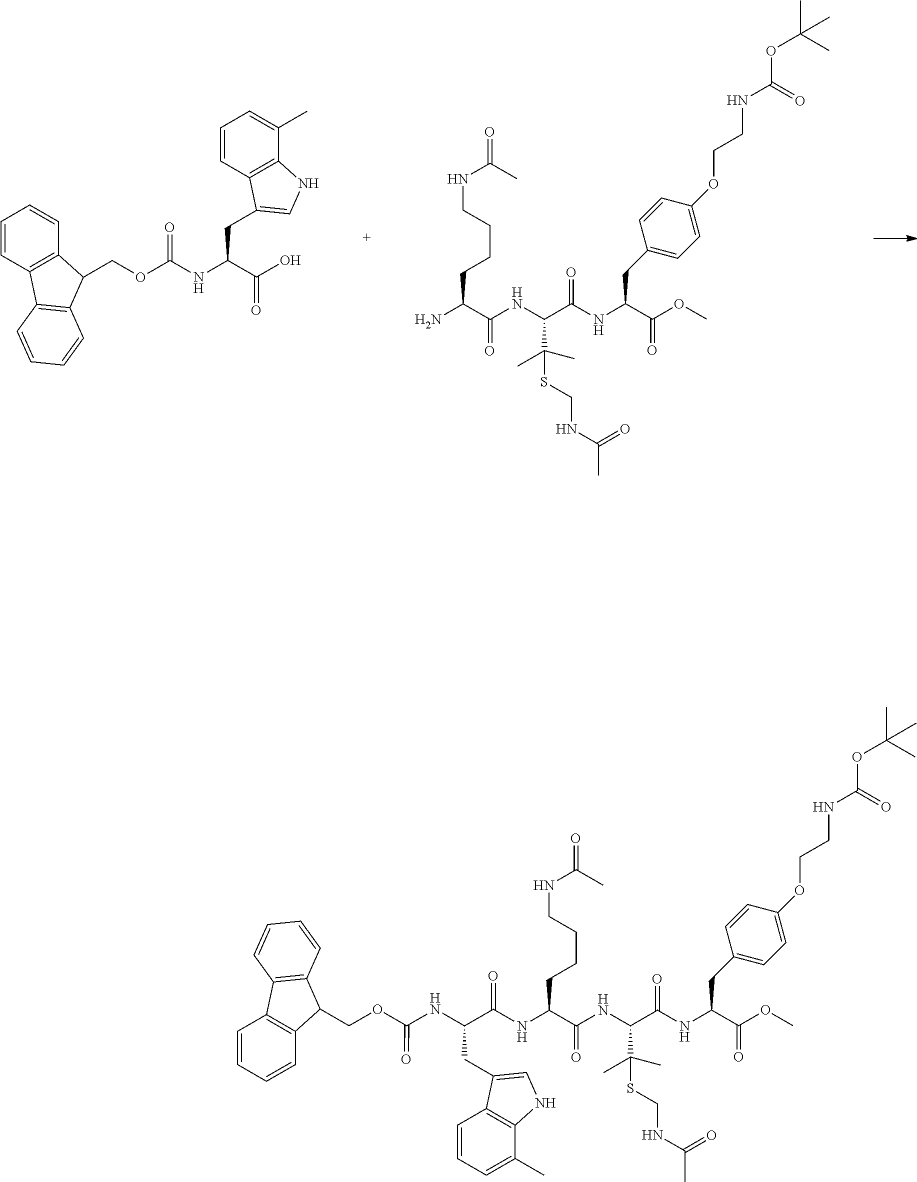 [SMILES: Cc1cccc2c(C[C@H](NC(=O)OCC3c4ccccc4-c4ccccc43)C(=O)O)c[nH]c12 ; COC(=O)[C@H](Cc1ccc(OCCNC(=O)OC(C)(C)C)cc1)NC(=O)[C@@H](NC(=O)[C@@H](N)CCCCNC(C)=O)C(C)(C)SCNC(C)=O ; COC(=O)[C@H](Cc1ccc(OCCNC(=O)OC(C)(C)C)cc1)NC(=O)[C@@H](NC(=O)[C@H](CCCCNC(C)=O)NC(=O)[C@H](Cc1c[nH]c2c(C)cccc12)NC(=O)OCC1c2ccccc2-c2ccccc21)C(C)(C)SCNC(C)=O]

To the mixture of H-Lys(Ac)-Pen(Acm)-Tyr(2-Boc-ea)-OMe (0.512 mol, 1.0 eq.) in EtOAc/DMSO (8:2; v:v) (2.6 L) was added Fmoc-Trp(7Me)-OH (0.415 mol, 0.81 eq.) and TBTU (0.486 mol, 0.95 eq.) and the reaction mixture was stirred for 2 minutes at r.t.. DIPEA (1.024 mol, 2.00 eq.) was added and the reaction mixture was stirred at r.t. IPC-HPLC after 1 h showed 0.41% H-Lys(Ac)-Pen(Acm)-Tyr(2-Boc-ea)-OMe, n.d. Fmoc-Trp(7Me)-OH. After 2 hours the reaction mixture was diluted with EtOAc and washed twice with 5% NaHCO₃ aq. The aqueous phases were washed with EtOAc. The organic phases evaporated at 40° C. The residue was azeotroped twice with EtOAc. The residue was stored at 4-5° C. overnight. The residue was diluted with EtOAc and slowly added to IPE/heptane (1:1; v:v) over 10 minutes. The suspension was filtered and washed three times with IPE. The solid was dried under high vacuum at 35° C. overnight. 487.67 g of a white solid, Fmoc-Trp(7Me)-Lys(Ac)-Pen(Acm)-Tyr(2-Boc-ea)-OMe, were obtained (HPLC-Purity 95.04%, ESI-MS 1133.33 m/z [M+H]⁺, Yield 76.48% over both steps).

### Example II-5f: Synthesis of H-Trp(7Me)-Lys(Ac)-Pen(Acm)-Tyr(2-Boc-ea)-OMe

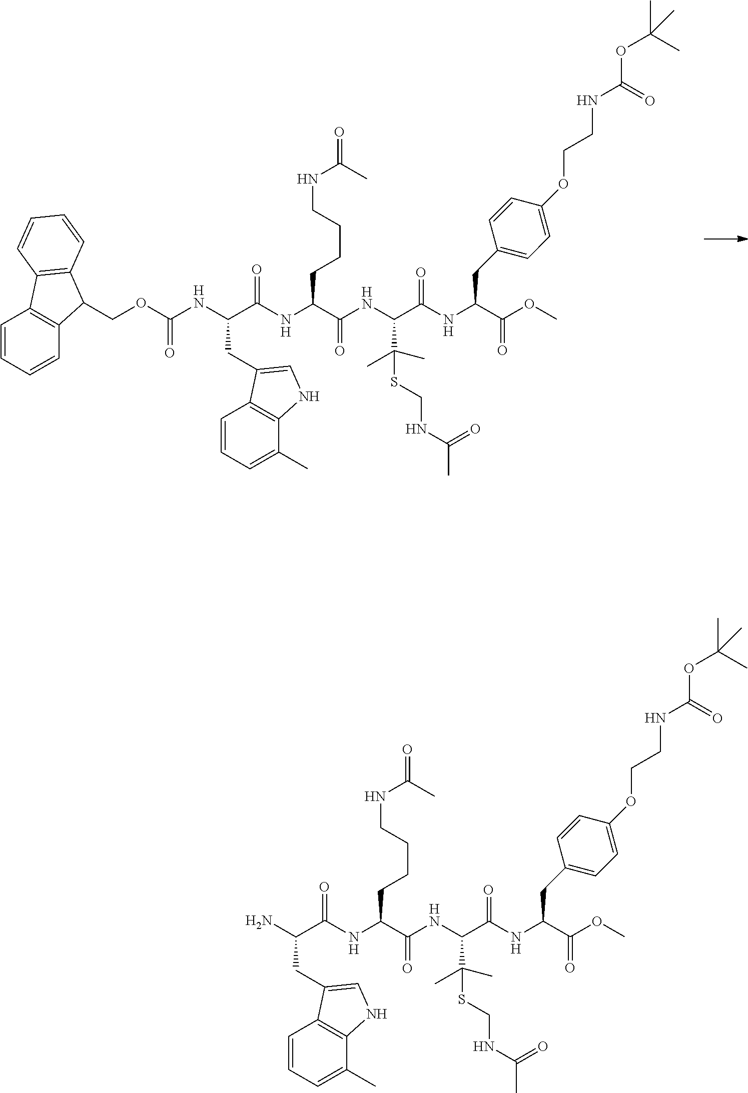 [SMILES: COC(=O)[C@H](Cc1ccc(OCCNC(=O)OC(C)(C)C)cc1)NC(=O)[C@@H](NC(=O)[C@H](CCCCNC(C)=O)NC(=O)[C@H](Cc1c[nH]c2c(C)cccc12)NC(=O)OCC1c2ccccc2-c2ccccc21)C(C)(C)SCNC(C)=O ; COC(=O)[C@H](Cc1ccc(OCCNC(=O)OC(C)(C)C)cc1)NC(=O)[C@@H](NC(=O)[C@H](CCCCNC(C)=O)NC(=O)[C@@H](N)Cc1c[nH]c2c(C)cccc12)C(C)(C)SCNC(C)=O]

To a solution of Fmoc-7Me-Trp-Lys(Ac)-Pen(Acm)-Tyr(2-Boc-ea)-OMe (450 g, 397 mmol, 1.0 eq.) in EtOAc/DMSO (8:2, v:v) (4.5 L) at r.t. was added DBU (29.63 mL, 198.5 mmol, 0.5 eq.) and the reaction mixture stirred at r.t. IPC-HPLC after 1 h showed 0.01% Fmoc-7-Me-Trp-Lys(Ac)-Pen(Acm)-Tyr(2-Boc-ea)-OMe. After 1.5 h the reaction mixture was diluted with EtOAc/Me-THF 1:1 (9.0 L). The organic solution was washed twice with NaHCO₃(5%, aq.) (4.5 L) and H2O (4.5 L). The aqueous phases were washed with EtOAc/Me-THF 1:1 (4.5 L). The organic phases was evaporated under reduced pressure at 40° C. (AT). The residue co-evaporated with EtOAc (3×2 L). The residue was stored at 4-5° C. overnight.

The residue was taken up in EtOAc (1 L) to give in total 3 L volume. The diluted mixture was dropped onto IPE (30 L) over 5 minutes. The suspension was stirred at r.t. for 10 minutes. The suspension was filtered and washed twice with IPE (1.5 L). The white solid was dried under high vacuum at 35° C. overnight. The white solid was dissolved in EtOAc (3.5 L) for 10 minutes. The solution was added to IPE (35 L) over 10 minutes. The white suspension was stirred at r.t. over 30 minutes. The white suspension was filtered and washed twice with IPE (1.5 L). The white solid, H-Trp(7Me)-Lys(Ac)-Pen(Acm)-Tyr(2-Boc-ea)-OMe, was dried under high vacuum at 35° C. overnight. (HPLC-Purity: 95.59% ESI-MS: 911.56 m/z [M+H]⁺, Yield 92.9%)

### Example II-6: Synthesis of Ac-Pen(Trt)-Asn-Thr(tBu)-Trp(7Me)-Lys(Ac)-Pen(acm)-Tyr(2-Boc-ea)-OMe (Ac-FG1-OMe)

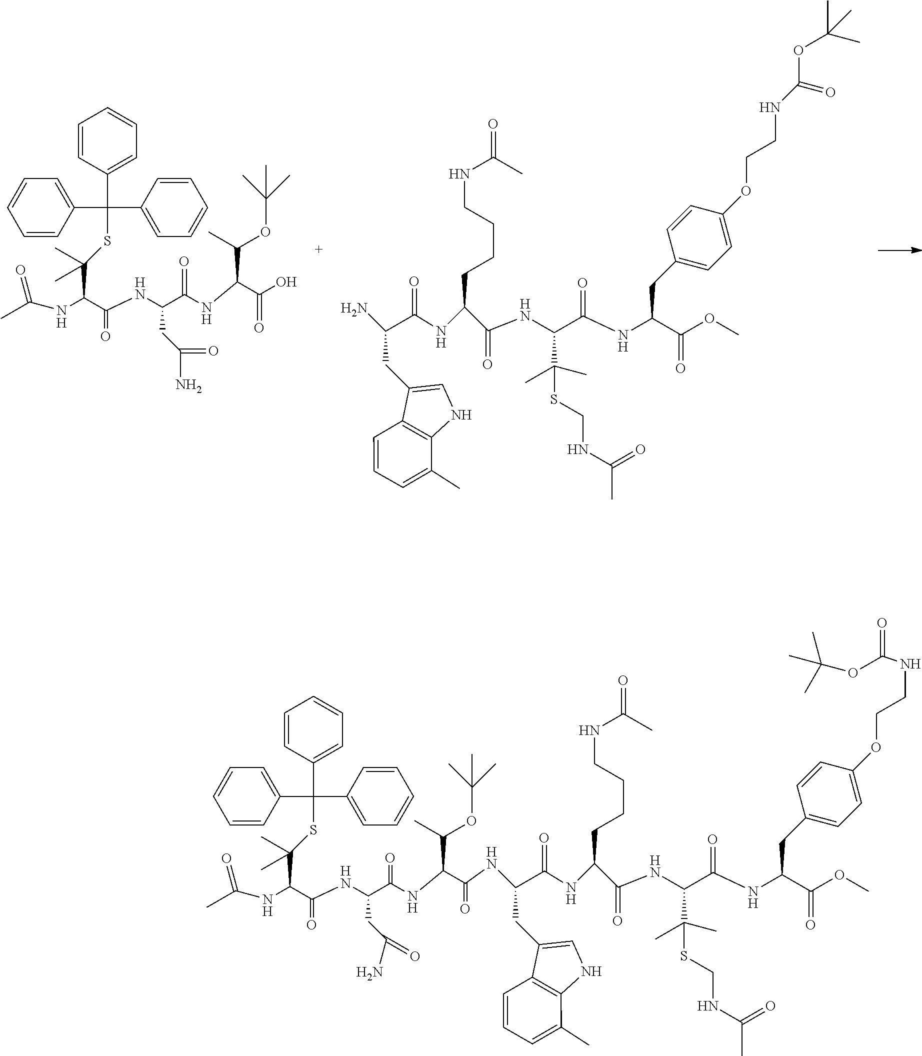 [SMILES: CC(=O)N[C@H](C(=O)N[C@@H](CC(N)=O)C(=O)N[C@H](C(=O)O)C(C)OC(C)(C)C)C(C)(C)SC(c1ccccc1)(c1ccccc1)c1ccccc1 ; COC(=O)[C@H](Cc1ccc(OCCNC(=O)OC(C)(C)C)cc1)NC(=O)[C@@H](NC(=O)[C@H](CCCCNC(C)=O)NC(=O)[C@@H](N)Cc1c[nH]c2c(C)cccc12)C(C)(C)SCNC(C)=O ; COC(=O)[C@H](Cc1ccc(OCCNC(=O)OC(C)(C)C)cc1)NC(=O)[C@@H](NC(=O)[C@H](CCCCNC(C)=O)NC(=O)[C@H](Cc1c[nH]c2c(C)cccc12)NC(=O)[C@@H](NC(=O)[C@H](CC(N)=O)NC(=O)[C@@H](NC(C)=O)C(C)(C)SC(c1ccccc1)(c1ccccc1)c1ccccc1)C(C)OC(C)(C)C)C(C)(C)SCNC(C)=O]

In a double jacketed 10 L glass reactor were loaded Ac-Pen(Trt)-Asn-Thr(tBu)-OH (212.0 g, 273.692 mmol, 1.0 eq.), H-7Me-Trp-Lys(Ac)-Pen(Acm)-Tyr(2-Boc-ea)-OMe (275.4 g, 281.903 mmol, 1.03 eq.), Oxyma B® (50.7 g, 273.692 mmol, 1.0 eq.) and Acetonitrile (3.23 kg, 4.2 L). The mixture was stirred at 25° C. for 30 minutes (red-fine suspension) and DIC (38.0 g, 301.061 mmol, 1.1 eq.) was added over 5 minutes and the reaction mixture stirred at 25° C.. IPC-HPLC after 3 hours, shown completeness of the reaction. A white to beige suspension was formed. The suspension was stirred at 25° C. for 21 hours more and the product separated by filtration through a pore 3 glass filter and the filter cake washed with AcN (3×0.3 L). The wet product was re-suspended in AcN (2.0 L, 1.55 kg) and the suspension heated to 40° C. within 30 minutes and stirred at this temperature for 1.5 hours. The suspension was cooled to 15° C. within 1 hour and stirred at this temperature for 6 hours.

The product was separated by filtration through a pore 3 glass filter. The filter cake was washed with AcN (3×0.3 L mL) and dried under reduced pressure at 35° C. to give 364.0 g of an off-white to beige solid, Ac-Pen(Trt)-Asn-Thr(tBu)-Trp(7Me)-Lys(Ac)-Pen(acm)-Tyr(2-Boc-ea)-OMe, (Yield: 83.2%, HPLC purity: 97.0%, ESI-MS: m/z=1597.32 [M+H⁺]⁺).

### Example II-7: Synthesis of Ac-Pen(Trt)-Asn-Thr(tBu)-Trp(7Me)-Lys(Ac)-Pen(acm)-Tyr(2-Boc-ea)-OH (Ac-FG1-OH (linear))

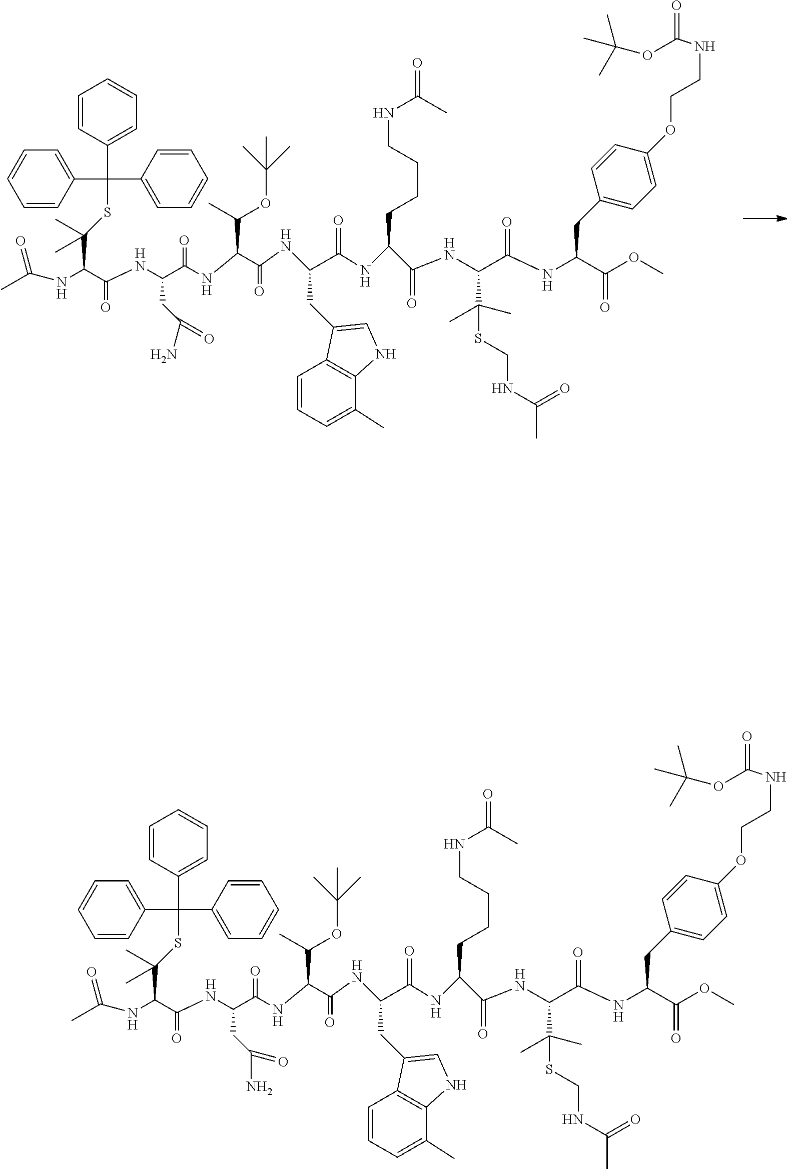 [SMILES: COC(=O)[C@H](Cc1ccc(OCCNC(=O)OC(C)(C)C)cc1)NC(=O)[C@@H](NC(=O)[C@H](CCCCNC(C)=O)NC(=O)[C@H](Cc1c[nH]c2c(C)cccc12)NC(=O)[C@@H](NC(=O)[C@H](CC(N)=O)NC(=O)[C@@H](NC(C)=O)C(C)(C)SC(c1ccccc1)(c1ccccc1)c1ccccc1)C(C)OC(C)(C)C)C(C)(C)SCNC(C)=O ; COC(=O)[C@H](Cc1ccc(OCCNC(=O)OC(C)(C)C)cc1)NC(=O)[C@@H](NC(=O)[C@H](CCCCNC(C)=O)NC(=O)[C@H](Cc1c[nH]c2c(C)cccc12)NC(=O)[C@@H](NC(=O)[C@H](CC(N)=O)NC(=O)[C@@H](NC(C)=O)C(C)(C)SC(c1ccccc1)(c1ccccc1)c1ccccc1)C(C)OC(C)(C)C)C(C)(C)SCNC(C)=O]

Ac-FG1-OMe (150.0 g, 89.1% w/w, 83.637 mmol, 1.0 eq.), THF (0.75 L, 0.657 Kg) and water (0.525 L, 0.525 kg) were loaded into a 1.5 L glass jacketed reactor with overhead stirrer. The mixture was stirred at 25° C. until a solution was obtained and cooled to 0° C. within 30 minutes. A solution of LiOH×H₂O (5.264 g, 125.455 mmol, 1.5 eq.) in water (0.225 L, 0.225 kg) was added within one hour to the reaction mixture under stirring and keeping the temperature at 0-2° C. IPC-HPLC 2 h after complete LiOH solution. addition shown completeness of the reaction. The pH of the reaction mixture was adjusted to 4.9 with HCl (conc.) and heated to 25° C. within 30 min. The reaction mixture was diluted with MeTHF (1.5 L) and the phases left to separate. The solvent was evaporated under reduced pressure at 40° C. to ca. 300 mL and the residue co-evaporated with EtOAc (3×1 L). The product starts to crystallize after the 2^{nd}. co-evaporation. The residue was taken up in EtOAc (820 mL), stirred at 60° C. for 30 minutes, cooled to 25° C. within 4 hours and stirred at this temperature overnight. The product was separated by filtration through a pore 3 glass filter and the filter cake washed with EtOAc (200 mL). The product was dried under vacuum at 35° C. for 24 h to give 135.7 g of a beige to white-of solid, Ac-Pen(Trt)-Asn-Thr(tBu)-Trp(7Me)-Lys(Ac)-Pen(acm)-Tyr(2-Boc-ea)-OH. (Yield: 82.8%, HPLC purity: 96.1% ESI-MS: m/z=1583.36 [M+H⁺]⁺)

### Example II-8: Synthesis of Ac-Pen(Trt)-Asn-Thr(tBu)-Trp(7Me)-Lys(Ac)-Pen(acm)-Tyr(2-Boc-ea)-OH (S—S, 1-6) (Ac-FG1-OH (cyclic))

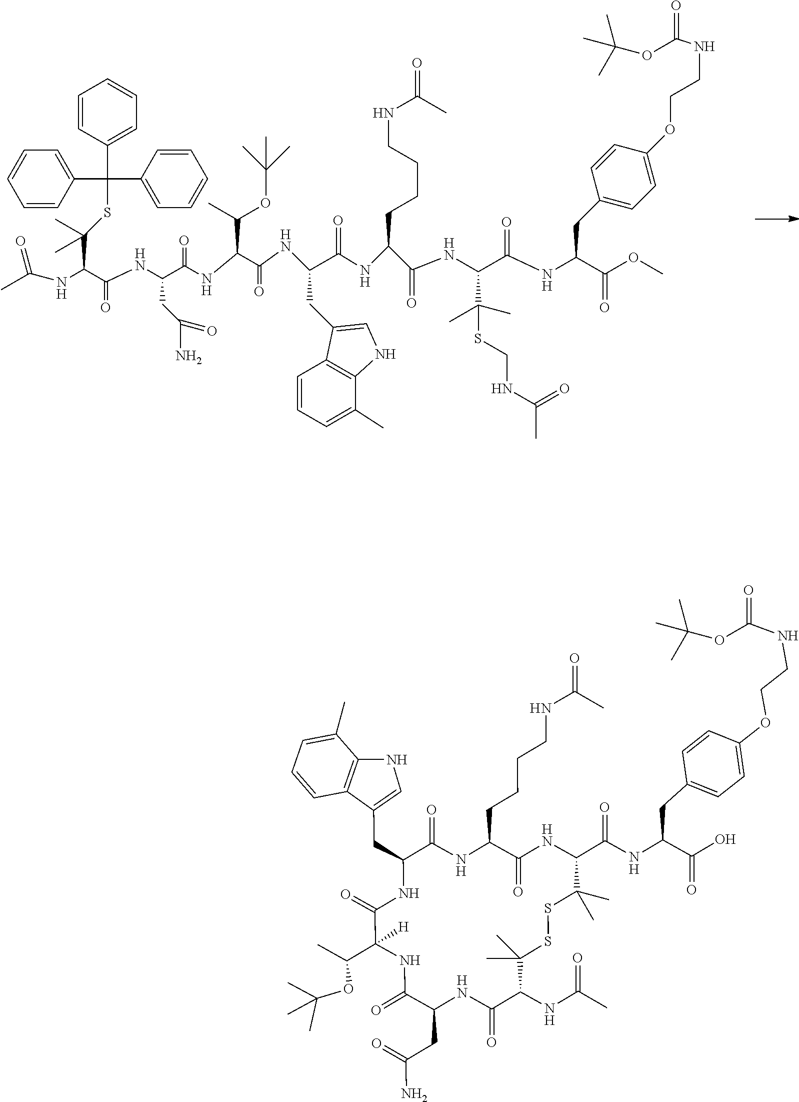 [SMILES: COC(=O)[C@H](Cc1ccc(OCCNC(=O)OC(C)(C)C)cc1)NC(=O)[C@@H](NC(=O)[C@H](CCCCNC(C)=O)NC(=O)[C@H](Cc1c[nH]c2c(C)cccc12)NC(=O)[C@@H](NC(=O)[C@H](CC(N)=O)NC(=O)[C@@H](NC(C)=O)C(C)(C)SC(c1ccccc1)(c1ccccc1)c1ccccc1)C(C)OC(C)(C)C)C(C)(C)SCNC(C)=O ; CC(=O)NCCCC[C@@H]1NC(=O)[C@H](Cc2c[nH]c3c(C)cccc23)NC(=O)[C@H]([C@@H](C)OC(C)(C)C)NC(=O)[C@H](CC(N)=O)NC(=O)[C@@H](NC(C)=O)C(C)(C)SSC(C)(C)[C@@H](C(=O)N[C@@H](Cc2ccc(OCCNC(=O)OC(C)(C)C)cc2)C(=O)O)NC1=O]

In a 20 L glass reactor a solution of iodine (123.2 g, 3.40 Eq, 485.5 mmol) and potassium iodide (80.6 g, 3.40 Eq, 485.5 mmol) in acetone/water (8:2; 12.0 L) was stirred for 30 min at 25° C. A solution (slightly turbid to thin suspension) of Ac-FG1-OH (linear) (273.7 g, HPLC-assay: 82.65%, 1 Eq, 142.8 mmol) in acetone/water (8:2; 2.28 L) was added with a dosage pump over 8 hour under nitrogen atmosphere at 25° C. (Conc: 10 mM). The reaction mixture was stirred at 25° C., overnight (15 h), cooled to 15° C. and a solution of Sodium thiosulfate pentahydrate (241.0 g, 6.80 Eq, 971.0 mmol) in water (714 mL) was added within 15 min yielding a clear, yellowish solution. The pH was adjusted from 2.8 to 5.0 with addition of NaHCO₃-5% (500 mL). The reaction mixture got slightly turbid and rose. The reaction mixture was transferred into a 20 L rotoevaporator, all equipment was flushed with acetone (250 mL) and also transferred.

The mixture was evaporated under reduced pressure at 40° C. to remove the acetone. The residue (rose, clear solution with oily product on the wall) was diluted with brine (1.3 L) and Me-THF (2.3 L). The pH 6.2 was adjusted to 3.0 with NaHSO₄-5%. The mixture was stirred for 20 min until all was dissolved. The phases were separated and the aqueous phase (pH 3.8) was extracted with Me-THF (1×1.5 L) and NaHSO₄-5% (20 mL). pH (UP2): 3.6. The combined organic phases were washed with brine 10% (3×0.80 L). The last separation needed 30 min to have full separation.

The organic phase (3.85 L) was evaporated under reduced pressure at 40° C. to a turbid solution (2.50 L). The thin suspension was azeotropicly distilled with Me-THF (4×500 mL) to form a white thick but stirrable suspension during the 3rd portion (Residue: 2.50 L/2.32 kg). The suspension was diluted with MTBE (2000 mL) and heptane (500 mL) and stirred for 15 min at 25° C. Filtrated and washed with MTBE (4×250 mL). The product was dried in a vacuum oven at 40° C. for 18 h.

This product (215.6 g) was dissolved in EtOAc (6.00 L) and water (210 mL) at temperature of 65° C. on the rotoevaporater. The slightly turbid, rose solution was clear filtrated over a Por3 glass filter and washed with EtoAc (500 mL, 50° C.). All transferred into a 20 L glass reactor with AT=55° C. The rose solution already started to get turbid (55° C.) and was seeded also at this point. Stirred for 15 min at =45° C. Then 1 h at =35° C. Then 2 h at 25° C. and 18 h at 20° C. Then cooled with at=0° C. and stirred for 2 h. The white suspension was filtrated and washed with EtOAc (3×350 mL). The product, Ac-Pen(Trt)-Asn-Thr(tBu)-Trp(7Me)-Lys(Ac)-Pen(acm)-Tyr(2-Boc-ea)-OH (S—S, 1-6) (Ac-FG1-OH (cyclic)), was dried in a vacuum oven at 40° C. (ESI-MS: [M+H]⁺: 1268.55)

### Example II-9: Synthesis of Ac-Pen(Trt)-Asn-Thr(tBu)-Trp(7Me)-Lys(Ac)-Pen(acm)-Tyr(2-Boc-ea)-2Nal-αMe-Lys(Boc)-Lys(Ac)-Asn-D-Leu-NH₂ (S—S, 1-6)

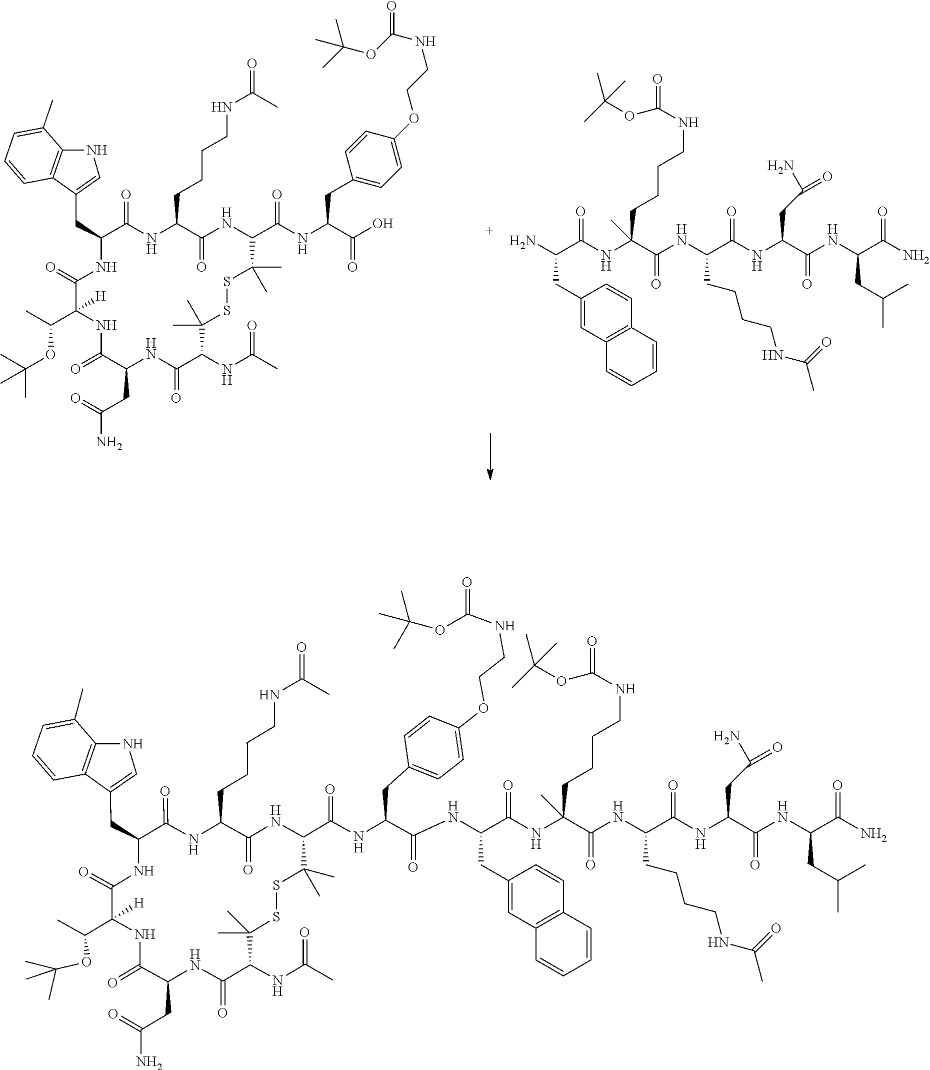

In a 400 mL flask Ac-FG1[S—S]—OH (15.00 g, 1 Eq, 10.61 mmol, HPLC: 97.4%, assay: 89.7%), H-FG2+3-NH2 (10.12 g, 1.03 Eq, 10.92 mmol, HPLC: 97.6%, assay: 92.2%) and Oxyma-B (2.160 g, 1.10 Eq, 11.67 mmol) were dissolved in DMF (60 mL, Synthesis Quality). The dark violet solution was diluted with Me-THF (240 mL). The violet solution was stirred at 25° C. DIC (1.339 g, 1.66 mL, 1.00 Eq, 10.61 mmol) was added at once and stirred at 25° C. for 6 h. (The addition of DIC showed no exothermic reaction). Added DIC (334.6 mg, 415 μL, 0.25 Eq, 2.652 mmol) and stirred further at 25° C.

After 24 h the reaction mixture was diluted with Me-THF (240 mL). The red solution was washed with NaHSO₄-5% solution (1×200 mL and 2×60 mL), NaHCO₃-5% solution (1×200 mL and 2×60 mL), NaCl-10% (2×60 ml) and water (1×60 mL). HPLC-4: 93.3% product.

The organic phase was evaporated under reduced pressure at 45° C. to a orange solution (92 g). The solution was co-distillated with MeTHF/MeOH (9:1; 2×100 mL). To a solution of 90 g. (Test precipitation showed that the solution was to be concentrated). Diluted with Me-THF to 140 g. The product oiled out. Added MeOH (5 mL) to form an orange solution. The solution was added within 20 min at RT to EtOAc (800 mL). A fine, white suspension was formed. Stirred for 1 h.

Several additional batches of Ac-Pen(Trt)-Asn-Thr(tBu)-Trp(7Me)-Lys(Ac)-Pen(acm)-Tyr(2-Boc-ea)-2Nal-αMe-Lys(Boc)-Lys(Ac)-Asn-D-Leu-NH2 (S—S, 1-6) were added to the material prepared above, (1.56 g, (0.499 g and 1.353 g) and the mixture was stirred for 15 min. The suspension was filtrated and washed with EtOAc (3×). The product was dried at 40° C. in a vacuum oven for 18 h to yield, Ac-Pen(Trt)-Asn-Thr(tBu)-Trp(7Me)-Lys(Ac)-Pen(acm)-Tyr(2-Boc-ea)-2Nal-αMe-Lys(Boc)-Lys(Ac)-Asn-D-Leu-NH2 (S—S, 1-6), 26.17 g/100%.

### Example II-10: Synthesis of Ac-Pen-Asn-Thr-Trp(7Me)-Lys(Ac)-Pen-Tyr(2-ea)-2Nal-αMe-Lys-Lys(Ac)-Asn-D-Leu-NH2 (S—S, 1-6)

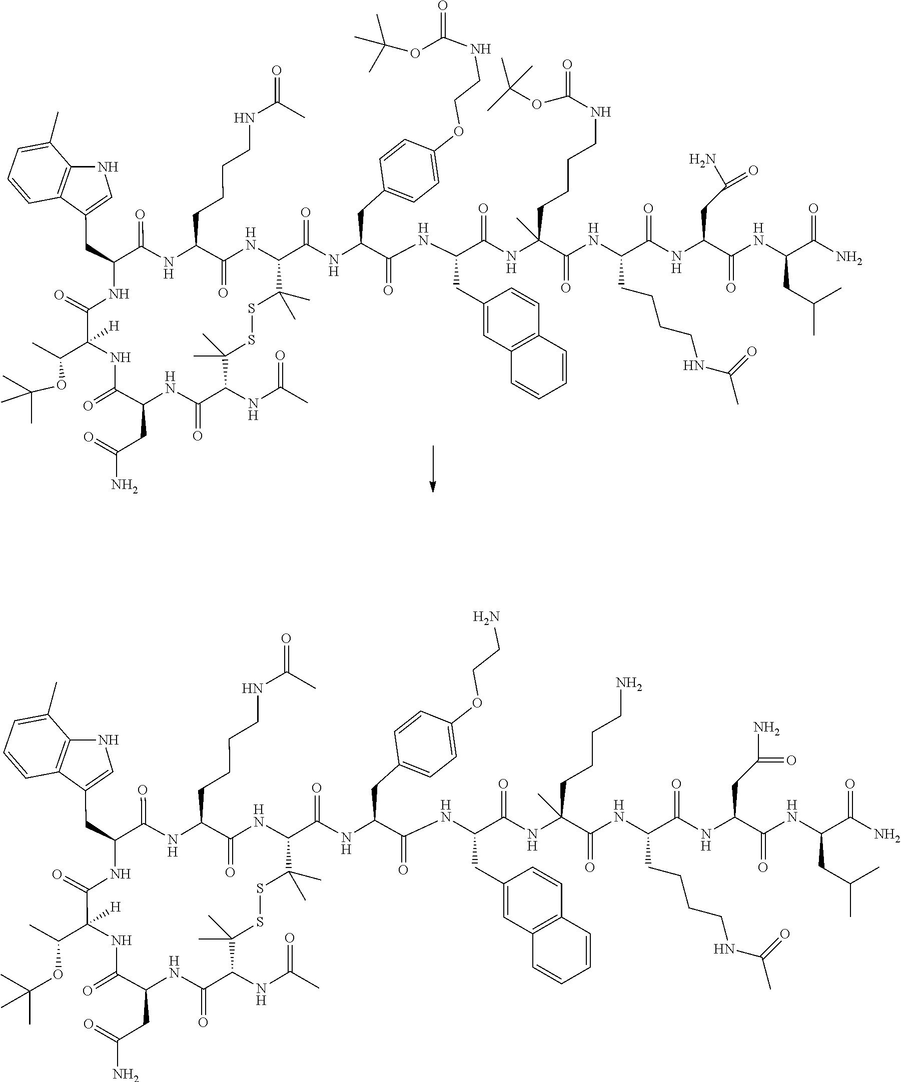

Ac-Pen(Trt)-Asn-Thr(tBu)-Trp(7Me)-Lys(Ac)-Pen(acm)-Tyr(2-Boc-ea)-2Nal-αMe-Lys(Boc)-Lys(Ac)-Asn-D-Leu-NH2 (S—S, 1-6)((12.6 g, 5.987 mmol, 1.0 eq.), HFIP (100 mL) and DODT (5 mL) were loaded into a 250 mL Systag reactor and the mixture stirred at 25° C. until a solution was formed. 4 N HCl in EtOAc (25 mL) pre-cooled at 4° C. was added to the reaction mixture over 5 minutes and the reaction mixture was stirred at 25-30° C. (initial increase of the temperature to 33° C. was observed). IPC-HPLC 45 min after complete addition of shown complete reaction. 1 h after complete addition of 4 N HCl the product was precipitated by adding EtOAc (130 mL) pre-cooled at 5° C. slowly to the reaction mixture. The white suspension formed was cooled to 4° C. within 10 minutes and stirred at this temperature for 15 minutes. The product was separated by filtration through a pore 3 glass filter and the filter cake washed with EtOAc (2×20 mL). The product was dried under vacuum at 35° C. for 18 h to give 12.94 g of a white solid Ac-Pen-Asn-Thr-Trp(7Me)-Lys(Ac)-Pen-Tyr(2-ea)-2Nal-αMe-Lys-Lys(Ac)-Asn-D-Leu-NH2 (S—S, 1-6)(HPLC purity: 92.3%, ESI-MS: m/z=924.88 Da [M+2H⁺]²⁺, Assay: 75.6%, Yield: 112.5%, not assay-corrected).

### Example II-10a: Preparation of HCl salt of Ac-Pen-Asn-Thr-Trp(7Me)-Lys(Ac)-Pen-Tyr(2-ea)-2Nal-αMe-Lys-Lys(Ac)-Asn-D-Leu-NH2 (S—S, 1-6)

5.0 g of the crude product was dissolved in 1-PrOH:Water (80:20, v:v) (25 mL) at 40° C. and the hazy solution filtered through a pore 3 glass filter. The solution was cooled to 15° C. within 45 minutes, seeded with 50 mg (1%) of crystals and stirred at this temperature for 1 hour.

1-PrOH (75 mL, 62 g) was added to the reaction mixture over 3 hours (a white precipitate is formed). The temperature was adjusted to 0° C. over 4 h and stirred at this temperature overnight.

The product was separated by filtration through a pore 3 glass filter (slow filtration) and the filter cake washed with 1-PrOH (5 mL) at 0° C. The product dried under vacuum at 35° C. over the weekend to give 3.2 g of a white powder, Ac-Pen-Asn-Thr-Trp(7Me)-Lys(Ac)-Pen-Tyr(2-ea)-2Nal-αMe-Lys-Lys(Ac)-Asn-D-Leu-NH2 (S—S, 1-6)×HCl. (HPLC purity: 95.8%, ESI-MS: m/z=924.91 Da [M+2H⁺]²⁺, HPLC Assay: 93.1%, Yield: 78.8%).

### Example II-10b: Preparation of Acetate salt of Ac-Pen-Asn-Thr-Trp(7Me)-Lys(Ac)-Pen-Tyr(2-ea)-2Nal-αMe-Lys-Lys(Ac)-Asn-D-Leu-NH2 (S—S, 1-6)

A solution of Ac-Pen-Asn-Thr-Trp(7Me)-Lys(Ac)-Pen-Tyr(2-ea)-2Nal-αMe-Lys-Lys(Ac)-Asn-D-Leu-NH2 (S—S, 1-6)x HCl salt (5.0 g) in 100 mL MeOH:H₂O (9:1, v:v) (hazy solution) was percolated through a Lewatit MP64 (acetate form) (25 g) column and the column washed with water (300 mL). The eluate was collected in fractions of 50 mL that were analysed by TLC to detect the presence of product. The fractions of interest (ca. 250 mL) were mixed and the solution filtered through a 0.45 μm TPP filter. The filtered solution was frozen and freeze-dried to give 5.02 g of a white powder, Ac-Pen-Asn-Thr-Trp(7Me)-Lys(Ac)-Pen-Tyr(2-ea)-2Nal-αMe-Lys-Lys(Ac)-Asn-D-Leu-NH2 (S—S, 1-6)×acetate (HPLC purity: 96.9%, ESI-MS: m/z=924.88 Da [M+2H⁺]²⁺, HPLC Assay: 92.9%, Yield: 98.0%).

The following reaction scheme may aid in understanding the reactions discussed throughout the following examples.

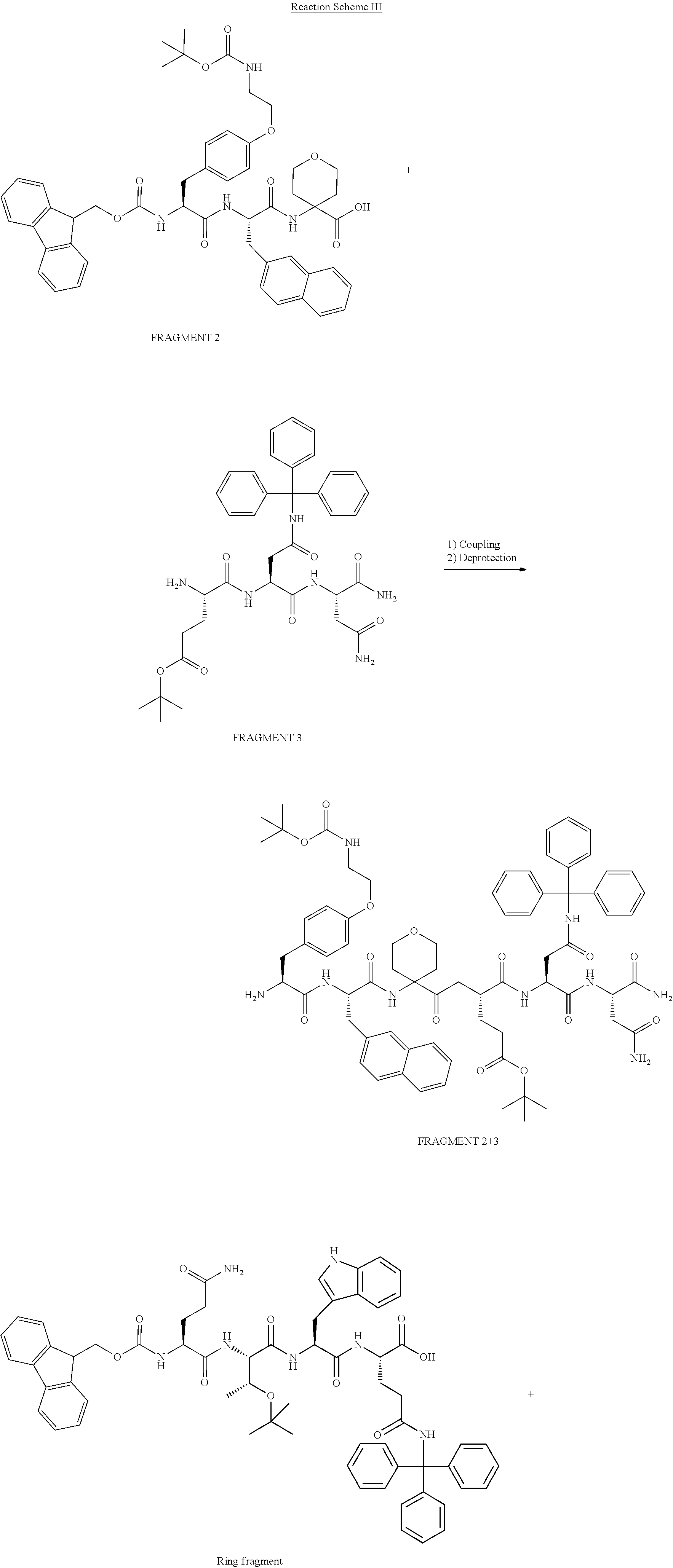

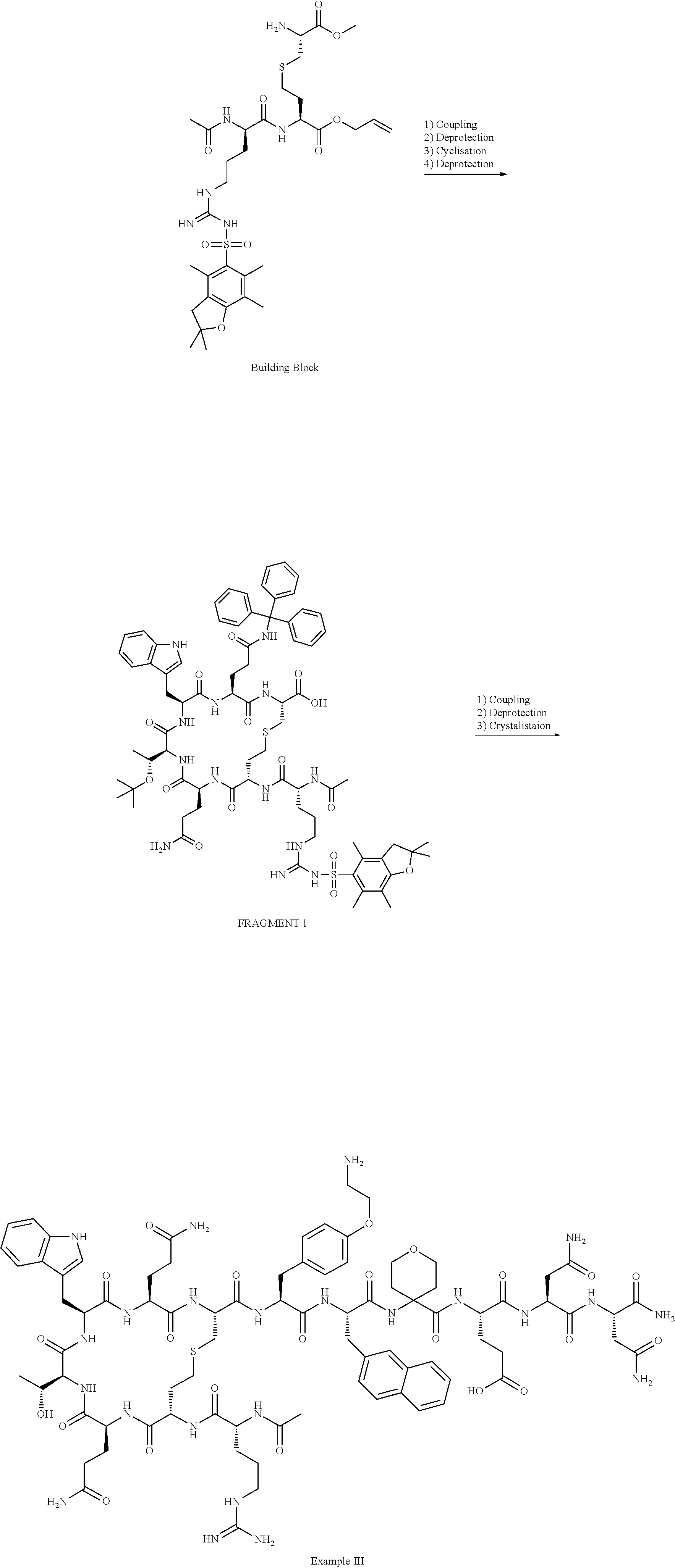

### Example III-1: Synthesis of Fragment 2

### Example III-1a: Synthesis H-THPGly-OMe*HCl

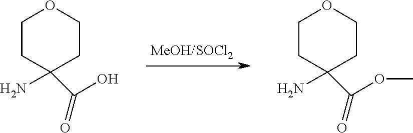

In a 500 mL flask, MeOH (300 ml) was cooled to −5° C. Thionylchloride (31.7 g/2.0 eq.) was added within 20 min at −5 to 0° C. and stirred for 15 min. To the reaction mixture at −2 to 0° C., H-THPGly-OH (20.00 g/1.00 eq.) was added portion wise within 2 min and stirred for 15 min at 0° C. Then heated to reflux with 75° C. (jacket temperature) and stirred at reflux. The solution was evaporated to an oil. IPE (250 mL) was added and a white suspension was formed. After stirring for 15 min at r.t. the suspension was filtered and washed with 4×20 ml IPE. The product was dried under reduced pressure in a vacuum oven at 40° C. to yield 24.49 g (91%).

### Example III-1b: Synthesis Boc-2-Nal-THPGly-OMe

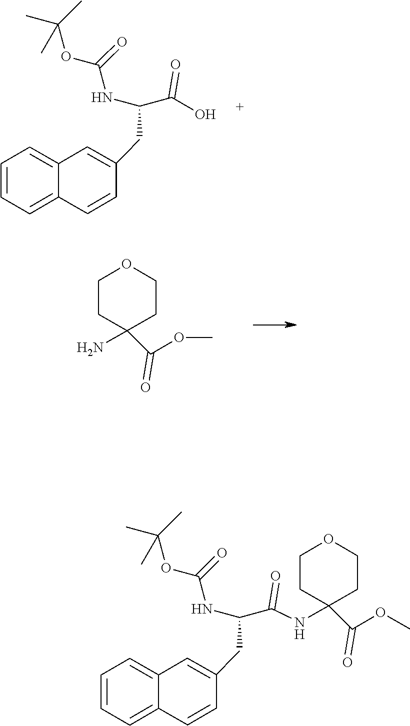 [SMILES: CC(C)(C)OC(=O)N[C@@H](Cc1ccc2ccccc2c1)C(=O)O ; COC(=O)C1(N)CCOCC1 ; COC(=O)C1(NC(=O)[C@H](Cc2ccc3ccccc3c2)NC(=O)OC(C)(C)C)CCOCC1]

In a 2 L flask, Boc-2-Nal-OH (70.00 g/1.0 eq.) and H-THPGly-OMe*HCl (46.60 g/1.05 eq.) were dissolved in DMF (700 mL). The clear, yellow solution was cooled to 10° C. and NMM (76 ml/3.1 eq.) was added. The formed white thin suspension was cooled to 0° C. TBTU (78.40 g/1.1 eq.) was added in one portion. The suspension was stirred in the ice bath for 2 h. Then warmed to 20° C. in 30 min and stirred further. The reaction mixture was stirred overnight at room temperature (25° C.). The reaction mixture was diluted with EtOAc (1400 mL) and washed with NaHCO₃-solution (2%, 800 ml). The aq. layer was extracted with EtOAc (300 ml). The combined organic layers were washed with NaHCO₃-solution (2%)/NaCl solution (5% aq.) (800 ml) and NaCl solution 10% (2×400 mL). The organic layer was dried over Na₂SO₄ and clear filtered. The filtrate was evaporated under reduced pressure at 45° C. to an oil and co-distillated with EtOAc (100 mL) to a residue of 180 g. The EtOAc oil was diluted with IPE (245 ml) and brought to turbidity with heptane (180 ml). The turbid solution was seeded and after 10 min a white, very thick suspension was formed. The suspension was diluted with heptane (820 mL) and stirred for an additional 30 min. The product was filtered, washed with heptane (4×80 mL) and dried in a vacuum oven at 40° C. to yield 86.61 g/85%.

### Example III-1c: Synthesis Boc-2-Nal-THPGly-OH

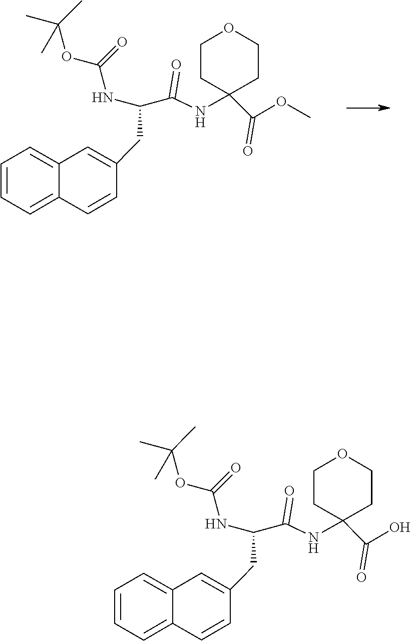 [SMILES: COC(=O)C1(NC(=O)[C@H](Cc2ccc3ccccc3c2)NC(=O)OC(C)(C)C)CCOCC1 ; CC(C)(C)OC(=O)N[C@@H](Cc1ccc2ccccc2c1)C(=O)NC1(C(=O)O)CCOCC1]

In a flask Boc-2-Nal-THPGly-OMe (86.50 g/1.0 eq.) was stirred in dioxane (680 ml) and water (1020 ml). NaOH 30% (29.1 mL/1.5 eq.) was added and stirred at room temperature (pH 14.0). After 15 min a turbid solution with some lumps was formed. Additional dioxane (120 mL) and water (180 mL) were added. After 4 h the nearly clear solution turned into a jelly suspension. Water (200 ml) was added the suspension got thinner; the temperature was raised from 26° C. to 40° C. At 35° C. the reaction mixture was a clear, yellowish solution. After 5 h, the solution was evaporated under reduced pressure at 50° C. (dioxane removed, 850 ml distillate). The aqueous solution formed a thick gel. The gel was diluted with water (700 ml) and cooled to 20° C. The pH adjusted to 3 with NaHSO₄-solution (5%, 300 mL) and NaHSO₄-solution (20%, 50 mL). The gel started to form a fine white suspension. The suspension was stirred for 15 min. The thin white suspension was filtered and washed with water (3×150 mL). The product was dried in a vacuum oven at 45° C. to yield 83.05 g/99%.

### Example III-1d: Synthesis H-2-Nal-THPGly-OH*HCl

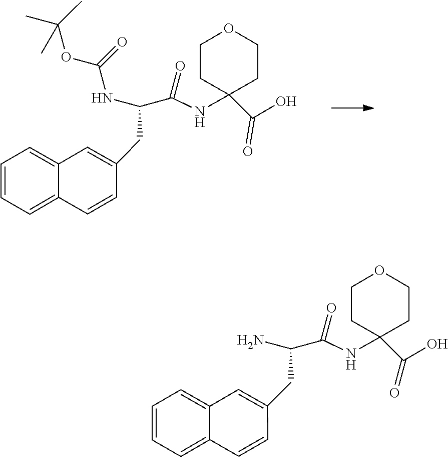 [SMILES: CC(C)(C)OC(=O)N[C@@H](Cc1ccc2ccccc2c1)C(=O)NC1(C(=O)O)CCOCC1 ; N[C@@H](Cc1ccc2ccccc2c1)C(=O)NC1(C(=O)O)CCOCC1]

In a flask HCl/dioxane solution (4 M, 415 mL) was cooled to 0° C. Boc-2-Nal-THPGly-OH (83.0 g) was added as a solid within 10 min at max. 12° C. and rinsed with dioxane (15 mL). The suspension was stirred in an ice-bath. After 45 min, the reaction mixture was precipitated onto IPE (2.1 L). The thin white suspension was filtered and washed with IPE (4×150 mL). The product was dried in a vacuum oven at 40° C. to yield 72.97 g.

### Example III-1e: Synthesis Fmoc-Tyr(2-Boc-ea)-OSu

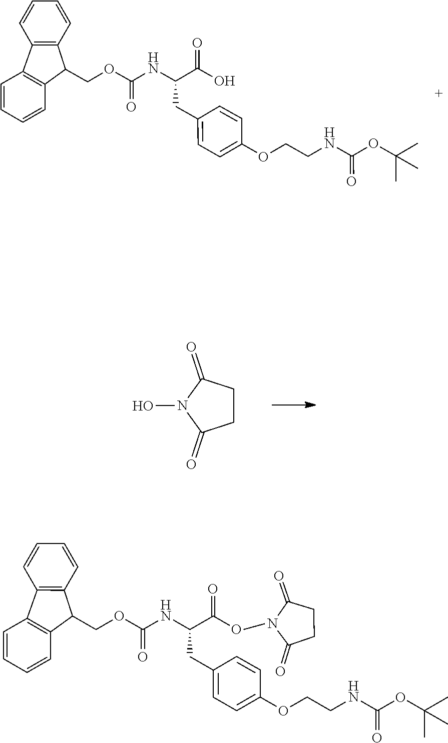 [SMILES: CC(C)(C)OC(=O)NCCOc1ccc(C[C@H](NC(=O)OCC2c3ccccc3-c3ccccc32)C(=O)O)cc1 ; O=C1CCC(=O)N1O ; CC(C)(C)OC(=O)NCCOc1ccc(C[C@H](NC(=O)OCC2c3ccccc3-c3ccccc32)C(=O)ON2C(=O)CCC2=O)cc1]

In a 250 mL flask Fmoc-Tyr(2-Boc-ea)-OH (10.00 g/1.00 eq.) and HOSu (2.32 g/1.1 eq.) were dissolved in THF (100 mL dry, over molecular sieve) at 20° C. under N₂ atmosphere. The clear, colourless solution was cooled to 0° C. A solution of DCC (4.17 g/1.1 eq.) in THF (50 mL dry, over molecular sieve) was added over 120 min at 0±2° C. (dosage speed 0.5 g/min). The dosage unit was flushed with THF (10 mL; dry over molecular sieve) into the reactor. The white suspension was stirred at 0±2° C. for 30 min. Then the temperature was raised to 15° C. over 600 min. (constant ramp). The suspension was clear filtrated and the white residue was washed with THF (2×20 mL). The clear colourless filtrate was evaporated under reduced pressure at 40° C. to a thick oil (20 g). The oil was diluted with IPA (150 mL) and stirred 5 min at 40° C. until a slightly yellow solution appears. Then stirred at r.t.. After 10 min of stirring a white, thick suspension was formed. The suspension was cooled to 5-10° C., stirred for 30 min, filtered and washed with IPA (4×5 mL). The product was dried in a vacuum oven at 35° C. to yield 10.99 g/93%.

### Example III-1f: Synthesis Fmoc-Tyr(2-Boc-ea)-2-Nal-THPGly-OH (Fragment 2)

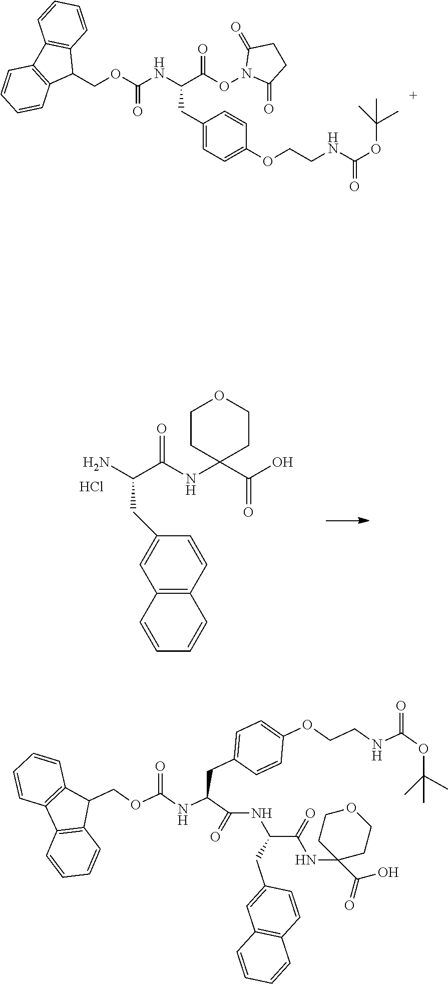 [SMILES: CC(C)(C)OC(=O)NCCOc1ccc(C[C@H](NC(=O)OCC2c3ccccc3-c3ccccc32)C(=O)ON2C(=O)CCC2=O)cc1 ; Cl ; N[C@@H](Cc1ccc2ccccc2c1)C(=O)NC1(C(=O)O)CCOCC1 ; CC(C)(C)OC(=O)NCCOc1ccc(C[C@H](NC(=O)OCC2c3ccccc3-c3ccccc32)C(=O)N[C@@H](Cc2ccc3ccccc3c2)C(=O)NC2(C(=O)O)CCOCC2)cc1]

In a 400 mL flask Fmoc-Tyr(2-Boc-ea)-OSu (20.00 g/1.00 eq.) and H-2-Nal-THPGly-OH*HCl (12.36/1.05 eq.) were dissolved in THF (dry over molecular sieve, 250 ml) at 20° C. The slightly turbid solution was cooled to 15° C. A solution of DIPEA (10.04 g/2.50 eq.) in THF (dry over molecular sieve 50 ml) was added over 3 h at 15° C. The reaction mixture was heated to 20° C. over 4 h; DIPEA (1.35 mL/0.25 eq.) was added. The reaction mixture was heated to 30° C. within 20 min. The reaction mixture was stored in the fridge (5° C.) over the weekend. The reaction mixture was evaporated under reduced pressure at 40° C. to an oil. The oil was dissolved in EtOAc (300 mL) and washed with NaHSO₄ (5%, 2×100 mL) and with NaCl-solution (10%, 2×50 ml). The organic phase was evaporated under reduced pressure at 40° C. to a residue of 140 g. The solution was diluted with IPE (60 mL) to turbidity. The turbid solution was stirred until a thick suspension was formed. The suspension was diluted with IPE (200 mL) and stirred for 1 h. The white suspension was filtrated and washed with IPE (3×50 mL). The product was dried in a vacuum oven at 40° C. to yield 25.87 g/96%. The product was dissolved in EtOAc (200 mL) at 25° C. With IPE (120 mL) the solution was turned into a very fine suspension and stirred further at room temperature until it crystalized. The suspension was diluted with IPE (200 mL), filtered and washed with IPE (3×60 mL). The product was dried in a vacuum oven at 40° C. to yield 23.60 g/87%

### Example III-2: Synthesis of Fragment 3

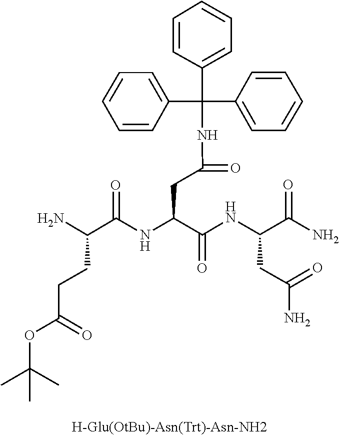 [SMILES: "H-Glu(OtBu)-Asn(Trt)-Asn-NH2" ; CC(C)(C)OC(=O)CC[C@H](N)C(=O)N[C@@H](CC(=O)NC(c1ccccc1)(c1ccccc1)c1ccccc1)C(=O)N[C@@H](CC(N)=O)C(N)=O]

### Example III-2a: Synthesis of H-Asn-NH₂*HCl

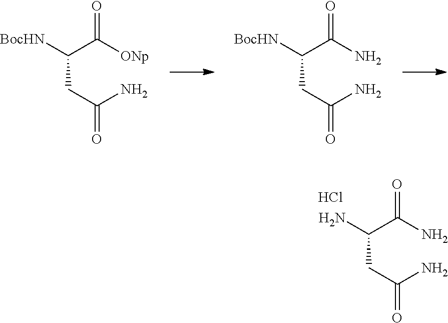

Boc-Asn-ONp (100.0 g, 1.0 eq.) was suspended in Me-THF (2.0 L) and NH₃/MeOH solution (100.0 mL) was added. The reaction mixture turned immediately yellow and formed a suspension. After 15 min IPE (2.5 L) was added to the yellow suspension. The suspension was filtered and washed with IPE (10×250 mL). The white product was dried overnight under reduced pressure in a vacuum oven at 40° C. Boc-Asn-NH2: 50 g (65%).

Boc-Asn-NH2 (4.5 g) was suspended in DMF (35 mL). Then HCl solution in dioxane (˜4 M, 75 mL) was added. The mixture dissolved within 2 min and after 40 s a white suspension was formed. The suspension was stirred for 15 min then filtered and washed with DMF (3×20 mL). The product was dried at 40° C./HV.H-Asn-NH₂*HCl: 3.0 g (92%)

Boc-Asn-NH2 (50.0 g, 1.0 eq.) was suspended in DMF (500 mL). Then HCl solution in dioxane (ca. 4 M, 400 mL) was added. The temperature raised up to 50° C. The mixture dissolved within 2 min. After 10 min, when the temperature cooled down to 44° C., the product started to crystallize. Stirred for 2 h until the temperature reached 20-25° C., then filtered and washed with DMF (3×200 mL). The product was dried at 40° C./HV. H-Asn-NH₂*HCl: 17.5 g (48.3%).

### Example III-2b: Synthesis of Z-Asn(Trt)-Asn-NH₂

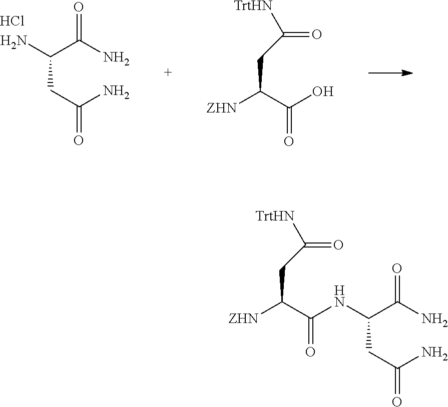

Z-Asn(Trt)-OH (10.93 g, 0.90 eq) was dissolved in DMF (95 mL). Then TBTU (1.0 eq) was added and stirred again until a colourless solution was obtained. In another flask H-Asn(Trt)-NH₂*HCl (4.12 g, 1.0eq. assay: 102.94%) was added and dissolved in DMSO (25 mL). This turbid solution was added to the first mixture. Then NMM was added (15 mL, 5.65 eq.) and the reaction mixture was stirred at room temperature (the pH reached pH 7.5-8.0). After an hour the mixture was evaporated at 60° C./HV. The thick oil (+DMSO) was dissolved with EtOAc (25 mL) and precipitated into water (800 mL, 1/16 v:v). The product was filtered, washed with water (2×20 mL) and EtOAc (2×20 mL) and dried at 40° C./HV to yield Z-Asn(Trt)-Asn-NH2: 11.6 g-78.2%.

### Example III-2c: Synthesis of H-Asn(Trt)-Asn-NH₂*HCl

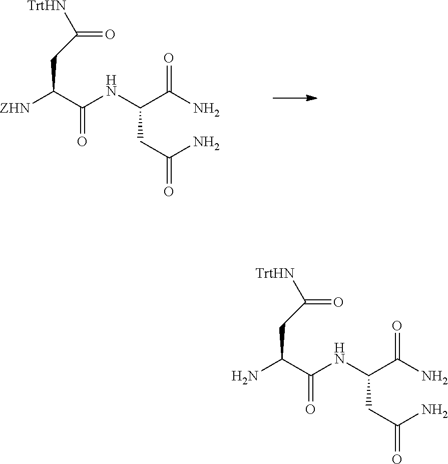

In a hydrogenator Z-Asn(Trt)-Asn-NH₂ (0.200 g, 1.0 eq.) was suspended in MeOH/H₂O (95:5) (10 mL) and Pd/C 5% (0.023 g) was added. The hydrogenation was started at 45° C./3.5 bar H₂ pressure. After 30 min a solution (with black catalyst particles) was formed. The reaction mixture was clear filtrated and washed with MeOH. The filtrate was evaporated under reduced pressure at 45° C. to a white, oily solid. The solid was co-distillated with EtOAc (2×10 ml) and dissolved in MeOH (2 mL). The solution was precipitated on IPE (15 mL). The suspension was centrifuged and the product was washed once with IPE (20 mL). The product was dried in a vacuum oven at 40° C. to yield a white powder.

### Example III-2d: Synthesis of Z-Glu(OtBu)-OH

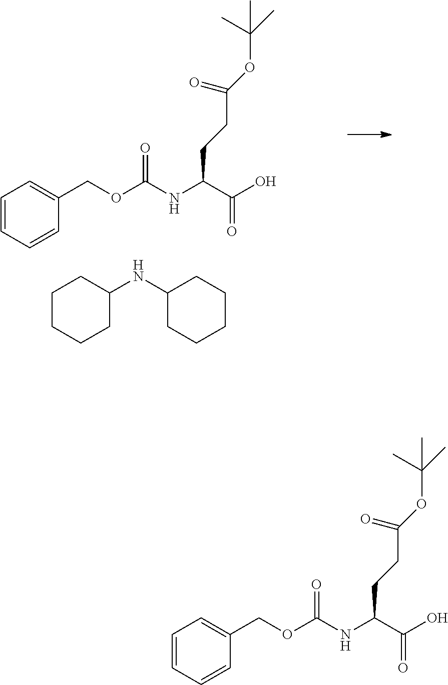 [SMILES: CC(C)(C)OC(=O)CC[C@H](NC(=O)OCc1ccccc1)C(=O)O ; C1CCC(NC2CCCCC2)CC1 ; CC(C)(C)OC(=O)CC[C@H](NC(=O)OCc1ccccc1)C(=O)O]

Z-Glu(OtBu)-OH*DCHA (400.0 g) was weighed into a 3 L flask following the addition of MTBE (1.5 L) and water (0.25 L) to the flask. To the white suspension, H₂SO₄-solution (50%, 100 mL) was added until the pH reached 1.5. When the pH reached pH 2.0, the suspension started to dissolve. The layers were separated and the water layer was washed with MTBE (400 mL). The organic layer was evaporated at 60° C./HV to an oil and dried by azeotropic distillation with EtOAc (3×300 mL). To the oil IPE (1 L) and seed-crystals were added and stirred overnight at r.t.. The white suspension was filtered, washed with IPE (2×200 mL) and dried at 40° C./HV. Z-Glu(OtBu)-OH 99.5 g/37%.

### Example III-2e: Synthesis of Z-Glu(OtBu)-Asn(Trt)-Asn-NH2

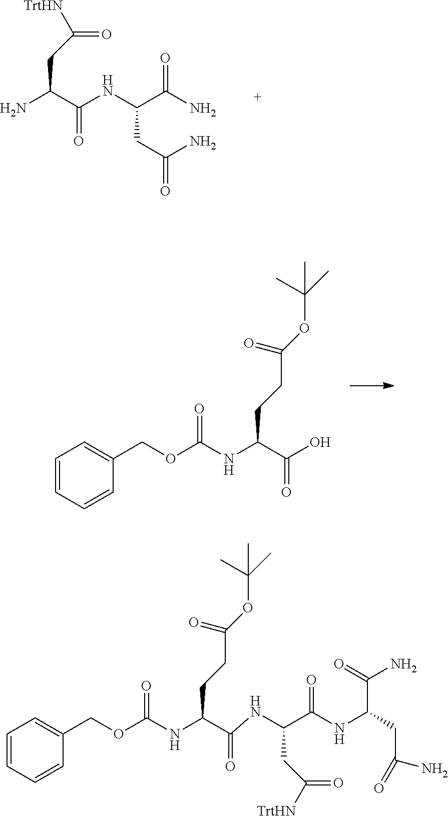

In a flask, Z-Glu(OtBu)-OH (5.73 g, 1.00 eq.) and H-Asn(Trt)-Asn-NH₂ (8.28 g, 1.00 eq.) was dissolved in DMF (85 mL). The solution was cooled to 0° C. DIPEA (9.2 mL, 3.10eq.) was added followed by TBTU (6.00 g, 1.1 eq.) all in the ice bath (AT=0° C.). IT raised to 10° C.→clear, slightly yellowish solution formed. The solution was stirred 1 h at 0° C. then at room temperature (25° C.). The reaction mixture was precipitated by adding on NaHCO₃ (2%) solution (400 ml). The white suspension was filtered and washed with water (4×100 mL) and EtOAc (3×80 mL). The product was dried in a vacuum oven at 45° C. to yield 12.08 g/88%.

### Example III-2f: Synthesis of H-Glu(OtBu)-Asn(Trt)-Asn-NH₂ (Fragment 3)

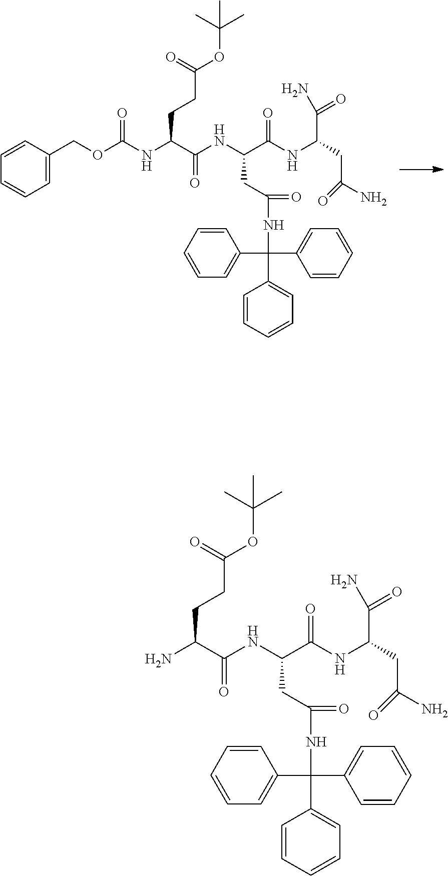 [SMILES: CC(C)(C)OC(=O)CC[C@H](NC(=O)OCc1ccccc1)C(=O)N[C@@H](CC(=O)NC(c1ccccc1)(c1ccccc1)c1ccccc1)C(=O)N[C@@H](CC(N)=O)C(N)=O ; CC(C)(C)OC(=O)CC[C@H](N)C(=O)N[C@@H](CC(=O)NC(c1ccccc1)(c1ccccc1)c1ccccc1)C(=O)N[C@@H](CC(N)=O)C(N)=O]

In a hydrogenation flask Z-Glu(OtBu)-Asn(Trt)-Asn-NH₂ (15.00 g), Pd/C 5% (1.50 g) were suspended in methanol (135 mL) and water (15 mL). The hydrogenation was started at 45° C./3.5 bar H₂. After 1 h a black solution was formed. The suspension was clear filtrated and washed with methanol. Water (100 mL) was added to the filtrate. The filtrate was evaporated under reduced pressure at 40° C. (80 mL distillate). A thick suspension was formed. Additional water (200 mL) was added. A thick, stirrable suspension was formed. With NaHCO₃-5%-solution (approx. 20 mL) the pH was raised to 8.5. The suspension was filtered and washed with water (3×80 mL) and EtOAc (3×40 ml). The product was dried in a vacuum oven at 45° C. to yield 11.16 g/89%.

### Example III-3: Synthesis of Fragment 2+3

### Example III-3a: Fmoc-Tyr(2-Boc-ea)-2-Nal-THPGly-Glu(OtBu)-Asn(Trt)-Asn-NH₂ (Fmoc-FG2+3-NH2)

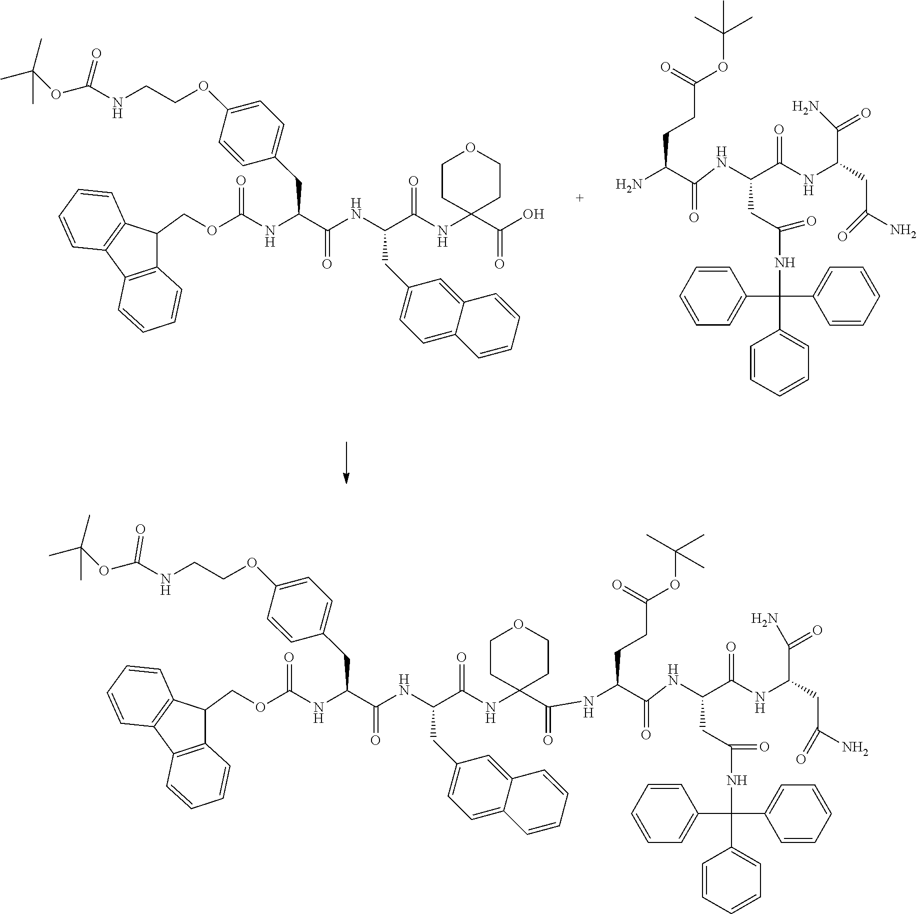 [SMILES: CC(C)(C)OC(=O)NCCOc1ccc(C[C@H](NC(=O)OCC2c3ccccc3-c3ccccc32)C(=O)N[C@@H](Cc2ccc3ccccc3c2)C(=O)NC2(C(=O)O)CCOCC2)cc1 ; CC(C)(C)OC(=O)CC[C@H](N)C(=O)N[C@@H](CC(=O)NC(c1ccccc1)(c1ccccc1)c1ccccc1)C(=O)N[C@@H](CC(N)=O)C(N)=O ; CC(C)(C)OC(=O)CC[C@H](NC(=O)C1(NC(=O)[C@H](Cc2ccc3ccccc3c2)NC(=O)[C@H](Cc2ccc(OCCNC(=O)OC(C)(C)C)cc2)NC(=O)OCC2c3ccccc3-c3ccccc32)CCOCC1)C(=O)N[C@@H](CC(=O)NC(c1ccccc1)(c1ccccc1)c1ccccc1)C(=O)N[C@@H](CC(N)=O)C(N)=O]

In a 500 mL flask H-Glu(OtBu)-Asn(Trt)-Asn-NH₂ (13.96 g/1.00 eq.) and Fmoc-Tyr(2-Boc-ea)-2-Nal-THPGly-OH (18.3 g/1.00 eq) were suspended in 2-Me-THF (324 mL) and DMSO (36 mL)(9/1). To the white, thick suspension 2,4,6-collidine (8.14 mL/3.00 eq.) was added. To the still thick suspension PyAOP (14.4 g/1.05 eq.) was added at once. After 2 min of stirring a yellow, clear solution was formed. The reaction mixture was stirred at room temperature (25° C.), 2,4,6-collidine (2 mL) was added (pH raised to 7.5) and then stirred overnight. Afterwards another portion of PyAOP (0.7 g) was added and the reaction mixture was stirred further at room temperature. The reaction mixture was washed with NaHCO₃-5%-solution (2×100 mL) and brine (2×100 mL). Na₂SO₄ was added to the organic layer, it was stirred for 5 min and clear filtrated. The mother liquor was evaporated at 55° C./HV to an oil. The oil was dissolved in methanol (360 mL). The clear solution was stirred at room temperature. After 3 min it started to crystalize and formed a white suspension (1 hour). The suspension was cooled to 18° C. and stirred for an additional 30 min. The suspension was filtrated and washed with methanol (4×50 ml). The product was dried in a vacuum oven at 35° C. to yield 28.6 g/91.6%.

### Example III-3b: Synthesis of Fragment 2+3 (H-FG2+3-NH₂)

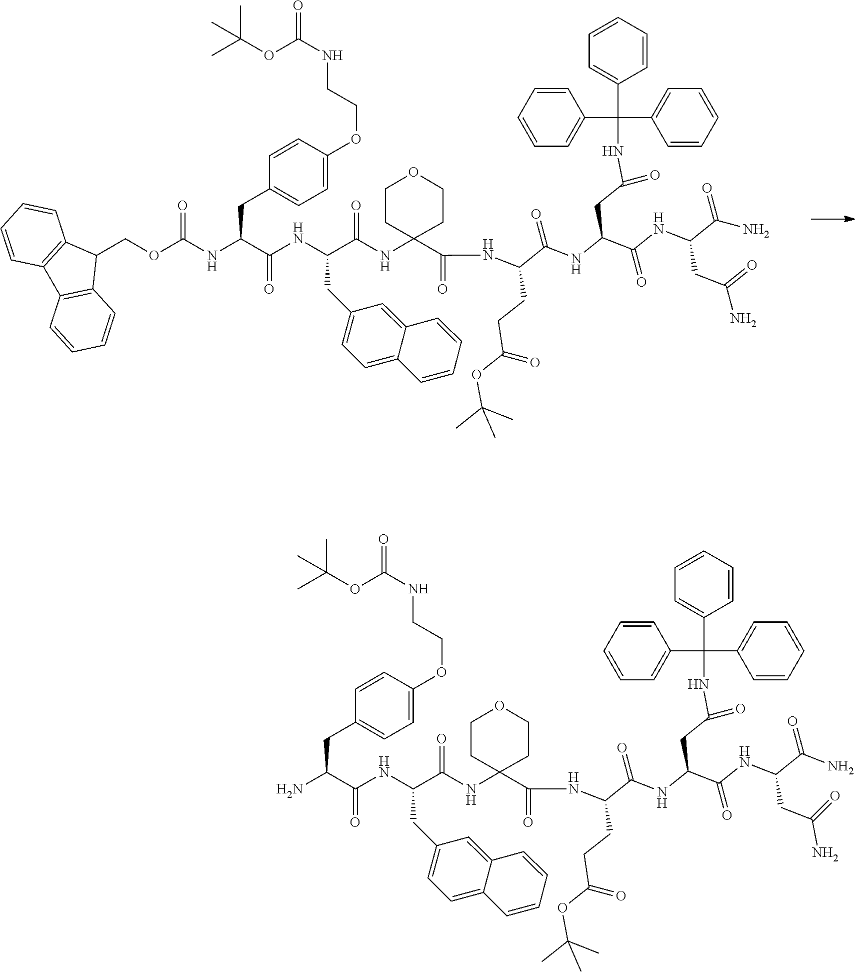 [SMILES: CC(C)(C)OC(=O)CC[C@H](NC(=O)C1(NC(=O)[C@H](Cc2ccc3ccccc3c2)NC(=O)[C@H](Cc2ccc(OCCNC(=O)OC(C)(C)C)cc2)NC(=O)OCC2c3ccccc3-c3ccccc32)CCOCC1)C(=O)N[C@@H](CC(=O)NC(c1ccccc1)(c1ccccc1)c1ccccc1)C(=O)N[C@@H](CC(N)=O)C(N)=O ; CC(C)(C)OC(=O)CC[C@H](NC(=O)C1(NC(=O)[C@H](Cc2ccc3ccccc3c2)NC(=O)[C@@H](N)Cc2ccc(OCCNC(=O)OC(C)(C)C)cc2)CCOCC1)C(=O)N[C@@H](CC(=O)NC(c1ccccc1)(c1ccccc1)c1ccccc1)C(=O)N[C@@H](CC(N)=O)C(N)=O]

Fmoc-Tyr(2-Boc-ca)-2-Nal-THPGly-Glu(OtBu)-Asn(Trt)-Asn-NH₂ (Fmoc-FG2+3-NH2, 28.5 g) was dissolved in 2-Me-THF (280 mL) and DBU (4.18 mL) was added. The reaction mixture was stirred at room temperature for 1.0 h and precipitated onto 2.8 L IPE to form a white suspension. The suspension was filtered, washed twice with IPE (200 mL). and dried at 35° C./HV overnight to yield 25.1 g-103.1% (not assay corrected, the product contains dibenzofulvene). FG 2+3 (25.1 g) was weighed in a 500 mL round flask And MeOH (100 mL) was added. To dissolve it was warmed up to 45° C. and then stirred at r.t. It started to crystallize. The suspension was stirred for 1 h at r.t., IPE (100 mL) was added, then cooled to 0-5° C. and stirred for 5 h at this temperature. Filtered the suspension and washed it twice with IPE (100 mL). Then the product was dried at 40° C./HV to yield 20.8 g-85.4%.

### Example III-4: Synthesis of Building Block

### Example III-4a: Synthesis of Boc-Hse-OAll

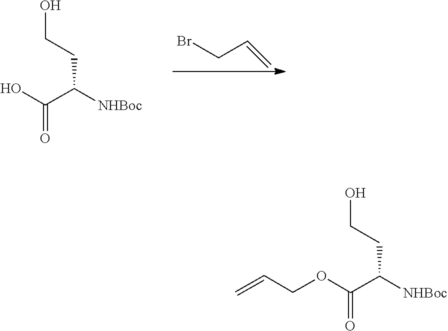

In a 10 L glass reactor were loaded acetonitrile (3.0 L) and Boc-Hse-OH (300.0 g, 1.368 mol, 1.0 eq). The suspension formed was stirred at r.t. and DIPEA (530.61 g, 4.105 mol, 3.0 eq) was added at once. The reaction mixture was stirred at r.t. until a clear solution was obtained. To this solution, ally bromide (331.13 g, 2.736 mol, 2.0 eq) was added and the reaction mixture stirred at r.t. IPC-HPLC after 48 hours showed that the reaction is finished. At this point the solvent was evaporated under reduced pressure at 35° C. until a white suspension starts to form. The residue was taken up in EtOAc (3.0 L) and the white suspension formed stirred at r.t. for 10 minutes until a homogeneous white suspension was formed. The precipitate was separated by filtration and the filter cake washed with EtOAc (0.1 L). The organic phase was then washed with 5% NaHCO₃ (3×1.5 L), brine (1.5 L) and dried with Na₂SO₄ anhydrous, followed by evaporation of the solvent under reduced pressure at 35° C. to an oil. The oil was resuspended in IPE:heptane (1:1, v:v) (3.0 L) and the suspension formed stirred for 0.5 hours at r.t. until a clear fine white suspension was obtained. The solid was separated by filtration through a pore 3 glass filter and the filter cake washed with IPE:heptane (1:1, v:v) (2×0.2 L). The solvent of the filtrate was evaporated under reduced pressure at 35° C. to an oil and dried under high vacuum at r.t. for 18 hours to give 260 g of a yellow oil, Yield: 73.3%.

### Example III-4b: Synthesis of Boc-Abu(4-Cl)-OAll

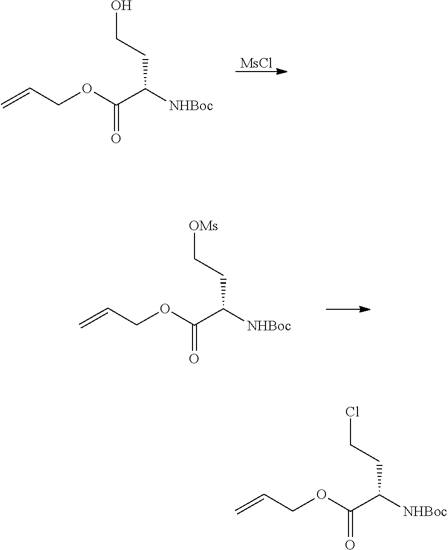 [SMILES: "MsCl" ; C=CCOC(=O)[C@H](CCO)NC(=O)OC(C)(C)C ; C=CCOC(=O)[C@H](CCOS(C)(=O)=O)NC(=O)OC(C)(C)C ; C=CCOC(=O)[C@H](CCCl)NC(=O)OC(C)(C)C]

To a stirred solution of Boc-Hse-OAll (255.0 g, 0.983 mol, 1.0 eq.) in DCM (2.0 L) at 0-5° C. was added triethylamine (129.37 g, 1.278 mol, 1.3 eq.) and the reaction mixture stirred at 0-5° C. for 5-10 minutes. Mesyl chloride (135.2 g, 1.180 mol, 1.2 eq.) was added dropwise over 10 minutes. The reaction mixture was stirred at 0-5° C. for 10 minutes after complete addition of mesyl chloride and heated to 20-25° C. over 30 minutes. IPC-HPLC two hours after complete addition of MsCl, showed completeness of the reaction. The reaction mixture was filtered through a pore 3 glass filter to remove the solid formed and the solvent was evaporated to an oil under reduced pressure at 35° C. The oil was redissolved in DMF (2.0 L), LiCl (208.5 g, 4.917 mol, 5 eq.) was added and the reaction mixture was stirred at 20-25° C. IPC-HPLC after 18 hours, showed almost quantitative conversion of the starting material.

At this point the reaction mixture was filtered to separate the solid and the filtrate concentrated under reduced pressure at 60° C. to an oil. The oil was redissolved in a mixture of IPE:water (2:1, v:v) (6.0 L) and stirred until no solid was present. The phases were left to separate and the organic phase was washed with water (2.0 L), NaHCO₃ (2%) (2×2.0 L), brine (1.5 L) and dried with Na₂SO₄ anhydrous. The solvent was then evaporated under reduced pressure at 40° C. to give a yellowish oil. The oil was dried under high vacuum at r.t. for 18 hours to give 235 g of a whitish solid. (HPLC purity: 98.3%, ESI-MS: m/z=178.07/180.05 [M-Boc+H⁺]⁺). The crude product (225 g) was diluted with heptane (1500 mL) at 50° C. The product solution was cooled to 0-5° C. in an ice water bath. A thick suspension was formed slowly. This suspension was stirred at 0-5° C. for 30 minutes and the product separated by filtration through a pore 3 glass filter. The filter cake was then dried under vacuum at 35° C. to give 208.0 g of a white solid. (HPLC: 98.6%, yield: 76.2%).

### Example III-4c: Synthesis of H-Abu(4-Cl)-OAll x HCl

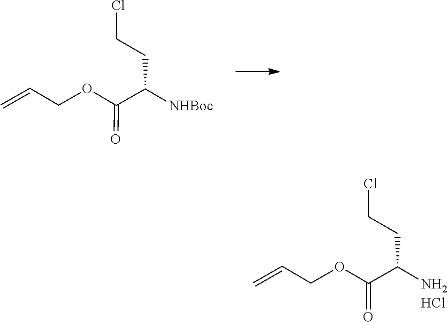 [SMILES: C=CCOC(=O)[C@H](CCCl)NC(=O)OC(C)(C)C ; C=CCOC(=O)[C@@H](N)CCCl ; Cl]

To a round-bottom flask with overhead stirring containing Boc-Abu(4-Cl)-OAll (208 g, 748.9 mmol) was added 4 N HCl in dioxane (1.5 L) pre-cooled at 4° C. The reaction mixture was stirred while left warm to r.t. TLC-IPC after 60 minutes showed completeness of the reaction. At this point the solvent was evaporated under reduced pressure at 35° C. The product precipitated during the concentration. The solid was re-dissolved in dioxane (0.5 L) and the solvent evaporated under reduced pressure at 35° C. again until the product started to precipitate. IPE (3.5 L) was added and the suspension was stirred in an ice-water bath for 30 minutes. The product was then separated by filtration, the filter cake washed with IPE (2×200 mL) and the product dried under vacuum at 35° C. for 18 hours to give 158.5 g of a white solid (: 98.9%).

### Example III-4d: Synthesis of Boc-D-Arg(Pbf)-Abu(4-Cl)-OAll

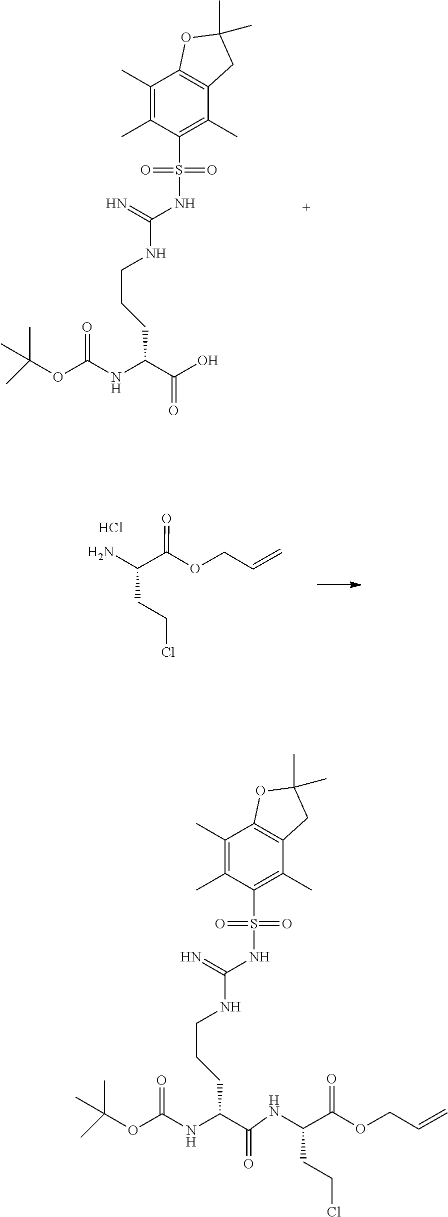 [SMILES: Cc1c(C)c(S(=O)(=O)NC(=N)NCCC[C@@H](NC(=O)OC(C)(C)C)C(=O)O)c(C)c2c1OC(C)(C)C2 ; Cl ; C=CCOC(=O)[C@@H](N)CCCl ; C=CCOC(=O)[C@H](CCCl)NC(=O)[C@@H](CCCNC(=N)NS(=O)(=O)c1c(C)c(C)c2c(c1C)CC(C)(C)O2)NC(=O)OC(C)(C)C]

To a stirred solution of Boc-D-Arg(Pbf)-OH (400.0 g, 91% assay, 691.16 mmol, 1.0 eq.), H-D-Abu(4-Cl)—OH (148.0 g, 99.4% assay, 691.16 mmol, 1.0 eq.) in DMF:AcN (1:4, v:v) (2.5 L) at 2° C., was added DIPEA (268.0 mL, 2073.48 mmol, 3.0 eq.) followed by TBTU (233.0 g, 725.72 mmol, 1.05 eq.) and the reaction mixture stirred at 2° C. for 10 min, warmed to 25° C. within 30 minutes and stirred at this temperature. IPC-HPLC after 8 hours showed that the reaction was complete (S.M: ≤0.1%). The reaction mixture was cooled to 0° C. and stirred for 12 hours (overnight). The solvent was evaporated under reduced pressure at 40° C. until no more solvent distillate was observed and the residue was taken up in EtOAc (12.0 L). The organic phase was washed with 5% NaHCO₃ aq. (6 L), 5% NaHCO₃ aq.: brine (5:1, v:v) (6 L), brine (5 L) and dried with Na₂SO₄ anh. The organic phase was concentrated by evaporation under reduced pressure to an oil and the residue was taken up in MTBE (1.0 L). The product was precipitated by adding the concentrated product solution slowly to IPE:heptane (1:1, v:v) (10.0 L) at 0-5° C. The white suspension formed was stirred at 0-5° C. for 15 minutes and the product separated by filtration through a pore 3 glass filter. The filter cake was washed with IPE:heptane (1:1, v:v ) (2×250 mL) and dried under vacuum at 35° C. to give 470.0 g of a white solid. (HPLC purity: 96.6%, (Boc-D-Arg(Pbf)-Hse(lactone): 1.4%, ESI-MS: 686.48 (M+H⁺)+, yield: 99.1%).

### Example III-4f: Synthesis of H-D-Arg(Pbf)-Abu(4-Cl)-OAll x HCl

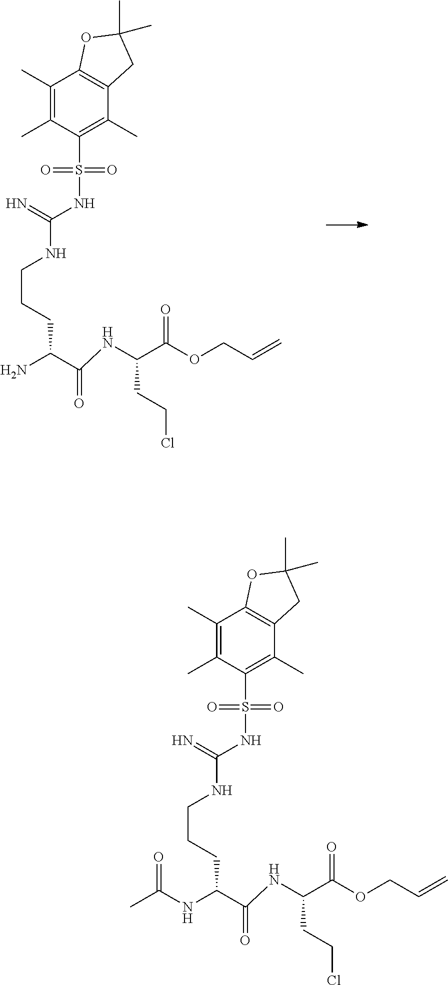

To a stirred solution of Boc-D-Arg(Pbf)-Abu(4-Cl)-OAll (220.0 g, 320.6 mmol, 1.0 eq) in EtOAc (550 mL) at 0-5° C. was added 4 N HCl in EtOAc (1.65 L) pre-cooled at 0-5° C. and the reaction mixture was stirred at 0-5° C. IPC-HPLC after 2 hours showed complete reaction. IPE (6.0 L) was added and the suspension formed stirred for 15 minutes at 0-5° C. The product was separated by filtration, the filter cake washed with IPE (2×500 mL) and dried under reduced pressure at 35° C. to give 199 g of a white solid. (HPLC purity: 95.2%, yield: 99.7%).

### Example III-4 g: Synthesis of Ac-D-Arg(Pbf)-Abu(4-Cl)-OAll

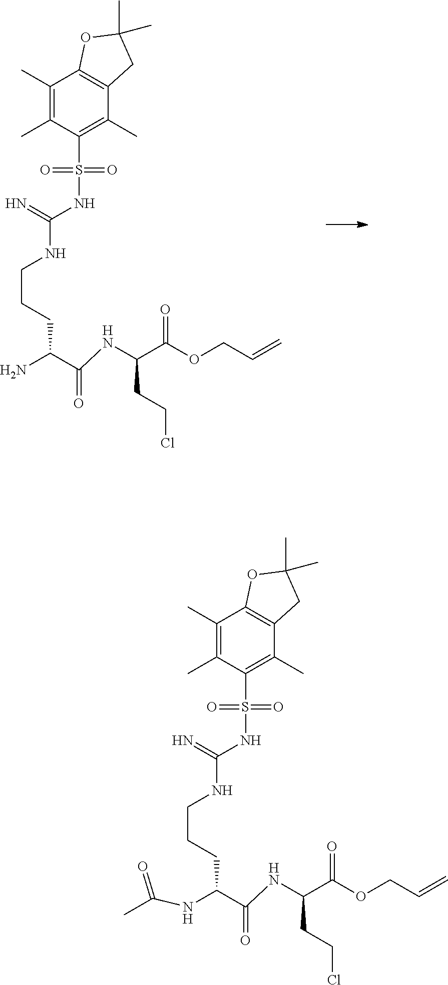

To a stirred solution of H-D-Arg(Pbf)-Abu(4-Cl)-OAll x HCl (380.0 g, 610.34 mmol, 1.0 eq) in MeOH (4.0 L) at 20-25° C., acetic anhydride (86.6 mL, 915.5 mmol, 1.5 eq.) and 2,4,6-collidine (166.4 mL, 1373.3 mmol, 2.25 eq.) were added and the reaction mixture stirred at 20-25° C. IPC-HPLC after 1 hour showed completeness of the reaction. The solvent was evaporated under reduced pressure at 40° C. and the residue taken up in EtOAc (6.0 L). The white suspension formed was stirred at r.t. for 5-10 min and the solid separated by filtration. The filtrate was then washed with NaHSO₄(5% aq.) (2×3.0 L), NaHCO₃ (5% aq.) (2×3.0 L), brine (3.0 L) and dried over Na₂SO₄ anhydrous. The solvent was evaporated under reduced pressure at 40° C. to an oil and the oil was taken up in a mixture of EtOAc:MtBE (1:1, v:v) (1 L). The product was precipitated by adding the concentrated product solution to IPE:heptane (1:1, v:v ) (12 L) slowly at 0-5° C. The suspension formed was stirred at 0-5° C. for 30 minutes and the product separated by filtration through a pore 3 glass filter. The filter cake was then washed with IPE:heptane (1:1, v:v) (2×0.5 L) and dried under vacuum at 35° C. to give 320.0 g of a white solid (HPLC purity: 96.0%, ESI-MS: m/z=628.3 [M+H⁺]⁺, yield: 83.5%.

### Example III-4 h: Synthesis of Ac-D-Arg(Pbf)-Abu(4-(S)-Cys-OMe)-OAll (Building block)

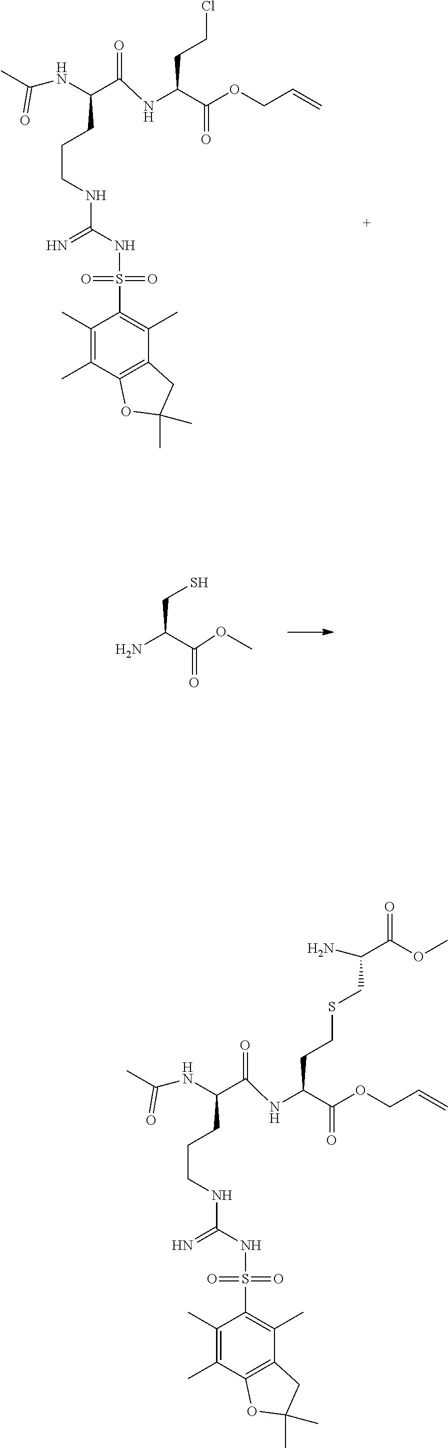

A solution of Ac-D-Arg(Pbf)-Abu(4-Cl)-OAll (180.0 g, 286.5 mmol, 1.0 eq.) and H-Cys-OMe x HCl (123.0 g, 716.3 mmol, 2.5 eq.) in degassed DMSO (0.5 L) was prepared (Sln A). This solution was added to a stirred suspension of Cs₂CO₃ (280.1 g, 859.6 mmol, 3.0 eq) and degassed DMSO (0.4 L) over 1 hour at 23±2° C. IPC-HPLC 2 hours after complete addition of Sln A showed completeness of the reaction. The reaction mixture was added to a mixture of Me-THF:NaHCO₃ (5%, aq): brine (6:3:1, v:v:v) (15 L) and stirred for 5-10 minutes. The phases were left to separate and the organic phase was washed with NaHCO₃ (5%, aq): brine (3:1, v:v ) (6.0 L), brine (4.0 L). The aqueous phase from the first wash was extracted with MeTHF (2×3.0 L) and the organic phases were mixed with the original and dried with Na₂SO₄ anhydrous. The solvent was then evaporated under reduced pressure at 40° C. to ca 0.9 L. This solution was added to IPE (9.0 L). and the product precipitated. The solid was filtered off and the filter cake was washed IPE (2×0.5 L) and dried under vacuum at 35° C. to give 166.0 g of a white solid (HPLC purity: 91.5%, yield: 79.7%).

218 g of this crude product was dissolved in THF (1.5 kg) at 40° C. The solution was cooled to 0° C. within 40 min and the solution stirred at this temperature for 30 minutes. IPE (0.5 kg) was added over 30 minutes at 0° C. and the suspension formed stirred at 0° C. for one hour more. The product was separated by filtration through a pore 3 glass filter, the filter cake was washed with THF:IPE (3:1, w.w) precooled at −5° C. (2×300 mL) and dried under vacuum at 35° C. for 18 hours to give 192 g of a white solid. HPLC purity: 96.9% ESI-MS: m/z=727.53 [M+H⁺]⁺, Yield: 88.0%.

### Example III-5: Ring Fragment Synthesis

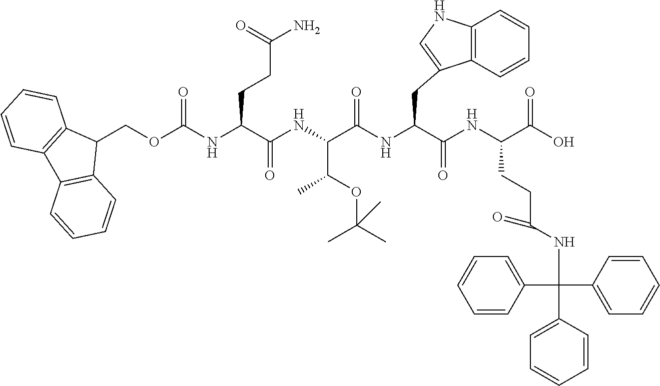 [SMILES: C[C@@H](OC(C)(C)C)[C@H](NC(=O)[C@H](CCC(N)=O)NC(=O)OCC1c2ccccc2-c2ccccc21)C(=O)N[C@@H](Cc1c[nH]c2ccccc12)C(=O)N[C@@H](CCC(=O)NC(c1ccccc1)(c1ccccc1)c1ccccc1)C(=O)O]

### Example III-5a: Synthesis of Z-Trp-Gln(Trt)-OMe

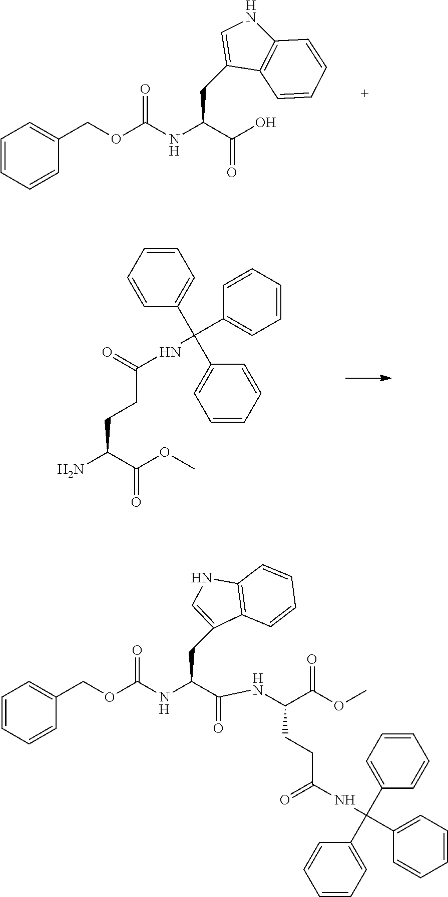 [SMILES: O=C(N[C@@H](Cc1c[nH]c2ccccc12)C(=O)O)OCc1ccccc1 ; COC(=O)[C@@H](N)CCC(=O)NC(c1ccccc1)(c1ccccc1)c1ccccc1 ; COC(=O)[C@H](CCC(=O)NC(c1ccccc1)(c1ccccc1)c1ccccc1)NC(=O)[C@H](Cc1c[nH]c2ccccc12)NC(=O)OCc1ccccc1]

In a flask H-Gln(Trt)-OMe (4.00 g/1.00 eq.) and Z-Trp-OH (3.36 g/1.00 eq.) were dissolved in DMF (40 mL). To the yellow, clear solution NMM (4.37 ml/4.00 eq.) was added at room temperature. The solution was cooled to 0° C. in an ice bath and TBTU (3.51 g/1.10 eq.) was added in one portion. The reaction mixture was stirred at 0° C. for 2 h than warmed to room temperature. HPLC-2 (18 h): 90.7% product; n.d. H-Gln(Trt)-OMe; 5.4% Z-Trp-OH. The resultant yellow solution (reaction mixture) was precipitated with 450 mL aq. NaHCO₃ (2%) solution. The suspension was filtered and washed with aq. NaHCO₃ (5%) solution (4×25 ml) and water (4×2 5 ml). The product was dried under reduced pressure in a vacuum oven at 45° C. to provide 7.34 g of raw product (102%).

The product was recrystallized by adding MTBE (425 mL) to the raw product (7.34 g). The suspension was stirred at 40° C. The product partially dissolved in the solvent but started to crystallize at the same time. A white fine suspension was obtained which was stirred for 30 min at 40° C. The suspension was stirred for 1 h at 10° C. The suspension was filtered and washed with MTBE (2×25 mL). The product was dried under reduced pressure in a vacuum oven at 45° C. to yield 6.75 g (98%).

### Example III-5b: Synthesis of H-Trp-Gln(Trt)-OMe*HCl

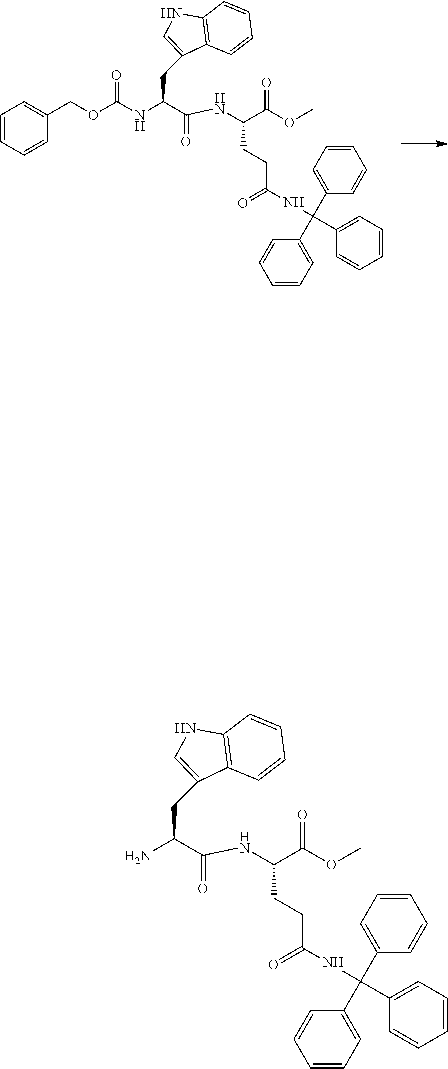 [SMILES: COC(=O)[C@H](CCC(=O)NC(c1ccccc1)(c1ccccc1)c1ccccc1)NC(=O)[C@H](Cc1c[nH]c2ccccc12)NC(=O)OCc1ccccc1 ; COC(=O)[C@H](CCC(=O)NC(c1ccccc1)(c1ccccc1)c1ccccc1)NC(=O)[C@@H](N)Cc1c[nH]c2ccccc12]

In a hydrogenation reactor (20 L), Z-Trp-Gln(Trt)-OMe (625.8 g/1.00 eq.) was suspended in MeOH/H₂O (10.0 L; 95/5). Pd/C 5% (47.0 g) and HCl 36.4% (77.4 mL/1.05 eq.) were added. The suspension was hydrogenolysed at 30° C. and 2.5 bar H₂ pressure. The reaction mixture was clear filtered and washed with MeOH (2×500 mL). The filtrate was stored at 5° C. overnight. The strong orange filtrate was evaporated under reduced pressure at 45° C. to an orange solution (9.0 L distillate). EtOAc (3×1.0 L) was added, evaporated again to an orange solution (3.0 L distillate) and then to a thick oil (followed by azeotropic distillation EtOAc 2×1.0 L). EtOAc (3.0 L) was added and the reaction mixture was precipitated by adding on IPE (9.0 L). The pink suspension was filtered and washed with IPE (3×1.0 L). The product was dried in a vacuum oven at 40° C. to yield 556.8 g/103%.

### Example III-5c: Synthesis Z-Thr(tBu)-OH

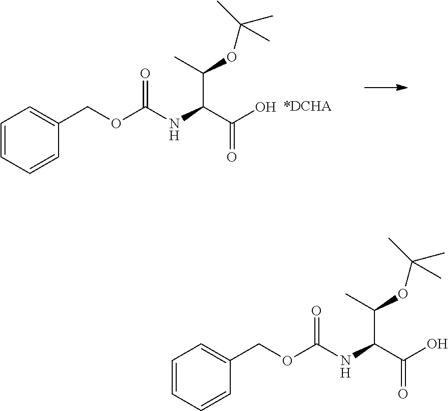 [SMILES: C[C@@H](OC(C)(C)C)[C@H](NC(=O)OCc1ccccc1)C(=O)O ; C[C@@H](OC(C)(C)C)[C@H](NC(=O)OCc1ccccc1)C(=O)O]

In a 10 L flask Z-Thr(tBu)-OH*DCHA (400.0 g) was suspended in EtOAc (3 L) und water (1 L): a thick, white suspension was formed. Under strong stirring the pH was adjusted to 1.5-2 with sulphuric acid (10% aprox. 1.1 L). The mixture was stirred for 10 min and a clear solution was formed. The layers were separated, and the organic layer was washed with water (3×1.5 L). The organic layer was evaporated under reduced pressure at 40° C. to an oil. The residual oil was diluted with EtOAc (1 L) and again concentrated under reduced pressure at 40° C. to an oil. The residual oil was diluted with EtOAc (1 L) and concentrated under reduced pressure at 40° C. to an oil. The oil was dissolved with DMF (1 L). The product solution was evaporated under reduced pressure at 40° C. for 15 min to remove residual EtOAc to yield 616.8 g/100%.

### Example III-5d: Synthesis Z-Thr(tBu)-Trp-Gln(Trt)-OMe

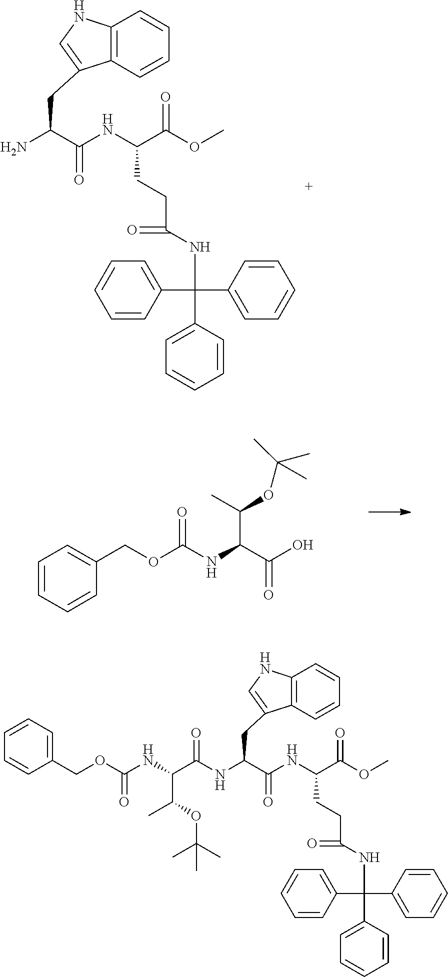 [SMILES: COC(=O)[C@H](CCC(=O)NC(c1ccccc1)(c1ccccc1)c1ccccc1)NC(=O)[C@@H](N)Cc1c[nH]c2ccccc12 ; C[C@@H](OC(C)(C)C)[C@H](NC(=O)OCc1ccccc1)C(=O)O ; COC(=O)[C@H](CCC(=O)NC(c1ccccc1)(c1ccccc1)c1ccccc1)NC(=O)[C@H](Cc1c[nH]c2ccccc12)NC(=O)[C@@H](NC(=O)OCc1ccccc1)[C@@H](C)OC(C)(C)C]

In a 10 L reactor H-Trp-Gln(Trt)-OMe*HCl (517.0 g/1.00 eq.), Z-Thr(tBu)-OH oil 41.3% (596.3 g/0.96 eq.) and Z-Thr(tBu)-OH oil 46.6% (20.5 g/0.04 eq.) and were dissolved in DMF (synthesis quality (5.0 L)). The mixture was stirred at 20° C. for 15 min until a red solution was formed. The solution was cooled to 0° C. with jacket temperature of −5° C. TBTU (292.lg/1.10 eq.) was added in one portion. Jacket temperature was raised to 0° C. NMM (273 mL/3.00 eq.) was added dropwise over 2 h. The reaction mixture was stirred at jacket temperature 0° C. for additional 2 h (pH=5.5). The jacket temperature was warmed first to 5° C., after 1-2 h to 15° C. and the reactions mixture was stirred overnight. The reaction mixture was evaporated under reduced pressure at 65° C. until DMF was removed almost completely and a red to yellowish oil was obtained. The residual oil was dissolved in EtOAc (2.5 L). The organic layer was washed with NaHCO₃-solution (2%, 3×1.5), and water (3×2 L). The organic layer was evaporated under reduced pressure at 40° C. to an oil and dried by azeotropical distillation with EtOAc (2×1 L). The residual oil was dissolved in MeTHF (1.5 L) at 40° C.

The MeTHF solution was diluted with IPE (aprox. 1 L) till a turbidity was observed and stirred for 68 h at 20° C.: an oil was formed. The solvent was evaporated and dissolved again in MeTHF (1.5 L) at 40° C. (approx. 2 L solution). The solution was added over 50 min at room temperature to IPE (12 L): a fine yellowish suspension was formed. The suspension was stirred for 1 h at room temperature (18-20° C.). The suspension was filtered and washed with IPE (3×1.5 L). The product was dried in a vacuum oven at 40° C. to yield 630.6 g/87%.

### Example III-5e: Synthesis H-Thr(tBu)-Trp-Gln(Trt)-OH

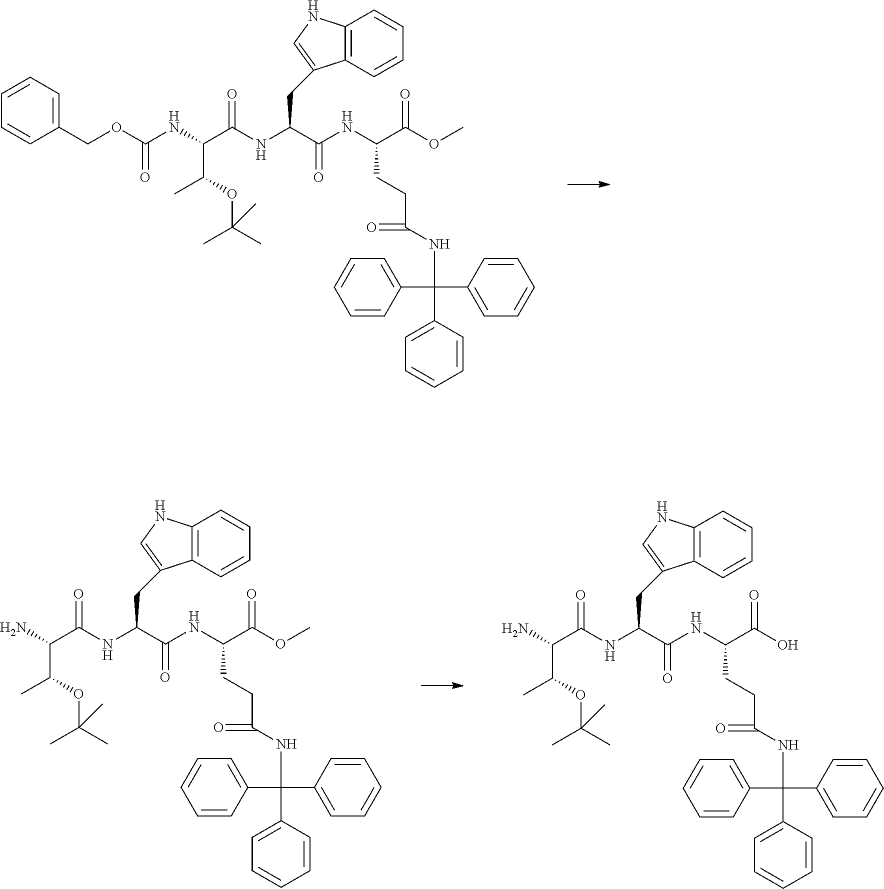 [SMILES: COC(=O)[C@H](CCC(=O)NC(c1ccccc1)(c1ccccc1)c1ccccc1)NC(=O)[C@H](Cc1c[nH]c2ccccc12)NC(=O)[C@@H](NC(=O)OCc1ccccc1)[C@@H](C)OC(C)(C)C ; COC(=O)[C@H](CCC(=O)NC(c1ccccc1)(c1ccccc1)c1ccccc1)NC(=O)[C@H](Cc1c[nH]c2ccccc12)NC(=O)[C@@H](N)[C@@H](C)OC(C)(C)C ; C[C@@H](OC(C)(C)C)[C@H](N)C(=O)N[C@@H](Cc1c[nH]c2ccccc12)C(=O)N[C@@H](CCC(=O)NC(c1ccccc1)(c1ccccc1)c1ccccc1)C(=O)O]

In a 20 L hydrogenator Z-Thr(tBu)-Trp-Gln(Trt)-OMe (607.0 g/1.0 eq.) was dissolved in MeOH/H₂O (9/1; 6070 mL). Pd/C 5% (67.0 g) was added. The hydrogenolysis was started at 25° C./3.0 bar H₂ pressure. The hydrogenolysis was stopped after 5 h. The catalyst was filtrated off and washed with methanol (2×200 mL). The yellowish solution was evaporated under reduced pressure to a slimy substance (removal of methanol). The slime was dissolved in THF (4.2 L) and water (1.2 L). The mixture was stored overnight at 5° C. After warming up to 15° C. a clear solution was obtained. To this mixture, a solution of LiOH*H₂O (43.3 g/1.50 eq.) in water (1.77 L) was added over 3 h at 5° C. The solution was stirred for another 1 h/5° C. Then methanol (3 L) was added and the yellow solution was heated to 20° C. With HCl 10% (320 mL) the pH was adjusted to 7.0. The yellow solution turned into a suspension and was stirred for 1 h at 20° C. The suspension was filtrated and washed with methanol (3×1 L). The product was dried in a vacuum oven at 40° C. to yield 486.9 g/96.5%.

### Example III-5f: Synthesis Fmoc-Gln-ONp

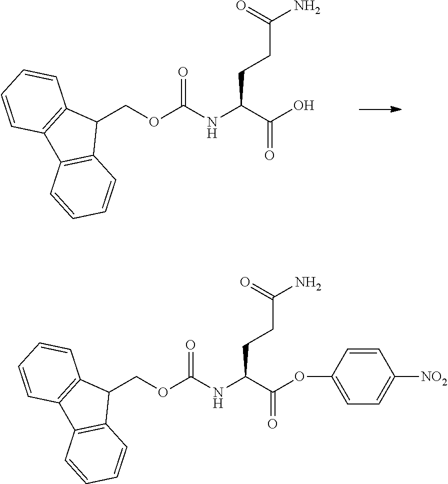 [SMILES: NC(=O)CC[C@H](NC(=O)OCC1c2ccccc2-c2ccccc21)C(=O)O ; NC(=O)CC[C@H](NC(=O)OCC1c2ccccc2-c2ccccc21)C(=O)Oc1ccc([N+](=O)[O-])cc1]

In 10 L reactor Fmoc-Gln-OH (600.0 g/1.00 eq.) and 4-nitrophenol (226.6 g/1.00 eq.) were suspended in DMF (6 L). The suspension was heated to 35° C. and stirred for 1 h. Then after addition of another portion of DMF (2 L) and stirring for 20 min a clear, yellow solution was formed. The solution was cooled to −5° C. and a solution of DCC (370 g/1.10 eq.) in DMF (2 L) was added dropwise over 3 h at 0-(−5) ° C. The yellow, thin suspension was stirred overnight at 0° C. The reaction mixture was slowly heated up to r.t. while it stirred. Then it was clear filtrated and washed with DMF 2×1 L. The filtrate was precipitated on water (35 L) and a yellowish suspension was formed. The suspension was filtrated and washed with water (3×6.5 L) and IPE (3×5 L). The product was dried in a vacuum oven at 40° C. to yield 870 g.

### Example III-5 g: Synthesis Fmoc-Gln-Thr(tBu)-Trp-Gln(Trt)-OH (Ring fragment)

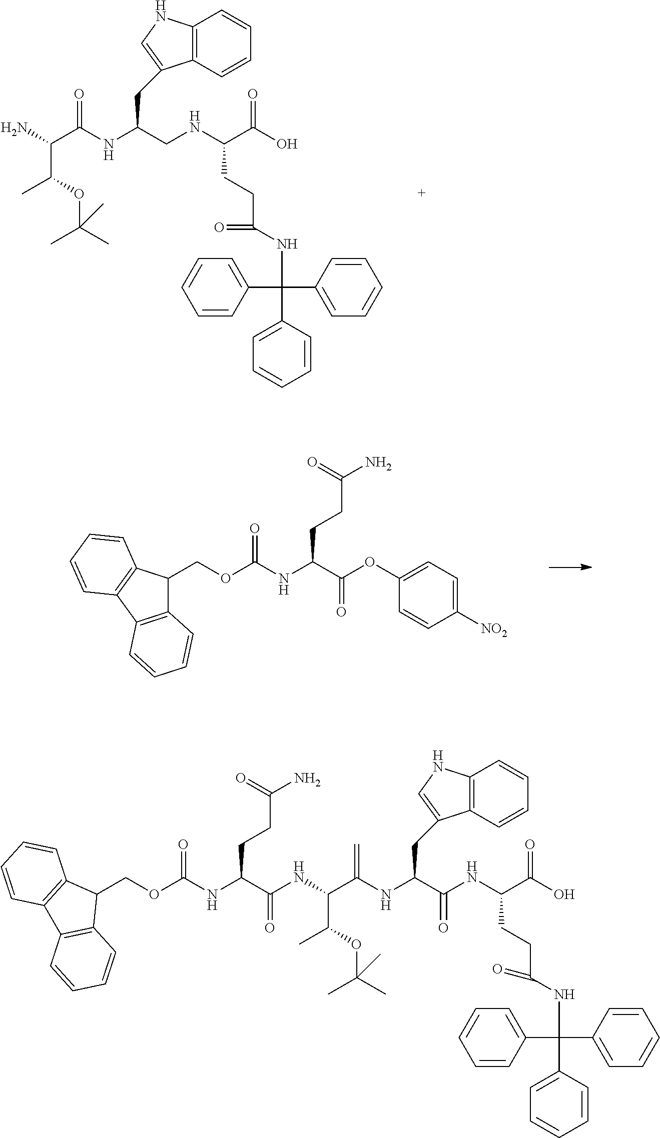 [SMILES: C[C@@H](OC(C)(C)C)[C@H](N)C(=O)N[C@H](CN[C@@H](CCC(=O)NC(c1ccccc1)(c1ccccc1)c1ccccc1)C(=O)O)Cc1c[nH]c2ccccc12 ; NC(=O)CC[C@H](NC(=O)OCC1c2ccccc2-c2ccccc21)C(=O)Oc1ccc([N+](=O)[O-])cc1 ; C=C(N[C@@H](Cc1c[nH]c2ccccc12)C(=O)N[C@@H](CCC(=O)NC(c1ccccc1)(c1ccccc1)c1ccccc1)C(=O)O)[C@@H](NC(=O)[C@H](CCC(N)=O)NC(=O)OCC1c2ccccc2-c2ccccc21)[C@@H](C)OC(C)(C)C]

In 10 L reactor Fmoc-Gln-ONp (210.7 g/0.90 eq.) was dissolved in DMF (synthesis quality, 2 L) to form a clear, colourless solution. In a flask H-Thr(tBu)-Trp-Gln(Trt)-OH (350 g/1.0 eq.) was suspended in DMF (synthesis quality, 3.5 L). Collidine (202.8 g/3.50 eq.) was added. The suspension was stirred 45 min at 45° C. and a turbid solution was formed. This thin, yellow suspension was added at 22° C. over 6.75 h to the Fmoc-Gln-ONp-solution and stirred at r.t. overnight. The reaction mixture was split in two portions (3 L). The first portion (3 L) was diluted with EtOAc (4.5 L) and NaHSO₄-5% (5 L). The phases were separated and the aqueous phase was extracted with EtOAc (2.7 L). The second portion was treated in the same way. The combined organic phases were washed with water (4×1.5 L). The organic layer was evaporated under reduced pressure at 45° C. to a thin suspension approx. 5 L. The mixture was co-distilled with EtOAc (5×3 L) again to 5 L residue. The thin suspension was stirred in an ice bath for 30 min, filtrated and washed with EtOAc (3×1 L). The product was dried in a vacuum oven at 40° C. to yield 366.8 g/71%.

The raw product was dissolved in EtOAc (5.7 L), DMF (980 mL) and water (350 mL). After stirring at room temperature for 30 min a clear, yellow solution was formed. The solution was washed with NaCl-10% solution (2×1.0 L) and water (2×1.0 L). The organic phase was diluted with EtOAc (2.0 L) and evaporated under reduced pressure at 45° C. After 2.0 L of distillate the thin suspension was co-distilled with EtOAc (3×1.5 L). During evaporation the product crystalized to a white suspension. The suspension was stirred 1 h in the ice bath. and filtrated. The filter cake was washed with EtOAc (4×0.60 L). The product was dried in a vacuum oven 40° C. The product was dried in a vacuum oven 40° C. to yield 356.90 g/69%.

### Example III-6: Synthesis of Fragment 1

### Example III-6a: Coupling of Fmoc-Gln(Trt)-Thr(tBu)-Gln(Trt)-OH and Ac-D-Arg(Pbf)-Abu(4-(S)-Cys-OMe)-OAll

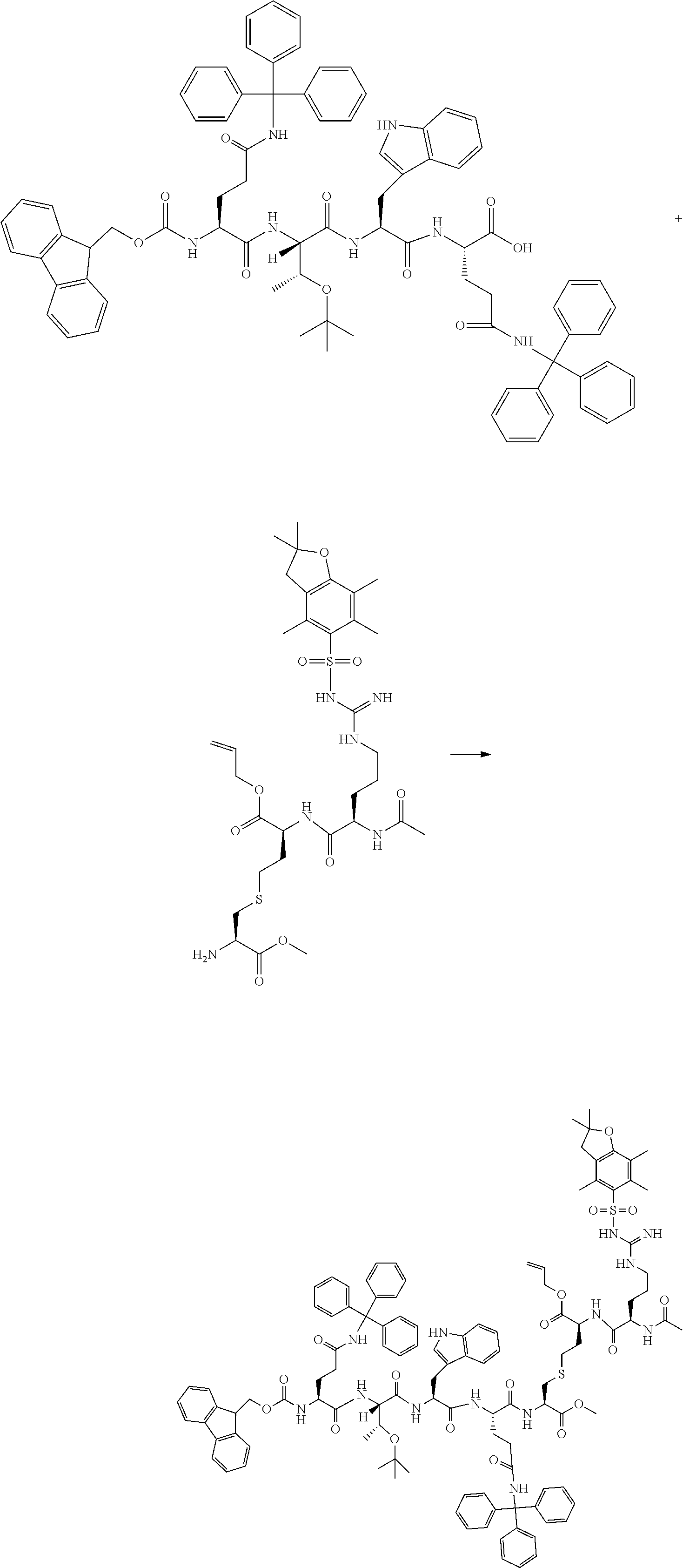

To a solution of Ac-D-Arg(Pbf)-Abu(4-(S)-Cys-OMe)-OAll (1.12 g, 1.54 mmol, 1.02 eq), Fmoc-Gln(Trt)-Thr(tBu)-Trp-Gln(Trt)-OH (2.0 g, 1.15 mmol, 1.0 eq) and 2,4,6-collidine (0.796 mL, 6.04 mmol, 4.0 eq.) in acetonitrile (40 mL), PyAOP (0.905 g, 1.74 mmol, 1.15 eq.) was added and the reaction mixture was stirred at 20° C. After 16 hours, HPLC-IPC showed that the reaction was almost complete (BB: 0.6%, FG1 Ring: 0.2%). The solvent was evaporated under reduced pressure at 35° C. and the residue was taken up in EtOAc (100 mL). The organic phase was washed with NaHSO₄ (aq. 5%) (50 mL), NaHCO₃ (aq. 5%) (50 mL), NaHCO₃ (aq. 5%): brine (3:1) (60 mL), brine (50 mL) and dried with MgSO₄ anhydrous. The product solution was concentrated to 20 mL and the product precipitated by adding the concentrated solution to IPE (200 mL) at 0-5° C. The white suspension formed was stirred at 0 5° C. for 10 minutes and the product separated by filtration through a pore 3 glass filter. The filter cake was washed with IPE (2×10 mL) and dried under reduced pressure at 35° C. to give 3.12 g of a white solid (HPLC purity: 81.5%, ESI-MS: m/z=1972.59 [M-Trt+H⁺]⁺, m/z=1018.45 [M+2H⁺]²⁺, yield: 101.4%).

### Example III-6b: Allyl ester and Fmoc Cleavage

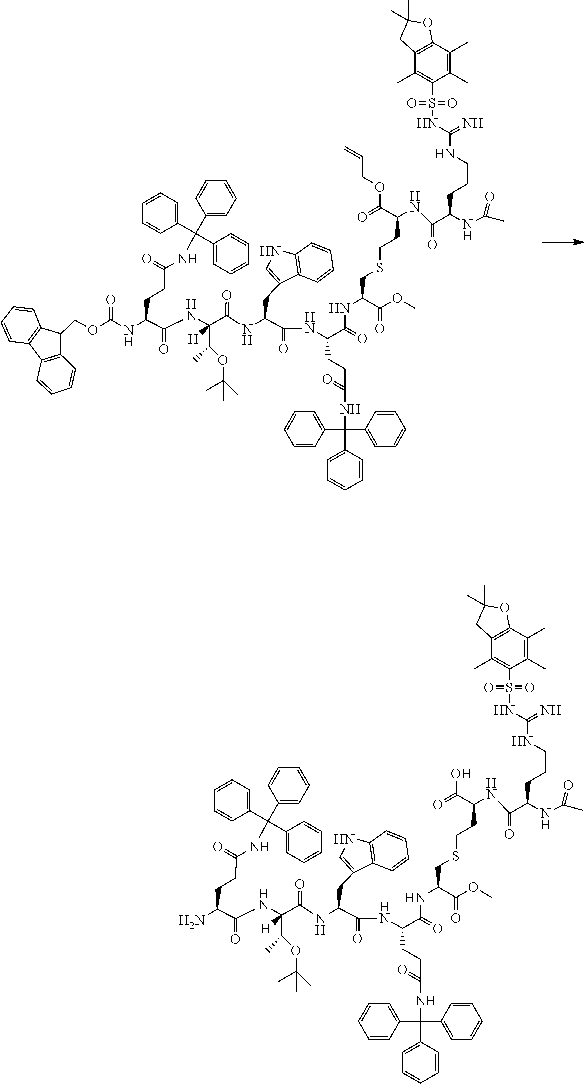

To a solution of Fmoc-Gln(Trt)-Thr(tBu)-Trp-Gln(Trt)-Cys-OMe-((S)-4-Abu-D-Arg(Pbf)-Ac)-OAll (2.9 g, 1.421 mmol, 1.0 eq.) and phenylsilane (1.75 mL, 0.14.21 mmol, 10.0 eq.) in anhydrous THF (30.0 mL), was added Pd(PPh₃)₄ (0.082 g, 0.071 mmol, 0.05 eq.) and the reaction mixture stirred at r.t. The reaction was finished (IPC-HPLC: SM: n.d.). The product was then precipitated by adding the reaction mixture to IPE (300 mL) at 0-5° C. The suspension formed was stirred at 0-5° C. for 10 minutes and the product separated by filtration. The filter cake was washed with IPE (3×10 mL) and dried under vacuum at 35° C. to give 2.67 g of a gray solid. (HPLC purity: 84.6%, ESI-MS: m/z=1993.97 [M+H⁺]⁺, yield: 97.2%).

Fmoc deprotection: To a solution of Fmoc-Gln(Trt)-Thr(tBu)-Trp-Gln(Trt)-Cys-OMe-((S)-4-Abu-D-Arg(Pbf)-Ac)—OH (2.5 g, 1.293 mmol, 1.0 eq) in DMF (25 mL) at r.t. was added DBU (0.25 mL, 1% v:v) and the reaction mixture stirred at r.t. IPC-HPLC after 180 minutes showed that the reaction was finished (SM: n.d.). The solvent was evaporated under reduced pressure at 40° C. to an oil and the residue was taken up in EtOAc (100 mL) and a suspension was formed. The suspension was diluted with IPE (200 mL) and stirred for 10 minutes at r.t. The product was then separated by filtration, washed with IPE (2×10 mL) and dried under reduced pressure at 35° C. to give 2.28 g of a yellowish solid (HPLC purity: 82.5%, ESI-MS: m/z=1771.83 [M+H⁺]⁺, yield: 90.6%).

### Example III-6c: Cyclization of the linear precursor

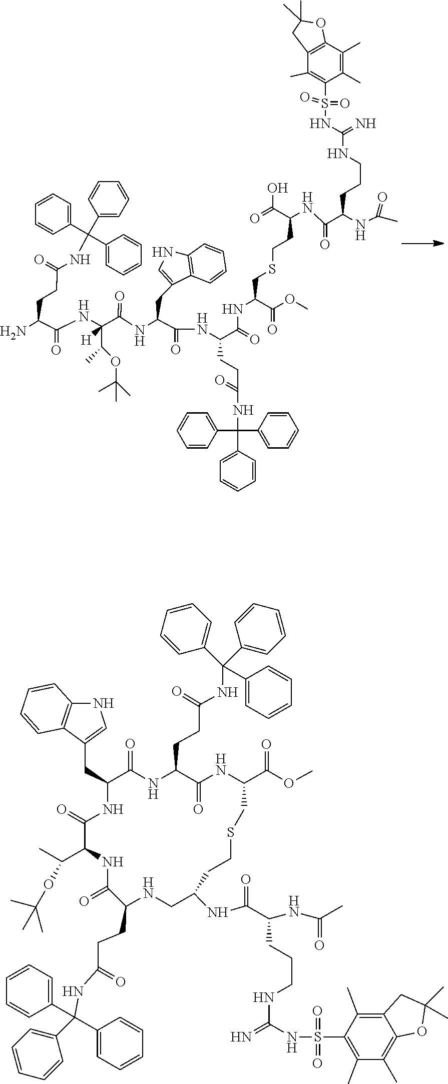

The following solutions were prepared:

Sln. A: The linear protected peptide (1.2 g, 0.678 mmol, 1.0 eq) and DIPEA (0.461 mL, 2.71 mmol, 4.0 eq.) were dissolved in 20 mL of DMF.

Sln. B.: PyOxim (0.536 g, 1.016 mmol, 1.5 eq) and 30 mL of DMF were dissolved in a 100 mL reactor vessel at r.t.

Sln. A was added to Sln. B over 2.0 hours at r.t. The final concentration was 13.5 mM. 30 minutes after complete addition of Sln. B, a sample was taken to HPLC-IPC and it was determined that the reaction was complete. (SM: n.d). At this point the solvent was evaporated to an oil and the oil re-dissolved in EtOAc (100 mL). The organic phase washed with 5% NaHCO₃ (2×50 mL), brine (50 mL) and dried with Na₂SO₄ anhydrous. The solvent evaporated under reduced pressure at 35° C. to an oil. The product was precipitated by adding the oil to IPE:heptane (1:1, v:v) (200 mL), separated by filtration through a pore 3 glass filter and dried under vacuum at 35° C. for 18 hours to give 1.11 g of a whitish solid. (HPLC purity: 81.3%, ESI-MS: 1753.85 [M+H⁺]⁺, yield: 92.6%).

### Example III-6d: Methyl Ester De-Protection

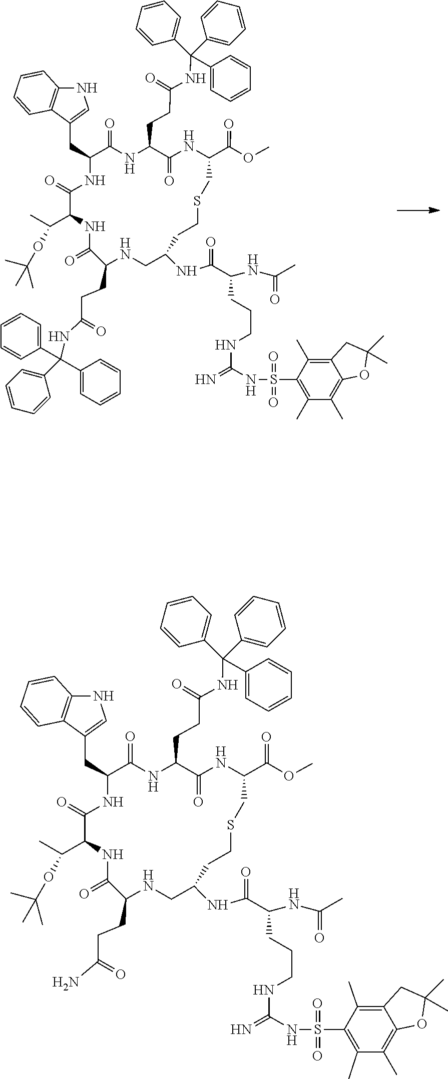

To a solution of the cyclic peptide, prepared in Example III-6c, (3.6 g, 2.383 mmol, 1.0 eq) in THF:water (3:1, v:v) (40 mL) was added LiOH×H₂O (0.198 g, 4.71 mmol, 2.0 eq) and the reaction mixture was stirred at r.t. IPC-HPLC after 1 hour showed completeness of the reaction (S.M: n.d). At this point, the solvent was evaporated and the remaining taken up in MeTHF (50 mL) and stirred until a fine suspension was formed. 5% NaHSO₄ (40 mL) was added and the mixture stirred until a clear biphasic solution was obtained. The phases were separated and the organic phase washed with water (40 mL), brine (40 mL) and dried over anhydrous sodium sulfate. The solvent was evaporated to approx. 15 mL and the product precipitated by addition of the product solution into IPE (150 mL). The product was then separated by filtration, the filter cake washed with IPE (2×10 mL) and dried under vacuum at 35° C. to give 3.2 g of a whitish solid. (HPLC purity: 82.2%, ESI-MS: m/z=1496.91 [M+H⁺]⁺, yield: 90.8%).

### Synthesis of Example III

### Example III-7: Coupling of Fragment 1 and Fragment 2+3 to prepare protected-Example III

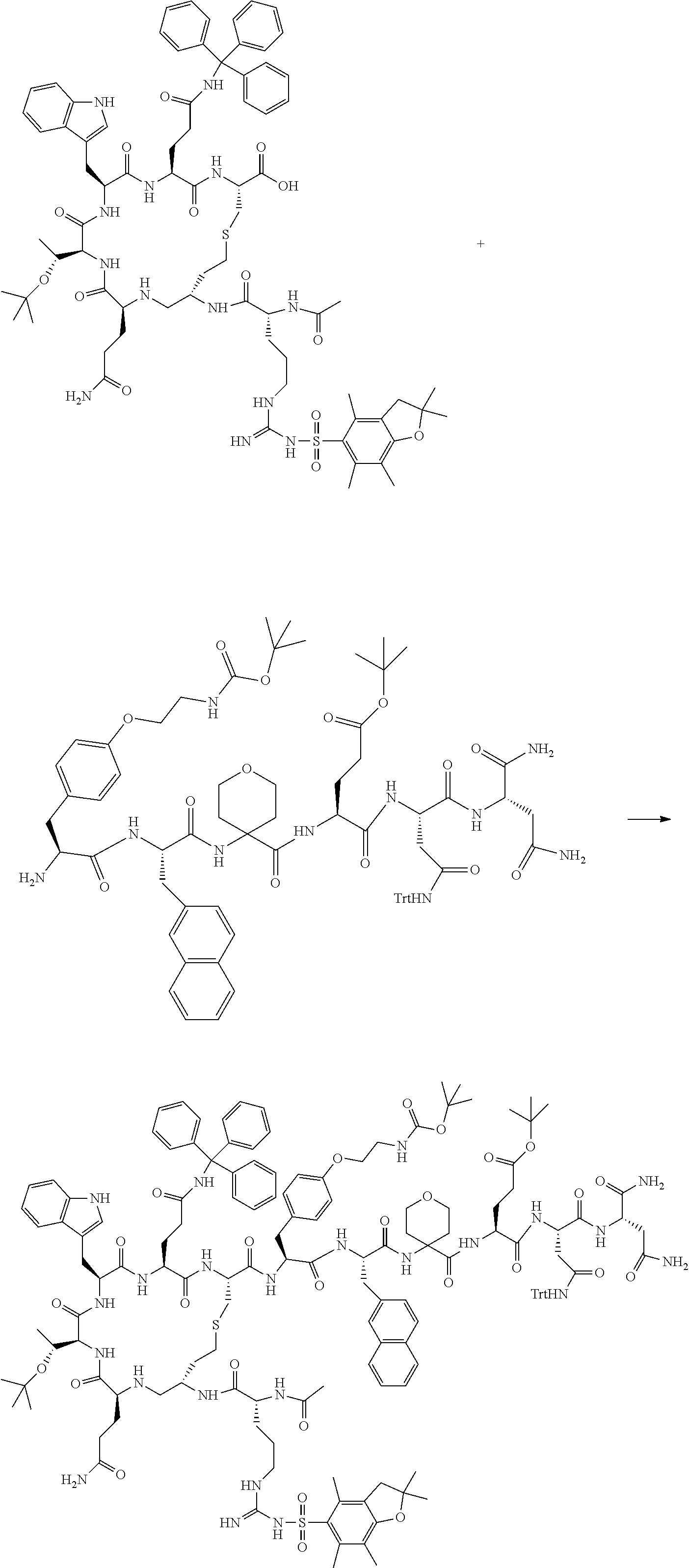

Fragment 1(2.745 g; 1.00 eq.) and Fragment 2+3 (2.369 g; 0.99 eq.) were dissolved in DMF (synthesis quality, 28 mL). The yellow solution was cooled in an ice bath, TBTU (0.620 g; 1.05 eq.) was added followed by DIPEA (936 μL) and the reaction mixture was stirred 1 h at 0° C. Then the mixture was stirred further at room temperature, additional Fragment 2+3 (24 mg/0.01 eq.) and TBTU (29 mg/0.05 eq.) were added and stirred overnight. The reaction mixture was precipitated onto NaHCO₃-2% (280 ml) and stirred for 5 min. The white suspension was filtrated and washed with water (3×25 mL). The product, protected-Example III, was dried in a vacuum oven at 40° C. to yield 4.435 g/87% ESI-MS: [M+2H]²: 1392.10.

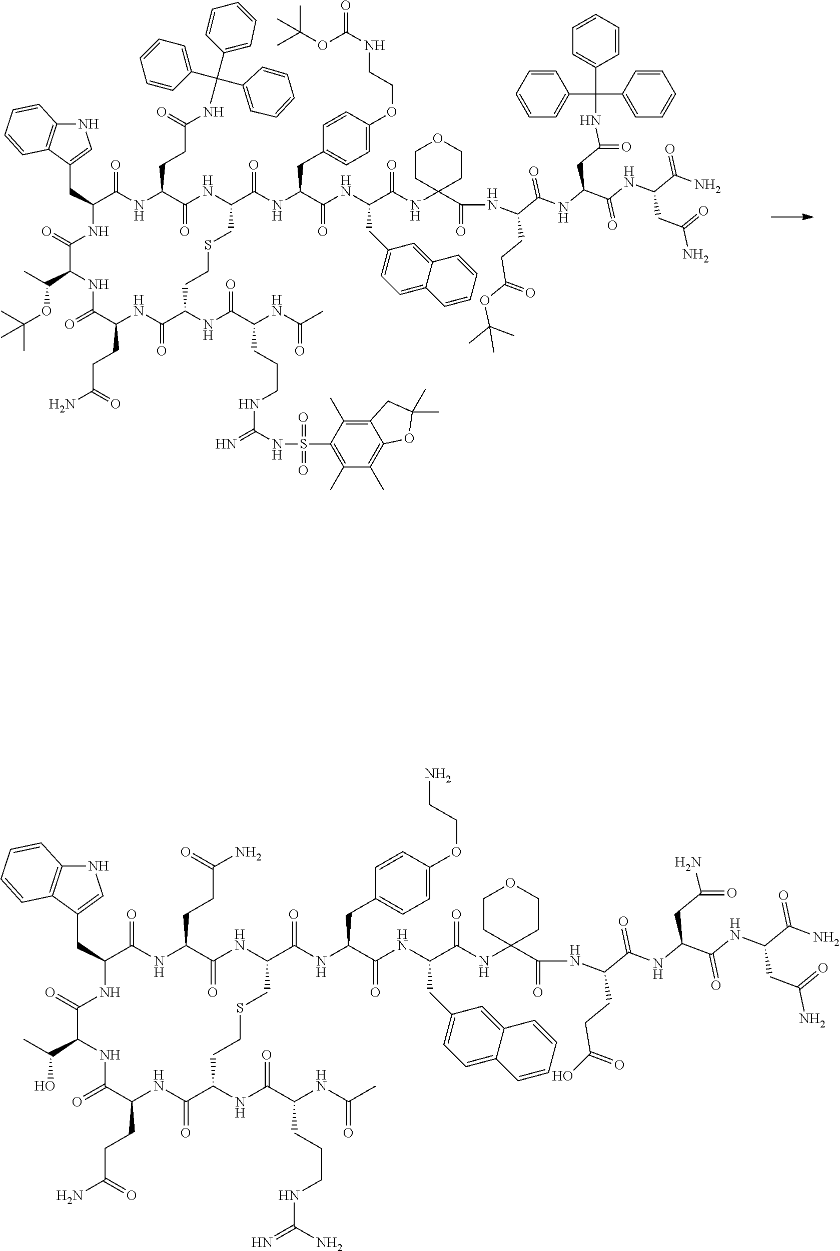

In a as protected-Example III (1.000 g was dissolved in HFIP (30.0 m e slightly turbid solution was clear filtrated and washed with HFIP (6.0 mL). To the clear, orange solution TIS (400 μL) and DODT (400 μL) were added. The solution was stirred at 22° C. and aq. HCl (36%, 4.00 mL) was added in two portions within 2 min. The solution was stirred at 22° C. After 1 h 15 min of stirring the reaction mixture was precipitated by adding on IPE (1000 mL). The product was washed three times with IPE. The product was dried in a vacuum oven at 40° C. to yield 675.4 mg/99% of Example III.

Alternatively, protected-Example III was converted to Example III using the following procedure: In a flask protected-Example III (11.33 g) was dissolved in HFIP (368.0 mL). The slightly turbid solution was clear filtrated and washed with HFIP (40.0 mL). To the clear, orange solution TIS (4.53 mL) and DODT (4.53 mL) were added. The solution was stirred at 23° C. and HCl (36%, 45.00 mL) was added in 2 min. The solution was stirred at (22° C.). After 45 min of stirring the reaction mixture was precipitated by adding on IPE (10.0 L). The beige suspension was stirred for 5 min, then filtrated and washed with IPE (5×100 mL). The product was dried in a vacuum oven at 35° C. to provide 7.992 g/103% (not assay corrected) MS: [M+2H]2+917.45; [M+H]⁺: 1834.26. The product was purified using semi-prep HPLC on a Daisopack SP-100-8-C8-PK (250×20 mm) column and gradient elution with water and MeOH+0.2% AcOH as eluents. Fractions with HPLC purity≥98.0% from all runs were mixed and concentrated to the half of the volume under vacuum at 35° C. The product solution was then injected into the Daisopack SP-100-8-C8-PK (250×20 mm) column, the column washed with a solution of 0.2 M ammonium acetate (100 mL) and 5% acetonitrile in water+0.2% AcOH (100 mL). The product was finally eluted using 25% acetonitrile in water+0.2% AcOH. Fractions of interest were tested by analytical HPLC (IPC-HPLC≥98.0%). Fractions fulfilling IPC-HPLC were mixed, diluted with water (1:1), frozen and lyophilized to give 0.46 g of the product as a white powder (HPLC purity: 98.9%, ESI-MS: m/z=917.38 [M+2H⁺]⁺).

Protected-Example III (1.5 g) was dissolved in a mixture of water and IPA (9.9 mL, 1:1 v/v) at 35° C. Then 130 μl water was added. The solution was cooled to 23° C. within 20 min and seed crystal were added (10 mg). Afterwards the mixture was stirred 2 h at 23° C., cooled to 5° C. with 0.1 K/min, stirred 3 h at 5° C., warmed to 22° C. within 30 min, stirred at 22° C. for 3 h, cooled to 5° C. with 0.1 K/min and stirred at 5° C. for 9 h. Then the suspension was filtered, washed with a mixture of water and IPA (1.5 mL, pre-cooled, 1:1, v/v) followed by IPA (0.75 mL) and dried under vacuum overnight at 40° C. to yield 603 mg of Example III (HPLC purity: 92.8%).

The following reaction scheme may aid in understanding the reactions discussed throughout the following examples.

### Reaction Scheme IV-1

The following reaction schemes (Schemes IV-1 to IV-3) are only meant to represent examples of the invention and are in no way meant to be a limit of the invention. The following liquid phase synthetic route to Compound 26 was developed.

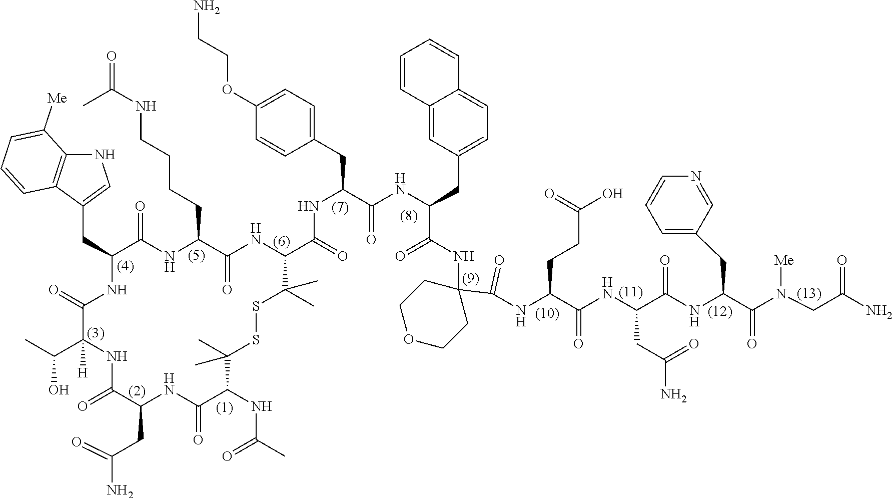 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

Compound 26 can be prepared by a convergent liquid phase peptide synthesis approach, as illustrated by Schemes IV-1, IV-2 and IV-3. In this approach, the target molecule is built by the condensation of the cyclic N-terminal heptapeptide fragment with the hexapeptide C-terminal peptide fragment.
To this end, a suitably protected tyrosine derivative residue (Pos7) can be reacted with a suitably protected penicillamine residue (Pos6) to form the dipeptide (7).
The dipeptide (7) can be deprotected at the terminal amine and reacted with a suitably protected lysine derivative residue (Pos5) to form tripeptide (9).
The tripeptide (9) can be deprotected at the terminal amine and reacted with a suitably protected tryptophan derivative residue (Pos4) to form tetrapeptide (11).
The tetrapeptide (11) can be deprotected at the terminal amine to form compound (20).
A suitably protected threonine derivative residue (Pos3) can be reacted with a suitably protected asparagine residue (Pos2) to form the dipeptide (1).
The dipeptide (1) can be deprotected at the terminal amine and reacted with a suitably protected penicillamine (Pos1) to form tripeptide (3).
The tripeptide (3) can be deprotected at the terminal amine and reacted with a suitable acylating reagent to form compound (5) which can be deprotected at the C-terminal to form compound (6).
The peptide fragments (6) and (20) are coupled together by a suitable coupling reagent to form peptide fragment (21).
The peptide fragment (21) can be deprotected at the C-terminal to form peptide fragment (22).
The peptide fragment (22) can be transformed into the cyclic peptide fragment (23).
A suitably protected sarcosine derivative residue (Pos13) can be reacted with a suitably protected 3-pyridyl-alanine derivative residue (Pos12) to form the dipeptide (12).
The dipeptide (12) can be deprotected at the terminal amine and reacted with a suitably protected asparagine derivative residue (Pos11) to form tripeptide (14).
The tripeptide (14) can be deprotected at the terminal amine and reacted with a suitably protected glutamic acid derivative residue (Pos10) to form tetrapeptide (16).
The tetrapeptide (16) can be deprotected at the terminal amine to form compound (17).
A suitably protected tetrahydropyran-glycine derivative residue (Pos9) can be reacted with a suitably protected naphthyl-alanine derivative residue (Pos8) to form the dipeptide (18).
The dipeptide (18) and the compound (17) can be coupled together by a suitable coupling reagent to form peptide fragment (19).
The peptide fragment (19) can be deprotected at the terminal amine to form peptide fragment (24).
The peptide fragments (23) and (24) are coupled together by a suitable coupling reagent to form the protected peptide intermediate (25), which is subsequently deprotected to form Compound 26.

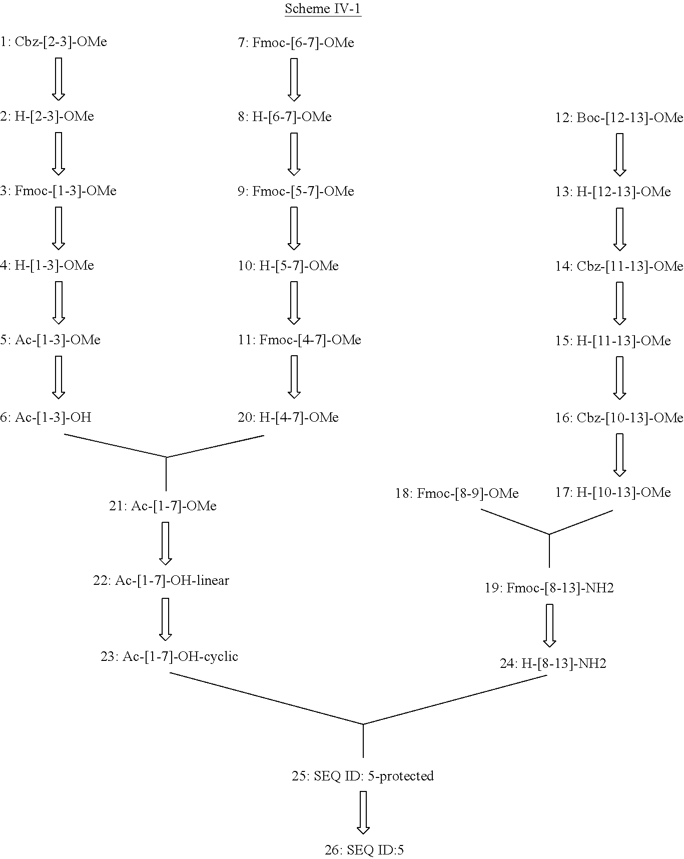

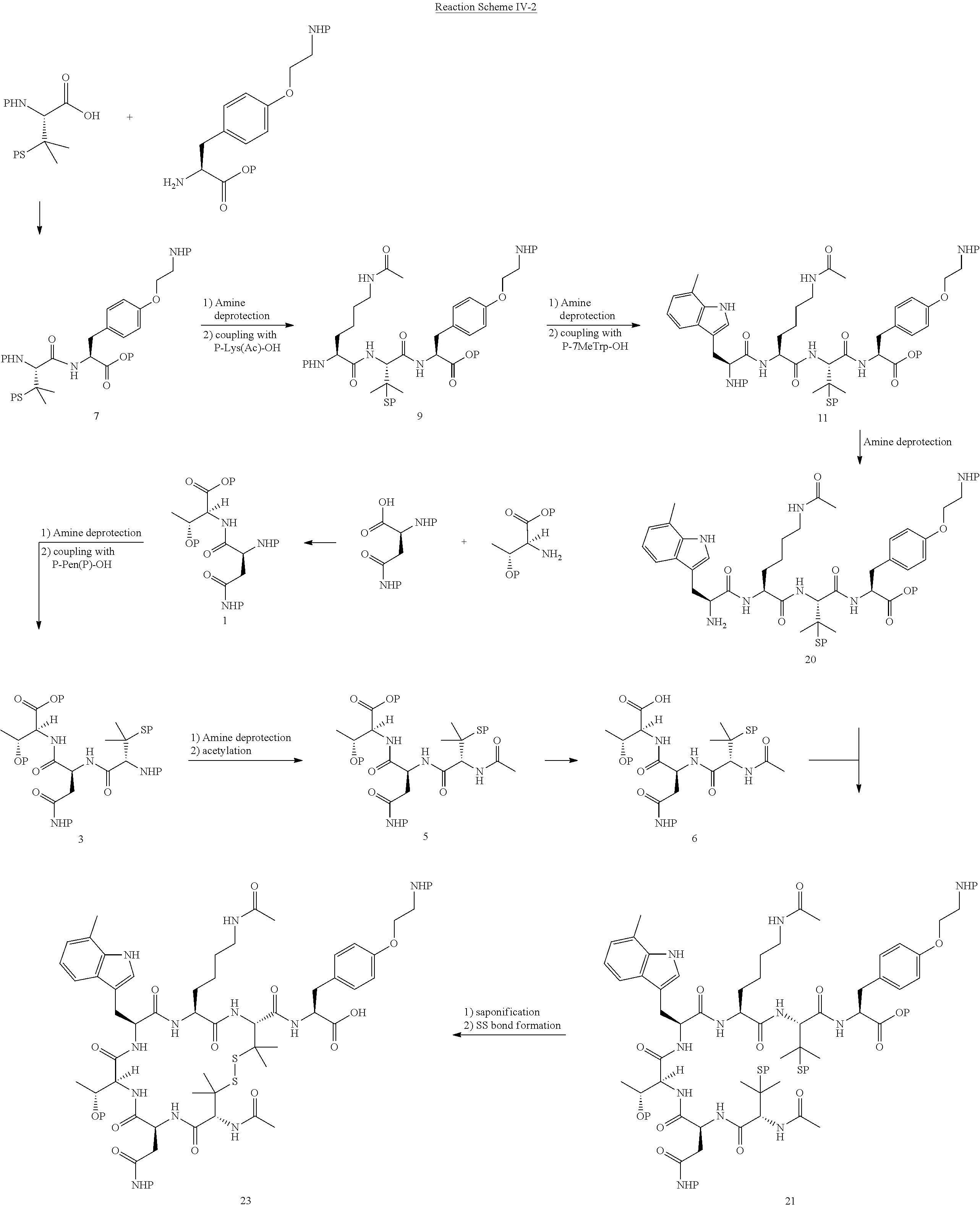

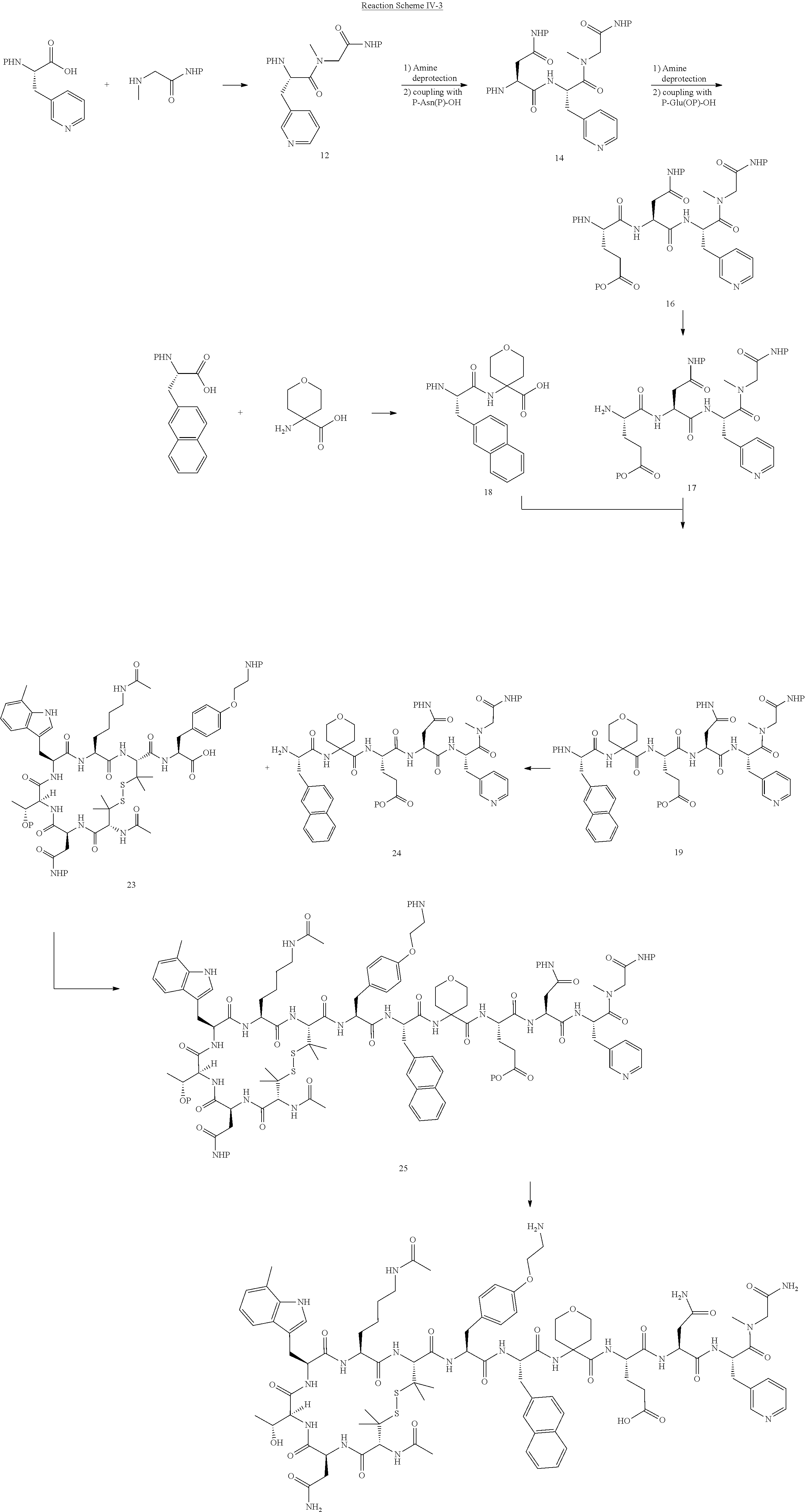

### Example IV-1: Synthesis of Cbz-[2-3]-OMe (Cbz-Asn-Thr(tBu)-OMe)

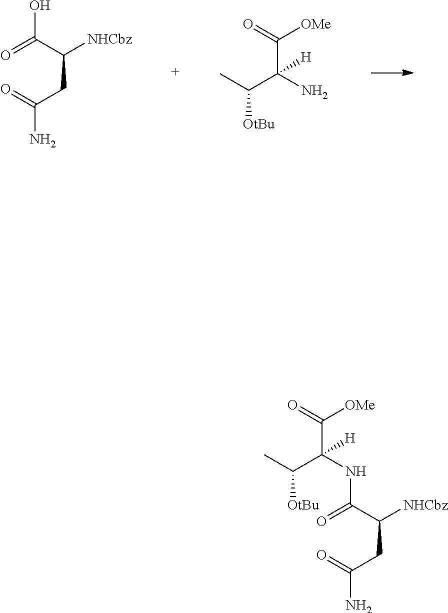 [SMILES: NC(=O)C[C@H](NC(=O)OCc1ccccc1)C(=O)O ; COC(=O)[C@@H](N)[C@@H](C)OC(C)(C)C ; COC(=O)[C@@H](NC(=O)[C@H](CC(N)=O)NC(=O)OCc1ccccc1)[C@@H](C)OC(C)(C)C]

To a solution of Z-Asn-OH (500.00 g, 1.877 mol, 1.0 eq.), H-Thr(tBu)-OMe (445.06 g, 1.971 mol, 1.05 eq.) and Oxyma Pure (266.85 g, 1.877 mol, 1.0 eq.) in EtOAc:DMSO (1:1, v:v) (3 L) was added triethylamine (572.6 mL, 4.311 mol, 2.2 eq.) and the mixture stirred at 25-30° C. for 10 minutes. EDC×HCl (468.00 g, 2.441 mol, 1.3 eq.) was added in four portions within one hour to the reaction mixture and stirred overnight until completion of the reaction.

The reaction mixture was then diluted with EtOAc (6 L) and washed with NaHSO₄ (5% aq.) (2×5 L), NaHCO₃ (5% aq.) (4×5 L) and water (5 L). The organic layer was evaporated under reduced pressure at 40° C. and the residue co-evaporated with EtOAc (3×3 L). The residue (ca. 25% w/v) was cooled to 20° C. and seeded with seed crystals. The product crystallized and the suspension formed was stirred at 0-5° C. for 2 h before the product was separated by filtration. The filter cake was washed with pre-cooled EtOAc (3×1 L) and the product dried under reduced pressure at 30° C. overnight to give 653 g (80% yield, 99% HPLC purity) of the desired product (Cbz-[2-3]-OMe) as a white solid.

### Example IV-2: Catalytic hydrogenolysis to produce H-[2-3]-OMe (H-Asn-Thr(tBu)-OMe)

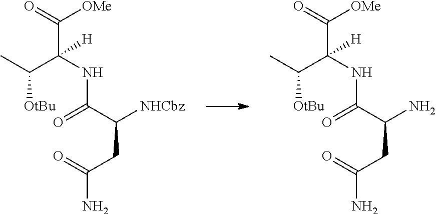 [SMILES: COC(=O)[C@@H](NC(=O)[C@H](CC(N)=O)NC(=O)OCc1ccccc1)[C@@H](C)OC(C)(C)C ; COC(=O)[C@@H](NC(=O)[C@@H](N)CC(N)=O)[C@@H](C)OC(C)(C)C]

Z-Asn-Thr(tBu)-OMe (330.0 g, 0.754 mol, 1.0 eq.) and EtOAc (2.6 L) were loaded into an hydrogenation reactor and the mixture stirred until a clear solution was obtained. Pd(OH)₂ on activated charcoal (6.6 g, 2% (w:w)) was added and the reactor was closed, under nitrogen and the temperature set at 40° C. Hydrogen pressure (3 bar) was applied, and the reaction mixture stirred until completion of the reaction. The reaction mixture was cooled to 25° C. and filtered through a deep filter to remove the catalyst and stored at 4° C. overnight (Mass: 2.46 kg).

### Example IV-3: Synthesis of Fmoc-[1-3]-OMe (Fmoc-Pen(Trt)-Asn-Thr(tBu)-OMe)

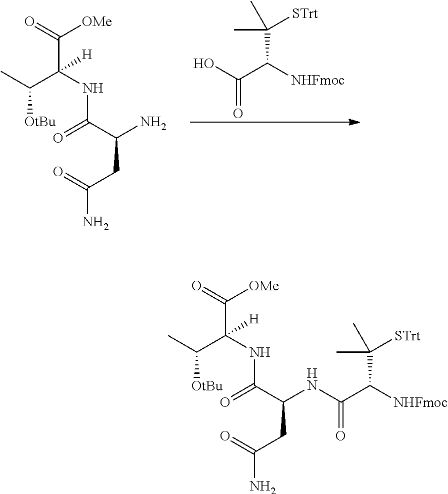

In a 15 L glass reactor with overhead stirrer were loaded Fmoc-Pen(Trt)-OH (387.0 g, 0.628 mol, 1.0 eq), TBTU (216.2 g, 0.673 mol, 1.07 eq.), DMSO (0.6 L) and the solution obtained in the step above (2.3 kg, 200.0 g H-Asn-Thr(tBu)-OMe, 0.659 mol, 1.05 eq.). The reaction mixture stirred at 22° C. until a clear solution was formed and DIPEA (305.2 mL, 1.795 mol, 2.0 eq) was added at once. The reaction mixture stirred at 20-25° C. for 1 h until completion of the reaction.

The reaction mixture was diluted with EtOAc (2.0 L) and washed with NaHCO₃ (5% aq.) (3×3 L) and water (2×3 L). The solvent of the organic phase was evaporated under reduced pressure at 35° C. and the residue co-evaporated with EtOAc (2×2.5 L).

The residue was taken in EtOAc (3.0 L) and the product precipitated by adding the product solution slowly to heptane (30 L). The white suspension formed was stirred at 20-25° C. for 30 minutes before the product was separated by filtration. The filter cake was washed with heptane (2×2 L) and dried under vacuum at 35° C. for 18 h to give 545 g (97% yield, 99% HPLC purity) of the desired product as a white solid.

### Example IV-4: Synthesis of H-[1-3]-OMe (H-Pen(Trt)-Asn-Thr(tBu)-OMe)

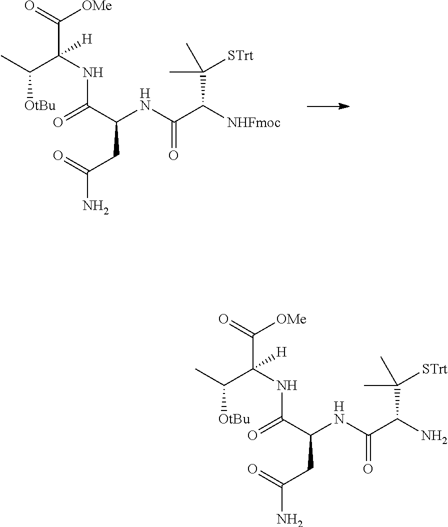

In a 15 L glass reactor with overhead stirrer were loaded Fmoc-Pen(Trt)-Asn-Thr(tBu)-OMe (500.0 g, 0.556 mol, 1.0 eq.) and EtOAc:DMSO (9:1, v:v) (5.0 L). The reaction mixture was stirred at 22° C. until a clear solution was formed. DBU (24.9 mL, 0.167 mol, 0.3 eq.) was added and the reaction mixture stirred at 20-25° C. for 90 min until completion of the reaction.

The reaction mixture was diluted with EtOAc (4.0 L) and washed with NaHCO₃ (5% aq.) (4 L) and water (4 L). The solvent of the organic phase was evaporated under reduced pressure at 40° C. and the residue co-evaporated with EtOAc (2×2.0 L). The residue was taken in EtOAc (2.0 L) and the product precipitated by adding the product solution to heptane (30 L). The white suspension formed was stirred at 20-25° C. for 15 minutes before the product was separated by filtration. The filter cake was washed with heptane (2×2 L) and dried under vacuum at 35° C. for 18 h to give 337 g (90% yield, 94% HPLC purity) of the desired product as a white solid.

### Example IV-5: Synthesis of Ac-[1-3]-OMe (Ac-Pen(Trt)-Asn-Thr(tBu)-OMe)

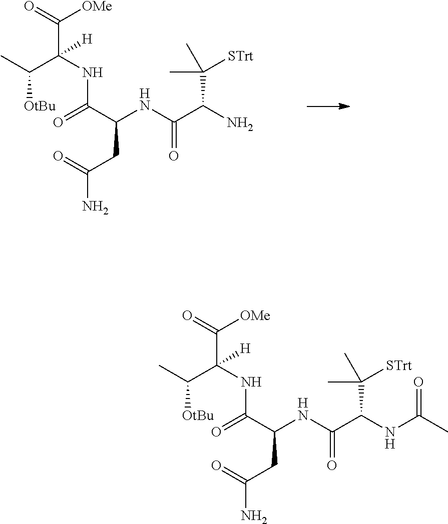

In a 15 L glass reactor with overhead stirrer were loaded H-Pen(Trt)-Asn-Thr(tBu)-OMe (318.2 g, 0.470 mol, 1.0 eq.) and MeOH (3.0 L) and the reaction mixture was stirred at 22° C. until a clear solution was formed. Acetic anhydride (55.6 mL, 0.588 mol, 1.25 eq.) was added and the reaction mixture stirred at 20-25° C. for 1 h until completion of the reaction. The reaction mixture was concentrated by evaporation of the solvent at 45° C. and the residue was taken up in EtOAc:Me-THF (1:1, v:v) (4.0 L). The organic phase was washed with NaHCO₃ (5% aq.) (2 L) and water (2 L) and the solvent of the organic phase evaporated under reduced pressure at 45° C. and the residue co-evaporated with EtOAc (2×1.5 L). The residue was taken in EtOAc (3.0 L) and the product precipitated by adding the product solution to heptane (30 L). The white suspension formed was stirred at 20-25° C. for 15 minutes before the product was separated by filtration. The filter cake was washed with heptane (4×1 L) and dried under vacuum at 35° C. for 18 h to give 328.2 g (97% yield, 99% HPLC purity) of the desired product as a white solid.

### Example IV-6: Synthesis of Ac-[1-3]—OH (Ac-Pen(Trt)-Asn-Thr(tBu)-OH)

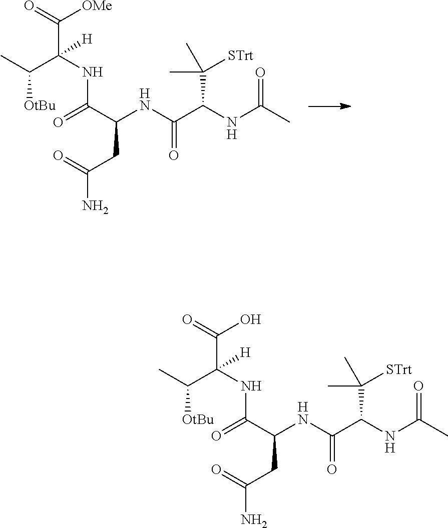

In a 10 L reactor with overhead stirring were loaded Ac-Pen(Trt)-Asn-Thr(tBu)-OMe (310.0 g, 0.431 mol, 1.0 eq.) and THF (1.5 L). The mixture was stirred until a clear solution was obtained. The product solution was diluted with water (1.5 L) and cooled to 5° C. A solution of LiOH×H₂O (25.35 g, 0.603 mol, 1.4 eq.) in water (0.5 L) was added to the reaction mixture within 90 minutes while stirring at 5° C. for 5 h until completion of the reaction. The reaction mixture was diluted with water (1.5 L) and Me-THF (5 L) and the layers separated. Me-THF (6 L) was added to the aqueous phase and the pH of the biphasic mixture was adjusted to 4.7 with HCl (10%, 100 mL). The phases were separated and the aqueous phase re-extracted with EtOAc (3.0 L). Both organic phases were mixed and the solvent evaporated under reduced pressure at 35° C. followed by co-evaporation of the residue with EtOAc (2×3.0 L). The residue was taken in EtOAc (3.0 L) and the product precipitated by adding the product solution to heptane (35 L). The white suspension formed was stirred at 20-25° C. for 15 minutes before the product was separated by filtration. The filter cake was washed with heptane (2×0.5 L) and dried under vacuum at 35° C. for 18 h to give 252.4 g (83% yield, 98% HPLC purity) of the desired product as a white solid.

### Example IV-7: Synthesis of Fmoc-[6-7]-OMe (Fmoc-Pen(Acm)-Tyr(2-Boc-ea)-OMe)

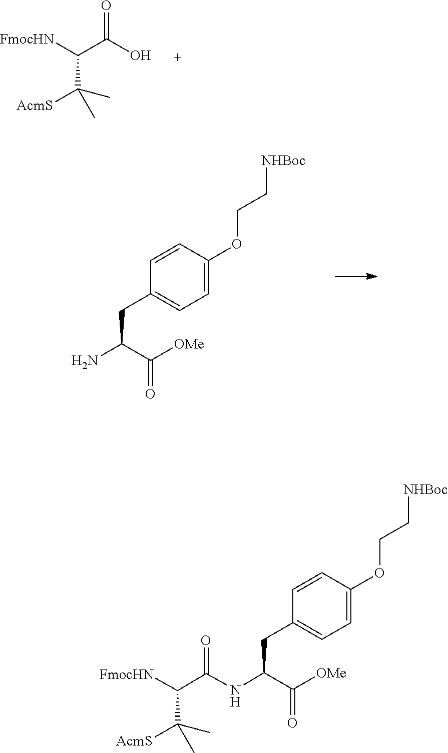

Fmoc-Pen(Acm)-OH (0.79 mol, 1.0 eq), H-Tyr(2-Boc-ea)-OMe (0.81 mol, 1.02 eq.) and Oxyma Pure (0.79 mol, 1.0 eq.) were dissolved in EtOAc (3.5 L). DIC (0.87 mol, 1.1 eq.) was added over 2 hours at 23° C. and the reaction stirred for 1 h until completion of the reaction. The reaction mixture was filtered and the filter cake was washed with EtOAc. The product solution was washed with NaHSO₄ aq., NaHCO₃ aq. and water. The solvent was evaporated under reduced pressure at 35° C. and the resulting residue was co-evaporated with EtOAc. The residue was taken up in Me-THF (2 L) and the product solution was precipitated by adding the solution to pentane (35 L) over 30 minutes. The white suspension was separated by filtration and the product washed with pentane. The off-white product was dried in a vacuum oven under reduced pressure at 35° C. over 48 h to give 637.8 g (106% yield, 94% HPLC purity) of the desired product as an off-white solid.

### Example IV-8: Synthesis of H-[6-7]-OMe (H-Pen(Acm)-Tyr(2-Boc-ea)-OMe)

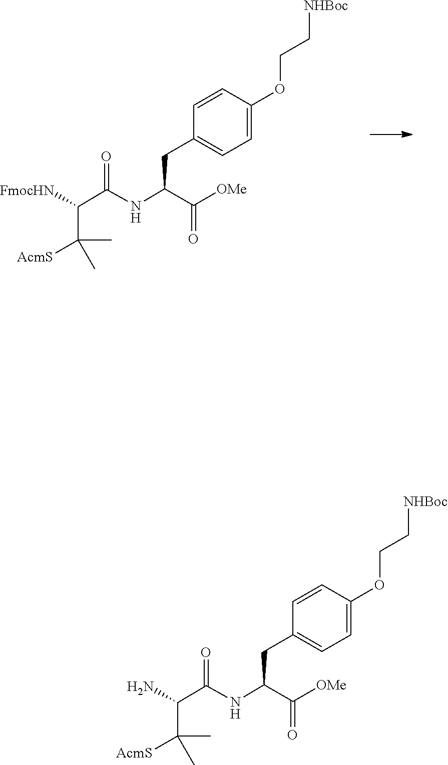

Fmoc-Pen(Acm)-Tyr(2-Boc-ea)-OMe (0.786 mol, 1.0 eq) was dissolved in EtOAc/DMSO (9:1; v:v) (6 L). To this solution, DBU (0.393 mol, 0.5 eq.) was added. The reaction mixture was stirred at RT for 2.5 hours until completion of the reaction. The organic product layer was diluted with EtOAc and washed with 5% NaHCO₃ aq. and H₂O. The aqueous phases were washed with EtOAc. The organic phases were combined and evaporated at 35° C. The residue was co-distillated twice with EtOAc. The residue was stored at 4-5° C. overnight and directly used as starting material for the following step (Example 9, synthesis of Fmoc-[5-7]-OMe).

### Example IV-9: Synthesis of Fmoc-[5-7]-OMe (Fmoc-Lys(Ac)-Pen(Acm)-Tyr(2-Boc-ea)-OMe)

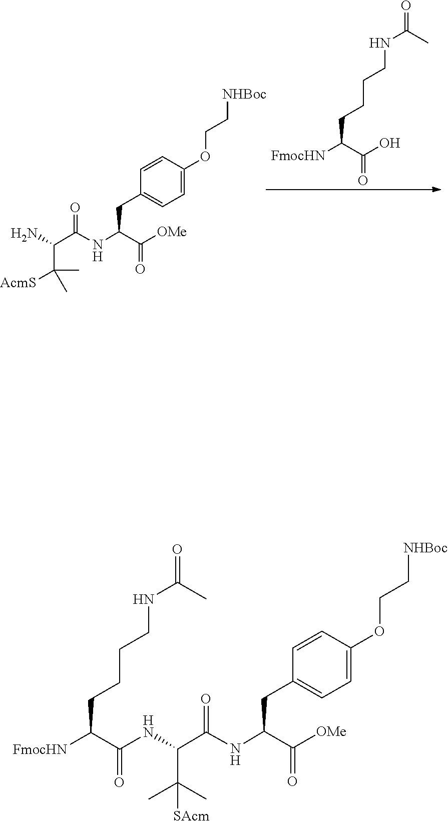

To the solution of H-Pen(Acm)-Tyr(2-Boc-ea)-OMe (0.732 mol, 1.0 eq.) in EtOAc/DMSO (8:2; v:v) (3 L) was added Fmoc-Lys(Ac)—OH (0.637 mol, 0.87 eq.) and TBTU (0.696 mol, 0.95 eq.) and the reaction mixture was stirred for 2 minutes at RT. DIPEA (1.391 mol, 1.90 eq.) was added and the reaction mixture was stirred at RT for 2 h until completion of the reaction. The reaction mixture was diluted with EtOAc and washed twice with 20% NaHSO₄ aq, H₂O, twice with 5% Na₂CO₃ aq. and again with water. The water phases were washed with EtOAc. The organic phase was evaporated at 35° C. and co-distilled twice with EtOAc. The residue was dissolved in EtOAc. The mixture was slowly added to IPE over 25 minutes. The suspension was filtered and washed three times with IPE. The solid was dried under high vacuum at 35° C. overnight. The white solid was dissolved in EtOAC. The solution was added to IPE over 5 minutes. The suspension was filtered and washed three times with IPE. The solid was dried under high vacuum at 35° C. overnight to give 554.9 g (76% yield over 2 steps, 97 HPLC purity) of the desired product as a white solid.

### Example IV-10: Synthesis of H-[5-7]-OMe (H-Lys(Ac)-Pen(Acm)-Tyr(2-Boc-ea)-OMe)

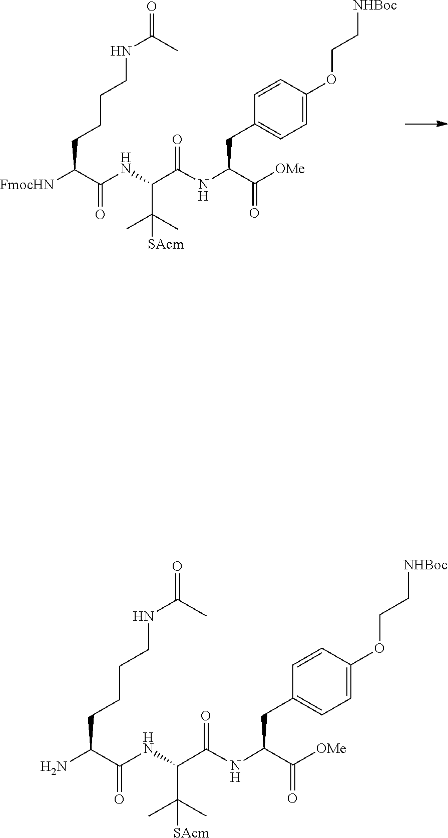

Fmoc-Lys(Ac)-Pen(Acm)-Tyr(2-Boc-ea)-OMe (0.563 mol, 1.0 eq) was dissolved in EtOAc/DMSO (9:1; v:v) (5.2 L). To this solution, DBU (0.281 mol, 0.5 eq.) was added. The reaction mixture was stirred at RT for 1.5 hours until completion of the reaction. The organic product layer was diluted with EtOAc and washed twice with H₂O. The aqueous phases were washed twice with EtOAc. The organic phases were combined and evaporated at 40° C. The residue was co-distillated twice with EtOAc. The residue was stored at 4-5° C. overnight and directly used as starting material for the following step (step 11, synthesis of Fmoc-[4-7]-OMe).

### Example IV-11: Synthesis of Fmoc-[4-7]-OMe (Fmoc-Trp(7Me)-Lys(Ac)-Pen(Acm)-Tyr(2-Boc-ea)-OMe)

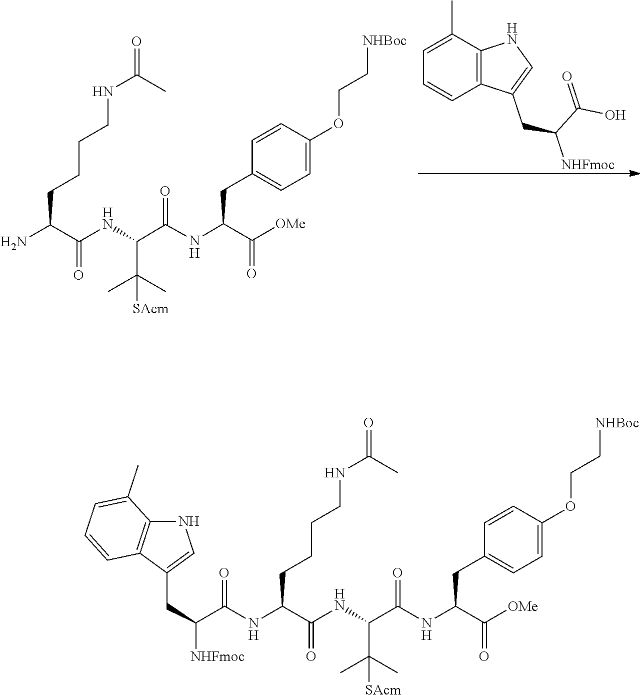

To the solution of H-Lys(Ac)-Pen(Acm)-Tyr(2-Boc-ea)-OMe (0.512 mol, 1.0 eq.) in EtOAc/DMSO (8:2; v:v) (2.6 L) was added Fmoc-Trp(7Me)-OH (0.415 mol, 0.81 eq.) and TBTU (0.486 mol, 0.95 eq.) and the reaction mixture was stirred for 2 minutes at RT. DIPEA (1.024 mol, 2.00 eq.) was added and the reaction mixture was stirred at RT for 2 h until completion of the reaction. The reaction mixture was diluted with EtOAc and washed twice with 5% NaHCO₃ aq. The aqueous phases were washed with EtOAc. The organic phases evaporated at 40° C. The residue was azeotroped twice with EtOAc. The residue was diluted with EtOAc and slowly added to IPE/heptane (1:1; v:v) over 10 minutes. The suspension was filtered and washed three times with IPE. The solid was dried under high vacuum at 35° C. overnight to give 487.67 g (76% yield over 2 steps, 95% HPLC purity) of the desired product as a white solid.

### Example IV-12: Synthesis of Boc-[12-13]-NH2 (Boc-3Pal-Sarc-NH2)

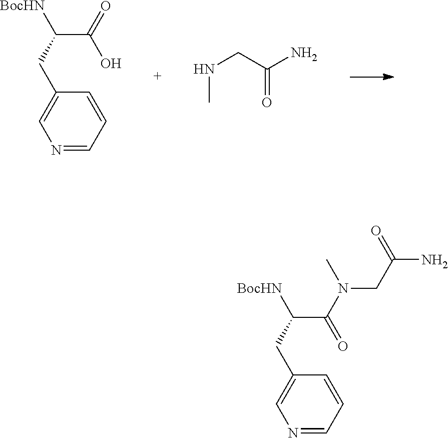

Boc-3-Pal-OH (106.5 g, 0.40 mol, 1.0 eq) was dissolved in DMF (0.5 L) and cooled to −6° C. Pyridine (32 mL, 1.0 eq.) and NMM (44 mL, 1.0 eq.) were added, and the mixture cooled down to −25° C. Pivaloyl chloride (49 mL, 1.0 eq.) was added, and the mixture stirred for 20 min at −10° C. In a parallel reactor, H-Sar-NH2·HCl (49.8 g, 1.0 eq.) was dissolved in DMF (250 mL) and water (50 mL) and the solution cooled to 0° C. After addition of NMM (44 mL), the resulting amine solution was added to the solution of the activated Boc-3-Pal-OH. The resulting suspension was stirred at RT for 14 h until completion of the reaction. The suspension was cooled to 3° C. and the precipitated product was filtered off, washed four times with 250 mL cold water (4° C.), and dried in vacuo at 35° C. for 14 h to give the desired product (116 g, 88% yield, 99% HPLC purity) as a white solid.

### Example IV-13: Synthesis of H-[12-13]-NH2. HCl (H-3Pal-Sarc-NH2)

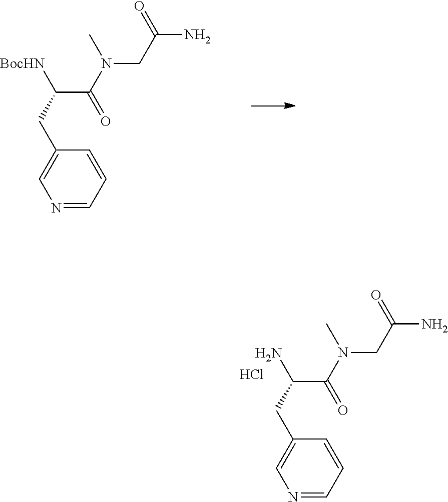 [SMILES: CN(CC(N)=O)C(=O)[C@H](Cc1cccnc1)NC(=O)OC(C)(C)C ; Cl ; CN(CC(N)=O)C(=O)[C@@H](N)Cc1cccnc1]

Boc-3-Pal-Sar-NH2 (115 g, 0.34 mol) was added under good stirring to HCl in EtOAc (3.4 N, 1.2 L). The reaction was complete after 1 h. The suspension was filtered, the residue was washed three times with 0.5 L EtOAc and dried in vacuo at 35° C. for 16 h to give the desired product (163 g, >100% yield, 99% HPLC purity) as a white solid.

### Example IV-14: Synthesis of Cbz-Asn-3Pal-Sarc-NH2

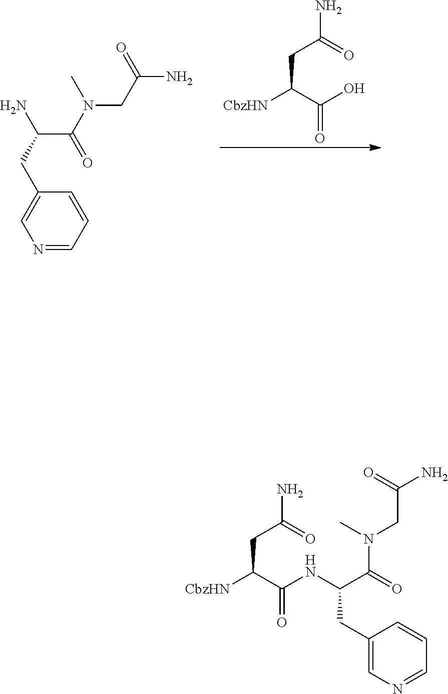 [SMILES: CN(CC(N)=O)C(=O)[C@@H](N)Cc1cccnc1 ; NC(=O)C[C@H](NC(=O)OCc1ccccc1)C(=O)O ; CN(CC(N)=O)C(=O)[C@H](Cc1cccnc1)NC(=O)[C@H](CC(N)=O)NC(=O)OCc1ccccc1]

### Pre-Activation of Z-Asn-OH

87.5 g Z-Asn-OH (0.33 mol, 1.1 eq.) and 41.6 g (1.2 eq.) HOSu were dissolved in 440 mL DMF, and the solution was cooled down to 0° C. DIC (54 mL, 1.1 eq.) was added within 5 min, and the mixture was stirred for 17 h at 0° C. The resulting suspension was clarified by filtration, and the filter cake was washed two times with 40 mL DMF each. The filtrate was directly used for the coupling reaction.

### Coupling and Workup

140.6 g (49.6% peptide content; 0.30 mol; 1.0 eq.) of H-3-Pal-Sar-NH2·HCl was added to the solution of Z-Asn-OSu at 10° C. and a total of 117 mL triethylamine was added within 1 h, under cooling (maximum temperature 32° C.) to adjust the pH to 7 resulting in a thick suspension. The suspension was stirred for an additional 30 min until completion of the reaction. 875 mL ACN was added to the thick product suspension, which was stirred for 1 h. The product was filtered off, washed five times with 1 L ACN each, and dried in vacuo at 35° C. for 18 h to give the desired product (113 g, 78% yield, 99.3% HPLC purity as a white solid.

### Example IV-15: Synthesis of H-[11-13]-NH2 (H-Asn-3Pal-Sarc-NH2)

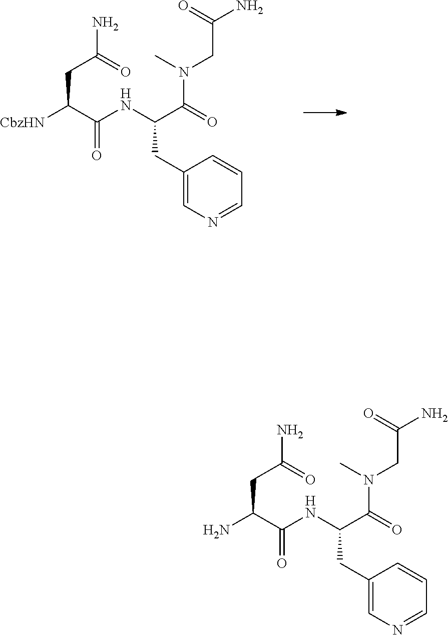 [SMILES: CN(CC(N)=O)C(=O)[C@H](Cc1cccnc1)NC(=O)[C@H](CC(N)=O)NC(=O)OCc1ccccc1 ; CN(CC(N)=O)C(=O)[C@H](Cc1cccnc1)NC(=O)[C@@H](N)CC(N)=O]

Z-Asn-3-Pal-Sar-NH2 (111.4 g, 0.23 mol) was suspended in DMF (0.9 L). After addition of 11 g Pd/C catalyst, hydrogen was passed through the mixture for 7 h resulting in dissolution of the starting material. The mixture was held over night at 4° C. leading to precipitation of the product. This suspension (1 L) was used for the next coupling step without filtering off the catalyst.

### Example IV-16: Synthesis of Cbz-[10-13]-NH2 (Cbz-Glu(tBu)-Asn-3Pal-Sarc-NH2)

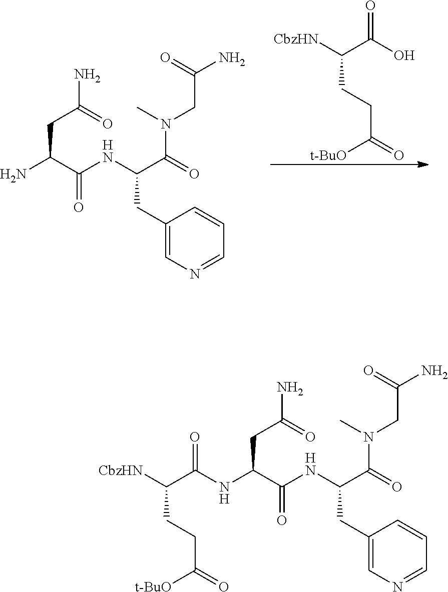 [SMILES: CN(CC(N)=O)C(=O)[C@H](Cc1cccnc1)NC(=O)[C@@H](N)CC(N)=O ; CC(C)(C)OC(=O)CC[C@H](NC(=O)OCc1ccccc1)C(=O)O ; CN(CC(N)=O)C(=O)[C@H](Cc1cccnc1)NC(=O)[C@H](CC(N)=O)NC(=O)[C@H](CCC(=O)OC(C)(C)C)NC(=O)OCc1ccccc1]

### Pre-Activation of Z-Glu(OtBu)-OH

Z-Glu(OtBu)-OH (77.4 g, 0.23 mol, 1.0 eq.) and HOSu (29.1 g, 1.1 eq.) were dissolved in DMF (200 mL) and the solution cooled to 0° C. DIC (37 mL, 1.04 eq) was added within 10 min. The reaction was heated to 25° C. within 10 h and stirred further for 8 h.

### Coupling and Workup

To 1.0 L suspension of H-Asn-3-Pal-Sar-NH2 (1.0 eq.) in DMF from the previous step (H-[11-13]-NH2), Z-Glu(OtBu)-OSu was added as suspension. The pH was adjusted to pH 8.5 by addition of Et₃N (16 mL, 0.5 eq.) and the mixture was stirred for 90 min.

The suspension was filtered. The filtrate was extracted consecutively with 2 L IPE/hexane 1:1, 2 L IPE, and 1 L IPE. The product started to precipitate during the extractions. The lower phase was evaporated to a thick suspension (0.5 L) and diluted with 1 L THF to obtain a thin suspension. The product was filtered off and washed with 1 L DMF/THF 1:3, as well as two times with 1 L THF each, and dried in vacuo at 35° C. for 16 h to give the crude product (109 g, 71% yield, 98% HPLC purity).

Purification by suspending in water: Z-Glu(OtBu)-Asn-3-Pal-Sar-NH2 (109 g) was suspended in water (550 mL) for 5 min at 65° C. and stirred for 30 min at 25° C. The product was filtered off, washed three times with 0.5 L water and dried in vacuo at 35° C. for 20 h to give the desired product (85 g, 55% overall yield, 99% HPLC purity).

### Example IV-17: Synthesis of H-[10-13]-NH2 (H-Glu(tBu)-Asn-3Pal-Sarc-NH2)

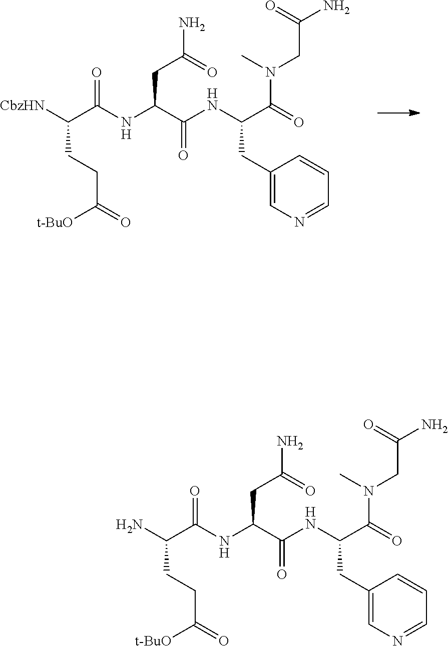 [SMILES: CN(CC(N)=O)C(=O)[C@H](Cc1cccnc1)NC(=O)[C@H](CC(N)=O)NC(=O)[C@H](CCC(=O)OC(C)(C)C)NC(=O)OCc1ccccc1 ; CN(CC(N)=O)C(=O)[C@H](Cc1cccnc1)NC(=O)[C@H](CC(N)=O)NC(=O)[C@@H](N)CCC(=O)OC(C)(C)C]

Z-Glu(OtBu)-Asn-3-Pal-Sar-NH2 (105 g, 1.0 eq.) was suspended in MeOH (1130 mL) and water (170 mL). The mixture was stirred at 40° C. until the starting material dissolved completely. Pd/C (10.5 g) was then added and the reaction mixture was stirred at 40° C. under a hydrogen pressure of 3.5 bar for 2 hours. The reaction suspension was filtered and the cake washed with MeOH (2×60 mL). The filtrate was dried with Na₂SO₄ and concentrated under reduced pressure at 45° C. The residue was poured onto IPE (4000 mL) and after stirring for 15 minutes at room temperature the suspension was filtered and the cake washed with IPE (2×100 mL). The white solid was dried in a vacuum oven at 35° C. for 18 hours to give 78.0 g (93% yield, 96% HPLC purity) of the desired product as a white solid.

### Example IV-18: Synthesis of Fmoc-[8-9]—OH (Fmoc-2Nal-Gly(THP)-OH)

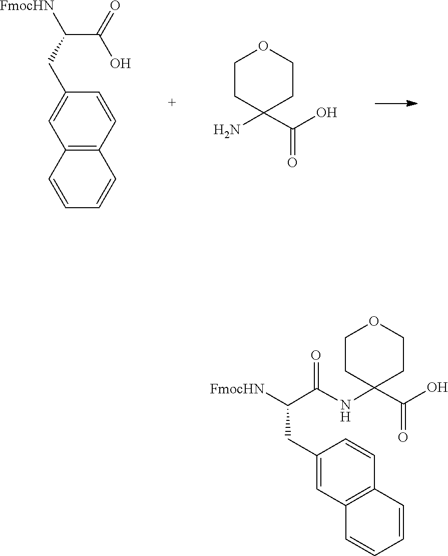

To a suspension of H-THPGly-OH (10.8 g, 1.2 eq) in acetonitrile (120 mL) under inert atmosphere was added N,O-bis(trimethylsilyl)acetamide (30.1 g, 2.4 eq) and the reaction mixture was heated to 70° C. and stirred at this temperature for 1 h. To a solution of Fmoc-2-Nal-OH (27.0 g, 1 eq) in 2-methyltetrahydrofuran (200 mL) cooled to 0° C. was added PivCl (8.2 g, 1.1 eq) and NMM (6.9 g, 1.1 eq). The solution was warmed up to 10° C. The H-THPGly-OH solution was added to the cold Fmoc-2-Nal-THPGly-OH suspension within 30 minutes and the reaction mixture was stirred overnight at RT. Me-THF (200 mL) was added to the reaction mixture. The organic phase was extracted twice with NaHSO₄ (˜5% eq.) and washed once with NaCl (˜3%, eq.). The solution was concentrated at 45° C. and azeotroped with Me-THF. The residue was diluted with Me-THF (90 mL) and was slowly precipitated onto heptane (900 mL). The white suspension was filtered and washed with heptane (100 mL). The solid was dried in a vacuum drying cabinet at 40° C. to give 35 g Fmoc-2-Nal-THPGly-OH (100% yield, 97% HPLC purity) as a white solid.

### Example IV-19: Synthesis of Fmoc-[8-13]-NH2 (Fmoc-2Nal-Gly(THP)-Glu(tBu)-Asn-3Pal-Sarc-NH2)

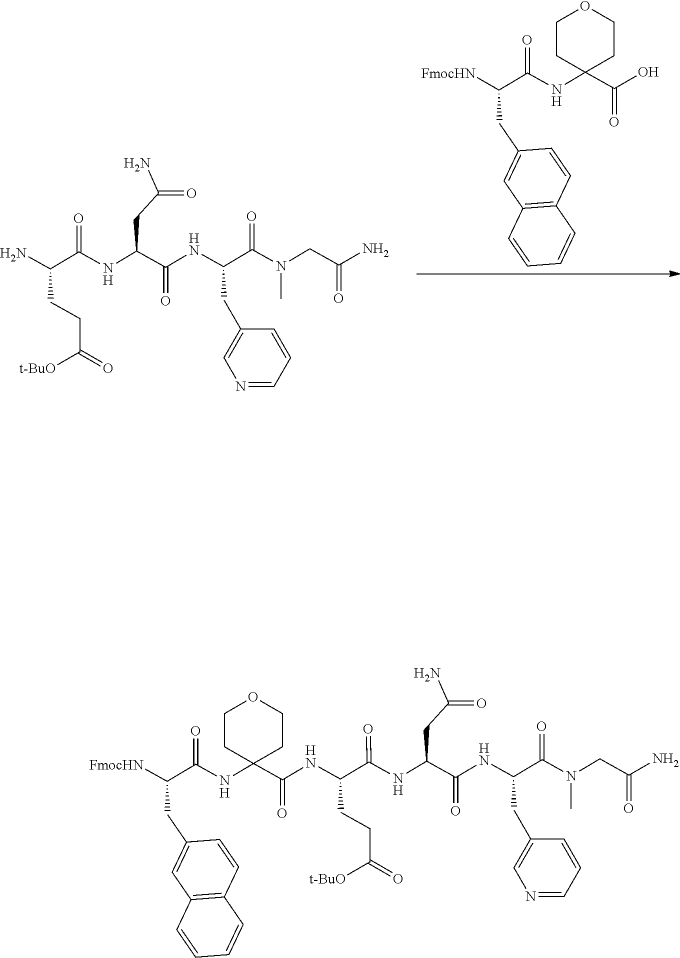

Fmoc-2-Nal-THPGly-OH (59.0 g, 1.0 eq.) and H-Glu(OtBu)-Asn-3-Pal-Sar-NH2 (67.2 g, 1.2 eq.) were suspended in MeTHF (1490 mL) and DMSO (310 mL) and the mixture was cooled to 0-5° C. PyAOP (59.9 g, 1.1 eq.) was added in one portion followed by the addition of DIPEA (36.4 mL, 2.0 eq.). The reaction mixture was then allowed to warm to room temperature and stirred for 16 hours. The reaction was then diluted with MeTHF (1500 mL) and extracted with 2% NaHCO₃ (2×2000 mL) and brine (4% NaCl aq) (1×2000 mL). The organic phase was evaporated under reduced pressure at 45° C. and the residual oil co-distilled with MeTHF (2×1000 mL). The residual thick suspension was diluted with MTBE (1250 mL) and stirred at 40° C. for 20 minutes and at room temperature for 30 minutes. The suspension was then filtered and the cake washed with MTBE (3×300 mL). The white solid was dried in a vacuum oven at 30° C. for 2 days to give 133 g (99% yield, 95% HPLC purity) of the desired product as a white solid.

### Example IV-20: Synthesis of H-[4-7]-OMe (H-Trp(7Me)-Lys(Ac)-Pen(Acm)-Tyr(2-Boc-ea)-OMe)

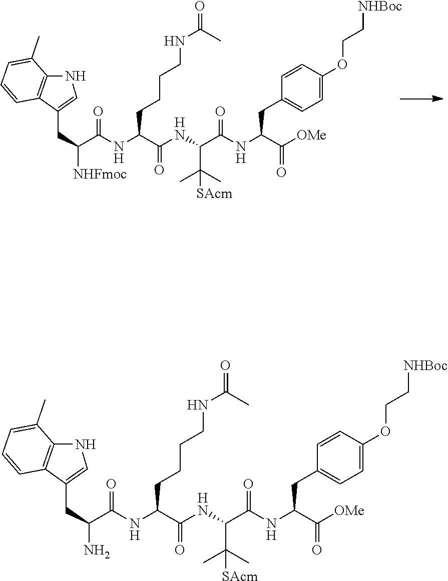

To a solution of Fmoc-7Me-Trp-Lys(Ac)-Pen(Acm)-Tyr(2-Boc-ea)-OMe (450 g, 397 mmol, 1.0 eq.) in EtOAc/DMSO (8:2, v:v) (4.5 L) at RT was added DBU (29.63 mL, 198.5 mmol, 0.5 eq.) and the reaction mixture was stirred at RT. IPC-HPLC after 1 h showed 0.01% Fmoc-7-Me-Trp-Lys(Ac)-Pen(Acm)-Tyr(2-Boc-ea)-OMe. 1.5 h after DBU addition the reaction mixture was diluted with EtOAc/Me-THF 1:1 (9.0 L). The organic solution was washed twice with NaHCO₃ (5%, aq.) (4.5 L) and once with H₂O (4.5 L). The aqueous phases were combined and were washed with EtOAc/Me-THF 1:1 (4.5 L). The organic phase was evaporated under reduced pressure at 40° C. The residue was co-evaporated with EtOAc (3×2 L). The residue was stored at 4-5° C. overnight. The residue was taken up in EtOAc (1 L) to give in total 3 L volume. The diluted mixture was added at RT onto IPE (30 L) over 5 minutes. The suspension was stirred at RT for 10 minutes. The suspension was filtered and the filter cake was washed twice with IPE (1.5 L). The white solid was dried under high vacuum at 35° C. overnight. The white solid (345.7 g) was re-dissolved in EtOAc (3.5 L) within 10 minutes. The solution was added to IPE (35 L) over 10 minutes. The white suspension was stirred at RT over 30 minutes. The white suspension was filtered and the filter cake was washed twice with IPE (1.5 L). The white solid was dried under high vacuum at 35° C. overnight to give H-Trp(7Me)-Lys(Ac)-Pen(Acm)-Tyr(2-Boc-ea)-OMe (336 g, 93%, assay (Q-NMR): 89%).

### Example IV-21: Synthesis of Ac-[1-7]-OMe (Ac-Pen(Trt)-Asn-Thr(tBu)-Trp(7Me)-Lys(Ac)-Pen(Acm)-Tyr(2-Boc-ea)-OMe)

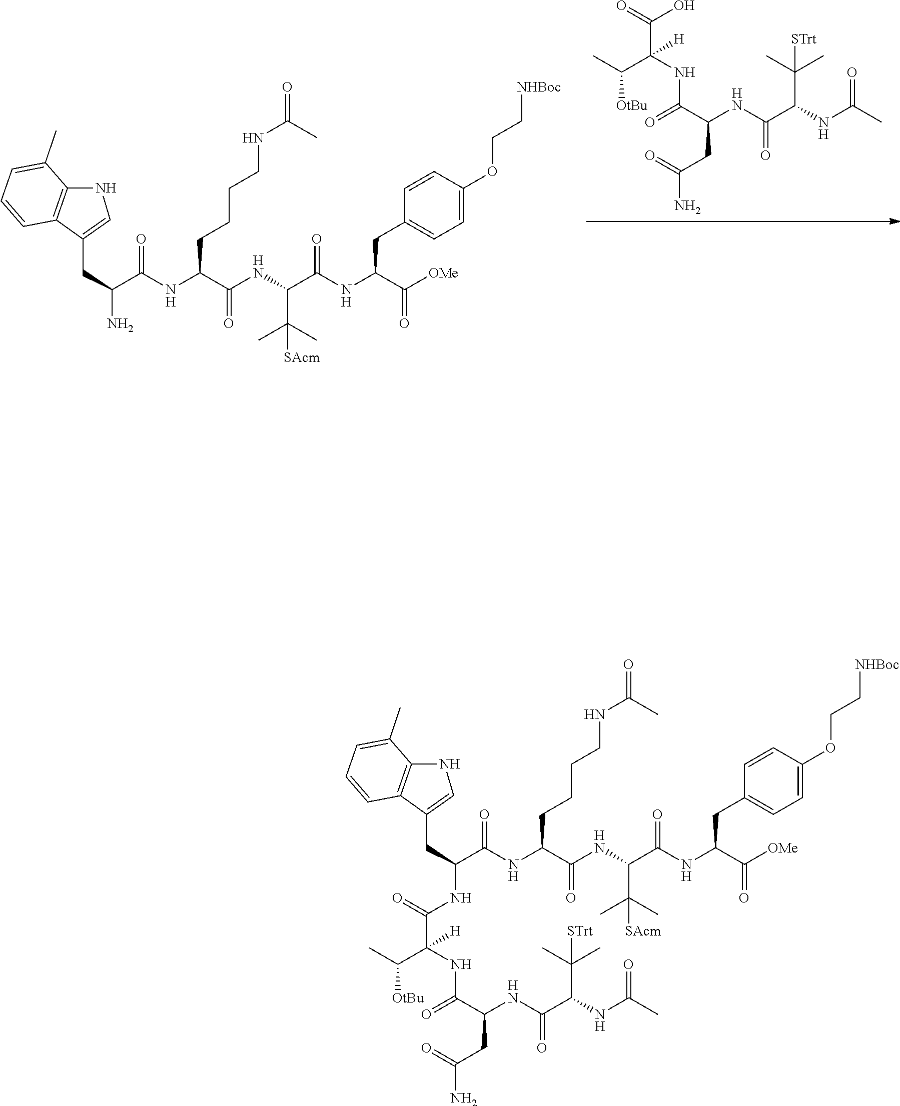

In a double-jacketed 10 L glass reactor were loaded Ac-Pen(Trt)-Asn-Thr(tBu)-OH (212.0 g, 273.692 mmol, 1.0 eq.), H-7Me-Trp-Lys(Ac)-Pen(Acm)-Tyr(2-Boc-ea)-OMe (275.4 g, 93.3w/w %, 281.903 mmol, 1.03 eq.), Oxyma B (50.7 g, 273.692 mmol, 1.0 eq.) and Acetonitrile (3.23 kg, 4.2 L). The mixture was stirred at 25° C. for 30 minutes (red-fine suspension) and DIC (38.0 g, 301.061 mmol, 1.1 eq.) was added over 5 minutes and the reaction mixture was stirred at 25° C. IPC-HPLC after 3 hours showed completeness of the reaction. A white to beige suspension was formed. The suspension was stirred at 25° C. for further 21 hours and the product separated by filtration through a pore 3 glass filter and the filter cake was washed with ACN (3×0.3 L). The wet product was re-suspended in ACN (2.0 L, 1.55 kg) and the suspension was heated to 40° C. within 30 minutes and stirred at this temperature for 1.5 hours. The suspension was cooled to 15° C. within 1 hour and was stirred at this temperature for 6 hours. The product was separated by filtration through a pore 3 glass filter. The filter cake was washed with ACN (3×0.3 L mL) and dried under reduced pressure at 35° C. to give 364.0 g of a off-white to beige solid (HPLC purity: 97.0% (D-allo-Thr: 0.08%), ESI-MS: m/z=1597.32 [M+H⁺], Yield: 83.2%, Peptide content (CAT): 91.7%, HPLC Assay: 89.1%).

### Example IV-22: Synthesis of Ac-[1-7]—OH-linear (Ac-Pen(Trt)-Asn-Thr(tBu)-Trp(7Me)-Lys(Ac)-Pen(Acm)-Tyr(2-Boc-ea)-OH-linear)

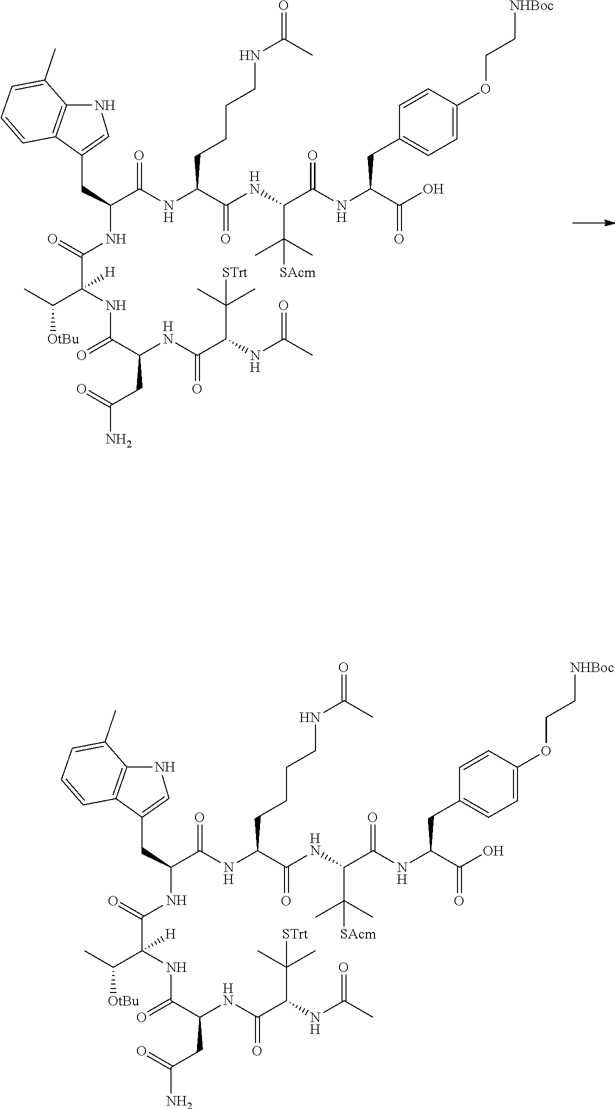

Ac-[1-7]-OMe (150.0 g, 89.1% w/w, 83.637 mmol, 1.0 eq.), THF (0.75 L, 0.657 kg) and water (0.525 L, 0.525 kg) were loaded into a 1.5 L glass jacketed reactor with overhead stirrer. The mixture was stirred at 25° C. until a solution was obtained and cooled to 0° C. within 30 minutes. A solution of LiOH×H₂O (5.264 g, 125.455 mmol, 1.5 eq.) in water (0.225 L, 0.225 kg) was added within one hour to the reaction mixture under stirring and the temperature was kept at 0-2° C. Two h after complete LiOH solution addition, IPC-HPLC showed completeness of the reaction (Product: 99.1%, D-Tyr: 0.9%, S.M: n.d). The pH of the reaction mixture was adjusted to 4.9 with HCl (conc.) and the mixture was heated to 25° C. within 30 min. The reaction mixture was diluted with MeTHF (1.5 L) and the phases left to separate. The solvent was evaporated from the organic layer under reduced pressure at 40° C. 300 mL and the residue was co-evaporated with EtOAc (3×1 L). The product started to precipitate after the 2nd. co-evaporation. The suspension was taken up in EtOAc (820 mL), stirred at 60° C. for 30 minutes, cooled to 25° C. within 4 hours and stirred at this temperature overnight. The product was separated by filtration through a pore 3 glass filter and the filter cake washed with EtOAc (200 mL). The product was dried under vacuum at 35° C. for 24 h to give 135.7 g of a beige to off-white solid. (HPLC purity: 96.1% (D-Tyr=0.9%), ESI-MS: m/z=1583.36 [M+H⁺]⁺, Yield: 82.8% (assay corrected), HPLC Assay: 80.7%).

### Example IV-23: Synthesis of Ac-[1-7]—OH-cyclic (Ac-Pen(Trt)-Asn-Thr(tBu)-Trp(7Me)-Lys(Ac)-Pen(Acm)-Tyr(2-Boc-ea)-OH-cyclic)

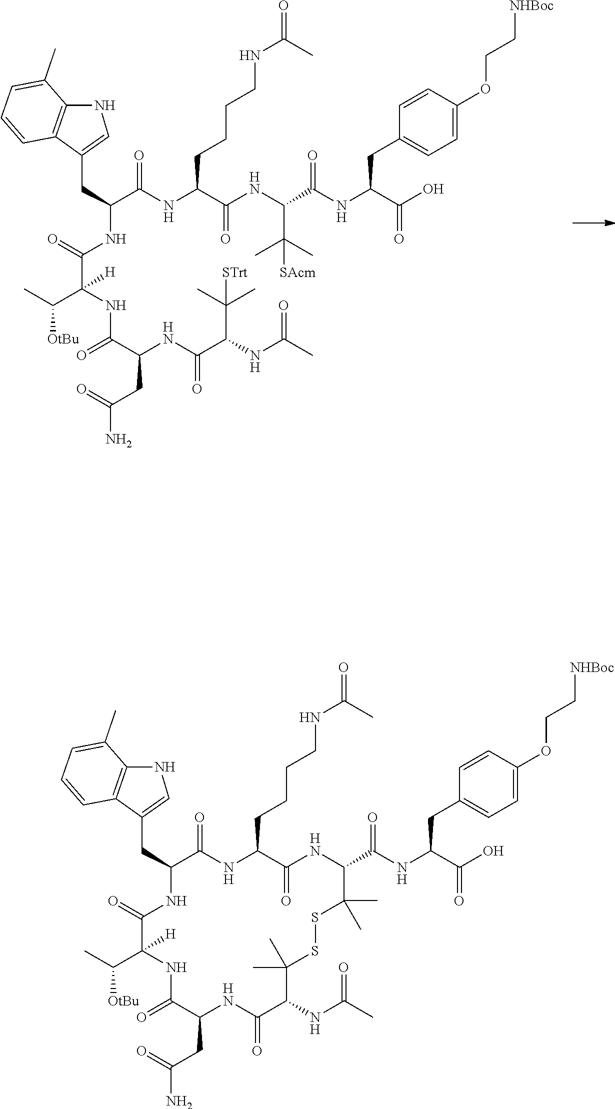

In a 400 mL glass reactor, a solution iodine (3.17 g, 5.00 equiv., 12.5 mmol) and potassium iodide (2.08 g, 5.00 equiv., 12.5 mmol) in ACN/NMP/water (11.81/5.91/7.59 vol, 101.2 mL) was stirred for 15 min at 25° C. A solution of Ac-[1-7]—OH-linear (4.00 g, NMR-assay: 74-90%, UPLC purity: 99.2%, 1 equiv., 2.5 mmol) in ACN/NMP/water (3.13/1.57/2.01 vol, 26.8 mL) was added with a dosage pump within 2 hours under nitrogen atmosphere at 25° C. (Conc: 20 mM). The reaction mixture was stirred at 25° C., overnight (20 h). The solution was cooled to 15° C. and a solution of ascorbic acid (3.3 g, 7.5 equiv., 18.8 mmol) in water (48 mL) was added within 15 min to give a clear, almost colourless solution. The pH was adjusted from 1.2 to 5.0 with addition of NaHCO₃ (10% aqueous solution) (20.3 g). The reaction mixture became turbid (precipitation of TrtOH). The solid was removed via filtration and the filtrate was transferred into a round-bottom flask. The mixture was evaporated under reduced pressure at 45° C. to remove the acetonitrile. The residue was diluted with Me-THF (100 mL). The pH was adjusted to 3.0 with NaHSO₄ (10% aqueous solution) and the mixture was stirred for 30 min (3.8 g). The phases were separated, and the aqueous phase was extracted with Me-THF (1×40 mL). The combined organic phases were washed with brine 10% (2×40 mL) and water (1×40 mL). The organic phase (76.4 g) was evaporated under reduced pressure at 45° C. to a clear, yellowish solution with some solid (30.4 g). The mixture was azeotropically distilled with Me-THF (3×15 mL) to form a white thick suspension. Even though suspension was thick, it was able to be stirred. The suspension was diluted with MTBE (30 mL) and heptane (6.8 mL) and stirred for at least 15 min at 25° C. It was filtered and washed with MTBE (2×5 mL). The product was dried in a vacuum oven at 40° C. for 18 h to give 3.052 g (82.8% yield, assay corrected) of a white solid. UPLC assay: 86.0%; Area %: 94.3%

### Example IV-24: Synthesis of H-[8-13]-NH2 (H-2Nal-Gly(THP)-Glu(tBu)-Asn-3-3Pal-Sarc-NH2)

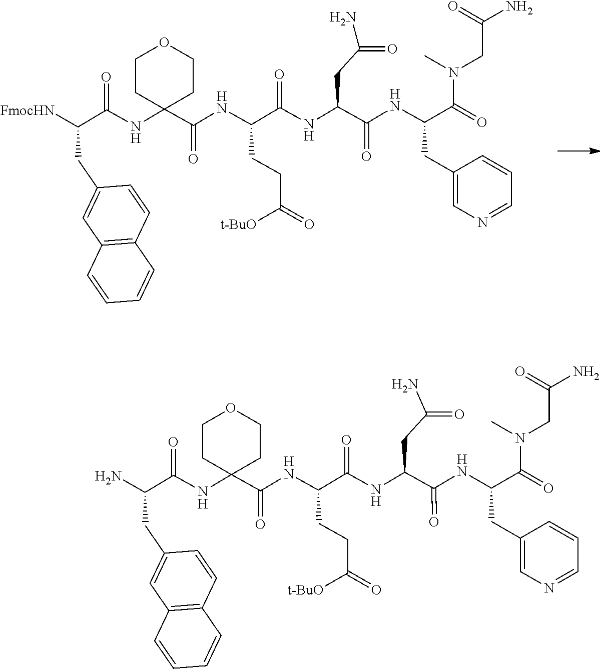

Fmoc-2-Nal-THPGly-Glu(OtBu)-Asn-3-Pal-Sar-NH2 (109.0 g, 1.0 eq.) was dissolved at room temperature in MeTHF (680 mL) and DMSO (172 mL). DBU (9.11 mL, 0.6 eq.) was slowly added to the solution and the mixture was stirred for 17 hours. The reaction mixture was extracted with 20% w/w (weight/weight) solution of NaCl in water (2000 mL) and the aqueous phase was back-extracted with IPA (3×700 mL). The combined IPA layers were concentrated under reduced pressure at 45° C. The residue was filtered to remove the precipitated salts. The filtrate was diluted with IPA (500 mL) and concentrated, this procedure was repeated twice. The oily residue was co-distilled with EtOAc (2×500 mL) and was poured onto a EtOAc/MTBE (9:1, 5000 mL) mixture and the suspension was stirred at room temperature for 15 minutes. The suspension was then filtered and the cake was washed with EtOAc/MTBE (1:1, 2×500 mL). The white solid was dried in a vacuum oven at 30° C. for 2 days to give 80.8 g (93% yield, 93.5% HPLC purity) of the desired product as a white solid.

### Example IV-25: Synthesis of Compound 25 (Ac-Pen(Trt)-Asn-Thr(tBu)-Trp(7Me)-Lys(Ac)-Pen(Acm)-Tyr(2-Boc-ea)-2Nal-Gly(THP)-Glu(tBu)-Asn-3Pal-Sarc-NH2)

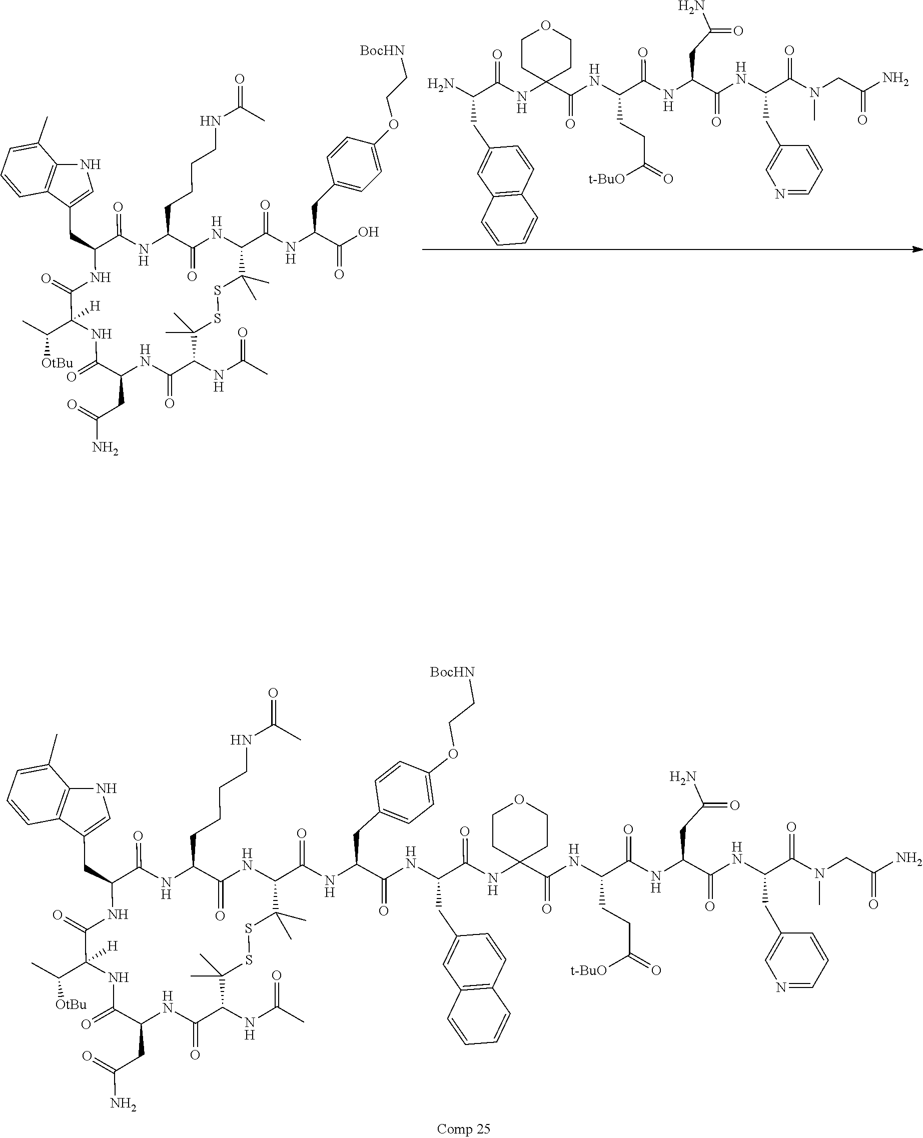

In a 10 L reactor Ac-[1-7]—OH-cyclic (90.00 g, 91.0% Wt, 1 Eq, 64.56 mmol), H-[8-13]-NH2 (75.01 g, 75.5% Wt, 1.02 Eq, 65.85 mmol) and Oxyma-B (13.15 g, 1.10 Eq, 71.02 mmol) were dissolved in Me-THF/DMF (4:1) (1350 mL) at 25° C. for 20 min. To the dark blue solution was added DIC (8.148 g, 10.1 mL, 1.00 Eq, 64.56 mmol) within 2 min and stirred for 2.5 h at 25° C. Additional DIC (2.037 g, 2.53 mL, 0.25 Eq, 16.14 mmol) was added and stirred further at 25° C. After completion of the reaction, the mixture was diluted with Me-THF (4.00 L) and was washed with NaHCO₃ (5% w/w solution of NaHCO₃ in water) (1×1.50 L) and stirred at 30° C. for 20 min. The phases were separated. There was approximately 100 mL of oily phase between. The organic phase (with oil) was diluted with MeTHF (1.00 L) and washed with NaHCO₃ (2.5% solution of NaHCO₃ in water) (2×1.25 L) and water (2×1.00 L). All phase separations were performed at 30° C. The organic phase was evaporated to a thin suspension and co distilled with MeTHF/MeOH (4:1; 2×1.20 L) to an orange oil. The oil was precipitated by dropping within 10 min on EtOAc (4.00 L) at 20° C. The white suspension was stirred for 30 min then filtered and washed with EtOAc (3×200 mL). The product was dried in a vacuum oven at 40° C. for 24 h and 3 days at 35° C. to give 133.1 g (86% yield assay corrected, HPLC purity 92%) of the desired product as a white solid.

### Example IV-26: Synthesis of Compound 26 (Ac-Pen-Asn-Thr-Trp(7Me)-Lys(Ac)-Pen-Tyr(2-ea)-2-Nal2Nal-Gly(THP)-Glu-Asn-3-Pal3Pal-Sarc-NH2)

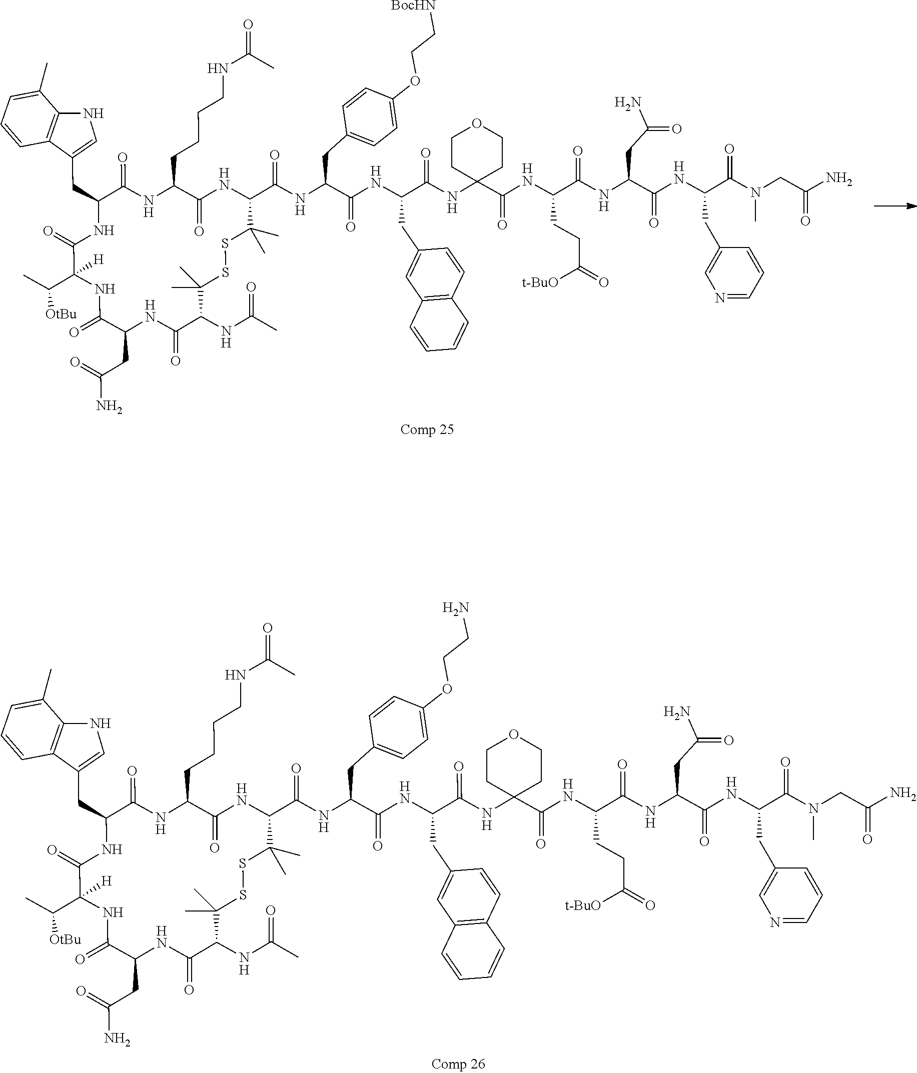

Compound 25 (60.0 g, 87.8% Wt (assay), 1 Eq, 25.0 mmol) was loaded in a 6 L double jacket reactor equipped with condenser, thermometer and stirrer. IProAc (150 mL) was added and stirred for 15 min at 15° C. The suspension was stirred at 15° C. and cold (5° C.) 3M HCl/IProAc (1050 mL) was added within 1 min and stirred at 15° C. for 5 min. The mixture was warmed up to 30° C. within 20 min and stirred for 5 h. After completion of the reaction, the mixture was cooled to −5° C. and NaOH (about 5% aqueous solution of NaOH) (1500 mL) was added in 40 min. Additional water (150 mL) was added and the pH was adjusted to 2.8 with NaOH (5% aqueous solution of NaOH) (145 mL) at 22° C. The mixture was stirred at 22° C. for 1 h until the solids were dissolved and the phases were separated. The organic phase was extracted with water (120 mL). The combined aqueous phases (approx. 2.2 L yellow solution) were stirred at 22° C. and the pH was adjusted to 4.25 within 20 min with NaOH (about 5% aqueous solution of NaOH) (30 mL) and stirred for 1 h to give a white suspension. The pH was adjusted to 4.50 with NaOH (5% aqueous solution of NaOH) (13 mL) and the suspension was stirred for 14 h at 20° C. The suspension was filtrated and washed with water 5° C. (2×50 mL). The wet cake was dried in a vacuum oven at 45° C. for 24 h to give compound 26 (42.8 g, 89% yield, 95% HPLC purity) as a white powder.

1. A process for preparing a monocyclic peptide or a salt thereof, comprising coupling a monocyclic peptide fragment with a linear chain peptide fragment,
wherein the monocyclic peptide fragment is a peptide containing between 4 and 11 amino acid residues;
wherein the monocyclic peptide fragment comprises a ring containing between 4 and 8 amino acid residues;
wherein the linear chain peptide fragment is a peptide containing between 4 and 10 amino acid residues; and wherein an amide bond is formed between the monocyclic peptide fragment and the linear chain peptide fragment.

2. The process of claim 1 wherein the coupling of the monocyclic peptide fragment with the linear chain peptide fragment is carried out in a solution phase without using a solid support.

3. (canceled)

4. (canceled)

5. (canceled)

6. (canceled)

7. (canceled)

8. The process of claim 1, wherein the monocyclic peptide fragment comprises a ring which is cyclized by a bond between side chains of two amino acid residues.

9. The process of claim 1, wherein the monocyclic peptide fragment comprises a ring which is cyclized via a disulfide-bridge or a thioether bond between side chains of two amino acid residues.

10. (canceled)

11. (canceled)

12. (canceled)

13. (canceled)

14. (canceled)

15. The process of claim 1, wherein the linear chain peptide fragment is an unbranched peptide.

16. The process of claim 1, wherein the monocyclic peptide fragment comprises a ring which is appended by at least one peptide chain containing at least 1 amino acid residue.

17. (canceled)

18. The process of claim 1, further comprising a step of cyclizing a second linear peptide fragment to form the monocyclic peptide fragment, wherein the second linear peptide fragment is a peptide containing between 4 and 11 amino acid residues.

19. (canceled)

20. (canceled)

21. (canceled)

22. (canceled)

23. (canceled)

24. The process of claim 1, wherein the monocyclic peptide fragment is a peptide of Formula (III-A), (III-B) or (III-C):
R¹-X1a-X2a-X3a-X4a-X5a-X6a-X7a-X8a-X9a-R³ (III-A);
R¹-X1a-X2a-X3a-X4a-X5a-X6a-X7a-X8a-X9a-X10a-R³ (III-B);
R¹-X1a-X2a-X3a-X4a-X5a-X6a-X7a-X8a-X9a-X10a-X11a-R³ (III-C);
wherein each of X1a, X2a, and X3a is independently absent or is an amino acid residue;
each of X4a, X5a, X6a, X7a, X8a, X9a, X10a and X11a is an amino acid residue;
R¹ is H, or C₁₋₂₀ alkanoyl;
R³ is OH or OP²; and P² is a carboxyl protecting group, and wherein the peptide of each of Formulas (III-A), (III-B) or (III-C) is cyclized via a bond between two amino acid residues to form a ring containing between 4 and 8 amino acid residues.

25. The process of claim 24, wherein the peptide of each of Formulas (III-A), (III-B) or (III-C) is cyclized via a bond between X4a and X9a.

26. The process of claim 1, wherein the linear peptide fragment is a peptide of Formula (IV-A), (IV-B), or (IV-C):
R²-X10a-X11a-X12a-X13a-X14a-X15a-X16a-X17a-X18a-X19a-R⁴ (IV-A);
R²-X11a-X12a-X13a-X14a-X15a-X16a-X17a-X18a-X19a-R⁴ (IV-B);
R²-X12a-X13a-X14a-X15a-X16a-X17a-X18a-X19a-R⁴ (IV-C);
wherein each of X10a, X11a, X12a, X13a, X14a, and X15a is an amino acid residue;
each of X16a, X17a, X18a, and X19a is independently absent or is an amino acid residue;
R² is H;
R⁴ is NHP¹, OP², NH₂, or OH,
P¹ is an amino protecting group; and
P² is a carboxyl protecting group.

27. The process of claim 1, wherein the monocyclic peptide fragment is a peptide of Formula (III-A):
R¹-X1a-X2a-X3a-X4a-X5a-X6a-X7a-X8a-X9a-R³ (III-A);
wherein each of X1a, X2a, and X3a is independently absent or is an amino acid residue;
each of X4a, X5a, X6a, X7a, X8a, and X9a is an amino acid residue;
R¹ is H, or C₁₋₂₀ alkanoyl;
R³ is OH or OP²; and wherein the peptide of Formula (III-A) is cyclized via a bond between two amino acid residues to form a ring containing between 4 and 8 amino acid residues; and
wherein the linear peptide fragment is of Formula (IV-A):
R²-X10a-X11a-X12a-X13a-X14a-X15a-X16a-X17a-X18a-X19a-R⁴ (IV-A);
wherein each of X10a, X11a, X12a, X13a, X14a, and X15a is an amino acid residue;
each of X16a, X17a, X18a, and X19a is independently absent or is an amino acid residue;
R² is H;
R⁴ is NHP¹, OP², NH₂, or OH;
P¹ is an amino protecting group; and
each P² is independently a carboxyl protecting group;
and wherein the amide bond is formed between X9a of Formula (III-A) and X10a of Formula (IV-A).

28. (canceled)

29. The process of claim 1, wherein the monocyclic peptide fragment is a peptide of Formula (III-B):
R¹-X1a-X2a-X3a-X4a-X5a-X6a-X7a-X8a-X9a-X10a-R³ (III-B);
wherein each of X1a, X2a, and X3a is independently absent or is an amino acid residue;
each of X4a, X5a, X6a, X7a, X8a, X9a and X10a is an amino acid residue;
R¹ is H, or C₁₋₂₀ alkanoyl;
R³ is OH or OP²; and wherein the peptide of each of Formula (III-B) is cyclized via a bond between two amino acid residues to form a ring containing between 4 and 8 amino acid residues; and
wherein the linear peptide fragment is of Formula (IV-B):
R²-X11a-X12a-X13a-X14a-X15a-X16a-X17a-X18a-X19a-R⁴ (IV-B);
wherein each of X1a, X12a, X13a, X14a, and X15a is an amino acid residue;
each of X16a, X17a, X18a, and X19a is independently absent or is an amino acid residue;
R² is H;
R⁴ is NHP¹, OP², NH₂, or OH;
P¹ is an amino protecting group; and
each P² is independently a carboxyl protecting group;
and wherein the amide bond is formed between X10a of Formula (III-B) and X11a of Formula (IV-B).

30. (canceled)

31. The process of claim 1, wherein the monocyclic peptide fragment is a peptide of Formula (III-C):
R¹-X1a-X2a-X3a-X4a-X5a-X6a-X7a-X8a-X9a-X10a-X11a-R³ (III-C);
wherein each of X1a, X2a, and X3a is independently absent or is an amino acid residue;
each of X4a, X5a, X6a, X7a, X8a, X9a, X10a, and X11a is an amino acid residue;
R¹ is H, or C₁₋₂₀ alkanoyl;
R³ is OH or OP²;and wherein the peptide of each of Formula (III-C) is cyclized via a bond between two amino acid residues to form a ring containing between 4 and 8 amino acid residues; and
wherein the linear peptide fragment is of Formula (IV-C):
R²-X12a-X13a-X14a-X15a-X16a-X17a-X18a-X19a-R⁴ (IV-C);
wherein each of X12a, X13a, X14a, and X15a is an amino acid residue;
wherein each of X16a, X17a, X18a, and X19a is independently absent or is an amino acid residue;
R² is H; and
R⁴ is NHP¹, OP², NH₂, or OH;
P¹ is an amino protecting group; and
each P² is independently a carboxyl protecting group;
and wherein the amide bond is formed between X11a of Formula (III-C) and X12a of Formula (IV-C).

32-54. (canceled)

55. The process of claim 1, wherein the monocyclic peptide fragment is a compound of Formula (III): 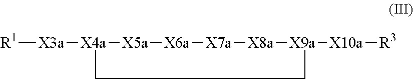
and the linear chain peptide fragment is a compound of Formula (IV):
R²-X11a-X12a-X13a-X14a-X15a-X16a-R⁴ (IV)
and wherein an amide bond is formed between X10a and X1a to form a compound of Formula (II): 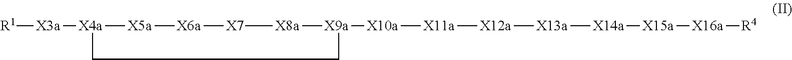
wherein
R¹ is H, or C₁₋₂₀ alkanoyl;
R² is H;
R³ is OH or OP²;
R⁴ is NHP¹, or NH₂;
P¹ is an amino protecting group;
P² is a carboxyl protecting group;
X3a is absent or any amino acid residue;
X4a is Abu, Cys, (D)Cys, alpha-MeCys, (D)Pen, Pen, or Pen(sulfoxide);
X5a is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys;
X6a is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val;
X7a is unsubstituted Trp, or Trp substituted with cyano, halo, alkyl, substituted or unsubstituted aryl, haloalkyl, hydroxy, or alkoxy;
X8a is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1Nal, 2-Nal, or Trp;
X9a is Abu, Cys, (D)Cys, alpha-MeCys, (D)Pen, Pen, or Pen(sulfoxide);
X10a is unsubstituted Phe, or Phe substituted with halo, alkyl, haloalkyl, hydroxy, alkoxy, carboxy, carboxamido, 2-aminoethoxy, or 2-acetylaminoethoxy;
X11a is 2Nal, unsubstituted Trp, or Trp substituted with cyano, halo, alkyl, haloalkyl, hydroxy, alkoxy, Phe(2-Me), Phe(3-Me), Phe(4-Me), Phe(3,4-dimethoxy), or 1Nal;
X12a is 4-amino-4-carboxy-tetrahydropyran (Gly(THP)), alpha-MeLys, alpha-MeLeu, alpha-MeArg, alpha-MePhe, alpha-MeLeu, alpha-MeLys, alpha-MeAsn, alpha-MeTyr, Ala, cyclohexylAla, Lys, or 2-aminoisobutyric acid;
X13a is 2-aminoisobutyric acid, Glu, Cit, Gln, Lys(Ac), alpha-MeArg, alpha-MeGlu, alpha-MeLeu, alpha-MeLys, alpha-Me-Asn, alpha-MeLys(Ac), Dab(Ac), Dap(Ac), homo-Lys(Ac), or Lys; or X13a is Lys, pegylated Lys, b-homoGlu, or Lys(Y2-Ac), wherein Y2 is an amino acid residue;
X14a is Asn, 2Nal, 2-aminoirobutyric acid, Arg, Cit, Asp, Phe, Gly, Lys, Leu, Ala, (D)Ala, beta-Ala, His, Thr, n-Leu, Gln, Ser, (D)Ser, Tic, Trp, alpha-MeGln, alpha-MeAsn, alpha-MeLys(Ac), Dab(Ac), Dap(Ac), homo-Lys(Ac), or Lys(Ac);
X15a is Ala, beta-Ala, Arg, Asn, Asp, Cit, Cys, Glu, Gln, Gly, substituted or unsubstituted His, (D)His, Ile, Lue, (D)Lue, Lys, (D)Lys, Met, 2Pal, 3Pal, or 4Pal, Phe, Pro, 5-Pyal, 2Quin, 3Quin, Ser, Thr, Trp, Tyr, Val, Leu;
X16a is absent or Sarc, aMeLeu, (D)NMeTyr, His, (D)Thr, bAla, Pro, or (D)Pro;
and the compound is cyclized via a Pen-Pen disulfide bond between X4a and X9a; or the compound is cyclized via a Abu-Cys or Abu-Pen thioether bond between X4a and X9a.

56. The process of claim 55, wherein the amino, carboxyl, hydroxyl and thiol groups on the amino acid side chains of each of X3a-X19a are independently protected with a protecting group.

57. The process of claim 56, wherein the protecting group for each amino group on the amino acid side chains are independently selected from Fmoc, Cbz and Boc.

58. (canceled)

59. (canceled)

60. (canceled)

61. (canceled)

62. (canceled)

63. (canceled)

64. (canceled)

65. (canceled)

66. The process of claim 55, further comprising deprotecting the compound of Formula (II) to form a compound of Formula (I): 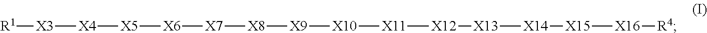
wherein R¹ is H, or C₁₋₂₀ alkanoyl;
R⁴ is NH₂;
X3 is absent or any amino acid residue;
X4 is Abu, Cys, (D)Cys, alpha-MeCys, (D)Pen, Pen, or Pen(sulfoxide);
X5 is Cit, Glu, Gly, substituted Gly, Leu, Ile, beta-Ala, Ala, Lys, Asn, Pro, alpha-MeGln, alpha-MeLys, alpha-MeLeu, alpha-MeAsn, Lys(Ac), alpha-MeLys(Ac), Dab(Ac), Dap(Ac), homo-Lys(Ac), Gln, Asp, or Cys;
X6 is Thr, 2-aminoisobutyric acid, Asp, Dab, Gly, Pro, Ser, alpha-MeGln, alpha-MeLys, alpha-MeLeu, alpha-MeAsn, alpha-MeThr, alpha-MeSer, or Val;
X7 is unsubstituted Trp, or Trp substituted with cyano, halo, alkyl, substituted or unsubstituted aryl, haloalkyl, hydroxy, or alkoxy;
X8 is Gln, alpha-Me-Lys, alpha-MeLeu, alpha-MeLys(Ac), beta-homoGln, Cit, Glu, Phe, Asn, Thr, Val, 2-aminoisobutyric acid, alpha-MeGln, alpha-MeAsn, Lys(Ac), Dab(Ac), Dap(Ac), homo-Lys(Ac), 1Nal, 2-Nal, or Trp;
X9 is Abu, Cys, (D)Cys, alpha-MeCys, (D)Pen, Pen, or Pen(sulfoxide);
X10 is unsubstituted Phe, or Phe substituted with halo, alkyl, haloalkyl, hydroxy, alkoxy, carboxy, carboxamido, 2-aminoethoxy, or 2-acetylaminoethoxy;
X11 is 2Nal, unsubstituted Trp, or Trp substituted with cyano, halo, alkyl, haloalkyl, hydroxy, alkoxy, Phe(2-Me), Phe(3-Me), Phe(4-Me), Phe(3,4-dimethoxy), or 1Nal;
X12 is 4-amino-4-carboxy-tetrahydropyran (Gly(THP)), alpha-MeLys, alpha-MeLeu, alpha-MeArg, alpha-MePhe, alpha-MeLeu, alpha-MeLys, alpha-MeAsn, alpha-MeTyr, Ala, cyclohexylAla, Lys, or 2-aminoisobutyric acid;
X13 is 2-aminoisobutyric acid, Glu, Cit, Gln, Lys(Ac), alpha-MeArg, alpha-MeGlu, alpha-MeLeu, alpha-MeLys, alpha-Me-Asn, alpha-MeLys(Ac), Dab(Ac), Dap(Ac), homo-Lys(Ac), or Lys; or X13 is Lys, pegylated Lys, b-homoGlu, or Lys(Y2-Ac), wherein Y2 is an amino acid residue;
X14 is Asn, 2Nal, 2-aminoirobutyric acid, Arg, Cit, Asp, Phe, Gly, Lys, Leu, Ala, (D)Ala, beta-Ala, His, Thr, n-Leu, Gln, Ser, (D)Ser, Tic, Trp, alpha-MeGln, alpha-MeAsn, alpha-MeLys(Ac), Dab(Ac), Dap(Ac), homo-Lys(Ac), or Lys(Ac);
X15 is Ala, beta-Ala, Arg, Asn, Asp, Cit, Cys, Glu, Gln, Gly, substituted or unsubstituted His, (D)His, Ile, Lue, (D)Lue, Lys, (D)Lys, Met, 2Pal, 3Pal, or 4Pal, Phe, Pro, 5-Pyal, 2Quin, 3Quin, Ser, Thr, Trp, Tyr, Val, Leu;
X16 is absent or Sarc, aMeLeu, (D)NMeTyr, His, (D)Thr, bAla, Pro, or (D)Pro;
and compound is cyclized via a Pen-Pen disulfide bond between X4 and X9; or the compound is cyclized via a Abu-Cys or Abu-Pen thioether bond between X4 and X9.

67. (canceled)

68. (canceled)

69. The process of claim 55, wherein the X4a is (D)Pen, Pen, or Pen(sulfoxide); and X9a is (D)Pen, Pen, or Pen(sulfoxide).

70. The process of claim 69, further comprising a step of preparing the compound of Formula (III) comprising cyclizing a compound of Formula (III′)
R¹-X3a-X4a-X5a-X6a-X7a-X8a-X9a-X10a-R⁵ (III′)
to form a Pen-Pen disulfide bond between X4a and X9a; wherein
R¹ is H, or C₁₋₂₀ alkanoyl;
R⁵ is OH, or OP²; and P² is a carboxyl protecting group.

71. The process of claim 70, wherein the thiol groups on X4a and X9a are deprotected prior to, or during, the cyclizing of the compound of Formula (III′).

72. (canceled)

73. (canceled)

74. (canceled)

75. (canceled)

76. (canceled)

77. The process of claim 55, wherein the step of preparing the compound of Formula (III) comprises reacting a compound of Formula (V)
R¹-X3a-X4a-X5a-X6a-R³ (V)
with a compound of Formula (VI)
R²-X7a-X8a-X9a-X10a-R⁵ (VI)
to form an amide bond between X6a and X7a;
wherein
R¹ is H, or C₁₋₂₀ alkanoyl;
R² is H;
R³ is OH or OP²;
R⁵ is OH, or OP²; and each P² is independently a carboxyl protecting group.

78-130. (canceled)

131. The process of claim 1, wherein the process yields the monocyclic peptide in an amount of greater than 1 Kg.

132-200. (canceled)
